(19) United States
(12) Patent Application Publication
(10) Pub. No.: US 2024/0277807 A1
(43) Pub. Date: Aug. 22, 2024

(54) ACTRII PROTEINS AND USES THEREOF

(71) Applicant: Acceleron Pharma Inc., Cambridge, MA (US)

(72) Inventors: Gang Li, Cambridge, MA (US); Patrick Andre, Cambridge, MA (US); Ravindra Kumar, Cambridge, MA (US)

(21) Appl. No.: 18/568,140

(22) PCT Filed: Jun. 10, 2022
(86) PCT No.: PCT/US2022/033008

§ 371 Date: Dec. 7, 2023

Related U.S. Application Data
(60) Provisional application No. 63/209,860, filed on Jun. 11, 2021.

(51) Int. Cl. A61K 38/17; A61P 11/00

(52) U.S. Cl. CPC A61K 38/179; A61P 11/00

(57) ABSTRACT

In some aspects, the disclosure relates to compositions and methods comprising ActRII polypeptides and TßRII polypeptides to treat, prevent, or reduce the progression rate and/or severity of pulmonary hypertension associated with lung disease (e.g., pulmonary hypertension associated with chronic obstructive pulmonary disease (COP)), interstitial lung disease (ILD), or combined pulmonary fibrosis and emphysema (CPFE)), particularly treating, preventing or reducing the progression rate and/or severity of one or more pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) associated complications.

### CROSS-REFERENCE TO RELATED APPLICATIONS

This application claims the benefit of and priority to U.S. Provisional Application No. 63/209,860, filed Jun. 11, 2021. The foregoing application is incorporated herein by reference in its entirety.

### FIELD OF INVENTION

This application relates to compositions and methods comprising ActRII polypeptides and TβRII polypeptides to treat, prevent, or reduce the progression rate and/or severity of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), particularly in treating, preventing or reducing the progression rate and/or severity of one or more pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) associated complications.

### BACKGROUND OF THE INVENTION

Pulmonary hypertension (PH) is a disease characterized by high blood pressure in lung vasculature, including pulmonary arteries, pulmonary veins, and pulmonary capillaries. In general, PH is defined as a mean pulmonary artery pressure (mPAP)≥20 mm Hg at rest or ≥30 mm Hg with exercise [Hill et al., Respiratory Care 54(7):958-68 (2009)]. One of the main PH symptoms is difficulty in breathing or shortness of breath, and other symptoms include fatigue, dizziness, fainting, peripheral edema (swelling in foot, legs or ankles), bluish lips and skin, chest pain, angina pectoris, light-headedness during exercise, non-productive cough, racing pulse and palpitations. PH can be a severe disease causing heart failure, which is one of the most common causes of death in people who have pulmonary hypertension. Postoperative pulmonary hypertension may complicate many types of surgeries or procedures, and present a challenge associated with a high mortality.

PH may be grouped based on different manifestations of the disease sharing similarities in pathophysiologic mechanisms, clinical presentation, and therapeutic approaches [Simonneau et al., JACC 54(1):S44-54 (2009)]. Clinical classification of PH was first proposed in 1973, and a recent updated clinical classification was endorsed by the World Health Organization (WHO) in 2018. According to the updated PH clinical classification, there are five main groups of PH: pulmonary arterial hypertension (PAH), characterized by a pulmonary artery wedge pressure (PAWP)≤15 mm Hg; PH due to left heart disease (also known as pulmonary venous hypertension or congestive heart failure), characterized by a PAWP>15 mm Hg; PH due to lung diseases and/or hypoxia; PH due to pulmonary artery obstructions; and PH with unclear and/or multifactorial etiologies [Simonneau et al., JACC 54(1):S44-54 (2009); Hill et al., Respiratory Care 54(7):958-68 (2009)]. PAH is further classified into idiopathic PAH (IPAH), a sporadic disease in which there is neither a family history of PAH nor an identified risk factor; heritable PAH; PAH induced by drugs and toxins; PAH associated with connective tissue diseases, HIV infection, portal hypertension, congenital heart diseases, schistosomiasis, and chronic hemolytic anemia; and persistent PH of newborns [Simonneau et al., (2019) Eur Respir J: 53:1801913]. Diagnosis of various types of PH requires a series of tests.

In general, PH treatment depends on the cause or classification of PH. Where PH is caused by a known medicine or medical condition, it is known as a secondary PH, and its treatment is usually directed at the underlying disease. Treatment of Group 3 pulmonary hypertension has traditionally been to optimize treatment of the underlying lung disease and give long-term oxygen therapy to those who are hypoxic. The efficacy of pulmonary vasodilators in this group of patients is unclear. Furthermore, there have been mixed results from meta-analysis assessing the effects of vasodilators on exercise tolerance and quality of life. More studies are required in order to establish the groups of patients who stand to most benefit from vasodilator therapy but the current advice is treat the lung, not the pressure. See, e.g., McGettrick M. et al., Glob Cardiol Sci Pract. 2020 Apr. 30; 2020(1).

There is a high, unmet need for effective therapies for treating pulmonary hypertension. Accordingly, it is an object of the present disclosure to provide methods for treating, preventing, or reducing the progression rate and/or severity of PH, particularly treating, preventing or reducing the progression rate and/or severity of one or more PH-associated complications.

### SUMMARY OF THE INVENTION

In certain aspects, the disclosure provides a method of treating pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

In certain aspects, the disclosure provides a method of treating pulmonary hypertension associated with obstructive lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with obstructive lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the obstructive lung disease is selected from the group consisting of chronic obstructive pulmonary disease (COPD), cystic fibrosis, asthma, emphysema, lymphangioleiomyomatosis, and chronic bronchitis. In some embodiments, the one or more complications of pulmonary hypertension associated with obstructive lung disease is selected from the group consisting of increased need for supplemental oxygen, reduced mobility, and decreased survival.

In certain aspects, the disclosure provides a method of treating pulmonary hypertension associated with restrictive lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with restrictive lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the restrictive lung disease is selected from the group consisting of pulmonary fibrosis, interstitial lung disease, sarcoidosis, idiopathic pulmonary fibrosis, pneumoconiosis, obesity, scoliosis, myasthenia gravis, and pleural effusion. In some embodiments, the one or more complications of pulmonary hypertension associated with restrictive lung disease is selected from the group consisting of shortness of breath with exertion, shortness of breath during rest, shortness of breath with minimal activity, cough, dry cough, productive cough, chronic cough, fatigue, weight loss, anxiety, depression, and fibrosis. In certain aspects, the disclosure provides a method of treating pulmonary hypertension associated with combined obstructive and restrictive lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the combined obstructive and restrictive lung disease is a pulmonary parenchymal disorder. In some embodiments, the pulmonary parenchymal disorder is selected from the group consisting of: Sarcoidosis, COPD and ILD, COPD and idiopathic pulmonary fibrosis, Pneumoconiosis, ILD, Langerhans cell histiocytosis, IPF, Pulmonary alveolar proteinosis, Lymphangioleiomyomatosis, and Bronchiolitis obliterans syndrome. In some embodiments, the pneumoconiosis is selected from the group consisting of silicosis, coal worker's lung, and berylliosis. In some embodiments, the ILD is associated with systemic lupus erythematosus, rheumatoid arthritis, connective tissue disease, interstitial pneumonitis, constrictive bronchiolitis, or cryptogenic organizing pneumonia.

In some embodiments, the combined obstructive and restrictive lung disease is a combination of pulmonary parenchymal disorder and a non-pulmonary disease. In some embodiments, the combination of pulmonary parenchymal disorder and non-pulmonary disease is selected from the group consisting of: COPD and other non-parenchymal diseases, CHF and other non-pulmonary diseases, asthma and other disorder, ILD and obesity, ILD and CHF, and lung hypoplasia and scoliosis.

In some embodiments, the COPD and other non-parenchymal disease is selected from the group consisting of: COPD and congestive heart failure (CHF), COPD and obesity, COPD and thoracic surgery, COPD and diaphragm paralysis, COPD and scoliosis, and COPD and pleurodesis. In some embodiments, the CHF and other non-pulmonary disease is selected from the group consisting of: CHF and scoliosis, CHF and lung resection, and CHF and obesity. In some embodiments, the asthma and other disorder are selected from the group consisting of: asthma and obesity, asthma and lung resection, asthma and radiation fibrosis, asthma and trapped lung, and asthma and CHF.

In certain aspects, the disclosure provides a method of treating pulmonary hypertension associated with interstitial lung disease (ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with interstitial lung disease (ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the ILD is associated with a condition selected from the group consisting of a connective tissue disease, sarcoidosis, vascular destruction due to progressive parenchymal fibrosis, vascular inflammation, perivascular fibrosis, thrombotic angiopathy, and endothelial dysfunction. In some embodiments, the connective tissue disease is selected from the group consisting of systemic sclerosis, rheumatoid arthritis, polymositis, dermatomyositis, and Sjogren syndrome.

In certain aspects, the disclosure provides a method of treating pulmonary hypertension associated with chronic obstructive pulmonary disease (COPD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with chronic obstructive pulmonary disease (COPD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the one or more complications of pulmonary hypertension associated with chronic obstructive pulmonary disease (COPD) is selected from the group consisting of wheezing, productive cough, frequent cough, tightness in the chest, shortness of breath without physical activity, shortness of breath with physical activity, respiratory infection, weight loss, weakness in the muscles of the lower extremities, swelling in the lower extremities, and heart disease. In some embodiments, the patient has COPD with Gold grade 1, Gold grade 2, Gold grade 3, or Gold grade 4 as recognized by the Global Initiative for Chronic Obstructive Lung Disease. In some embodiments, the patient has Group A COPD, Group B COPD, Group C COPD, or Group D COPD. In some embodiments, the patient has COPD selected from the group consisting of: Stage 1, Stage 2, Stage 3, and Stage 4. In some embodiments, the patient has alpha-1-antityrypsin deficiency.

In certain aspects, the disclosure provides a method of treating pulmonary hypertension associated with combined pulmonary fibrosis and emphysema (CPFE), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with combined pulmonary fibrosis and emphysema (CPFE), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating pulmonary hypertension associated with fibrotic idiopathic interstitial pneumonia (IIP), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with fibrotic idiopathic interstitial pneumonia (IIP), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the patient has one or more diagnostic parameters selected from the group consisting of a high fibrotic score and a low diffusing capacity for carbon monoxide (DL_{CO}).

In certain aspects, the disclosure provides a method of treating pulmonary hypertension associated with idiopathic pulmonary fibrosis (IPF), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with idiopathic pulmonary fibrosis (IPF), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the one or more complications of pulmonary hypertension associated with idiopathic pulmonary fibrosis (IPF) is selected from the group consisting of increased need for supplemental oxygen, reduced mobility, and decreased survival.

In certain aspects, the disclosure provides a method of treating pulmonary hypertension associated with non-idiopathic pulmonary fibrosis interstitial lung disease (non-IPF ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with non-idiopathic pulmonary fibrosis interstitial lung disease (non-IPF ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the non-IPF ILD is selected from the group consisting of smoking-related ILD, hypersensitivity pneumonitis related ILD, connective tissue-related ILD, occupation-related ILD, or medication-induced ILD. In some embodiments, the one or more complications of pulmonary hypertension associated with non-idiopathic pulmonary fibrosis interstitial lung disease (non-IPF ILD) is selected from the group consisting of increased need for supplemental oxygen, reduced mobility, and decreased survival.

In certain aspects, the disclosure provides a method of treating pulmonary hypertension associated with nonspecific interstitial pneumonia (NSIP), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure provides a method of treating, preventing, or reducing the progression rate and/or severity of one or more complications of pulmonary hypertension associated with nonspecific interstitial pneumonia (NSIP), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the patient has a right ventricular systolic pressure (RVSP) of greater than 35 mmHg. In some embodiments, the method decreases the RVSP in the patient. In some embodiments, the method reduces the RVSP in the patient by at least 10% at least 15%, at least 20%, at least 25%, at least 30%, at least 35%, at least 40%, at least 45%, or at least 50%. In some embodiments, the method reduces the RVSP in the patient to less than 25 mmHg.

In some embodiments, the patient has a pulmonary artery systolic pressure (PASP) of greater than 25 mmHg prior to treatment. In some embodiments, the patient has a PASP of at least 35 mmHg, 40 mmHg, 45 mmHg, 50 mmHg, 55 mmHg, or 60 mmHg prior to treatment. In some embodiments, the method decreases the PASP in the patient. In some embodiments, the method reduces the PASP in the patient by at least 10%, 15%, 20%, 25%, 30%, 35%, 40%, 45%, or at least 50%. In some embodiments, the method reduces the PASP in the patient by at least 5 mmHg (e.g., at least 5 mmHg, 10 mmHg, 15 mmHg, 20 mmHg, or 25 mmHg). In some embodiments, the method reduces the PASP in the patient to less than 25 mmHg. In some embodiments, the method reduces the PASP in the patient to less than 20 mmHg.

In some embodiments, the patient has a pulmonary vascular resistance (PVR) greater than or equal to 3 Wood Units prior to treatment. In some embodiments, the method decreases the PVR in the patient. In some embodiments, the method reduces the PVR in the patient by at least, 10%, 15%, 20%, 25%, 30%, 35%, 40%, 45%, or at least 50%. In some embodiments, the method reduces the PVR to less than 3 Woods Units.

In some embodiments, the patient has a mean pulmonary artery pressure (mPAP) selected from the group consisting of an mPAP of at least 17 mmHg, an mPAP of at least 20 mmHg, an mPAP of at least 25 mmHg, an mPAP of at least 30 mmHg, an mPAP of at least 35 mmHg, an mPAP of at least 40 mmHg, an mPAP of at least 45 mmHg, and an mPAP of at least 50 mmHg.

In some embodiments, the patient has an mPAP between 21-24 mmHg and a PVR of at least 3 Wood Units. In some embodiments, the patient has an mPAP of greater than 25 mmHg with a Cardiac Index (CI) of less than 2.0 L/min/m². In some embodiments, the patient has an mPAP of greater than 25 mmHg with a CI of less than 2.5 L/min/m². In some embodiments, the method reduces the mPAP in the patient by at least 10%, 15%, 20%, 25%, 30%, 35%, 40%, 45%, or at least 50%. In some embodiments, the method reduces the mPAP by at least 3 mmHg, 5, 7, 10, 12, 15, 20, or 25 mm Hg in the patient. In some embodiments, the method reduces the mPAP to a value selected from the group consisting of less than 17 mmHg, less than 20 mmHg, less than 25 mmHg, and less than 30 mmHg.

In some embodiments, the patient has a mean right atrial pressure (mRAP) selected from the group consisting of an mRAP of at least 5 mmHg, an mRAP of at least 6 mmHg, an mRAP of at least 8 mmHg, an mRAP of at least 10 mmHg, an mRAP of at least 12 mmHg, an mRAP of at least 14 mmHg, and an mRAP of at least 16 mmHg. In some embodiments, the method improves the mean right atrial pressure (mRAP) in the patient. In some embodiments, the improvement in the mRAP is a reduction in the mRAP. In some embodiments, the method reduces the mRAP in the patient by at least, 10%, 15%, 20%, 25%, 30%, 35%, 40%, 45%, or at least 50%. In some embodiments, the method reduces the mRAP by at least 1 mm Hg, 2, 3, 4, 5, 6, 7, 8, 9, 10, 11, 12, 13, 14, or 15 mm Hg in the patient.

In some embodiments, the patient has a cardiac output of less than 4 L/min. In some embodiments, the method increases the cardiac output in the patient by at least 10%, at least 15%, at least 20%, at least 25%, at least 30%, at least 35%, at least 40%, at least 45%, or at least 50%. In some embodiments, the method increases the cardiac output in the patient by at least 0.5 L/min, 1, 1.5, 2, 2.5, 3, 3.5, or 4 L/min in the patient. In some embodiments, the method increases the cardiac output in the patient to at least 4 L/min.

In some embodiments, the patient has a cardiac index (CI) of less than 2.5 L/min/m², 2.0, 1.5, or 1 L/min/m². In some embodiments, the method increases the CI in the patient by at least 10%, at least 15%, at least 20%, at least 25%, at least 30%, at least 35%, at least 40%, 45%, or at least 50%. In some embodiments, the method increases the CI in the patient by at least 0.2 L/min/m², 0.4, 0.6, 0.8, 1, 1.2, 1.4, 1.6, 1.8, or 2 L/min/m² in the patient. In some embodiments, the method increases the CI in the patient to at least 2.5 L/min/m².

In some embodiments, the method increases exercise capacity of the patient. In some embodiments, the patient has a Borg dyspnea index (BDI) at least 0.5, 1, 1.5, 2, 2.5, 3, 3.5, 4, 4.5, 5, 5.5, 6, 6.5, 7, 7.5, 8, 8.5, 9, 9.5, or 10 index points. In some embodiments, the method reduces the patient's Borg dyspnea index (BDI). In some embodiments, the method reduces the patient's BDI by at least 0.5, 1, 1.5, 2, 2.5, 3, 3.5, 4, 4.5, 5, 5.5, 6, 6.5, 7, 7.5, 8, 8.5, 9, 9.5, or 10 index points.

In some embodiments, the patient has a 6-minute walk distance (6 MWD) of less than 550 meters, 500, 450, 440, 400, 380, 350, 300, 250, 200, or 150 meters prior to treatment. In some embodiments, the method increases the patient's 6 MWD by at least 10 meters, 15, 20, 25, 30, 35, 40, 45, 50, 55, 60, 65, 70, 75, 80, 85, 90, 95, 100, 125, 150, 175, 200, 250, 300, or 400 meters.

In some embodiments, the method prevents or reduces pulmonary hypertension Functional Class progression as recognized by the World Health Organization (WHO). In some embodiments, the method prevents or reduces pulmonary hypertension Functional Class progression from Functional Class I to Class II pulmonary hypertension as recognized by the WHO. In some embodiments, the method prevents or reduces pulmonary hypertension Functional Class progression from Functional Class II to Class III pulmonary hypertension as recognized by the WHO. In some embodiments, the method prevents or reduces pulmonary hypertension Functional Class progression from Functional Class III to Class IV pulmonary hypertension as recognized by the WHO.

In some embodiments, the method promotes or increases pulmonary hypertension Functional Class regression as recognized by the WHO. In some embodiments, the method promotes or increases pulmonary hypertension Functional Class regression from Class IV to Class III pulmonary hypertension as recognized by the WHO. In some embodiments, the method promotes or increases pulmonary hypertension Functional Class regression from Class III to Class II pulmonary hypertension as recognized by the WHO. In some embodiments, the method promotes or increases pulmonary hypertension Functional Class regression from Class II to Class I pulmonary hypertension as recognized by the WHO.

In some embodiments, the patient has elevated NT-proBNP levels as compared to a healthy patient prior to treatment. In some embodiments, the patient has normal NT-proBNP levels after treatment. In some embodiments, the patient has a NT-proBNP level of at least 100 pg/mL, 150, 200, 300, 400, 500, 1000, 3000, 5000, 10,000, 15,000, or 20,000 pg/mL.

In some embodiments, the method decreases NT-proBNP levels in the patient. In some embodiments, the method decreases NT-proBNP levels in the patient by at least 10%, 15%, 20%, 25%, 30%, 35%, 40%, 45%, 50%, 55%, 60%, 65%, 70%, 75%, or at least 80%. In some embodiments, the method decreases NT-proBNP levels in the patient by at least 30%. In some embodiments, the method decreases NT-proBNP levels to normal levels. In some embodiments, the normal level of NT-proBNP is <100 pg/ml.

In some embodiments, the patient has elevated brain natriuretic peptide (BNP) levels as compared to a healthy patient. In some embodiments, the patient has normal BNP levels after treatment. In some embodiments, the patient has a BNP level of at least 100 pg/mL, 150, 200, 300, 400, 500, 1000, 3000, 5000, 10,000, 15,000, or 20,000 pg/mL. In some embodiments, the method decreases BNP levels in the patient by at least 10%, 15%, 20%, 25%, 30%, 35%, 40%, 45%, 50%, 55%, 60%, 65%, 70%, 75%, or at least 80%. In some embodiments, the method decreases BNP levels to normal levels (i.e., <100 pg/ml).

In some embodiments, the patient has a diastolic pressure gradient (DPG) of greater than 7 mmHg prior to treatment. In some embodiments, the patient has a DPG of at least 7 mmHg (e.g., at least 7, 10, 15, 20, 25, 30, 35, 40, 45, or 50 mmHg) prior to treatment. In some embodiments, the method decreases the DPG in the patient. In some embodiments, the method reduces the DPG in the patient by at least 10% (e.g., 10%, 15%, 20%, 25%, 30%, 35%, 40%, 45%, or at least 50%). In some embodiments, the method reduces the DPG in the patient to less than 7 mmHg.

In some embodiments, the method increases the patient's quality of life by at least 10% (e.g., 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 100%). In some embodiments, the patient's quality of life is improved as measured using the Cambridge Pulmonary Hypertension Outcome Review (CAMPHOR).

In some embodiments, the patient has pulmonary fibrosis. In some embodiments, the method decreases the pulmonary fibrosis in the patient. In some embodiments, the method reduces the pulmonary fibrosis in the patient by at least 10%, at least 15%, at least 20%, at least 25%, at least 30%, at least 35%, at least 40%, at least 45%, or at least 50%.

In some embodiments, the patient has a diffusing capacity of carbon monoxide (DL_{CO}) less than 60%, 55%, 50%, 45%, 40%, 35%, 30%, 25%, or 20% prior to treatment. In some embodiments, the method increases the DL_{CO} in the patient. In some embodiments, the method increases the DL_{CO} in the patient by at least 5%, at least 10%, at least 15%, at least 20%, at least 25%, at least 30%, at least 35%, at least 40%, at least 45%, or at least 50%. In some embodiments, the method increases the DL_{CO} to at least 40%, 45%, 50%, 55%, 60%, or 65%.

In some embodiments, the patient has a carbon monoxide transfer coefficient (K_{CO}) less than 60% of predicted values, less than 55% of predicted values, less than 50% of predicted values, less than 45%, of predicted values less than 40% of predicted values, less than 35% of predicted values, less than 30% of predicted values, less than 25% of predicted values, or less than 20% of predicted values. In some embodiments, the method increases the K_{CO} in the patient. In some embodiments, the method increases the K_{CO} in the patient by at least 5%, at least 10%, at least 15%, at least 20%, at least 25%, at least 30%, at least 35%, at least 40%, at least 45%, or at least 50%. In some embodiments, the method increases the K_{CO} to at least 40%, 45%, 50%, 55%, 60%, or 65%.

In some embodiments, the patient has a composite physiologic index (CPI) greater than 15, 20, 25, 30, 35, 40, 45, 50, 55, 60, 65, 70, 75, and 80 prior to treatment. In some embodiments, the method decreases the CPI in the patient. In some embodiments, the method decreases the CPI in the patient by at least 10%, at least 15%, at least 20%, at least 25%, at least 30%, at least 35%, at least 40%, at least 45%, or at least 50%. In some embodiments, the method decreases the CPI to less than 70, 65, 60, 55, 50, 45, 40, 35, 30, 25, 20, 15, 10, or 5.

In some embodiments, the patient has an arterial oxygen saturation of less than 95%, 90%, 85%, 80%, 75%, 70%, 65%, 60%, 55%, 50%, 45%, 40%, 35%, or 30% prior to treatment. In some embodiments, the method increases the arterial oxygen saturation in the patient. In some embodiments, the method increases the arterial oxygen saturation in the patient by at least 5%, at least 10%, at least 15%, at least 20%, at least 25%, at least 30%, at least 35%, at least 40%, at least 45%, or at least 50%. In some embodiments, the method increases the arterial oxygen saturation to at least 85%, 90%, or 95%. In some embodiments, the arterial oxygen saturation is measured at rest.

In some embodiments, the patient has a TAPSE of less than 20 mm, 18, 16, 14, or 12 mm prior to treatment. In some embodiments, the method increases the TAPSE to at least 20 mm, 22, 24, 26, 28, or 30 mm.

In some embodiments, the patient has a forced expiratory volume in one second (FEV₁) prior to treatment selected from the group consisting of greater than 70%, between 60% to 69%, between 50% to 59%, between 35% to 49%, and less than 35%. In some embodiments, the method increases the FEV₁ in the patient. In some embodiments, the method increases the FEV₁ in the patient by at least 5%, at least 10%, at least 15%, at least 20%, at least 25%, at least 30%, at least 35%, at least 40%, at least 45%, or at least 50%. In some embodiments, the method increases the FEV₁ to at least 60%, 65%, 70%, 75%, 80%, 85%, 90%, or 95%.

In some embodiments, the patient has a forced vital capacity (FVC) selected from the group consisting of greater than 80%, greater than 70%, between 60% to 69%, between 50% to 59%, between 35% to 49%, and less than 35%. In some embodiments, the method increases the FVC in the patient. In some embodiments, the method increases the FVC in the patient by at least 5%, at least 10%, at least 15%, at least 20%, at least 25%, at least 30%, at least 35%, at least 40%, at least 45%, or at least 50%. In some embodiments, the method increases the FVC to at least 60%, 65%, 70%, 75%, 80%, 85%, 90%, or 95%.

In some embodiments, the method improves right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in right ventricular function is due to an increase in ejection fraction. In some embodiments, the improvement in right ventricular function is due to an increase in right ventricular fractional area change and ejection fraction. In some embodiments, the method decreases right ventricular hypertrophy in the patient. In some embodiments, the method decreases right ventricular hypertrophy in the patient by at least 10%, at least 15%, at least 20%, at least 25%, at least 30%, at least 35%, at least 40%, at least 45%, or at least 50%.

In some embodiments, the method decreases smooth muscle hypertrophy in the patient. In some embodiments, the method decreases smooth muscle hypertrophy in the patient by at least 10%, 15%, 20%, 25%, 30%, 35%, 40%, 45%, or at least 50%.

In some embodiments, the method reduces the risk of death. In some embodiments, the method reduces the risk of death associated with pulmonary arterial hypertension by at least 10%, at least 15%, at least 20%, at least 25%, at least 30%, at least 35%, at least 40%, at least 45%, or at least 50%.

In some embodiments, the method increases transplant free survival in the patient. In some embodiments, the method increases transplant free survival in the patient by at least 10%, at least 15%, at least 20%, at least 25%, at least 30%, at least 35%, at least 40%, at least 45%, or at least 50%.

In some embodiments, the method treats one or more comorbidities of pulmonary hypertension associated with lung disease. In some embodiments, the one or more comorbidities of pulmonary hypertension associated with lung disease are selected from the group consisting of systemic hypertension, decreased renal function, diabetes mellitus, hyperlipidemia, obesity, coronary artery disease (CAD), obstructive sleep apnea, pulmonary embolism, heart failure, atrial fibrillation and anemia.

In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the sequence of amino acids corresponding to residues 30-110 of SEQ ID NO: 1. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence SEQ ID NO: 2. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 3.

In some embodiments, the ActRII polypeptide is a fusion protein further comprising an Fc domain of an immunoglobulin. In some embodiments, the Fc domain of the immunoglobulin is an Fc domain of an IgG1 immunoglobulin.

In some embodiments, the Fc fusion protein further comprises a linker domain positioned between the ActRII polypeptide domain and the Fc domain of the immunoglobulin. In some embodiments, the linker domain is selected from the group consisting of: TGGG (SEQ ID NO: 20), TGGGG (SEQ ID NO: 18), SGGGG (SEQ ID NO: 22), GGGGS (SEQ ID NO: 22), GGG (SEQ ID NO: 16), GGGG (SEQ ID NO: 17), and SGGG (SEQ ID NO: 21).

In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 23. In some embodiments, the ActRII polypeptide comprises an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to the amino acid sequence of SEQ ID NO: 41.

In some embodiments, the ActRII polypeptide comprises an amino acid sequence that is at least 90% identical to an amino acid sequence corresponding to residues 30-110 of SEQ ID NO: 1, wherein the polypeptide binds to activin and/or GDF11. In some embodiments, the polypeptide comprises an amino acid sequence that is at least 90% identical to an amino acid sequence corresponding to residues 21-135 of SEQ ID NO: 1, wherein the polypeptide binds to activin and/or GDF11.

In some embodiments, the polypeptide is lyophilized. In some embodiments, the polypeptide is soluble. In some embodiments, the polypeptide is administered using subcutaneous injection. In some embodiments, the polypeptide is administered approximately every 3 weeks. In some embodiments, the polypeptide is administered approximately every 4 weeks.

In some embodiments, the polypeptide is part of a homodimer protein complex. In some embodiments, the polypeptide is glycosylated. In some embodiments, the polypeptide has a glycosylation pattern obtainable by expression in a Chinese hamster ovary cell.

In some embodiments, the ActRII polypeptide binds to one or more ligands selected from the group consisting of: activin A, activin B, and GDF11. In some embodiments, the ActRII polypeptide further binds to one or more ligands selected from the group consisting of: BMP10, GDF8, and BMP6.

In some embodiments, the ActRII polypeptide is administered at a dose from 0.1 mg/kg to 2.0 mg/kg. In some embodiments, the ActRII polypeptide is administered at a dose of 0.3 mg/kg. In some embodiments, the ActRII polypeptide is administered at a dose of 0.7 mg/kg.

In some embodiments, the TβRII polypeptide comprises a TβRII extracellular domain, wherein the TβRII extracellular domain comprises an amino acid sequence at least 80% identical to: i) an amino acid sequence that begins at any one of amino acids 23, 24, 25, 26, 27, 28, 29, 30, 31, 32, 33, 34, or 35 of SEQ ID NO: 43 and ends at any one of amino acids 153, 154, 155, 156, 157, 158, or 159 of SEQ ID NO: 43; or ii) an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the TβRII extracellular domain comprises an amino acid sequence at least 90% identical to an amino acid sequence that begins at any one of amino acids 23, 24, 25, 26, 27, 28, 29, 30, 31, 32, 33, 34, or 35 of SEQ ID NO: 43 and ends at any one of amino acids 153, 154, 155, 156, 157, 158, or 159 of SEQ ID NO: 43. In some embodiments, the TβRII extracellular domain comprises an amino acid sequence at least 90% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the TβRII extracellular domain comprises an amino acid sequence at least 95% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the TβRII extracellular domain comprises an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the TβRII extracellular domain comprises an amino acid sequence at least 90% identical to SEQ ID NO: 44. In some embodiments, the TβRII extracellular domain comprises an amino acid sequence at least 95% identical to SEQ ID NO: 44. In some embodiments, the TβRII extracellular domain comprises the amino acid sequence of SEQ ID NO: 44.

In some embodiments, the TβRII extracellular domain consists of an amino acid sequence at least 90% identical to an amino acid sequence that begins at any one of amino acids 23, 24, 25, 26, 27, 28, 29, 30, 31, 32, 33, 34, or 35 of SEQ ID NO: 43 and ends at any one of amino acids 153, 154, 155, 156, 157, 158, or 159 of SEQ ID NO: 43. In some embodiments, the TβRII extracellular domain consists of an amino acid sequence at least 95% identical to an amino acid sequence that begins at any one of amino acids 23, 24, 25, 26, 27, 28, 29, 30, 31, 32, 33, 34, or 35 of SEQ ID NO: 43 and ends at any one of amino acids 153, 154, 155, 156, 157, 158, or 159 of SEQ ID NO: 43. In some embodiments, the TβRII extracellular domain consists of an amino acid sequence that begins at any one of amino acids 23, 24, 25, 26, 27, 28, 29, 30, 31, 32, 33, 34, or 35 of SEQ ID NO: 43 and ends at any one of amino acids 153, 154, 155, 156, 157, 158, or 159 of SEQ ID NO: 43.

In some embodiments, the TβRII extracellular domain consists of an amino acid sequence at least 90% identical to a sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the TβRII extracellular domain consists of an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the TβRII extracellular domain consists of an amino acid sequence at least 90% identical to SEQ ID NO: 46. In some embodiments, the TβRII extracellular domain consists of an amino acid sequence at least 95% identical to SEQ ID NO: 46. In some embodiments, the TβRII extracellular domain portion consists of the amino acid sequence of SEQ ID NO: 46.

In some embodiments, the TβRII extracellular domain consists of an amino acid sequence at least 90% identical to SEQ ID NO: 68. In some embodiments, the TβRII extracellular domain consists of an amino acid sequence at least 95% identical to SEQ ID NO: 68. In some embodiments, the TβRII extracellular domain portion consists of the amino acid sequence of SEQ ID NO: 68.

In some embodiments, the TβRII extracellular domain consists of an amino acid sequence at least 90% identical to SEQ ID NO: 70. In some embodiments, the TβRII extracellular domain consists of an amino acid sequence at least 95% identical to SEQ ID NO: 70. In some embodiments, the TβRII extracellular domain portion consists of the amino acid sequence of SEQ ID NO: 70.

In some embodiments, the TβRII polypeptide is a fusion protein further comprising a heterologous domain. In some embodiments, the heterologous domain comprises an immunoglobulin Fc domain. In some embodiments, the immunoglobulin Fc domain is a human immunoglobulin Fc domain. In some embodiments, the heterologous domain comprises an amino acid sequence that is at least 90% identical to SEQ ID NO: 11. In some embodiments, the heterologous domain comprises an amino acid sequence that is at least 95% identical to SEQ ID NO: 11. In some embodiments, the heterologous domain comprises the amino acid sequence of SEQ ID NO: 11. In some embodiments, the TβRII polypeptide or fusion polypeptide further comprises a linker.

In some embodiments, the linker comprises the amino acid sequence of SEQ ID NO: 82. In some embodiments, the TβRII polypeptide or fusion protein does not include amino acids 185-592 of SEQ ID NO: 42.

In some embodiments, the fusion protein consists of or consists essentially of: a) a TβRII polypeptide portion comprising an amino acid sequence that is at least 85%, 90%, 95%, 97%, or 99% identical to the amino acid sequence of SEQ ID NO: 46 and no more than 10, 9, 8, 7, 6, 5, 4, 3, 2 or 1 additional amino acids; b) a linker portion comprising an amino acid sequence that is at least 85%, 90%, 95%, 97%, or 99% identical to the amino acid sequence of SEQ ID NO: 82 and no more than 5, 4, 3, 2, or 1 additional amino acids; c) a heterologous portion comprising an amino acid sequence that is at least 85%, 90%, 95%, 97%, or 99% identical to the amino acid sequence of SEQ ID NO: 11 and no more than 25, 20, 15, 10, 5, 4, 3, 2, or 1 additional amino acids; and d) optionally a leader sequence (e.g., SEQ ID NO: 25).

In some embodiments, the fusion protein consists of or consists essentially of: a) a TβRII polypeptide portion comprising the amino acid sequence of SEQ ID NO: 46 and no more than 10, 9, 8, 7, 6, 5, 4, 3, 2, or 1 additional amino acids; b) a linker portion comprising the amino acid sequence of SEQ ID NO: 82 and no more than 5, 4, 3, 2 or 1 additional amino acids; c) a heterologous portion comprising the amino acid sequence of SEQ ID NO: 11 and no more than 25, 20, 15, 10, 5, 4, 3, 2, or 1 additional amino acids; and d) optionally a leader sequence (e.g., SEQ ID NO: 25).

In some embodiments, the fusion protein comprises: a) an extracellular domain of a TβRII portion; wherein the extracellular domain comprises an amino acid sequence that is at least 85%, 90%, 95%, 97%, or 99% identical to the sequence of SEQ ID NO: 46; b) a heterologous portion, wherein the heterologous portion comprises an amino acid sequence that is at least 85%, 90%, 95%, 97%, or 99% identical to the sequence of SEQ ID NO: 11; and c) a linker portion connecting the extracellular domain and the heterologous portion; wherein the linker comprises an amino acid sequence that is at least 85%, 90%, 95%, 97%, or 99% identical to the amino acid sequence of SEQ ID NO: 82.

In some embodiments, the fusion protein comprises: a) an extracellular domain of a TβRII portion; wherein the extracellular domain comprises the amino acid sequence of SEQ ID NO: 46; b) a heterologous portion, wherein the heterologous portion comprises the amino acid sequence of SEQ ID NO: 11; and c) a linker portion connecting the extracellular domain and the heterologous portion; wherein the linker comprises the amino acid sequence of SEQ ID NO: 82.

In some embodiments, the methods disclosed herein comprise further administering to the patient an additional active agent and/or supportive therapy. In some embodiments, the additional active agent and/or supportive therapy is selected from the group consisting of: beta-blockers, angiotensin-converting enzyme inhibitors (ACE inhibitors), angiotensin receptor blockers (ARBs), diuretic agents, lipid-lowering medications, endothelin blockers, PDE5 inhibitors, and prostacyclins. In some embodiments, the additional active agent and/or supportive therapy is selected from the group consisting of: prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); digoxin, diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), oxygen therapy, and lung and/or heart transplantation.

In some embodiments, the patient has been treated with one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In some embodiments, the method further comprises administration of one or more agents selected from the group consisting of: phosphodiesterase type 5 inhibitors, soluble guanylate cyclase stimulators, prostacyclin receptor agonist, and endothelin receptor antagonists. In some embodiments, the one or more agents is selected from the group consisting of: bosentan, sildenafil, beraprost, macitentan, selexipag, epoprostenol, treprostinil, iloprost, ambrisentan, and tadalafil.

In some embodiments, the patient has been treated with one or more vasodilators prior to administration of the polypeptide. In some embodiments, the method further comprises administration of one or more vasodilators. In some embodiments, the one or more vasodilators is selected from the group consisting of prostacyclin, epoprostenol, and sildenafil. In some embodiments, the vasodilator is prostacyclin.

In some embodiments, the patient has been receiving one or more therapies for pulmonary hypertension associated with lung disease. In some embodiments, the one or more therapies for pulmonary hypertension associated with lung disease is selected from the group consisting of: treprostinil, pirfenidone, nintedanib, prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1-*3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate); a left ventricular assist device (LVAD), oxygen therapy, and lung and/or heart transplantation.

In some embodiments, the ActRII polypeptide is administered to the patient about every week, about every two weeks, about every three weeks, or about every four weeks. In some embodiments, the ActRII polypeptide is administered to the patient every about three weeks. In some embodiments, the TβRII polypeptide is administered to the patient about every week, about every two weeks, about every three weeks, or about every four weeks. In some embodiments, the TβRII polypeptide is administered to the patient about every three weeks.

### BRIEF DESCRIPTION OF THE DRAWINGS

The file of this patent contains at least one drawing/photograph executed in color. Copies of this patent with color drawing(s)/photograph(s) will be provided by the Office upon request and payment of the necessary fee.

FIG. 1 shows an alignment of extracellular domains of human ActRIIB (SEQ ID NO: 31) and human ActRIIA (SEQ ID NO: 2) with the residues that are deduced herein, based on composite analysis of multiple ActRIIB and ActRIIA crystal structures, to directly contact ligand indicated with boxes.

FIG. 2 shows a multiple sequence alignment of various vertebrate ActRIIA proteins and human ActRIIA (SEQ ID NOs: 6-10 and 36-38).

FIG. 3 shows multiple sequence alignment of Fc domains from human IgG isotypes using Clustal 2.1. Hinge regions are indicated by dotted underline. Double underline indicates examples of positions engineered in IgG1 Fc (SEQ ID NO: 32) to promote asymmetric chain pairing and the corresponding positions with respect to other isotypes IgG2 (SEQ ID NO: 33), IgG3 (SEQ ID NO: 34) and IgG4 (SEQ ID NO: 35).

FIGS. 4A and 4B show the purification of ActRIIA-hFc expressed in CHO cells. The protein purifies as a single, well-defined peak as visualized by sizing column (FIG. 4A) and Coomassie stained SDS-PAGE (FIG. 4B) (left lane: molecular weight standards; right lane: ActRIIA-hFc).

FIGS. 5A and 5B show the binding of ActRIIA-hFec to activin (FIG. 5A) and GDF-11 (FIG. 5B), as measured by Biacore™ assay.

FIGS. 6A-6D show the effect of ActRIIA-mFc treatment of pulmonary hypertension and RV hypertrophy in Bleo-MCT PH-ILD rat model. Rx: ActRIIA-mFc s.c. 5 mpk, BIW; Bleo: bleomycin; MCT: Monocrotaline.

FIGS. 7A-7C show the effect of ActRIIA-mFc treatment of pulmonary hypertension and RV hypertrophy in Bleo/Su/Hx PH-ILD rat model. Rx: ActRIIA-mFc s.c. 5 mpk, BIW; Bleo: bleomycin; MCT: Monocrotaline.

FIG. 8 shows the amino acid sequence of native precursor for the B (short) isoform of human TGFβ receptor type II (hTβRII) (NP_003233.4) (SEQ ID NO: 43). Solid underline indicates the mature extracellular domain (ECD) (residues 23-159), and double underline indicates valine that is replaced in the A (long) isoform. Dotted underline denotes leader (residues 1-22).

FIG. 9 shows the amino acid sequence of native precursor for the A (long) isoform of human TβRII (NP_001020018.1) (SEQ ID NO: 42). Solid underline indicates the mature ECD (residues 23-184), and double underline indicates the splice-generated isoleucine substitution. Dotted underline denotes leader (residues 1-22).

FIG. 10 shows a comparison of the linker sequences of five different TβRII constructs (SEQ ID NOs 173-177, respectively, in order of appearance).

FIGS. 11A and 11B show in tabular form the binding affinity between TGFβ1 and TGFβ3 and one of several different TβRII-Fc fusion protein constructs.

FIGS. 12A and 12C graph the results from reporter gene assays testing the affinity of TGFβ1 for one of several different TβRII-Fc fusion protein constructs. FIGS. 12B and 12D graph the results from reporter gene assays testing the affinity of the TGFβ3 for one of several different TβRII-Fc fusion protein constructs. FIGS. 12E and 12F provide IC₅₀ data from these same experiments in tabular form.

### DETAILED DESCRIPTION

### 1. Overview

The present disclosure relates to compositions and methods of treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) comprising administering to a patient in need thereof a combination of an effective amount of an ActRII polypeptide and an effective amount of a TβRII polypeptide as described herein. In certain embodiments, the present disclosure provides methods of treating or preventing pulmonary hypertension associated with lung disease (e.g., pulmonary hypertension associated with COPD, ILD, or CPFE) in an individual in need thereof through administering to the individual a combination of a therapeutically effective amount of an ActRII polypeptide and an effective amount of a TβRII polypeptide as described herein.

Most lung diseases can be categorized as either obstructive or restrictive. Lung diseases that are characterized by both obstruction and restriction occur infrequently and are commonly caused by a combination of pulmonary parenchymal and non-pulmonary disorders. Obstructive lung diseases (e.g., COPD, chronic bronchitis, asthma, bronchiectasis, bronchiolitis, and cystic fibrosis) are characterized by an obstruction in the air passages and defined by exhalation that is slower and shallower than in a healthy individual. Restrictive lung diseases (e.g., adult respiratory distress syndrome (ARDS), pneumoconioses, pneumonia, eosinophilic pneumonia, tuberculosis, sarcoidosis, pulmonary fibrosis and idiopathic pulmonary fibrosis, pleural effusion, and pleurisy) are characterized by a reduced total lung capacity and defined by inhalation that fill the lungs far less than what is expected in a healthy individual. One of the prominent complications of lung disease is pulmonary hypertension. Pulmonary hypertension associated with lung disease (e.g., pulmonary hypertension associated with COPD, ILD, or CPFE) [World Health Organization Group 3 PH] is a progressive disease marked by inflammation and irreversible scarring of the lung tissue. Chronic lung disease is the second leading cause for pulmonary hypertension. The mortality rates for patients with pulmonary hypertension associated with lung disease (e.g., pulmonary hypertension associated with COPD, ILD, or CPFE) is the highest reported of any of the five diagnostic groups of pulmonary hypertension. Currently there is only one U.S. Food and Drug Administration-approved treatment for pulmonary hypertension associated with lung disease, treprostinil, which is also approved for the treatment of pulmonary arterial hypertension (PAH; WHO Group 1 pulmonary hypertension). All other treatments in clinical practice of pulmonary hypertension associated with lung disease are based on management of the underlying lung disease, as well as off-label use of certain treatments approved for pulmonary arterial hypertension (PAH) [World Health Organization (WHO) Group 1 PH].

Pulmonary hypertension associated with lung disease (e.g., pulmonary hypertension associated with COPD, ILD, or CPFE) can be definitively diagnosed using right heart catheterization, however echocardiography remains a good screening and monitoring tool for patients thought to be at risk. Echocardiography is used to detect elevated pulmonary artery systolic pressures (ePASP) as well as altered right-sided ventricle structure or dysfunction and evidence of left-sided heart disease. Other assessments and/or tools (e.g., the 6-min walk test (6 MWT), computed tomography (CT) scans, and pulmonary function tests). Despite being the only definitive test for pulmonary hypertension associated with lung disease, right heart catheterization is not required for every patient suspected of having this disease. However, in cases of suspected moderate or severe pulmonary hypertension, as well as suspected alternate etiologies for pulmonary hypertension, right heart catheterization is recommended.

In certain aspects, the disclosure relates to methods related to treating, preventing, or reducing the progression rate and/or severity of one or more complications of pulmonary hypertension associated with lung disease (e.g., an obstructive lung disease, a restrictive lung disease, or a combined obstructive and restrictive lung disease), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. The one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

The terms used in this specification generally have their ordinary meanings in the art, within the context of this disclosure and in the specific context where each term is used. Certain terms are discussed below or elsewhere in the specification to provide additional guidance to the practitioner in describing the compositions and methods of the disclosure and how to make and use them. The scope or meaning of any use of a term will be apparent from the specific context in which it is used.

The term “sequence similarity,” in all its grammatical forms, refers to the degree of identity or correspondence between nucleic acid or amino acid sequences that may or may not share a common evolutionary origin.

“Percent (%) sequence identity” with respect to a reference polypeptide (or nucleotide) sequence is defined as the percentage of amino acid residues (or nucleic acids) in a candidate sequence that are identical to the amino acid residues (or nucleic acids) in the reference polypeptide (nucleotide) sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid (nucleic acid) sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

“Agonize”, in all its grammatical forms, refers to the process of activating a protein and/or gene (e.g., by activating or amplifying that protein's gene expression or by inducing an inactive protein to enter an active state) or increasing a protein's and/or gene's activity.

“Antagonize”, in all its grammatical forms, refers to the process of inhibiting a protein and/or gene (e.g., by inhibiting or decreasing that protein's gene expression or by inducing an active protein to enter an inactive state) or decreasing a protein's and/or gene's activity.

The terms “about” and “approximately” as used in connection with a numerical value throughout the specification and the claims denotes an interval of accuracy, familiar and acceptable to a person skilled in the art. In general, such interval of accuracy is ±10%. Alternatively, and particularly in biological systems, the terms “about” and “approximately” may mean values that are within an order of magnitude, preferably ≤5-fold and more preferably ≤2-fold of a given value.

Numeric ranges disclosed herein are inclusive of the numbers defining the ranges. The term “between” as used in the present application is inclusive of the numbers defining the ranges. Moreover, all ranges disclosed herein are to be understood to encompass any and all subranges subsumed therein. For example, a stated range of “1 to 10” or “between 1 to 10” should be considered to include any and all subranges between and inclusive of the minimum value of 1 or more, e.g., 1 to 6.1, and ending with a maximum value of 10 or less, e.g., 5.5 to 10.

The terms “a” and “an” include plural referents unless the context in which the term is used clearly dictates otherwise. The terms “a” (or “an”), as well as the terms “one or more,” and “at least one” can be used interchangeably herein. Furthermore, “and/or” where used herein is to be taken as specific disclosure of each of the two or more specified features or components with or without the other. Thus, the term “and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following aspects: A, B, and C; A, B, or C; A or C; A or B; B or C; A and C; A and B; B and C; A (alone); B (alone); and C (alone).

Throughout this specification, the word “comprise” or variations such as “comprises” or “comprising” will be understood to imply the inclusion of a stated integer or groups of integers but not the exclusion of any other integer or group of integers.

### 2. ActRII Polypeptides

In certain aspects, the disclosure relates to ActRII polypeptides and uses thereof (e.g., of treating, preventing, or reducing the progression rate and/or severity of pulmonary hypertension associated with lung disease (e.g., pulmonary hypertension associated with COPD, ILD, or CPFE) or one or more complications of pulmonary hypertension associated with lung disease (e.g., pulmonary hypertension associated with COPD, ILD, or CPFE). As used herein, the term “ActRII” refers to the family of type II activin receptors. This family includes activin receptor type IIA (ActRIIA) and activin receptor type IIB (ActRIIB).

In certain embodiments, the present disclosure relates to ActRII polypeptides having an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence as set forth in anyone of SEQ ID NOs: 1, 2, 3, 23, 27, 30, and 41. As used herein, the term “ActRII” refers to a family of activin receptor type IIA (ActRIIA) proteins, a family of activin receptor type IIB (ActRIIB) proteins, or combinations and/or variants thereof. The ActRII polypeptides can be derived from any species and include variants derived from such ActRII proteins by mutagenesis or other modification. Reference to ActRII herein is understood to be a reference to any one of the currently identified forms. Members of the ActRII family are generally transmembrane proteins, composed of a ligand-binding extracellular domain comprising a cysteine-rich region, a transmembrane domain, and a cytoplasmic domain with predicted serine/threonine kinase activity.

The term ActRII polypeptide includes polypeptides comprising any naturally occurring polypeptide of an ActRII family member as well as any variants thereof (including mutants, fragments, fusions, and peptidomimetic forms) that retain a useful activity. Numbering of amino acids for all ActRII-related polypeptides described herein is based on the numbering of the human ActRII precursor protein sequence provided below (SEQ ID NO: 1), unless specifically designated otherwise.

The canonical human ActRII precursor protein sequence is as follows:

(SEQ ID NO: 1)

| | |
|---|---|
| 1 | MGAAAKLAFA VFLISCSSGA ILGRSETQEC LFFNANWEKD |
| | RTNQTGVEPC |
| 51 | YGDKDKRRHC FATWKNISGS IEIVKQGCWL DDINCYDRTD |
| | CVEKKDSPEV |
| 101 | YFCCCEGNMC NEKFSYFPEM EVTQPTSNPV TPKPPYYNIL |
| | LYSLVPLMLI |
| 151 | AGIVICAFWV YRHHKMAYPP VLVPTQDPGP PPPSPLLGLK |
| | PLQLLEVKAR |
| 201 | GREGCVWKAQ LLNEYVAVKI FPIQDKQSWQ NEYEVYSLPG |
| | MKHENILQFI |
| 251 | GAEKRGTSVD VDLWLITAFH EKGSLSDFLK ANVVSWNELC |
| | HIAETMARGL |
| 301 | AYLHEDIPGL KDGHKPAISH RDIKSKNVLL KNNLTACIAD |
| | FGLALKFEAG |
| 351 | KSAGDTHGQV GTRRYMAPEV LEGAINFQRD AFLRIDMYAM |
| | GLVLWELASR |
| 401 | CTAADGPVDE YMLPFEEEIG QHPSLEDMQE VVVHKKKRPV |
| | LRDYWQKHAG |
| 451 | MAMLCETIEE CWDHDAEARL SAGCVGERIT QMORLTNIIT |
| | TEDIVTVVTM |
| 501 | VTNVDFPPKE SSL |

The signal peptide is indicated by a single underline; the extracellular domain is indicated in bold font; and the potential, endogenous N-linked glycosylation sites are indicated by a double underline.

A processed (mature) extracellular human ActRII polypeptide sequence is as follows:

(SEQ ID NO: 2)
ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPP

The C-terminal “tail” of the extracellular domain is indicated by single underline. The sequence with the “tail” deleted (a A15 sequence) is as follows: ILGRSETQECLFFNANWEKDRTNQTGVEPCYGDKDKRRHCFATWKNISGSIEIVKQGCWLDD INCYDRTDCVEKKDSPEVYFCCCEGNMCNEKFSYFPEM (SEQ ID NO: 3) A nucleic acid sequence encoding human ActRII precursor protein is shown below (SEQ ID NO: 4), corresponding to nucleotides 159-1700 of Genbank Reference Sequence NM_001616.4. The signal sequence is underlined.

(SEQ ID NO: 4)

| | |
|---|---|
| 1 | ATGGGAGCTG CTGCAAAGTT GGCGTTTGCC GTCTTTCTTA |
| | TCTCCTGTTC |
| 51 | TTCAGGTGCTATACTTGGTA GATCAGAAAC TCAGGAGTGT |
| | CTTTTCTTTA |
| 101 | ATGCTAATTG GGAAAAAGAC AGAACCAATC AAACTGGTGT |
| | TGAACCGTGT |
| 151 | TATGGTGACA AAGATAAACG GCGGCATTGT TTTGCTACCT |
| | GGAAGAATAT |
| 201 | TTCTGGTTCC ATTGAAATAG TGAAACAAGG TTGTTGGCTG |
| | GATGATATCA |
| 251 | ACTGCTATGA CAGGACTGAT TGTGTAGAAA AAAAAGACAG |
| | CCCTGAAGTA |
| 301 | TATTTTTGTT GCTGTGAGGG CAATATGTGT AATGAAAAGT |
| | TTTCTTATTT |
| 351 | TCCGGAGATG GAAGTCACAC AGCCCACTTC AAATCCAGTT |
| | ACACCTAAGC |
| 401 | CACCCTATTA CAACATCCTG CTCTATTCCT TGGTGCCACT |
| | TATGTTAATT |
| 451 | GCGGGGATTG TCATTTGTGC ATTTTGGGTG TACAGGCATC |
| | ACAAGATGGC |
| 501 | CTACCCTCCT GTACTTGTTC CAACTCAAGA CCCAGGACCA |
| | CCCCCACCTT |
| 551 | CTCCATTACT AGGTTTGAAA CCACTGCAGT TATTAGAAGT |
| | GAAAGCAAGG |
| 601 | GGAAGATTTG GTTGTGTCTG GAAAGCCCAG TTGCTTAACG |
| | AATATGTGGC |
| 651 | TGTCAAAATA TTTCCAATAC AGGACAAACA GTCATGGCAA |
| | AATGAATACG |
| 701 | AAGTCTACAG TTTGCCTGGA ATGAAGCATG AGAACATATT |
| | ACAGTTCATT |
| 751 | GGTGCAGAAA AACGAGGCAC CAGTGTTGAT GTGGATCTTT |
| | GGCTGATCAC |
| 801 | AGCATTTCAT GAAAAGGGTT CACTATCAGA CTTTCTTAAG |
| | GCTAATGTGG |
| 851 | TCTCTTGGAA TGAACTGTGT CATATTGCAG AAACCATGGC |
| | TAGAGGATTG |
| 901 | GCATATTTAC ATGAGGATAT ACCTGGCCTA AAAGATGGCC |
| | ACAAACCTGC |
| 951 | CATATCTCAC AGGGACATCA AAAGTAAAAA TGTGCTGTTG |
| | AAAAACAACC |
| 1001 | TGACAGCTTG CATTGCTGAC TTTGGGTTGG CCTTAAAATT |
| | TGAGGCTGGC |
| 1051 | AAGTCTGCAG GCGATACCCA TGGACAGGTT GGTACCCGGA |
| | GGTACATGGC |
| 1101 | TCCAGAGGTA TTAGAGGGTG CTATAAACTT CCAAAGGGAT |
| | GCATTTTTGA |
| 1151 | GGATAGATAT GTATGCCATG GGATTAGTCC TATGGGAACT |
| | GGCTTCTCGC |
| 1201 | TGTACTGCTG CAGATGGACC TGTAGATGAA TACATGTTGC |
| | CATTTGAGGA |
| 1251 | GGAAATTGGC CAGCATCCAT CTCTTGAAGA CATGCAGGAA |
| | GTTGTTGTGC |
| 1301 | ATAAAAAAAA GAGGCCTGTT TTAAGAGATT ATTGGCAGAA |
| | ACATGCTGGA |
| 1351 | ATGGCAATGC TCTGTGAAAC CATTGAAGAA TGTTGGGATC |
| | ACGACGCAGA |
| 1401 | AGCCAGGTTA TCAGCTGGAT GTGTAGGTGA AAGAATTACC |
| | CAGATGCAGA |
| 1451 | GACTAACAAA TATTATTACC ACAGAGGACA TTGTAACAGT |
| | GGTCACAATG |
| 1501 | GTGACAAATG TTGACTTTCC TCCCAAAGAA TCTAGTCTA |

A nucleic acid sequence encoding processed soluble (extracellular) human ActRII polypeptide is as follows:

(SEQ ID NO: 5)

| | |
|---|---|
| 1 | ATACTTGGTA GATCAGAAAC TCAGGAGTGT CTTTTCTTTA |
| | ATGCTAATTG |
| 51 | GGAAAAAGAC AGAACCAATC AAACTGGTGT TGAACCGTGT |
| | TATGGTGACA |
| 101 | AAGATAAACG GCGGCATTGT TTTGCTACCT GGAAGAATAT |
| | TTCTGGTTCC |
| 151 | ATTGAAATAG TGAAACAAGG TTGTTGGCTG GATGATATCA |
| | ACTGCTATGA |
| 201 | CAGGACTGAT TGTGTAGAAA AAAAAGACAG CCCTGAAGTA |
| | TATTTTTGTT |
| 251 | GCTGTGAGGG CAATATGTGT AATGAAAAGT TTTCTTATTT |
| | TCCGGAGATG |
| 301 | GAAGTCACAC AGCCCACTTC AAATCCAGTT ACACCTAAGC |
| | CACCC |

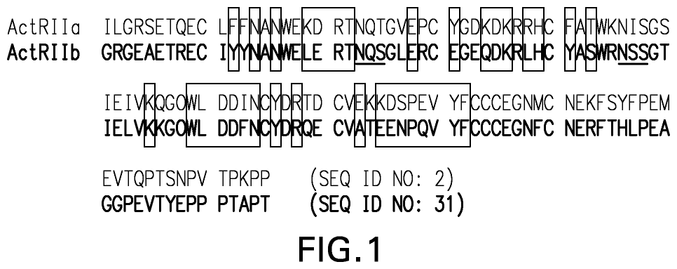

An alignment of the amino acid sequences of human ActRIIA extracellular domain and human ActRIIB extracellular domain is illustrated in FIG. 1. This alignment indicates amino acid residues within both receptors that are believed to directly contact ActRII ligands. For example, the composite ActRII structures indicated that the ActRIIA-ligand binding pocket is defined, in part, by residues F31, N33, N35, K38 through T41, E47, Y50, K53 through K55, R57, H58, F60, T62, K74, W78 through N83, Y85, R87, E92, and K94 through F101. At these positions, it is expected that conservative mutations will be tolerated.

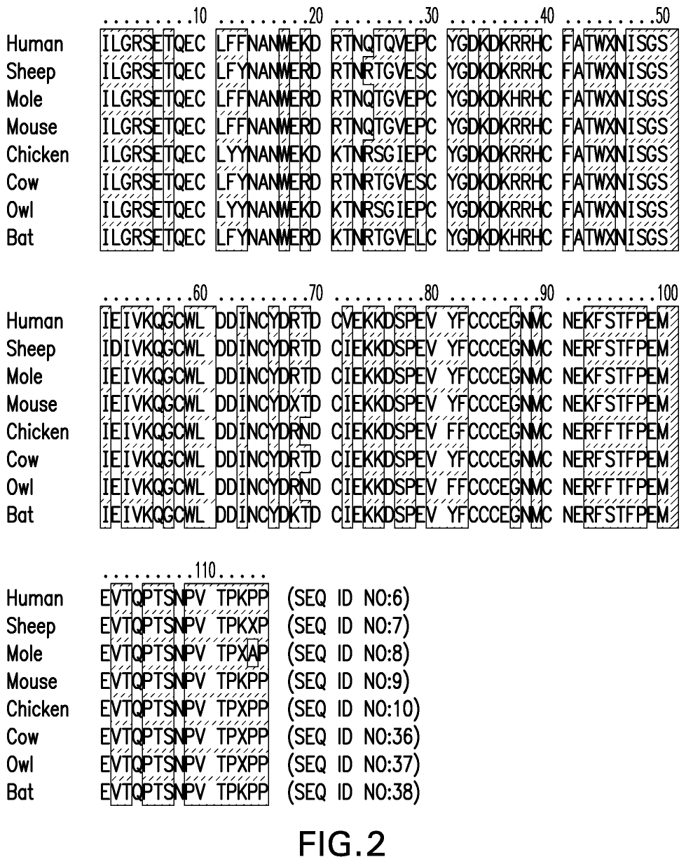

ActRII is well-conserved among vertebrates, with large stretches of the extracellular domain completely conserved. For example, FIG. 2 depicts a multi-sequence alignment of a human ActRIIA extracellular domain compared to various ActRIIA orthologs. Many of the ligands that bind to ActRIIA are also highly conserved. Accordingly, from these alignments, it is possible to predict key amino acid positions within the ligand-binding domain that are important for normal ActRII-ligand binding activities as well as to predict amino acid positions that are likely to be tolerant to substitution without significantly altering normal ActRII-ligand binding activities. Therefore, an active, human ActRII variant polypeptide useful in accordance with the presently disclosed methods may include one or more amino acids at corresponding positions from the sequence of another vertebrate ActRII, or may include a residue that is similar to that in the human or other vertebrate sequences.

Without meaning to be limiting, the following examples illustrate this approach to defining an active ActRII variant. As illustrated in FIG. 2, F13 in the human extracellular domain is Y in Ovis aries (SEQ ID NO: 7), Gallus gallus (SEQ ID NO: 10), Bos Taurus (SEQ ID NO: 36), Tyto alba (SEQ ID NO: 37), and Myotis davidii (SEQ ID NO: 38) ActRIIA, indicating that aromatic residues are tolerated at this position, including F, W, and Y. Q24 in the human extracellular domain is R in Bos Taurus ActRIIA, indicating that charged residues will be tolerated at this position, including D, R, K, H, and E. S95 in the human extracellular domain is F in Gallus gallus and Tyto alba ActRIIA, indicating that this site may be tolerant of a wide variety of changes, including polar residues, such as E, D, K, R, H, S, T, P, G, Y, and probably hydrophobic residue such as L, I, or F. E52 in the human extracellular domain is D in Ovis aries ActRIIA, indicating that acidic residues are tolerated at this position, including D and E. P29 in the human extracellular domain is relatively poorly conserved, appearing as S in Ovis aries ActRIIA and L in Myotis davidii ActRIIA, thus essentially any amino acid should be tolerated at this position.

Moreover, as discussed above, ActRII proteins have been characterized in the art in terms of structural/functional characteristics, particularly with respect to ligand binding [Attisano et al. (1992) Cell 68(1):97-108; Greenwald et al. (1999) Nature Structural Biology 6(1): 18-22; Allendorph et al. (2006) Proc Natl Acad Sci USA 103(20: 7643-7648; Thompson et al. (2003) The EMBO Journal 22(7): 1555-1566; as well as U.S. Pat. Nos. 7,709,605, 7,612,041, and 7,842,663]. For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. In addition to the teachings herein, these references provide ample guidance for how to generate ActRII variants that retain one or more desired activities (e.g., ligand-binding activity).

For example, a defining structural motif known as a three-finger toxin fold is important for ligand binding by type I and type II receptors and is formed by conserved cysteine residues located at varying positions within the extracellular domain of each monomeric receptor [Greenwald et al. (1999) Nat Struct Biol 6:18-22; and Hinck (2012) FEBS Lett 586:1860-1870]. Accordingly, the core ligand-binding domains of human ActRII, as demarcated by the outermost of these conserved cysteines, corresponds to positions 30-110 of SEQ ID NO: 1 (ActRII precursor). Therefore, the structurally less-ordered amino acids flanking these cysteine-demarcated core sequences can be truncated by about 1, 2, 3, 4, 5, 6, 7, 8, 9, 10, 11, 12, 13, 14, 15, 16, 17, 18, 19, 20, 21, 22, 23, 24, 25, 26, 27, 28, or 29 residues at the N-terminus and by about 1, 2, 3, 4, 5, 6, 7, 8, 9, 10, 11, 12, 13, 14, 15, 16, 17, 18, 19, 20, 21, 22, 23, 24, or 25 residues at the C-terminus without necessarily altering ligand binding. Exemplary ActRII extracellular domains truncations include SEQ ID NOs: 2 and 3.

Accordingly, a general formula for an active portion (e.g., ligand binding) of ActRII is a polypeptide that comprises, consists essentially of, or consists of amino acids 30-110 of SEQ ID NO: 1. Therefore ActRII polypeptides may, for example, comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to any one of amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118, 119, 120, 121, 122, 123, 124, 125, 126, 127, 128, 129, 130, 131, 132, 133, 134, or 135) of SEQ ID NO: 1. Other examples include constructs that begin at a position selected from 21-30 (e.g., beginning at any one of amino acids 21, 22, 23, 24, 25, 26, 27, 28, 29, or 30), 22-30 (e.g., beginning at any one of amino acids 22, 23, 24, 25, 26, 27, 28, 29, or 30), 23-30 (e.g., beginning at any one of amino acids 23, 24, 25, 26, 27, 28, 29, or 30), 24-30 (e.g., beginning at any one of amino acids 24, 25, 26, 27, 28, 29, or 30) of SEQ ID NO: 1, and end at a position selected from 111-135 (e.g., ending at any one of amino acids 111, 112,113, 114, 115, 116, 117, 118, 119, 120, 121, 122, 123, 124, 125, 126, 127, 128, 129, 130, 131, 132, 133, 134 or 135), 112-135 (e.g., ending at any one of amino acids 112, 113, 114, 115, 116, 117, 118, 119, 120, 121, 122, 123, 124, 125, 126, 127, 128, 129, 130, 131, 132, 133, 134 or 135), 113-135 (e.g., ending at any one of amino acids 113, 114, 115, 116, 117, 118, 119, 120, 121, 122, 123, 124, 125, 126, 127, 128, 129, 130, 131, 132, 133, 134 or 135), 120-135 (e.g., ending at any one of amino acids 120, 121, 122, 123, 124, 125, 126, 127, 128, 129, 130, 131, 132, 133, 134 or 135), 130-135 (e.g., ending at any one of amino acids 130, 131, 132, 133, 134 or 135), 111-134 (e.g., ending at any one of amino acids 110, 111, 112, 113, 114, 115, 116, 117, 118, 119, 120, 121, 122, 123, 124, 125, 126, 127, 128, 129, 130, 131, 132, 133, or 134), 111-133 (e.g., ending at any one of amino acids 110, 111, 112, 113, 114, 115, 116, 117, 118, 119, 120, 121, 122, 123, 124, 125, 126, 127, 128, 129, 130, 131, 132, or 133), 111-132 (e.g., ending at any one of amino acids 110, 111, 112, 113, 114, 115, 116, 117, 118, 119, 120, 121, 122, 123, 124, 125, 126, 127, 128, 129, 130, 131, or 132), or 111-131 (e.g., ending at any one of amino acids 110, 111, 112, 113, 114, 115, 116, 117, 118, 119, 120, 121, 122, 123, 124, 125, 126, 127, 128, 129, 130, or 131) of SEQ ID NO: 1. Variants within these ranges are also contemplated, particularly those comprising, consisting essentially of, or consisting of an amino acid sequence that has at least 70%, 75%, 80%, 85%, 86%, 87%, 88%, 89%, 90%, 91%, 92%, 93%, 94%, 95%, 96%, 97%, 98%, 99%, or 100% identity to the corresponding portion of SEQ ID NO: 1. Thus, in some embodiments, an ActRII polypeptide may comprise, consist essentially of, or consist of a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1. Optionally, ActRII polypeptides comprise a polypeptide that is at least 70%, 75%, 80%, 85%, 86%, 87%, 88%, 89%, 90%, 91%, 92%, 93%, 94%, 95%, 96%, 97%, 98%, 99%, or 100% identical to amino acids 30-110 of SEQ ID NO: 1, and comprising no more than 1, 2, 5, 10 or 15 conservative amino acid changes in the ligand-binding pocket. In some embodiments, the ActRII polypeptide is part of a homodimer protein complex.

In certain embodiments, the disclosure relates to an ActRII polypeptide (e.g., ActRIIA polypeptides, ActRIIB polypeptides, or combinations thereof), which includes fragments, functional variants, and modified forms thereof as well as uses thereof (e.g., treating, preventing, or reducing the pulmonary hypertension associated with lung disease (e.g., pulmonary hypertension associated with COPD, ILD, or CPFE). In some embodiments, ActRII polypeptides are soluble (e.g., an extracellular domain of ActRII). In some embodiments, ActRII polypeptides inhibit (e.g., Smad signaling) one or more GDF/BMP ligands [e.g., GDF11, GDF8, activin A, activin B, GDF3, BMP4, BMP6, BMP10, and/or BMP15]. In some embodiments, ActRII polypeptides bind to one or more GDF/BMP ligands [e.g., GDF11, GDF8, activin A, activin B, GDF3, BMP4, BMP6, BMP10, and/or BMP15]. In some embodiments, ActRII polypeptides of the disclosure comprise, consist essentially of, or consist of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to a portion of ActRII beginning at a residue corresponding to amino acids 21-30 (e.g., beginning at any one of amino acids 21, 22, 23, 24, 25, 26, 27, 28, 29, or 30) of SEQ ID NO: 1 and ending at a position corresponding to any one amino acids 110-135 (e.g., ending at any one of amino acids 110, 111, 112, 113, 114, 115, 116, 117, 118,119, 120, 121, 122, 123, 124, 125, 126, 127, 128, 129, 130, 131, 132, 133, 134 or 135) of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 30-110 of SEQ ID NO: 1. In certain embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical amino acids 21-135 of SEQ ID NO: 1. In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of any one of SEQ ID NOs: 1, 2, 3, 23, 27, 30, and 41.

In some embodiments, ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some alternative embodiments, the ActRII polypeptide (e.g., SEQ ID NO: 23) lacks the C-terminal lysine. In some embodiments, the ActRII polypeptide lacking the C-terminal lysine is SEQ ID NO: 41. In some embodiments, the ActRII polypeptides comprise, consist, or consist essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 23. In some embodiments, a patient is administered an ActRII polypeptide comprising, consisting, or consisting essentially of an amino acid sequence that is at least 70%, 75%, 80%, 85%, 86%, 87%, 88%, 89%, 90%, 91%, 92%, 93%, 94%, 95%, 97%, 98%, 99%, or 100% identical to the amino acid sequence of SEQ ID NO: 41. In some embodiments, a patient is administered a combination of SEQ ID NO: 23 and SEQ ID NO: 41.

In certain aspects, the present disclosure relates to ActRII polypeptides (e.g., ActRIIA polypeptides, ActRIIB polypeptides, or combinations thereof). In some embodiments, ActRII traps of the present disclosure are variant ActRII polypeptides (e.g., ActRIIA polypeptides, ActRIIB polypeptides, or combinations thereof) that comprise one or more mutations (e.g., amino acid additions, deletions, substitutions, and combinations thereof) in the extracellular domain (also referred to as the ligand-binding domain) of an ActRII polypeptide (e.g., a “wild-type” or unmodified ActRII polypeptide) such that the variant ActRII polypeptide has one or more altered ligand-binding activities than the corresponding wild-type ActRII polypeptide. In some embodiments, variant ActRII polypeptides of the present disclosure retain at least one similar activity as a corresponding wild-type ActRII polypeptide. For example, in some embodiments ActRII polypeptides bind to and inhibit (e.g. antagonize) the function of activin, GDF11 and/or GDF8. In some embodiments, ActRII polypeptides of the present disclosure further bind to and inhibit one or more of ligand of the GDF/BMP [e.g., GDF11, GDF8, activin A, activin B, GDF3, BMP4, BMP6, BMP10, and/or BMP15]. Accordingly, the present disclosure provides ActRII polypeptides that have an altered binding specificity for one or more ActRII ligands.

To illustrate, one or more mutations may be selected that increase the selectivity of the altered ligand-binding domain for GDF11 and/or GDF8 over one or more ActRII-binding ligands such as activins (activin A or activin B), particularly activin A. Optionally, the altered ligand-binding domain has a ratio of K_{d} for activin binding to K_{d} for GDF11 and/or GDF8 binding that is at least 2-, 5-, 10-, 20-, 50-, 100- or even 1000-fold greater relative to the ratio for the wild-type ligand-binding domain. Optionally, the altered ligand-binding domain has a ratio of IC₅₀ for inhibiting activin to IC₅₀ for inhibiting GDF11 and/or GDF8 that is at least 2-, 5-, 10-, 20-, 50-, 100- or even 1000-fold greater relative to the wild-type ligand-binding domain. Optionally, the altered ligand-binding domain inhibits GDF11 and/or GDF8 with an IC₅₀ at least 2-, 5-, 10-, 20-, 50-, 100- or even 1000-times less than the IC₅₀ for inhibiting activin.

### 3. TβRII Polypeptides

In certain aspects, the disclosure relates to TβRII polypeptides and uses thereof (e.g., of treating, preventing, or reducing the progression rate and/or severity of pulmonary hypertension associated with lung disease (e.g., pulmonary hypertension associated with COPD, ILD, or CPFE) or one or more complications of pulmonary hypertension associated with lung disease (e.g., pulmonary hypertension associated with COPD, ILD, or CPFE).

As described herein, a TβRII polypeptide may be used in combination with one or more additional active agents to treat pulmonary hypertension associated with lung disease (e.g., pulmonary hypertension associated with COPD, ILD, or CPFE) or one or more complications of pulmonary hypertension associated with lung disease (e.g., pulmonary hypertension associated with COPD, ILD, or CPFE).

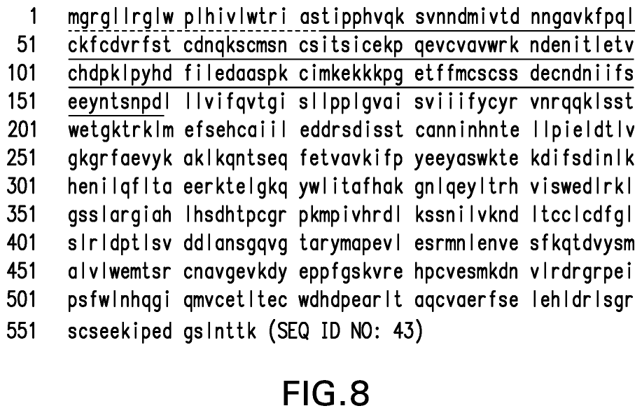

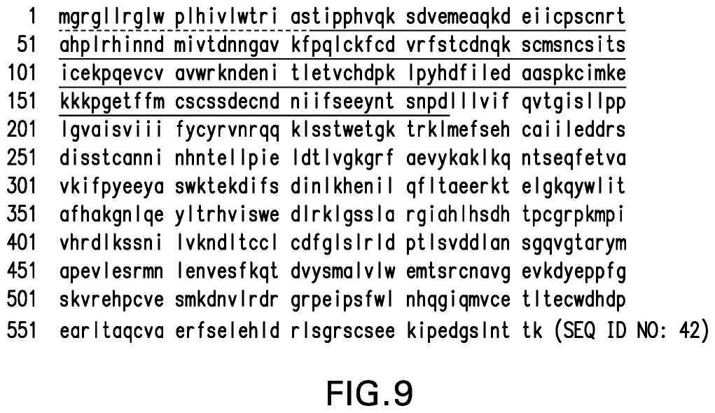

Naturally occurring TβRII proteins are transmembrane proteins, with a portion of the protein positioned outside the cell (the extracellular portion) and a portion of the protein positioned inside the cell (the intracellular portion). Aspects of the present disclosure encompass variant TβRII polypeptides comprising mutations within the extracellular domain and/or truncated portions of the extracellular domain of TβRII. As described above, human TβRII occurs naturally in at least two isoforms—A (long) and B (short)—generated by alternative splicing in the extracellular domain (ECD) (The short isoform is shown in FIG. 8, SEQ ID NO: 43; the long isoform is shown in FIG. 9, SEQ ID NO: 42). SEQ ID NO: 45, which corresponds to residues 23-159 of SEQ ID NO: 43, depicts the native full-length extracellular domain of the short isoform of TβRII. SEQ ID NO: 46, which corresponds to residues 23-184 of SEQ ID NO: 42, depicts the native full-length extracellular domain of the long isoform of TβRII. Unless noted otherwise, amino acid position numbering with regard to variants based on the TβRII short and long isoforms refers to the corresponding position in the native precursors, SEQ ID NO: 43 and SEQ ID NO: 42, respectively.

In certain embodiments, the disclosure provides variant TβRII polypeptides. A TβRII polypeptide of the disclosure may bind to and inhibit the function of a TGFβ superfamily member, such as but not limited to, TGFβ1 or TGFβ3. TβRII polypeptides may include a polypeptide consisting of, or comprising, an amino acid sequence at least 80% identical, and optionally at least 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 153-159 of SEQ ID NO: 43. TβRII polypeptides may include a polypeptide consisting of, or comprising, an amino acid sequence at least 80% identical, and optionally at least 85%, 90%, 91%, 92%, 93%, 94%, 95%, 96%, 97%, 98%, 99%, or 100% identical to a truncated ECD domain of a naturally occurring TβRII polypeptide, whose C-terminus occurs at any of amino acids 178-184 of SEQ ID NO: 42. In particular embodiments, the TβRII polypeptides comprise an amino acid sequence at least 80% identical, and optionally at least 85%, 90%, 910%, 92%, 93%, 94%, 95%, 96%, 97%, 98%, 99%, or 100% identical to the amino acid sequence of SEQ ID NO: 46. In particular embodiments, the TβRII polypeptides comprise an amino acid sequence at least 80% identical, and optionally at least 85%, 90%, 91%, 92%, 93%, 94%, 95%, 96%, 97%, 98%, 99%, or 100% identical to the amino acid sequence of SEQ ID NO: 45. Optionally, a TβRII polypeptide does not include more than 5 consecutive amino acids, or more than 10, 20, 30, 40, 50, 52, 60, 70, 80, 90, 100, 150 or 200 or more consecutive amino acids from a sequence consisting of amino acids 160-567 of SEQ ID NO: 43 or from a sequence consisting of amino acids 185-592 of SEQ ID NO: 42. In some embodiments, the TβRII polypeptide does not include amino acids 160-567 of SEQ ID NO: 43. In some embodiments, the TβRII polypeptide does not include amino acids 1-22 of SEQ ID NO: 43. In some embodiments, the TβRII polypeptide does not include amino acids 1-22 and 160-567 of SEQ ID NO: 43. In some embodiments, the TβRII polypeptide does not include amino acids 185-592 of SEQ ID NO: 42. In some embodiments, the TβRII polypeptide does not include amino acids 1-22 of SEQ ID NO: 42. In some embodiments, the TβRII polypeptide does not include amino acids 1-22 and 185-592 of SEQ ID NO: 42. The unprocessed TβRII polypeptide may either include or exclude any signal sequence, as well as any sequence N-terminal to the signal sequence. As elaborated herein, the N-terminus of the mature (processed) TβRII polypeptide may occur at any of amino acids 23-35 of SEQ ID NO: 43 or 23-60 of SEQ ID NO: 42. Examples of mature TβRII polypeptides include, but are not limited to, amino acids 23-159 of SEQ ID NO: 43 (set forth in SEQ ID NO: 45), amino acids 29-159 of SEQ ID NO: 43 (set forth in SEQ ID NO: 47), amino acids 35-159 of SEQ ID NO: 43 (set forth in SEQ ID NO: 48), amino acids 23-153 of SEQ ID NO: 43 (set forth in SEQ ID NO: 49), amino acids 29-153 of SEQ ID NO: 43 (set forth in SEQ ID NO: 50), amino acids 35-153 of SEQ ID NO: 43 (set forth in SEQ ID NO: 51), amino acids 23-184 of SEQ ID NO: 42 (set forth in SEQ ID NO: 46), amino acids 29-184 of SEQ ID NO: 42 (set forth in SEQ ID NO: 52), amino acids 60-184 of SEQ ID NO: 42 (set forth in SEQ ID NO: 53), amino acids 23-178 of SEQ ID NO: 42 (set forth in SEQ ID NO: 54), amino acids 29-178 of SEQ ID NO: 42 (set forth in SEQ ID NO: 55), and amino acids 60-178 of SEQ ID NO: 42 (set forth in SEQ ID NO: 56). It will be understood by one of skill in the art that corresponding variants based on the long isoform of TβRII will include nucleotide sequences encoding the 25-amino acid insertion along with a conservative Val-Ile substitution at the flanking position C-terminal to the insertion. The TβRII polypeptides accordingly may include isolated extracellular portions of TβRII polypeptides, including both the short and the long isoforms, variants thereof (including variants that comprise, for example, no more than 2, 3, 4, 5, 10, 15, 20, 25, 30, or 35 amino acid substitutions in the sequence corresponding to amino acids 23-159 of SEQ ID NO: 43 or amino acids 23-184 of SEQ ID NO: 42), fragments thereof, and fusion proteins comprising any of the foregoing, but in each case preferably any of the foregoing TβRII polypeptides will retain substantial affinity for at least one of, or both of, TGFβ1 or TGFβ3. Generally, a TβRII polypeptide will be designed to be soluble in aqueous solutions at biologically relevant temperatures, pH levels, and osmolarity.

In some embodiments, the variant TβRII polypeptides of the disclosure comprise one or more mutations in the extracellular domain that confer an altered ligand binding profile. A TβRII polypeptide may include one, two, five or more alterations in the amino acid sequence relative to the corresponding portion of a naturally occurring TβRII polypeptide. In some embodiments, the mutation results in a substitution, insertion, or deletion at the position corresponding to position 70 of SEQ ID NO: 43. In some embodiments, the mutation results in a substitution, insertion, or deletion at the position corresponding to position 110 of SEQ ID NO: 43. Examples include, but are not limited to, an N to D substitution or a D to K substitution in the positions corresponding to positions 70 and 110, respectively, of SEQ ID NO: 43. Examples of such variant TβRII polypeptides include, but are not limited to, the sequences set forth in SEQ ID NOs: 57-60. A TβRII polypeptide may comprise a polypeptide or portion thereof that is encoded by any one of SEQ ID NOs: 61-64, or silent variants thereof or nucleic acids that hybridize to the complement thereof under stringent hybridization conditions. In particular embodiments, a TβRII polypeptide comprises a polypeptide or portion thereof that is encoded by any one of SEQ ID NO: 62, or silent variants thereof or nucleic acids that hybridize to the complement thereof under stringent hybridization conditions.

In some embodiments, the variant TβRII polypeptides of the disclosure further comprise an insertion of 36 amino acids (SEQ ID NO: 65) between the pair of glutamate residues (positions 151 and 152 of SEQ ID NO: 43, or positions 176 and 177 of SEQ ID NO: 42) located near the C-terminus of the human TβRII ECD, as occurs naturally in the human TβRII isoform C (Konrad et al., BMC Genomics 8:318, 2007).

The disclosure further demonstrates that TβRII polypeptides can be modified to selectively antagonize TβRII ligands. The N70 residue represents a potential glycosylation site. In some embodiments, the TβRII polypeptides are aglycosylated. In some embodiments, the TβRII polypeptides are aglycosylated or have reduced glycosylation at position Asn157. In some embodiments, the TβRII polypeptides are aglycosylated or have reduced glycosylation at position Asn73.

In certain embodiments, a TβRII polypeptide binds to TGFβ1, and the TβRII polypeptide does not show substantial binding to TGFβ3. In certain embodiments, a TβRII polypeptide binds to TGFβ3, and the TβRII polypeptide does not show substantial binding to TGFβ1. Binding may be assessed using purified proteins in solution or in a surface plasmon resonance system, such as a Biacore™ system.

In certain embodiments, a TβRII polypeptide inhibits TGFβ1 cellular signaling, and the TβRII polypeptide has an intermediate or limited inhibitory effect on TGFβ3 signaling. In certain embodiments, a TβRII polypeptide inhibits TGFβ3 cellular signaling, and the TβRII polypeptide has an intermediate or limited inhibitory effect on TGFβ1 signaling. Inhibitory effect on cell signaling can be assayed by methods known in the art.

Taken together, an active portion of a TβRII polypeptide may comprise amino acid residues 23-153, 23-154, 23-155, 23-156, 23-157, or 23-158 of SEQ ID NO: 43, as well as variants of these amino acid residues starting at any of amino acids 24-35 of SEQ ID NO: 43. Similarly, an active portion of a TβRII polypeptide may comprise amino acid residues 23-178, 23-179, 23-180, 23-181, 23-182, or 23-183 of SEQ ID NO: 42, as well as variants of these amino acid residues starting at any of amino acids 24-60 of SEQ ID NO: 42. Exemplary TβRII polypeptides comprise amino acid residues 29-159, 35-159, 23-153, 29-153 and 35-153 of SEQ ID NO: 43 or amino acid residues 29-184, 60-184, 23-178, 29-178 and 60-178 of SEQ ID NO: 42. Variants within these ranges are also contemplated, particularly those having at least 80%, 85%, 90%, 95%, or 99% identity to the corresponding portion of SEQ ID NO: 43 or SEQ ID NO: 42. A TβRII polypeptide may be selected that does not include the sequence consisting of amino acid residues 160-567 of SEQ ID NO: 43 or amino acid residues 185-592 of SEQ ID NO: 42. In particular embodiments, the TβRII polypeptides comprise an amino acid sequence at least 80% identical, and optionally at least 85%, 90%, 91%, 92%, 93%, 94%, 95%, 96%, 97%, 98%, 99%, or 100% identical to the amino acid sequence of SEQ ID NO: 46.

In some embodiments, any of the TβRII polypeptides disclosed herein are at least 80%, 85%, 90%, 91%, 92%, 93%, 94%, 95%, 97%, 99% or 100% identical to the amino acid sequence of any one of SEQ ID NOs: 46, 45, 49, 54, 57, 58, 59, 60, 66, 67, 68, or 69, but lack one or more N-terminal amino acids as compared to the amino acid sequences of SEQ ID NO: 46, 45, 49, 54, 57, 58, 59, 60, 66, 67, 68, 69, or 70. In some embodiments, the TβRII polypeptide lacks the amino acid corresponding to the first amino acid (threonine) of any one of SEQ ID NOs: 46, 45, 49, 54, 57, 58, 59, 60, 66, 67, 68, 69, or 70. In some embodiments, the TβRII polypeptide lacks the amino acids corresponding to the first and second amino acids (threonine and isoleucine, respectively) of any one of SEQ ID NOs: 46, 45, 49, 54, 57, 58, 59, 60, 66, 67, 68, 69, or 70. In some embodiments, the TβRII polypeptide lacks the amino acids corresponding to the first, second and third amino acids (threonine, isoleucine, and proline, respectively) of any one of SEQ ID NOs: 46, 45, 49, 54, 57, 58, 59, 60, 66, 67, 68, 69, or 70. In some embodiments, the TβRII polypeptide lacks the amino acids corresponding to the first, second, third and fourth amino acids (threonine, isoleucine, proline, proline, respectively) of any one of SEQ ID NOs: 46, 45, 49, 54, 57, 58, 59, 60, 66, 67, 68, 69, or 70.

In some embodiments, any of the TβRII polypeptides disclosed herein are at least 80%, 85%, 90%, 91%, 92%, 93%, 94%, 95%, 97%, 99% or 100% identical to the amino acid sequence of any one of SEQ ID NOs: 46, 68, or 70, but lack the amino acid corresponding to the first amino acid (threonine) of SEQ ID NO: 46, 68, or 70. In some embodiments, the TβRII polypeptide lacks the amino acids corresponding to the first and second amino acids (threonine and isoleucine, respectively) of SEQ ID NO: 46, 68, or 70. In some embodiments, the TβRII polypeptide lacks the amino acids corresponding to the first, second and third amino acids (threonine, isoleucine, and proline, respectively) of SEQ ID NO: 46, 68, or 70. In some embodiments, the TβRII polypeptide lacks the amino acids corresponding to the first, second, third and fourth amino acids (threonine, isoleucine, proline, proline, respectively) of SEQ ID NO: 46, 68, or 70.

In some embodiments, the fusion polypeptide comprises an amino acid sequence that is at least 85%, 90%, 91%, 92%, 93%, 94%, 95%, 97%, or 99% identical to any of the TβRII polypeptide amino acid sequences disclosed herein (e.g., SEQ ID NO: 46), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation). In some embodiments, the fusion polypeptide comprises an amino acid sequence that is at least 85%, 90%, 91%, 92%, 93%, 94%, 95%, 97%, or 99% identical to any of the linker sequences disclosed herein (e.g., SEQ ID NO: 82), wherein the linker portion of the fusion polypeptide comprises no more than 5, 4, 3, 2, or 1 additional amino acids (but which may include further post-translational modifications, such as PEGylation). In some embodiments, the fusion polypeptide comprises an amino acid sequence that is at least 85%, 90%, 91%, 92%, 93%, 94%, 95%, 97%, or 99% identical to any of the heterologous portion sequences disclosed herein (e.g., SEQ ID NO: 11), wherein the heterologous portion of the fusion polypeptide comprises no more than 25, 20, 15, 10, 5, 4, 3, 2, or 1 additional amino acids (but which may include further post-translational modifications, such as PEGylation). In some embodiments, the fusion polypeptide comprises any of the TβRII polypeptide amino acid sequences disclosed herein (e.g., SEQ ID NO: 46), wherein the TβRII polypeptide portion of the fusion polypeptide comprises no more than 10, 9, 8, 7, 6, 5, 4, 3, 2, or 1 additional amino acids (but which may include further post-translational modifications, such as PEGylation). In some embodiments, the fusion polypeptide comprises any of the linker sequences disclosed herein (e.g., SEQ ID NO: 82), wherein the linker portion of the fusion polypeptide comprises no more than 5, 4, 3, 2, or 1 additional amino acids (but which may include further post-translational modifications, such as PEGylation). In some embodiments, the fusion polypeptide comprises any of the heterologous portion sequences disclosed herein (e.g., SEQ ID NO: 11 or 148), wherein the heterologous portion of the fusion polypeptide comprises no more than 25, 20, 15, 10, 5, 4, 3, 2, or 1 additional amino acids (but which may include further post-translational modifications, such as PEGylation).

In some embodiments, the disclosure provides a fusion polypeptide, wherein the fusion polypeptide consists or consists essentially of (and not necessarily in the following order): a) an amino acid sequence that is at least 85%, 90%, 91%, 92%, 93%, 94%, 95%, 97%, or 99% identical to any of the TβRII polypeptide amino acid sequences disclosed herein (e.g., SEQ ID NO: 46), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation); b) an amino acid sequence that is at least 85%, 90%, 91%, 92%, 93%, 94%, 95%, 97%, or 99% identical to any of the linker sequences disclosed herein (e.g., SEQ ID NO: 82), wherein the linker portion of the fusion polypeptide comprises no more than 5, 4, 3, 2 or 1 additional amino acids (but which may include further post-translational modifications, such as PEGylation); and c) an amino acid sequence that is at least 85%, 90%, 91%, 92%, 93%, 94%, 95%, 97%, or 99% identical to any of the heterologous portion sequences disclosed herein (e.g., SEQ ID NO: 11 or 148), wherein the heterologous portion of the fusion polypeptide comprises no more than 25, 20, 15, 10, 5, 4, 3, 2, or 1 additional amino acids (but which may include further post-translational modifications, such as PEGylation); and d) optionally a leader sequence (e.g., SEQ ID NO: 25). In some embodiments, the disclosure provides a fusion polypeptide, wherein the fusion polypeptide consists or consists essentially of (and not necessarily in the following order): a) any of the TβRII polypeptide amino acid sequences disclosed herein (e.g., SEQ ID NO: 46), wherein the TβRII polypeptide portion of the fusion polypeptide comprises no more than 10, 9, 8, 7, 6, 5, 4, 3, 2 or 1 additional amino acids (but which may include further post-translational modifications, such as PEGylation); b) any of the linker sequences disclosed herein (e.g., SEQ ID NO: 82), wherein the linker portion of the fusion polypeptide comprises no more than 5, 4, 3, 2 or 1 additional amino acids (but which may include further post-translational modifications, such as PEGylation); and c) any of the heterologous portion sequences disclosed herein (e.g., SEQ ID NO: 11 or 148), wherein the heterologous portion of the fusion polypeptide comprises no more than 25, 20, 15, 10, 5, 4, 3, 2, or 1 additional amino acids (but which may include further post-translational modifications, such as PEGylation); and d) optionally a leader sequence (e.g., SEQ ID NO: 25).

In some embodiments, the disclosure provides a fusion polypeptide consisting of or consisting essentially of (and not necessarily in the following order): a) a TβRII polypeptide portion consisting of an amino acid sequence that is at least 85%, 90%, 91%, 92%, 93%, 94%, 95%, 97%, or 99% identical to the amino acid sequence of SEQ ID NO: 46 and no more than 10, 9, 8, 7, 6, 5, 4, 3, 2 or 1 additional amino acids (but which may include further post-translational modifications, such as PEGylation); b) a linker portion consisting of an amino acid sequence that is at least 85%, 90%, 91%, 92%, 93%, 94%, 95%, 97%, or 99% identical to the amino acid sequence of SEQ ID NO: 82 and no more than 5, 4, 3, 2 or 1 additional amino acids (but which may include further post-translational modifications, such as PEGylation); and c) a heterologous portion consisting of an amino acid sequence that is at least 85%, 90%, 91%, 92%, 93%, 94%, 95%, 97%, or 99% identical to the amino acid sequence of SEQ ID NO: 11 or SEQ ID NO: 148 and no more than 25, 20, 15, 10, 5, 4, 3, 2, or 1 additional amino acids (but which may include further post-translational modifications, such as PEGylation); and d) optionally a leader sequence (e.g., SEQ ID NO: 25). In some embodiments, the disclosure provides a fusion polypeptide consisting or consisting essentially of (and not necessarily in the following order): a) a TβRII polypeptide portion consisting of the amino acid sequence of SEQ ID NO: 46 and no more than 10, 9, 8, 7, 6, 5, 4, 3, 2 or 1 additional amino acids (but which may include further post-translational modifications, such as PEGylation); b) a linker portion consisting of the amino acid sequence of SEQ ID NO: 82 and no more than 5, 4, 3, 2 or 1 additional amino acids (but which may include further post-translational modifications, such as PEGylation); and c) a heterologous portion consisting of the amino acid sequence of SEQ ID NO: 11 or SEQ ID NO: 148 and no more than 25, 20, 15, 10, 5, 4, 3, 2, or 1 additional amino acids (but which may include further post-translational modifications, such as PEGylation); and d) optionally a leader sequence (e.g., SEQ ID NO: 25).

In some embodiments, the disclosure provides a TβRII fusion polypeptide wherein the polypeptide does not comprise an additional ligand binding domain in addition to the TβRII domain. In some embodiments, the polypeptide comprises a linear amino acid sequence comprising a TβRII domain and a heterologous portion (e.g., an Fc portion), but the linear amino acid sequence does not comprise any additional ligand binding domains. In some embodiments, the polypeptide comprises a linear amino acid sequence comprising a TβRII domain and an Fc portion, but the linear amino acid sequence does not comprise any additional ligand binding domains. In some embodiments, the disclosure provides a TβRII fusion polypeptide wherein the polypeptide does not comprise multiple ligand binding domains in a single linear amino acid sequence. In some embodiments, the disclosure provides a TβRII fusion polypeptide wherein the polypeptide does not comprise more than one continuous linker sequence in a single linear amino acid sequence. In some embodiments, the polypeptide does not comprise multiple continuous glycine and/or serine linkers (e.g., a linker comprising (GGGGS)n, wherein n≥4 (SEQ ID NO: 180)) in a single linear amino acid sequence. In some embodiments, the disclosure provides a TβRII fusion polypeptide wherein the heterologous portion is an Fc domain, and wherein only one continuous linker is covalently bound to the Fc domain. In some embodiments, the only one continuous linker comprises or consists of a (GGGGS)n linker, wherein n≥4 (SEQ ID NO: 180).

### 4. Fusion Polypeptides

In certain aspects, the disclosure provides ActRII and TβRII fusion polypeptides. The fusion polypeptides may be prepared according to any of the methods disclosed herein or that are known in the art.

In some embodiments, any of the fusion polypeptides disclosed herein comprises the following components: a) any of the polypeptides disclosed herein (“A”) (e.g., an ActRII or TβRII polypeptide), b) any of the linkers disclosed herein (“B”), c) any of the heterologous portions disclosed herein (“C”) (e.g., an Fc immunoglobulin domain), and optionally a leader sequence (“X”) (e.g., a tissue plasminogen activator leader sequence). In such embodiments, the fusion polypeptide may be arranged in a manner as follows (N-terminus to C-terminus): A-B-C or C-B-A. In such embodiments, the fusion polypeptide may be arranged in a manner as follows (N-terminus to C-terminus): X-A-B-C or X-C-B-A. In some embodiments, the fusion polypeptide comprises each of A, B and C (and optionally a leader sequence), and comprises no more than 100, 90, 80, 70, 60, 50, 40, 30, 20, 10, 5, 4, 3, 2 or 1 additional amino acids (but which may include further post-translational modifications, such as glycosylation).

In some embodiments, the fusion polypeptide comprises a leader sequence positioned in a manner as follows (N-terminus to C-terminus): X-A-B-C, and the fusion polypeptide comprises 1, 2, 3, 4, or 5 amino acids between X and A. In some embodiments, the fusion polypeptide comprises a leader sequence positioned in a manner as follows (N-terminus to C-terminus): X-C-B-A, and the fusion polypeptide comprises 1, 2, 3, 4, or 5 amino acids between X and C. In some embodiments, the fusion polypeptide comprises a leader sequence positioned in a manner as follows (N-terminus to C-terminus): X-A-B-C, and the fusion polypeptide comprises an alanine between X and A. In some embodiments, the fusion polypeptide comprises a leader sequence positioned in a manner as follows (N-terminus to C-terminus): X-C-B-A, and the fusion polypeptide comprises an alanine between X and C. In some embodiments, the fusion polypeptide comprises a leader sequence positioned in a manner as follows (N-terminus to C-terminus): X-A-B-C, and the fusion polypeptide comprises a glycine and an alanine between X and A. In some embodiments, the fusion polypeptide comprises a leader sequence positioned in a manner as follows (N-terminus to C-terminus): X-C-B-A, and the fusion polypeptide comprises a glycine and an alanine between X and C. In some embodiments, the fusion polypeptide comprises a leader sequence positioned in a manner as follows (N-terminus to C-terminus): X-A-B-C, and the fusion polypeptide comprises a threonine between X and A. In some embodiments, the fusion polypeptide comprises a leader sequence positioned in a manner as follows (N-terminus to C-terminus): X-C-B-A, and the fusion polypeptide comprises a threonine between X and C. In some embodiments, the fusion polypeptide comprises a leader sequence positioned in a manner as follows (N-terminus to C-terminus): X-A-B-C, and the fusion polypeptide comprises a threonine between A and B. In some embodiments, the fusion polypeptide comprises a leader sequence positioned in a manner as follows (N-terminus to C-terminus): X-C-B-A, and the fusion polypeptide comprises a threonine between C and B.

In certain aspects, fusion proteins of the disclosure comprise at least a portion of an ActRII or TβRII polypeptide and one or more heterologous portions (e.g., an immunoglobulin Fc domain), optionally with one or more linker domain sequence positioned between the ActRII or TβRII polypeptide domain and the one or more heterologous portions. Well-known examples of such heterologous portions include, but are not limited to, polyhistidine, Glu-Glu, glutathione S transferase (GST), thioredoxin, protein A, protein G, an immunoglobulin heavy chain constant region (Fc), maltose binding protein (MBP), or human serum albumin.

A heterologous portion may be selected so as to confer a desired property. For example, some heterologous portions are particularly useful for isolation of the fusion proteins by affinity chromatography. For the purpose of affinity purification, relevant matrices for affinity chromatography, such as glutathione-, amylase-, and nickel- or cobalt-conjugated resins are used. Many of such matrices are available in “kit” form, such as the Pharmacia GST purification system and the QIAexpress™ system (Qiagen) useful with (HIS₆) fusion partners. As another example, a heterologous portion may be selected so as to facilitate detection of the fusion polypeptides. Examples of such detection domains include the various fluorescent proteins (e.g., GFP) as well as “epitope tags,” which are usually short peptide sequences for which a specific antibody is available. Well known epitope tags for which specific monoclonal antibodies are readily available include FLAG, influenza virus haemagglutinin (HA), and c-myc tags. In some cases, the heterologous portions have a protease cleavage site, such as for Factor Xa or Thrombin, which allows the relevant protease to partially digest the fusion proteins and thereby liberate the recombinant proteins therefrom. The liberated proteins can then be isolated from the heterologous portion by subsequent chromatographic separation.

In certain embodiments, an ActRII or TβRII polypeptide domain is fused, optionally with an intervening linker domain, to a heterologous domain that stabilizes the ligand trap domain in vivo (a “stabilizer” domain). In general, “stabilizing” is meant anything that increases serum half-life, regardless of whether this is because of decreased destruction, decreased clearance by the kidney, or other pharmacokinetic effect of the agent. Fusion polypeptides with the Fc portion of an immunoglobulin are known to confer desirable pharmacokinetic properties on a wide range of proteins. Likewise, fusions to human serum albumin can confer desirable properties. Other types of heterologous portions that may be selected include multimerizing (e.g., dimerizing, tetramerizing) domains and functional domains. In some embodiments, a stabilizing domain also functions as a multimerization domain. Such multifunctional domains include, for example, Fc immunoglobulin domains. Various examples of Fc immunoglobulin domains and Fc-fusion proteins comprising one or more polypeptide domains are described throughout the disclosure.

In some embodiments, fusion proteins of the disclosure additionally include any of various leader sequences at the N-terminus. Such a sequence would allow the peptides to be expressed and targeted to the secretion pathway in a eukaryotic system. See, e.g., Ernst et al., U.S. Pat. No. 5,082,783 (1992). Alternatively, a native signal sequence may be used to effect extrusion from the cell. Possible leader sequences include native leaders, tissue plasminogen activator (TPA) and honeybee melittin (SEQ ID NOs. 26, 25, and 24 respectively). Examples of fusion proteins incorporating a TPA leader sequence include SEQ ID NOs: 27 and 71-78. Processing of signal peptides may vary depending on the leader sequence chosen, the cell type used and culture conditions, among other variables, and therefore actual N-terminal start sites for mature (e.g., an ActRII or TβRII polypeptide) polypeptides may shift by 1, 2, 3, 4 or 5 amino acids in either the N-terminal or C-terminal direction.

In one embodiment, fusion proteins of the invention comprise the amino acid sequence set forth in any one of SEQ ID NOs.: 27 and 71-78. It will be understood by one of ordinary skill in the art that corresponding variants based on the long isoform of TβRII will include the 25-amino acid insertion along with a conservative Val-Ile substitution at the flanking position C-terminal to the insertion.

### A. Fc-Fusion Proteins

As specific examples of fusion polypeptides comprising a multimerization domain, the disclosure provides fusion polypeptides comprising an ActRII or TβRII polypeptide fused to a polypeptide comprising a constant domain of an immunoglobulin, such as a CH1, CH2, or CH3 domain of an immunoglobulin or an immunoglobulin Fc domain. As used herein, the term “immunoglobulin Fc domain” or simply “Fc” is understood to mean the carboxyl-terminal portion of an immunoglobulin chain constant region, preferably an immunoglobulin heavy chain constant region, or a portion thereof. For example, an immunoglobulin Fc region may comprise 1) a CH1 domain, a CH2 domain, and a CH3 domain, 2) a CH1 domain and a CH2 domain, 3) a CH1 domain and a CH3 domain, 4) a CH2 domain and a CH3 domain, or 5) a combination of two or more domains and an immunoglobulin hinge region. In one embodiment the immunoglobulin Fc region comprises at least an immunoglobulin hinge region a CH2 domain and a CH3 domain, and preferably lacks the CH1 domain. In some embodiments, the immunoglobulin Fc region is a human immunoglobulin Fc region. In some embodiments, the class of immunoglobulin from which the heavy chain constant region is derived is IgG (Igγ) (γ subclasses 1, 2, 3, or 4). In certain embodiments, the constant region is derived from IgG1. Other classes of immunoglobulin, IgA (Igα), IgD (Igδ), IgE (Igε) and IgM (Igμ), may be used. The choice of appropriate immunoglobulin heavy chain constant region is discussed in detail in U.S. Pat. Nos. 5,541,087 and 5,726,044, which is incorporated herein in its entirety. The choice of particular immunoglobulin heavy chain constant region sequences from certain immunoglobulin classes and subclasses to achieve a particular result is considered to be within the level of skill in the art. In some embodiments, portion of the DNA construct encoding the immunoglobulin Fc region preferably comprises at least a portion of a hinge domain, and preferably at least a portion of a CH3 domain of Fc gamma or the homologous domains in any of IgA, IgD, IgE, or IgM. Furthermore, it is contemplated that substitution or deletion of amino acids within the immunoglobulin heavy chain constant regions may be useful in the practice of the methods and compositions disclosed herein. One example would be to introduce amino acid substitutions in the upper CH2 region to create an Fc variant with reduced affinity for Fc receptors (Cole et al. (1997) J. Immunol. 159:3613). Fc domains derived from human IgG1, IgG2, IgG3, and IgG4 are provided herein.

An example of a native amino acid sequence that may be used for the Fc portion of human IgG1 (G1Fc) is shown below (SEQ ID NO.: 11). Dotted underline indicates the hinge region, and solid underline indicates positions with naturally occurring variants. In part, the disclosure provides polypeptides (e.g., ActRII and TβRII) comprising, consisting of, or consisting essentially of an amino acid sequence with 70%, 80%, 85%, 86%, 87%, 88%, 89%, 90%, 91%, 92%, 93%, 94%, 95%, 96%, 97%, 98%, 99%, or 100% identity to SEQ ID NO.: 11.

Naturally occurring variants in G1Fc would include E134D and M136L according to the numbering system used in SEQ ID NO: 11 (see Uniprot P01857).

| (SEQ ID NO: 11) | |
|---|---|
| 1 | 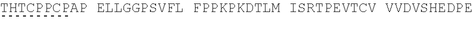 |
| 51 | VKFNWYVDGV EVHNAKTKPR EEQYNSTYRV VSVLTVLHQD WLNGKEYKCK |
| 101 | VSNKALPAPI EKTISKAKGQ PREPQVYTLP PSREEMTKNQ VSLTCLVKGF |
| 151 | YPSDIAVEWE SNGQPENNYK TTPPVLDSDG SFFLYSKLTV DKSRWQQGNV |
| 201 | FSCSVMHEAL HNHYTQKSLS LSPGK |

Optionally, the IgG1 Fc domain has one or more mutations at residues such as Asp-265, lysine 322, and Asn-434. In certain cases, the mutant IgG1 Fc domain having one or more of these mutations (e.g., Asp-265 mutation) has reduced ability of binding to the Fcγ receptor relative to a wild-type Fc domain. In other cases, the mutant Fc domain having one or more of these mutations (e.g., Asn-434 mutation) has increased ability of binding to the MHC class I-related Fc-receptor (FcRN) relative to a wild-type IgG1 Fc domain.

An example of a native amino acid sequence that may be used for the Fc portion of human IgG2 (G2Fc) is shown below (SEQ ID NO: 12). Dotted underline indicates the hinge region and double underline indicates positions where there are data base conflicts in the sequence (according to UniProt P01859). In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 12.

| (SEQ ID NO: 12) | |
|---|---|
| 1 | 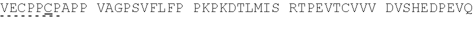 |
| 51 | FNWYVDGVEV HANKTKPREE QFNSTFRVVS VLTVVHQDWL NGKEYKCKVS |
| 101 | NKGLPAPIEK TISKTKGQPR EPQVYTLPPS REEMTKNQVS LTCLVKGFYP |
| 151 | SDIAVEWESN GQPENNYKTT PPMLDSDGSF FLYSKLTVDK SRWQQGNVFS |
| 201 | CSVMHEALHN HYTQKSLSLS PGK |

Two examples of amino acid sequences that may be used for the Fc portion of human IgG3 (G3Fc) are shown below. The hinge region in G3Fc can be up to four times as long as in other Fc chains and contains three identical 15-residue segments preceded by a similar 17-residue segment. The first G3Fc sequence shown below (SEQ ID NO: 13) contains a short hinge region consisting of a single 15-residue segment, whereas the second G3Fc sequence (SEQ ID NO: 14) contains a full-length hinge region. In each case, dotted underline indicates the hinge region, and solid underline indicates positions with naturally occurring variants according to UniProt P01859. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NOs: 13 and 14.

| (SEQ ID NO: 13) | |
|---|---|
| 1 | 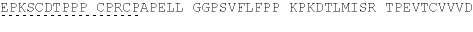 |
| 51 | VSHEDPEVQF KWYVDGVEVH NAKTKPREEQ YNSTFRVVSV LTVLHQDWLN |
| 101 | GKEYKCKVSN KALPAPIEKT ISKTKGQPRE PQVYTLPPSR EEMTKNQVSL |
| 151 | TCLVKGFYPS DIAVEWESSG QPENNYNTTP PMLDSDGSFF LYSKLTVDKS |
| 201 | RWQQGNIFSC SVMHEALHNR FTQKSLSLSP |
| (SEQ ID NO: 14) | |
| 1 | 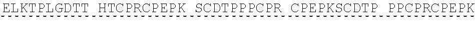 |
| 51 | 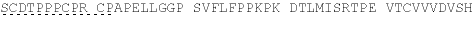 |
| 101 | EDPEVQFKXY VDGVEVHANK TKPREEQYNS TFRVVSVLTV LHQDWLNGKE |
| 151 | YKCKVSNKAL PAPIEKTISK TKGQPREPQV YTLPPSREEM TKNQVSLTCL |
| 201 | VKGFYPSDIA VEWESSGQPE NNYNTTPPML DSDGSFFLYS KLTVDKSRWQ |
| 251 | QGNIFSCSVM HEALHNRFTQ KSLSLSPGK |

Naturally occurring variants in G3Fc (for example, see Uniprot P01860) include E68Q, P76L, E79Q, Y81F, D97N, N100D, T124A, S169N, S169del, F221Y when converted to the numbering system used in SEQ ID NO: 13, and the present disclosure provides fusion proteins comprising G3Fc domains containing one or more of these variations. In addition, the human immunoglobulin IgG3 gene (IGHG3) shows a structural polymorphism characterized by different hinge lengths [see Uniprot P01859]. Specifically, variant WIS is lacking most of the V region and all of the CH1 region. It has an extra interchain disulfide bond at position 7 in addition to the 11 normally present in the hinge region. Variant ZUC lacks most of the V region, all of the CH1 region, and part of the hinge. Variant OMM may represent an allelic form or another gamma chain subclass. The present disclosure provides additional fusion proteins comprising G3Fc domains containing one or more of these variants.

An example of a native amino acid sequence that may be used for the Fc portion of human IgG4 (G4Fc) is shown below (SEQ ID NO: 15). Dotted underline indicates the hinge region. In part, the disclosure provides polypeptides comprising, consisting essential of, or consisting of amino acid sequences with 70%, 75%, 80%, 85%, 86%, 87%, 88%, 89%, 90%, 91%, 92%, 93%, 94%, 95%, 96%, 97%, 98%, 99%, or 100% identity to SEQ ID NO: 15.

| (SEQ ID NO: 15) | |
|---|---|
| 1 | 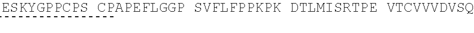 |
| 51 | EDPEVQFNWY VDGVEVHANK TKPREEQFNS TYRVVSVLTV LHQDWLNGKE |
| 101 | YKCKVSNKGL PSSIEKTISK AKGQPREPQV YTLPPSQEEM TKNQVSLTCL |
| 151 | VKGFYPSDIA VEWESNGQPE NNYKTTPPVL DSDGSFFLYS RLTVDKSRWQ |
| 201 | EGNVFSCSVM HEALHNHTYQ KSLSLSLGK |

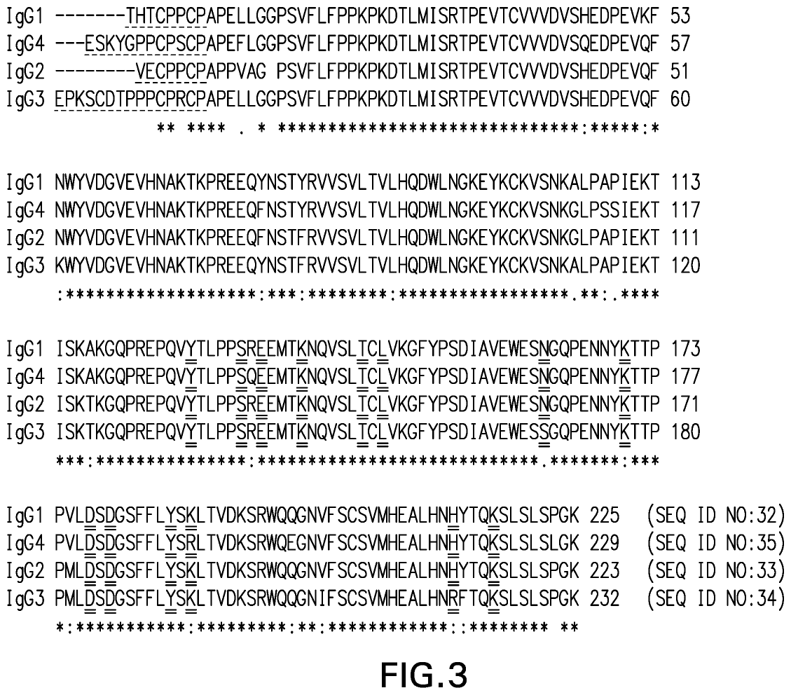

A variety of engineered mutations in the Fc domain are presented herein with respect to the G1Fc sequence (SEQ ID NO: 11), and analogous mutations in G2Fc, G3Fc, and G4Fc can be derived from their alignment with G1Fc in FIG. 3. Due to unequal hinge lengths, analogous Fc positions based on isotype alignment (FIG. 3) possess different amino acid numbers in SEQ ID NOs: 11, 12, 13, 14, and 15. It can also be appreciated that a given amino acid position in an immunoglobulin sequence consisting of hinge, C_{H}2, and C_{H}3 regions (e.g., SEQ ID NOs: 11, 12, 13, 14, and 15) will be identified by a different number than the same position when numbering encompasses the entire IgG1 heavy-chain constant domain (consisting of the C_{H}1, hinge, C_{H}2, and C_{H}3 regions) as in the Uniprot database. For example, correspondence between selected C_{H}3 positions in a human G1Fc sequence (SEQ ID NO: 11), the human IgG1 heavy chain constant domain (Uniprot P01857), and the human IgG1 heavy chain is as follows.

Correspondence of C_{H}3 Positions in Different Numbering Systems

| | IgG1 heavy chain | |
|---|---|---|
| G1Fc | constant domain | IgG1 heavy chain |
| (Numbering begins at first | (Numbering begins at | (EU numbering scheme |
| threonine in hinge region) | C_{H}1) | of Kabat et al., 1991*) |
| Y127 | Y232 | Y349 |
| S132 | S237 | S354 |
| E134 | E239 | E356 |
| T144 | T249 | T366 |
| L146 | L251 | L368 |
| K170 | K275 | K392 |
| D177 | D282 | D399 |
| Y185 | Y290 | Y407 |
| K187 | K292 | K409 |

*Kabat et al. (eds) 1991; pp. 688-696 in Sequences of Proteins of Immunological Interest, 5^{th} ed., Vol. 1, NIH, Bethesda, MD.

Various methods are known in the art that increase desired pairing of Fc-containing fusion polypeptide chains in a single cell line to produce an asymmetric fusion protein at acceptable yields [Klein et al (2012) mAbs 4:653-663; and Spiess et al (2015) Molecular Immunology 67(2A): 95-106]. Methods to obtain desired pairing of Fc-containing chains include, but are not limited to, charge-based pairing (electrostatic steering), “knobs-into-holes” steric pairing, SEEDbody pairing, and leucine zipper-based pairing [Ridgway et al (1996) Protein Eng 9:617-621; Merchant et al (1998) Nat Biotech 16:677-681; Davis et al (2010) Protein Eng Des Sel 23:195-202; Gunasekaran et al (2010); 285:19637-19646; Wranik et al (2012) J Biol Chem 287:43331-43339; U.S. Pat. No. 5,932,448; WO 1993/011162; WO 2009/089004, and WO 2011/034605].

In some embodiments, the disclosure provides Fc fusion proteins with engineered or variant Fc regions. Such Fc fusion proteins may be useful, for example, in modulating effector functions, such as, antigen-dependent cytotoxicity (ADCC) and complement-dependent cytotoxicity (CDC). Additionally, the modifications may improve the stability of the Fc fusion proteins. Amino acid sequence variants of the Fc fusion proteins are prepared by introducing appropriate nucleotide changes into the DNA, or by peptide synthesis. Such variants include, for example, deletions from, and/or insertions into and/or substitutions of, residues within the amino acid sequences of the antibodies and Fc fusion proteins disclosed herein. Any combination of deletion, insertion, and substitution is made to arrive at the final construct, provided that the final construct possesses the desired characteristics. The amino acid changes also may alter post-translational processes of the Fc fusion proteins, such as changing the number or position of glycosylation sites.

Fc fusion proteins with reduced effector function may be produced by introducing changes in the amino acid sequence, including, but are not limited to, the Ala-Ala mutation described by Bluestone et al. (see WO 94/28027 and WO 98/47531; also see Xu et al. 2000 Cell Immunol 200; 16-26). Thus, in certain embodiments, Fc fusion proteins of the disclosure with mutations within the constant region including the Ala-Ala mutation are used to reduce or abolish effector function. According to these embodiments, Fc fusion proteins comprise a mutation to an alanine at position 234 or a mutation to an alanine at position 235, or a combination thereof. In one embodiment, the Fc fusion protein comprises an IgG4 framework, wherein the Ala-Ala mutation would describe a mutation(s) from phenylalanine to alanine at position 234 and/or a mutation from leucine to alanine at position 235. In another embodiment, the Fc fusion protein comprises an IgG1 framework, wherein the Ala-Ala mutation would describe a mutation(s) from leucine to alanine at position 234 and/or a mutation from leucine to alanine at position 235. While alanine substitutions at these sites are effective in reducing ADCC in both human and murine antibodies, these substitutions are less effective at reducing CDC activity. Another single variant P329A, identified by a random mutagenesis approach to map the C1q binding site of the Fc, is highly effective at reducing CDC activity while retaining ADCC activity. A combination of L234A, L235A, and P329A (LALA-PG, Kabat positions) substitutions have been shown to effectively silence the effector function of human IgG1 antibodies. For a detailed discussion of LALA, LALA-PG, and other mutations, see Lo et al. (2017) 1 Biol. Chem. 292:3900-3908, the contents of which are hereby incorporated herein by reference in their entirety. In some embodiments, Fc fusion proteins of the disclosure comprise L234A, L235A, and P329G mutations (LALA-PG; Kabat positions) in the Fc region of the heavy chain. The Fc fusion protein may alternatively or additionally carry other mutations, including the point mutation K322A in the CH2 domain (Hezareh et al. 2001 J Virol. 75: 12161-8).

In particular embodiments, the Fc fusion protein is modified to either enhance or inhibit complement dependent cytotoxicity (CDC). Modulated CDC activity may be achieved by introducing one or more amino acid substitutions, insertions, or deletions in an Fc region (see, e.g., U.S. Pat. No. 6,194,551). Alternatively, or additionally, cysteine residue(s) may be introduced in the Fc region, thereby allowing interchain disulfide bond formation in this region. The Fc fusion protein thus generated may have improved or reduced internalization capability and/or increased or decreased complement-mediated cell killing. See Caron et al., J. Exp Med. 176:1191-1195 (1992) and Shopes, B. J. Immunol. 148:2918-2922 (1992), WO99/51642, Duncan & Winter Nature 322: 738-40 (1988); U.S. Pat. Nos. 5,648,260; 5,624,821; and WO94/29351.

### B. Linkers

The disclosure provides an ActRII polypeptide or a TβRII polypeptide (including variants thereof) that may be fused to an additional polypeptide disclosed herein including, for example, fused to a heterologous portion (e.g., an Fc portion). In these embodiments, the polypeptide portion (e.g., ActRII or TβRII polypeptides including variants thereof) is connected to the additional polypeptide (e.g., a heterologous portion such as an Fc domain) by means of a linker. In some embodiments, the linkers are glycine and serine rich linkers. In some embodiments, the linker is rich in glycine (e.g., 2-10, 2-5, 2-4, 2-3 glycine residues) or glycine and proline residues and may, for example, contain a single sequence of threonine/serine and glycines or repeating sequences of threonine/serine and/or glycines, e.g., GGG (SEQ ID NO: 16), GGGG (SEQ ID NO: 17), TGGGG (SEQ ID NO: 18), SGGGG (SEQ ID NO: 19), TGGG (SEQ ID NO: 20), or SGGG (SEQ ID NO: 21) singlets, or repeats. Other near neutral amino acids, such as, but not limited to, Thr, Asn, Pro and Ala, may also be used in the linker sequence. In some embodiments, the linker comprises various permutations of amino acid sequences containing Gly and Ser. In some embodiments, the linker is greater than 10 amino acids in length. In further embodiments, the linkers have a length of at least 12, 15, 20, 21, 25, 30, 35, 40, 45 or 50 amino acids. In some embodiments, the linker is less than 40, 35, 30, 25, 22 or 20 amino acids. In some embodiments, the linker is 10-50, 10-40, 10-30, 10-25, 10-21, 10-15, 10, 15-25, 17-22, 20, or 21 amino acids in length. In some embodiments, the linker comprises the amino acid sequence GlyGlyGlyGlySer (GGGGS) (SEQ ID NO: 22), or repetitions thereof (GGGGS)n, where n≥2. In particular embodiments n≥3, or n=3-10. In some embodiments, n≥4, or n=4-10. In some embodiments, n is not greater than 4 in a (GGGGS)n linker. In some embodiments, n=4-10, 4-9, 4-8, 4-7, 4-6, 4-5, 5-8, 5-7, or 5-6. In some embodiments, n=3, 4, 5, 6, or 7. In particular embodiments, n=4. In some embodiments, a linker comprising a (GGGGS)n sequence also comprises an N-terminal threonine. In some embodiments, the linker is any one of the following:

(SEQ ID NO: 79)
GGGGSGGGGS
(SEQ ID NO: 80)
TGGGGSGGGGS
(SEQ ID NO: 81)
TGGGGSGGGGSGGGGS
(SEQ ID NO: 82)
TGGGGSGGGGSGGGGSGGGGS
(SEQ ID NO: 83)
TGGGGSGGGGSGGGGSGGGGSGGGGS
(SEQ ID NO: 84)
TGGGGSGGGGSGGGGSGGGGSGGGGSGGGGS
(SEQ ID NO: 85)
TGGGGSGGGGSGGGGSGGGGSGGGGSGGGGSGGGGS
(SEQ ID NO: 178)
GGGGSGGGGS
(SEQ ID NO: 179)
GGGGSGGGGSGGGGS
(SEQ ID NO: 180)
GGGGSGGGGSGGGGSGGGGS
(SEQ ID NO: 181)
GGGGSGGGGSGGGGSGGGGSGGGGS
(SEQ ID NO: 182)
GGGGSGGGGSGGGGSGGGGSGGGGSGGGGS
or
(SEQ ID NO: 183)
GGGGSGGGGSGGGGSGGGGSGGGGSGGGGSGGGGS.

In some embodiments, the linker comprises the amino acid sequence of TGGGPKSCDK (SEQ ID NO: 86). In some embodiments, the linker is any one of SEQ ID NOs: 79-86 lacking the N-terminal threonine. In some embodiments, the linker does not comprise the amino acid sequence of SEQ ID NO: 84 or 85.

In some embodiments, a polypeptide described (e.g., ActRII, TβRII, and polypeptides including variants thereof) herein includes a polypeptide fused to a moiety by way of a linker. In some embodiments, the moiety increases stability of the polypeptide. In some embodiments, the moiety is selected from the group consisting of an Fc domain monomer, a wild-type Fc domain, an Fc domain with amino acid substitutions (e.g., one or more substitutions that reduce dimerization), an albumin-binding peptide, a fibronectin domain, or a human serum albumin. Suitable peptide linkers are known in the art, and include, for example, peptide linkers containing flexible amino acid residues such as glycine, alanine, and serine. In some embodiments, a linker can contain motifs, e.g., multiple or repeating motifs, of GA, GS, GG, GGA, GGS, GGG (SEQ ID NO: 16), GGGA (SEQ ID NO: 87), GGGS (SEQ ID NO: 88), GGGG (SEQ ID NO: 89), GGGGA (SEQ ID NO: 90), GGGGS (SEQ ID NO: 91), GGGGG (SEQ ID NO: 92), GGAG (SEQ ID NO: 93), GGSG (SEQ ID NO: 94), AGGG (SEQ ID NO: 95), or SGGG (SEQ ID NO: 21). In some embodiments, a linker can contain 2 to 12 amino acids including motifs of GA or GS, e.g., GA, GS, GAGA (SEQ ID NO: 96), GSGS (SEQ ID NO: 97), GAGAGA (SEQ ID NO: 98), GSGSGS (SEQ ID NO: 99), GAGAGAGA (SEQ ID NO: 100), GSGSGSGS (SEQ ID NO: 101), GAGAGAGAGA (SEQ ID NO: 102), GSGSGSGSGS (SEQ ID NO: 103), GAGAGAGAGAGA (SEQ ID NO: 104), and GSGSGSGSGSGS (SEQ ID NO: 105). In some embodiments, a linker can contain 3 to 12 amino acids including motifs of GGA or GGS, e.g., GGA, GGS, GGAGGA (SEQ ID NO: 106), GGSGGS (SEQ ID NO: 107), GGAGGAGGA (SEQ ID NO: 108), GGSGGSGGS (SEQ ID NO: 109), GGAGGAGGAGGA (SEQ ID NO: 110), and GGSGGSGGSGGS (SEQ ID NO: 111). In some embodiments, a linker can contain 4 to 12 amino acids including motifs of GGAG (SEQ ID NO: 112), GGSG (SEQ ID NO: 113), GGAGGGAG (SEQ ID NO: 114), GGSGGGSG (SEQ ID NO: 115), GGAGGGAGGGAG (SEQ ID NO: 116), and GGSGGGSGGGSG (SEQ ID NO: 117). In some embodiments, a linker can contain motifs of GGGGA (SEQ ID NO: 118) or GGGGS (SEQ ID NO: 119), e.g., GGGGAGGGGAGGGGA (SEQ ID NO: 120) and GGGGSGGGGSGGGGS (SEQ ID NO: 121). In some embodiments, an amino acid linker between a moiety (e.g., an Fc domain monomer, a wild-type Fc domain, an Fc domain with amino acid substitutions (e.g., one or more substitutions that reduce dimerization), an albumin-binding peptide, a fibronectin domain, or a human serum albumin) and a polypeptide (e.g., ActRII and TβRII polypeptides, including variants thereof) is GGG, GGGA (SEQ ID NO: 122), GGGG (SEQ ID NO: 17), GGGAG (SEQ ID NO: 123), GGGAGG (SEQ ID NO: 124), or GGGAGGG (SEQ ID NO: 125).

In some embodiments, a linker also contains amino acids other than glycine, alanine, and serine, e.g., AAAL (SEQ ID NO: 126), AAAK (SEQ ID NO: 127), AAAR (SEQ ID NO: 128), EGKSSGSGSESKST (SEQ ID NO: 129), GSAGSAAGSGEF (SEQ ID NO: 130), AEAAAKEAAAKA (SEQ ID NO: 131), KESGSVSSEQLAQFRSLD (SEQ ID NO: 132), GENLYFQSGG (SEQ ID NO: 133), SACYCELS (SEQ ID NO: 134), RSIAT (SEQ ID NO: 135), RPACKIPNDLKQKVMNH (SEQ ID NO: 136), GGSAGGSGSGSSGGSSGASGTGTAGGTGSGSGTGSG (SEQ ID NO: 137), AAANSSIDLISVPVDSR (SEQ ID NO: 138), or GGSGGGSEGGGSEGGGSEGGGSEGGGSEGGGSGGGS (SEQ ID NO: 139). In some embodiments, a linker can contain motifs, e.g., multiple or repeating motifs, of EAAAK (SEQ ID NO: 140). In some embodiments, a linker contains motifs, e.g., multiple or repeating motifs, of praline-rich sequences such as (XP)n, in which X is any amino acid (e.g., A, K, or E) and n is from 1-5, and PAPAP (SEQ ID NO: 141).

The length of the peptide linker and the amino acids used can be adjusted depending on the two polypeptides involved and the degree of flexibility desired in the final polypeptide fusion polypeptide. The length of the linker can be adjusted to ensure proper polypeptide folding and avoid aggregate formation.

### 5. Polypeptide Variants and Modifications

In part, the disclosure relates to ActRII and TβRII variant polypeptides. Variant polypeptides of the disclosure included, for example, variant polypeptides produced by one or more amino acid substitutions, deletions, additions or combinations thereof as well as variants of one or more post-translational modifications (e.g., including, but are not limited to, acetylation, carboxylation, glycosylation, phosphorylation, lipidation, and acylation). Methods for generating variant polypeptides comprising one or more amino acid modifications, particularly methods for generating variant polypeptides that have one or more desired properties, are described herein or otherwise well known in the art. Likewise, various methods for determining if a variant polypeptide has retained or developed one or more desired properties (e.g., alterations in ligand binding and/or antagonistic activities) are described herein or otherwise well known in the art. These methods can be used to generate variant polypeptides (e.g., variant ActRII or TβRII polypeptides) as well as validate their activity (or other properties) as described here.

As described above, the disclosure provides polypeptides (e.g., ActRII or TβRII polypeptides) sharing a specified degree of sequence identity or similarity to a naturally occurring polypeptide. To determine the percent identity of two amino acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues at corresponding amino acid positions are then compared. When a position in the first sequence is occupied by the same amino acid residue as the corresponding position in the second sequence, then the molecules are identical at that position (as used herein amino acid “identity” is equivalent to amino acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity and similarity between two sequences can be accomplished using a mathematical algorithm (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991).

In one embodiment, the percent identity between two amino acid sequences is determined using the Needleman and Wunsch (J Mol. Biol. (48):444-453 (1970)) algorithm which has been incorporated into the GAP program in the GCG software package (available at http://www.gcg.com). In a specific embodiment, the following parameters are used in the GAP program: either a Blosum 62 matrix or a PAM250 matrix, and a gap weight of 16, 14, 12, 10, 8, 6, or 4 and a length weight of 1, 2, 3, 4, 5, or 6. In yet another embodiment, the percent identity between two nucleotide sequences is determined using the GAP program in the GCG software package (Devereux, J., et al., Nucleic Acids Res. 12(1):387 (1984)) (available at http://www.gcg.com). Exemplary parameters include using a NWSgapdna.CMP matrix and a gap weight of 40, 50, 60, 70, or 80 and a length weight of 1, 2, 3, 4, 5, or 6. Unless otherwise specified, percent identity between two amino acid sequences is to be determined using the GAP program using a Blosum 62 matrix, a GAP weight of 10 and a length weight of 3, and if such algorithm cannot compute the desired percent identity, a suitable alternative disclosed herein should be selected.

In another embodiment, the percent identity between two amino acid sequences is determined using the algorithm of E. Myers and W. Miller (CABIOS, 4:11-17 (1989)) which has been incorporated into the ALIGN program (version 2.0), using a PAM120 weight residue table, a gap length penalty of 12 and a gap penalty of 4.

Another embodiment for determining the best overall alignment between two amino acid sequences can be determined using the FASTDB computer program based on the algorithm of Brutlag et al. (Comp. App. Biosci., 6:237-245 (1990)). In a sequence alignment the query and subject sequences are both amino acid sequences. The result of said global sequence alignment is presented in terms of percent identity. In one embodiment, amino acid sequence identity is performed using the FASTDB computer program based on the algorithm of Brutlag et al. (Comp. App. Biosci., 6:237-245 (1990)). In a specific embodiment, parameters employed to calculate percent identity and similarity of an amino acid alignment comprise: Matrix=PAM 150, k-tuple=2, Mismatch Penalty-1, Joining Penalty-20, Randomization Group Length=0, Cutoff Score=1, Gap Penalty=5 and Gap Size Penalty-0.05.

In some embodiments, the disclosure contemplates making functional variant polypeptides by modifying the structure of a polypeptide (e.g., an ActRII or TβRII polypeptide) for such purposes as enhancing therapeutic efficacy or stability (e.g., shelf-life and resistance to proteolytic degradation in vivo). Variants can be produced by amino acid substitution, deletion, addition, or combinations thereof. For instance, it is reasonable to expect that an isolated replacement of a leucine with an isoleucine or valine, an aspartate with a glutamate, a threonine with a serine, or a similar replacement of an amino acid with a structurally related amino acid (e.g., conservative mutations) will not have a major effect on the biological activity of the resulting molecule. Conservative replacements are those that take place within a family of amino acids that are related in their side chains. Whether a change in the amino acid sequence of a polypeptide of the disclosure results in a functional homolog can be readily determined by assessing the ability of the variant polypeptide to produce a response in cells in a fashion similar to the wild-type polypeptide, or to bind to one or more ActRII ligands including, for example, activin A, activin B, GDF8, GDF11, BMP6, and BMP10, or TβRII ligands including, for example, TGFβ1 and TGFβ3.

In certain embodiments, the disclosure contemplates specific mutations of a polypeptide (e.g., an ActRII or TβRII polypeptide) so as to alter the glycosylation of the polypeptide. Such mutations may be selected so as to introduce or eliminate one or more glycosylation sites, such as O-linked or N-linked glycosylation sites. Asparagine-linked glycosylation recognition sites generally comprise a tripeptide sequence, asparagine-X-threonine or asparagine-X-serine (where “X” is any amino acid) which is specifically recognized by appropriate cellular glycosylation enzymes. The alteration may also be made by the addition of, or substitution by, one or more serine or threonine residues to the sequence of the polypeptide (for O-linked glycosylation sites). A variety of amino acid substitutions or deletions at one or both of the first or third amino acid positions of a glycosylation recognition site (and/or amino acid deletion at the second position) results in non-glycosylation at the modified tripeptide sequence. Another means of increasing the number of carbohydrate moieties on a polypeptide is by chemical or enzymatic coupling of glycosides to the polypeptide. Depending on the coupling mode used, the sugar(s) may be attached to (a) arginine and histidine; (b) free carboxyl groups; (c) free sulfhydryl groups such as those of cysteine; (d) free hydroxyl groups such as those of serine, threonine, or hydroxyproline; (e) aromatic residues such as those of phenylalanine, tyrosine, or tryptophan; or (f) the amide group of glutamine. Removal of one or more carbohydrate moieties present on a polypeptide may be accomplished chemically and/or enzymatically. Chemical deglycosylation may involve, for example, exposure of a polypeptide to the compound trifluoromethanesulfonic acid, or an equivalent compound. This treatment results in the cleavage of most or all sugars except the linking sugar (N-acetylglucosamine or N-acetylgalactosamine), while leaving the amino acid sequence intact. Enzymatic cleavage of carbohydrate moieties on polypeptides can be achieved by the use of a variety of endo- and exo-glycosidases as described by Thotakura et al. [Meth. Enzymol. (1987) 138:350]. The sequence of a polypeptide may be adjusted, as appropriate, depending on the type of expression system used, as mammalian, yeast, insect, and plant cells may all introduce differing glycosylation patterns that can be affected by the amino acid sequence of the peptide. In general, polypeptides of the present disclosure for use in humans may be expressed in a mammalian cell line that provides proper glycosylation, such as HEK293 or CHO cell lines, although other mammalian expression cell lines are expected to be useful as well. In some embodiments, polypeptides of the disclosure (e.g., an ActRII or TβRII polypeptides) are glycosylated and have a glycosylation pattern obtainable from of the polypeptide in a CHO cell.

The disclosure further contemplates a method of generating mutants, particularly sets of combinatorial mutants of a polypeptide (e.g., an ActRII or TβRII polypeptide) as well as truncation mutants. Pools of combinatorial mutants are especially useful for identifying functionally active (e.g., ActRII or TβRII ligand binding) sequences. The purpose of screening such combinatorial libraries may be to generate, for example, polypeptides variants which have altered properties, such as altered pharmacokinetic or altered ligand binding. A variety of screening assays are provided below, and such assays may be used to evaluate variants. For example, polypeptide (e.g., an ActRII or TβRII polypeptide) variants may be screened for ability to bind to one or more ActRII or TβRII ligands (e.g., activin A, activin B, GDF8, GDF11, BMP6, and BMP10, or TGFβ1 and TGFβ3), to prevent binding of an ActRII ligand to an ActRII polypeptide or a TβRII ligand to a TβRII polypeptide, and/or to interfere with signaling caused by an ActRII or TβRII ligand.

The activity of a polypeptide (e.g., an ActRII or TβRII polypeptide), or variant thereof may also be tested in a cell-based or in vivo assay. For example, the effect of a polypeptide, or a variant thereof on the expression of genes involved in the pathogenesis of pulmonary hypertension associated with lung disease is assessed. This may, as needed, be performed in the presence of one or more recombinant ligand proteins (e.g., activin A, activin B, GDF8, GDF11, BMP6, BMP10, TGFβ1, and/or TGFβ3), and cells maybe transfected so as to produce polypeptide (e.g., an ActRII or TβRII polypeptide), and optionally, an ActRII or TβRII ligand. Likewise, a polypeptide, or a variant thereof may be administered to a mouse or other animal and effects on the pathogenesis of pulmonary hypertension associated with lung disease may be assessed using art-recognized methods. Similarly, the activity of a polypeptide, or variant thereof may be tested in blood cell precursor cells for any effect on growth of these cells, for example, by the assays as described herein and those of common knowledge in the art. A SMAD-responsive reporter gene may be used in such cell lines to monitor effects on downstream signaling.

In certain aspects, a polypeptide (e.g., an ActRII or TβRII polypeptide) of the disclosure bind to one or more ActRII or TβRII ligands. In some embodiments, a polypeptide of the disclosure bind to one or more ActRII or TβRII ligands with a K_{D} of at least 1×10⁻⁷ M. In some embodiments, the one or more ActRII or TβRII ligands is selected from the group consisting of: activin A, activin B, GDF8, GDF11, and BMP10, or TGFβ1 and TGFβ3.

In certain aspects, a polypeptide (e.g., an ActRII or TβRII polypeptide) of the disclosure inhibits one or more ActRII or TβRII family ligands. In some embodiments, a polypeptide of the disclosure inhibits signaling of one or more ActRII or TβRII ligands. In some embodiments, a polypeptide of the disclosure inhibits Smad signaling of one or more ActRII or TβRII ligands. In some embodiments, a polypeptide of the disclosure inhibits signaling of one or more ActRII or TβRII ligands in a cell-based assay. In some embodiments, a polypeptide of the disclosure inhibits one or more ActRII-ALK4 ligands selected from the group consisting of: activin A, activin B, GDF8, GDF11, and BMP10, or TGFβ1 and TGFβ3.

Combinatorial-derived variants can be generated which have increased selectivity or generally increased potency relative to a reference polypeptide (e.g., an ActRII or TβRII polypeptide). Such variants, when expressed from recombinant DNA constructs, can be used in gene therapy protocols. Likewise, mutagenesis can give rise to variants which have intracellular half-lives dramatically different than the corresponding unmodified a polypeptide. For example, the altered protein can be rendered either more stable or less stable to proteolytic degradation or other cellular processes which result in destruction, or otherwise inactivation, of an unmodified polypeptide. Such variants, and the genes which encode them, can be utilized to alter polypeptide complex levels by modulating the half-life of the polypeptide. For instance, a short half-life can give rise to more transient biological effects and, when part of an inducible expression system, can allow tighter control of recombinant polypeptide complex levels within the cell. In an Fc fusion protein, mutations may be made in the linker (if any) and/or the Fc portion to alter the half-life of the polypeptide.

A combinatorial library may be produced by way of a degenerate library of genes encoding a library of polypeptides which each include at least a portion of a polypeptide (e.g., an ActRII or TβRII polypeptide). For instance, a mixture of synthetic oligonucleotides can be enzymatically ligated into gene sequences such that the degenerate set of potential ActRII or TβRII encoding nucleotide sequences are expressible as individual polypeptides, or alternatively, as a set of larger fusion proteins (e.g., for phage display).

There are many ways by which the library of potential homologs can be generated from a degenerate oligonucleotide sequence. Chemical synthesis of a degenerate gene sequence can be carried out in an automatic DNA synthesizer, and the synthetic genes can then be ligated into an appropriate vector for expression. The synthesis of degenerate oligonucleotides is well known in the art [Narang, SA (1983) Tetrahedron 39:3; Itakura et al. (1981) Recombinant DNA, Proc. 3rd Cleveland Sympos. Macromolecules, ed. AG Walton, Amsterdam: Elsevier pp 273-289; Itakura et al. (1984) Annu. Rev. Biochem. 53:323; Itakura et al. (1984) Science 198:1056; and Ike et al. (1983) Nucleic Acid Res. 11:477]. Such techniques have been employed in the directed evolution of other proteins [Scott et al., (1990) Science 249:386-390; Roberts et al. (1992) Proc Natl Acad Sci USA 89:2429-2433; Devlin et al. (1990) Science 249: 404-406; Cwirla et al., (1990) Proc Natl Acad Sci USA 87: 6378-6382; as well as U.S. Pat. Nos. 5,223,409, 5,198,346, and 5,096,815].

Alternatively, other forms of mutagenesis can be utilized to generate a combinatorial library. For example, a polypeptide (e.g., an ActRII or TβRII polypeptide) of the disclosure can be generated and isolated from a library by screening using, for example, alanine scanning mutagenesis [Ruf et al. (1994) Biochemistry 33:1565-1572; Wang et al. (1994) J. Biol. Chem. 269:3095-3099; Balint et al. (1993) Gene 137:109-118; Grodberg et al. (1993) Eur. J. Biochem. 218:597-601; Nagashima et al. (1993) J. Biol. Chem. 268:2888-2892; Lowman et al. (1991) Biochemistry 30:10832-10838; and Cunningham et al. (1989) Science 244:1081-1085], by linker scanning mutagenesis [Gustin et al. (1993) Virology 193:653-660; and Brown et al. (1992) Mol. Cell Biol. 12:2644-2652; McKnight et al. (1982) Science 232:316], by saturation mutagenesis [Meyers et al., (1986) Science 232:613]; by PCR mutagenesis [Leung et al. (1989) Method Cell Mol Biol 1:11-19]; or by random mutagenesis, including chemical mutagenesis [Miller et al. (1992) A Short Course in Bacterial Genetics, CSHL Press, Cold Spring Harbor, NY; and Greener et al. (1994) Strategies in Mol Biol 7:32-34]. Linker scanning mutagenesis, particularly in a combinatorial setting, is an attractive method for identifying truncated (bioactive) forms of a polypeptide (e.g., an ActRII or TβRII polypeptide).

A wide range of techniques are known in the art for screening gene products of combinatorial libraries made by point mutations and truncations, and, for that matter, for screening cDNA libraries for gene products having a certain property. Such techniques will be generally adaptable for rapid screening of the gene libraries generated by the combinatorial mutagenesis of a polypeptide (e.g., an ActRII or TβRII polypeptide). The most widely used techniques for screening large gene libraries typically comprise cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected. Exemplary assays include ligand (e.g., activin A, activin B, GDF8, GDF11, BMP6, BMP10, TGFβ1, TGFβ3) binding assays and/or ligand-mediated cell signaling assays.

As will be recognized by one of skill in the art, most of the described mutations, variants or modifications described herein may be made at the nucleic acid level or, in some cases, by post-translational modification or chemical synthesis. Such techniques are well known in the art and some of which are described herein. In part, the present disclosure identifies functionally active portions (fragments) and variants of a polypeptide (e.g., an ActRII or TβRII polypeptide) that can be used as guidance for generating and using other variant polypeptides within the scope of the methods and uses described herein.

In certain embodiments, functionally active fragments of a polypeptide (e.g., an ActRII or TβRII polypeptide) of the disclosure can be obtained by screening polypeptides recombinantly produced from the corresponding fragment of the nucleic acid encoding polypeptides disclosed herein. In addition, fragments can be chemically synthesized using techniques known in the art such as conventional Merrifield solid phase f-Moc or t-Boc chemistry. The fragments can be produced (recombinantly or by chemical synthesis) and tested to identify those peptidyl fragments that can function as antagonists (inhibitors) of ActRII or TβRII receptors and/or one or more ligands (e.g., activin A, activin B, GDF8, GDF11, BMP6, BMP10, TGFβ1, or TGFβ3).

In certain embodiments, a polypeptide (e.g., an ActRII or TβRII polypeptide) or variants thereof of the disclosure further comprise post-translational modifications in addition to any that are naturally present in the polypeptide. Such modifications include, but are not limited to, acetylation, carboxylation, glycosylation, phosphorylation, lipidation, and acylation. As a result, the polypeptide may contain non-amino acid elements, such as polyethylene glycols, lipids, polysaccharide or monosaccharide, and phosphates. Effects of such non-amino acid elements on the functionality of a polypeptide may be tested as described herein for other polypeptide variants. When a polypeptide of the disclosure is produced in cells by cleaving a nascent form of the polypeptide, post-translational processing may also be important for correct folding and/or function of the protein. Different cells (e.g., CHO, HeLa, MDCK, 293, WI38, NIH-3T3 or HEK293) have specific cellular machinery and characteristic mechanisms for such post-translational activities and may be chosen to ensure the correct modification and processing of the polypeptides.

### 6. Nucleic Acids and Method of Manufacture

In certain aspects, the disclosure provides isolated and/or recombinant nucleic acids encoding any of the polypeptides disclosed herein including, for example, ActRII or TβRII polypeptides (e.g., soluble ActRII or TβRII polypeptides), as well as any of the variants disclosed herein. For example, SEQ ID NO: 4 encodes a naturally occurring ActRII precursor polypeptide, while SEQ ID NO: 5 encodes a soluble ActRII polypeptide, SEQ ID NOs: 61, 62, 63 and 64 encode variants of TβRII fusion polypeptides. The subject nucleic acids may be single-stranded or double stranded. Such nucleic acids may be DNA or RNA molecules. These nucleic acids may be used, for example, in methods for making ActRII or TβRII polypeptides or as direct therapeutic agents (e.g., in a gene therapy approach).

In certain aspects, the disclosure relates to isolated and/or recombinant nucleic acids comprising a coding sequence for one or more of the ActRII or TβRII polypeptide(s) as described herein. For example, in some embodiments, the disclosure relates to an isolated and/or recombinant nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 4, 5, 28, 61, 62, 63, or 64. In some embodiments, an isolated and/or recombinant polynucleotide sequence of the disclosure comprises a promoter sequence operably linked to a coding sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 4, 5, 28, 61, 62, 63, or 64). In some embodiments, the disclosure relates to vectors comprising an isolated and/or recombinant nucleic acid described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 4, 5, 28, 61, 62, 63, or 64). In some embodiments, the disclosure relates to a cell comprising an isolated and/or recombinant polynucleotide sequence described herein (e.g., a nucleic acid that is at least 75%, 80%, 85%, 90%, 91%, 92%, 93%, 94%, 95%, 96%, 97%, 98%, 99%, or 100% identical to the nucleic acid sequence corresponding to any one of SEQ ID Nos: 4, 5, 28, 61, 62, 63, or 64). In some embodiments, the cell is a CHO cell. In some embodiments, the cell is a COS cell.

In certain embodiments, nucleic acids encoding variant ActRII or TβRII polypeptides of the disclosure are understood to include nucleic acids that are variants of any one of SEQ ID NOs: 4, 5, 28, 61, 62, 63, or 64. Variant nucleotide sequences include sequences that differ by one or more nucleotide substitutions, additions, or deletions including allelic variants, and therefore, will include coding sequence that differ from the nucleotide sequence designated in any one of SEQ ID NOs: 4, 5, 28, 61, 62, 63, or 64.

In certain embodiments, variant ActRII polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to any one of SEQ ID NO: 4, 5, or 28. In certain embodiments, variant TβRII polypeptides of the disclosure are encoded by isolated and/or recombinant nucleic acid sequences that are at least 70%, 75%, 80%, 85%, 90%, 91%, 92%, 93%, 94%, 95%, 96%, 97%, 98%, 99%, or 100% identical to SEQ ID NO: 61-64.

In certain aspects, the subject nucleic acids encoding variant ActRII or TβRII polypeptides are further understood to include nucleic acids that are variants of SEQ ID NO: 4, 5, and 28 or 61-64, respectively. Variant nucleotide sequences include sequences that differ by one or more nucleotide substitutions, additions or deletions, such as allelic variants; and will, therefore, include coding sequences that differ from the nucleotide sequence of the coding sequence designated in SEQ ID NO: 4, 5, and 28 or 61-64, respectively.

In certain embodiments, the disclosure provides isolated or recombinant nucleic acid sequences that are at least 75%, 80%, 85%, 90%, 91%, 92%, 93%, 94%, 95%, 96%, 97%, 98%, 99%, or 100% identical to SEQ ID NO: 4, 5, or 28. In certain embodiments, the disclosure provides isolated or recombinant nucleic acid sequences that are at least 75%, 80%, 85%, 90%, 91%, 92%, 93%, 94%, 95%, 96%, 97%, 98%, 99%, or 100% identical to SEQ ID NO: 61, 62, 63, or 64. One of ordinary skill in the art will appreciate that nucleic acid sequences complementary to SEQ ID NO: 4, 5, 28, or 61-64, and variants of SEQ ID NO: 4, 5, 28, or 61-64 are also within the scope of this disclosure. In further embodiments, the nucleic acid sequences of the disclosure can be isolated, recombinant, and/or fused with a heterologous nucleotide sequence, or in a DNA library.

In other embodiments, nucleic acids of the disclosure also include nucleotide sequences that hybridize under highly stringent conditions to nucleic acids encoding ActRII or TβRII polypeptides of the disclosure, the complement sequence, or fragments thereof. As discussed above, one of ordinary skill in the art will understand readily that appropriate stringency conditions which promote DNA hybridization can be varied. For example, one could perform the hybridization at 6.0×sodium chloride/sodium citrate (SSC) at about 45° C., followed by a wash of 2.0×SSC at 50° C. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or temperature or salt concentration may be held constant while the other variable is changed. In one embodiment, the disclosure provides nucleic acids which hybridize under low stringency conditions of 6×SSC at room temperature followed by a wash at 2×SSC at room temperature.

Isolated nucleic acids which differ from the nucleic acids as set forth in the disclosure due to degeneracy in the genetic code are also within the scope of the disclosure. For example, a number of amino acids are designated by more than one triplet. Codons that specify the same amino acid, or synonyms (for example, CAU and CAC are synonyms for histidine) may result in “silent” mutations which do not affect the amino acid sequence of the polypeptide. However, it is expected that DNA sequence polymorphisms that do lead to changes in the amino acid sequences of the subject polypeptides will exist among mammalian cells. One skilled in the art will appreciate that these variations in one or more nucleotides (up to about 3-5% of the nucleotides) of the nucleic acids encoding a particular polypeptide may exist among individuals of a given species due to natural allelic variation. Any and all such nucleotide variations and resulting amino acid polymorphisms are within the scope of this disclosure.

In certain embodiments, the recombinant nucleic acids of the disclosure may be operably linked to one or more regulatory nucleotide sequences in an expression construct. Regulatory nucleotide sequences will generally be appropriate to the host cell used for expression. Numerous types of appropriate expression vectors and suitable regulatory sequences are known in the art for a variety of host cells. Typically, said one or more regulatory nucleotide sequences include, but are not limited to, promoter sequences, leader or signal sequences, ribosomal binding sites, transcriptional start and termination sequences, translational start and termination sequences, and enhancer or activator sequences. Constitutive or inducible promoters as known in the art are contemplated by the disclosure. The promoters may be either naturally occurring promoters, or hybrid promoters that combine elements of more than one promoter. An expression construct may be present in a cell on an episome, such as a plasmid, or the expression construct may be inserted in a chromosome. In one embodiment, the expression vector contains a selectable marker gene to allow the selection of transformed host cells. Selectable marker genes are well known in the art and will vary with the host cell used.

In certain aspects, the subject nucleic acid is provided in an expression vector comprising a nucleotide sequence encoding polypeptides of the disclosure (e.g., a variant ActRII or TβRII polypeptide), operably linked to at least one regulatory sequence. Regulatory sequences are art-recognized and are selected to direct expression of the polypeptides of the disclosure (e.g., a variant ActRII or TβRII polypeptide). Accordingly, the term regulatory sequence includes promoters, enhancers, and other expression control elements. Exemplary regulatory sequences are described in Goeddel; Gene Expression Technology: Methods in Enzymology, Academic Press, San Diego, CA (1990). For instance, any of a wide variety of expression control sequences that control the expression of a DNA sequence when operatively linked to it may be used in these vectors to express DNA sequences encoding polypeptides of the disclosure (e.g., a variant ActRII or TβRII polypeptide). Such useful expression control sequences, include, for example, the early and late promoters of SV40, tet promoter, adenovirus or cytomegalovirus immediate early promoter, RSV promoters, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. It should be understood that the design of the expression vector may depend on such factors as the choice of the host cell to be transformed and/or the type of polypeptide desired to be expressed. Moreover, the vector's copy number, the ability to control that copy number and the expression of any other polypeptide encoded by the vector, such as antibiotic markers, should also be considered.

A recombinant nucleic acid of the disclosure can be produced by ligating the cloned gene, or a portion thereof, into a vector suitable for expression in either prokaryotic cells, eukaryotic cells (yeast, avian, insect, or mammalian), or both. Expression vehicles for production of a recombinant variant ActRII or TβRII polypeptide include plasmids and other vectors. For instance, suitable vectors include plasmids of the types: pBR322-derived plasmids, pEMBL-derived plasmids, pEX-derived plasmids, pBTac-derived plasmids and pUC-derived plasmids for expression in prokaryotic cells, such as E. coli.

Some mammalian expression vectors contain both prokaryotic sequences to facilitate the propagation of the vector in bacteria, and one or more eukaryotic transcription units that are expressed in eukaryotic cells. The pcDNAI/amp, pcDNAI/neo, pRc/CMV, pSV2gpt, pSV2neo, pSV2-dhfr, pTk2, pRSVneo, pMSG, pSVT7, pko-neo and pHyg derived vectors are examples of mammalian expression vectors suitable for transfection of eukaryotic cells. Some of these vectors are modified with sequences from bacterial plasmids, such as pBR322, to facilitate replication and drug resistance selection in both prokaryotic and eukaryotic cells. Alternatively, derivatives of viruses such as the bovine papilloma virus (BPV-1), or Epstein-Barr virus (pHEBo, pREP-derived and p205) can be used for transient expression of polypeptides in eukaryotic cells. Examples of other viral (including retroviral) expression systems can be found below in the description of gene therapy delivery systems. The various methods employed in the preparation of the plasmids and in transformation of host organisms are well known in the art. For other suitable expression systems for both prokaryotic and eukaryotic cells, as well as general recombinant procedures, see Molecular Cloning A Laboratory Manual, 2nd Ed., ed. by Sambrook, Fritsch and Maniatis (Cold Spring Harbor Laboratory Press, 1989) Chapters 16 and 17. In some instances, it may be desirable to express the recombinant polypeptides by the use of a baculovirus expression system. Examples of such baculovirus expression systems include pVL-derived vectors (such as pVL1392, pVL1393 and pVL941), pAcUW-derived vectors (such as pAcUW1), and pBlueBac-derived vectors (such as the ß-gal containing pBlueBac III).

In one embodiment, a vector will be designed for production of the polypeptides of the disclosure (e.g., a variant ActRII or TβRII polypeptide) in CHO cells, such as a Pcmv-Script vector (Stratagene, La Jolla, Calif.), pcDNA4 vectors (Invitrogen, Carlsbad, Calif.) and pCI-neo vectors (Promega, Madison, Wisc.). As will be apparent, the subject gene constructs can be used to cause expression of the polypeptides of the disclosure (e.g., a variant ActRII or TβRII polypeptide) in cells propagated in culture, e.g., to produce polypeptides, including fusion polypeptides or polypeptides, for purification.

In certain embodiments, the disclosure relates to methods of making polypeptides of the disclosure (e.g., a variant ActRII or TβRII polypeptide) as described herein. Such a method may include expressing any of the nucleic acids disclosed herein in a suitable cell (e.g., a CHO cell or COS cell). Such a method may comprise: a) culturing a cell under conditions suitable for expression of the soluble polypeptides of the disclosure (e.g., a variant ActRII or TβRII polypeptide), wherein said cell comprises an expression construct of polypeptides of the disclosure (e.g., a variant ActRII or TβRII polypeptide). In some embodiments, the method further comprises recovering the expressed polypeptides of the disclosure (e.g., a variant ActRII or TβRII polypeptide). Polypeptides of the disclosure (e.g., a variant ActRII or TβRII polypeptide) may be recovered as crude, partially purified or highly purified fractions using any of the well-known techniques for obtaining protein from cell cultures.

This disclosure also pertains to a host cell transfected with a recombinant gene including a coding sequence for one or more polypeptides of the disclosure (e.g., a variant ActRII or TβRII polypeptide). The host cell may be any prokaryotic or eukaryotic cell. For example, polypeptides of the disclosure (e.g., a variant ActRII or TβRII polypeptide) may be expressed in bacterial cells such as E. coli, insect cells (e.g., using a baculovirus expression system), yeast, or mammalian cells. Other suitable host cells are known to those skilled in the art.

Accordingly, the present disclosure further pertains to methods of producing polypeptides of the disclosure (e.g., a variant ActRII or TβRII polypeptide). For example, a host cell transfected with an expression vector encoding polypeptides of the disclosure (e.g., a variant ActRII or TβRII polypeptide) can be cultured under appropriate conditions to allow expression of the polypeptides of the disclosure (e.g., a variant ActRII or TβRII polypeptide) to occur. The polypeptides of the disclosure (e.g., a variant ActRII or TβRII polypeptide) may be secreted and isolated from a mixture of cells and medium containing the polypeptides. Alternatively, the polypeptides of the disclosure (e.g., a variant ActRII or TβRII polypeptide) may be retained cytoplasmically or in a membrane fraction and the cells harvested, lysed and the protein isolated. A cell culture includes host cells, media and other byproducts. Suitable media for cell culture are well known in the art. The subject polypeptides of the disclosure (e.g., a variant ActRII or TβRII polypeptide) can be isolated from cell culture medium, host cells, or both, using techniques known in the art for purifying polypeptides, including ion-exchange chromatography, gel filtration chromatography, ultrafiltration, electrophoresis, and immunoaffinity purification with antibodies specific for particular epitopes of polypeptides of the disclosure (e.g., a variant ActRII or TβRII polypeptide). In one embodiment, the polypeptides of the disclosure (e.g., a variant ActRII or TβRII polypeptide) are fusion polypeptides containing a domain which facilitates purification.

In some embodiments, ActRII polypeptides and TβRII polypeptides to be used in accordance with the methods described herein are isolated polypeptides. As used herein, an isolated protein or polypeptide is one which has been separated from a component of its natural environment. In some embodiments, a polypeptide of the disclosure is purified to greater than 95%, 96%, 97%, 98%, or 99% purity as determined by, for example, electrophoretic (e.g., SDS-PAGE, isoelectric focusing (IEF), capillary electrophoresis) or chromatographic (e.g., ion exchange or reverse phase HPLC). Methods for assessment of purity are well known in the art [see, e.g., Flatman et al., (2007) J. Chromatogr. B 848:79-87]. In some embodiments, ActRII polypeptides and TβRII polypeptides to be used in accordance with the methods described herein are recombinant polypeptides.

In certain embodiments, ActRII or TβRII polypeptides of the disclosure can be produced by a variety of art-known techniques. For example, such ActRII or TβRII polypeptides can be synthesized using standard protein chemistry techniques such as those described in Bodansky, M. Principles of Peptide Synthesis, Springer Verlag, Berlin (1993) and Grant G. A. (ed.), Synthetic Peptides: A User's Guide, W. H. Freeman and Company, New York (1992). In addition, automated peptide synthesizers are commercially available (e.g., Advanced ChemTech Model 396; Milligen/Biosearch 9600). Alternatively, the ActRII or TβRII polypeptides, fragments or variants thereof may be recombinantly produced using various expression systems (e.g., E. coli, Chinese Hamster Ovary cells, COS cells, baculovirus) as is well known in the art (also see above). In a further embodiment, the ActRII or TβRII polypeptides may be produced by digestion of naturally occurring or recombinantly produced full-length ActRII or TβRII polypeptides by using, for example, a protease, e.g., trypsin, thermolysin, chymotrypsin, pepsin, or paired basic amino acid converting enzyme (PACE). Computer analysis (using a commercially available software, e.g., MacVector, Omega, PCGene, Molecular Simulation, Inc.) can be used to identify proteolytic cleavage sites. Alternatively, such ActRII or TβRII polypeptides may be produced from naturally occurring or recombinantly produced full-length ActRII or TβRII polypeptides such as standard techniques known in the art, such as by chemical cleavage (e.g., cyanogen bromide, hydroxylamine).

In another embodiment, a fusion gene coding for a purification leader sequence, such as a poly-(His)/enterokinase cleavage site sequence at the N-terminus of the desired portion of the recombinant polypeptides of the disclosure (e.g., a variant ActRII or TβRII polypeptide), can allow purification of the expressed fusion polypeptide by affinity chromatography using a Ni²⁺ metal resin. The purification leader sequence can then be subsequently removed by treatment with enterokinase to provide the purified polypeptides of the disclosure (e.g., a variant ActRII or TβRII polypeptide) (e.g., see Hochuli et al., (1987) J. Chromatography 411:177; and Janknecht et al., Proc Natl Acad Sci USA 88:8972).

Techniques for making fusion genes are well known. Essentially, the joining of various DNA fragments coding for different polypeptide sequences is performed in accordance with conventional techniques, employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers which give rise to complementary overhangs between two consecutive gene fragments which can subsequently be annealed to generate a chimeric gene sequence (see, for example, Current Protocols in Molecular Biology, eds. Ausubel et al., John Wiley & Sons: 1992).

### 7. Methods of Use

In part, the present disclosure relates to methods of treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) comprising administering to a patient in need thereof an effective amount of a combination of an ActRII polypeptide and a TβRII polypeptide as described herein. In some embodiments, the disclosure contemplates methods of treating, preventing, or reducing the progression rate and/or severity of one or more complications of pulmonary hypertension associate with lung disease (e.g., pulmonary hypertension associated with chronic obstructive pulmonary disease (COPD), interstitial lung disease (ILD), or combined pulmonary fibrosis and emphysema (CPFE)), comprising administering to a patient in need thereof an effective amount of a combination of an ActRII polypeptide and a TβRII polypeptide as described herein. In some embodiments, the ActRII polypeptide is administered at a dosing range of 0.1 mg/kg to 2.0 mg/kg (e.g., 0.3 mg/kg or 0.7 mg/kg). In some embodiments, the administration of the combination of an ActRII polypeptide and a TβRII polypeptide results in a change of one or more hemodynamic or functional parameters (e.g., a reduction in pulmonary vascular resistance (PVR); an increase in 6-minute walk distance (6 MWD); a decrease of the N-terminal pro B-type natriuretic peptide (NT-proBNP) levels; a prevention or delay in pulmonary hypertension Functional Class progression as recognized by the World Health Organization (WHO); a promotion or increase in pulmonary hypertension Functional Class regression as recognized by the WHO; an improvement in right ventricular function; and an improvement in pulmonary artery pressure).

In certain aspects, the disclosure relates to methods of treating pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the one or more complications of pulmonary hypertension associated with lung disease is selected from the group consisting of persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy.

These methods are particularly aimed at therapeutic and prophylactic treatments of animals, and more particularly, humans. The terms “subject,” an “individual,” or a “patient” are interchangeable throughout the specification and refer to either a human or a non-human animal. These terms include mammals, such as humans, non-human primates, laboratory animals, livestock animals (including bovines, porcines, camels, etc.), companion animals (e.g., canines, felines, other domesticated animals, etc.) and rodents (e.g., mice and rats). In particular embodiments, the patient, subject or individual is a human.

The terms “treatment”, “treating”, “alleviating”, “reducing the progression rate”, “reducing the severity of”, and the like are used herein to generally mean obtaining a desired pharmacologic and/or physiologic effect, and may also be used to refer to improving, alleviating, and/or decreasing the severity of one or more clinical complication of a condition being treated (e.g., pulmonary hypertension associated with lung disease). The effect may be prophylactic in terms of completely or partially delaying the onset or recurrence of a disease, condition, or complications thereof, and/or may be therapeutic in terms of a partial or complete cure for a disease or condition and/or adverse effect attributable to the disease or condition. “Treatment” as used herein covers any treatment of a disease or condition of a mammal, particularly a human. As used herein, a therapeutic that “prevents” a disorder or condition refers to a compound that, in a statistical sample, reduces the occurrence of the disorder or condition in a treated sample relative to an untreated control sample, or delays the onset of the disease or condition, relative to an untreated control sample.

In general, treatment or prevention of a disease or condition as described in the present disclosure (e.g., pulmonary hypertension associated with lung disease) is achieved by administering a combination of one or more ActRII polypeptides and TβRII polypeptides of the present disclosure in an “effective amount”. An effective amount of an agent refers to an amount effective, at dosages and for periods of time necessary, to achieve the desired therapeutic or prophylactic result. In the methods of the invention, an “effective amount” of an ActRII polypeptide is an amount that is useful in achieving a desired therapeutic or prophylactic result in combination with a TβRII polypeptide and an effective amount of a TβRII polypeptide is an amount that is useful in achieving a desired therapeutic or prophylactic result in combination with a ActRII polypeptide. An effective amount of an ActRII polypeptide or a TβRII polypeptide for use in the methods of the invention may be the same or different as an effective amount of such polypeptide when used in monotherapy methods (i.e., not in combination with any other agent). A “therapeutically effective amount” of an agent of the present disclosure may vary according to factors such as the disease state, age, sex, and weight of the individual, and the ability of the agent to elicit a desired response in the individual. A “prophylactically effective amount” refers to an amount effective, at dosages and for periods of time necessary, to achieve the desired prophylactic result.

In certain aspects, the disclosure contemplates the use of an ActRII polypeptide and a TβRII polypeptide, in combination with one or more additional active agents or other supportive therapy for treating or preventing a disease or condition (e.g., pulmonary hypertension associated with lung disease). As used herein, “in combination with”, “combinations of”, “combined with”, or “conjoint” administration refers to any form of administration such that additional active agents or supportive therapies (e.g., second, third, fourth, etc.) are still effective in the body (e.g., multiple compounds are simultaneously effective in the patient for some period of time, which may include synergistic effects of those compounds). Effectiveness may not correlate to measurable concentration of the agent in blood, serum, or plasma. For example, the different therapeutic compounds can be administered either in the same formulation or in separate formulations, either concomitantly or sequentially, and on different schedules. Thus, a subject who receives such treatment can benefit from a combined effect of different active agents or therapies. The combination of one or more ActRII polypeptides and TβRII polypeptides of the disclosure can be administered concurrently with, prior to, or subsequent to, one or more other additional agents or supportive therapies, such as those disclosed herein. In general, each active agent or therapy will be administered at a dose and/or on a time schedule determined for that particular agent. The particular combination to employ in a regimen will take into account compatibility of the ActRII polypeptide and TβRII polypeptide of the present disclosure with the additional active agent or therapy and/or the desired effect.

### WHO Classification Outline

Pulmonary hypertension conditions treated by methods describe herein, can comprise any one or more of the conditions recognized according to the World Health Organization (WHO). See, e.g., Simonneau (2019) Eur Respiri J: 53:1801913.

TABLE 1
Clinical Classification of Pulmonary Hypertension

```
Group 1: Pulmonary arterial hypertension (PAH)
1.1 Idiopathic PAH
1.2 Heritable PAH
  1.2.1 BMPR2
  1.2.2 ALK-1, ENG, SMAD9, CAV1, KCNK3
  1.2.3 Unknown
1.3 Drug and toxin induced PAH
1.4 Associated with:
  1.4.1 Connective tissue disease
  1.4.2 HIV infection
  1.4.3 Portal hypertension
  1.4.4 Congenital heart diseases
  1.4.5 Schistosomiasis
1.5 PAH long-term responders to calcium channel blockers
1.6 PAH with overt features of venous/capillaries (PVOD/PCH) involvement
1.7 Persistent PH of the newborn syndrome
Group 2: Pulmonary hypertension due to left heart disease
2.1 PH due to heart failure with preserved LVEF¹ (HFpEF)
2.2 PH due to heart failure with reduced LVEF (HFrEF)
2.3 Valvular heart disease
2.4 Congenital/acquired cardiovascular conditions leading to post-capillary PH
Group 3: Pulmonary hypertension due to lung disease and/or hypoxia
3.1 Obstructive lung disease
3.2 Restrictive lung disease
3.3 Other lung disease with mixed restrictive/obstructive pattern
3.4 Hypoxia without lung disease
3.5 Developmental lung disorders
Group 4: Pulmonary hypertension due to pulmonary artery obstructions
4.1 Chronic thromboembolic PH
4.2 Other pulmonary artery obstructions
4.2.1 Sarcoma (high or intermediate grade) or angiosarcoma
4.2.2 Other malignant tumours
  Renal carcinoma
  Uterine carcinoma
  Germ cell tumours of the testis
  Other tumours
4.2.3 Non-malignant tumours
  Uterine leiomyoma
4.2.4 Arteritis without connective tissue disease
4.2.5 Congenital pulmonary artery stenoses
4.2.6 Parasites
  Hydatidosis
Group 5: Pulmonary hypertension with unclear and/or multifactorial mechanisms.
5.1 Hematological disorders (e.g., Chronic hemolytic anaemia and myeloproliferative
disorders)
5.2 Systemic and metabolic disorders (e.g., Pulmonary Langerhans cell histiocytosis,
Gaucher disease, Glycogen storage disease, Neurofibromatosis, and Sarcoidosis)
5.3 Others (e.g., Chronic renal failure with or without haemodialysis and Fibrosing
mediastinitis)
5.4 Complex congenital heart disease
¹Left ventricular ejection fraction
```

As used herein, the term “pulmonary hemodynamic parameter” refers to any parameter used to describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO).

Many of the pulmonary hemodynamic parameters described above are interrelated. For example, PVR is related to mPAP, PCWP and CO according to the following equation:

PVR=(mPAP−PCWP)/CO [Woods Units]

The PVR measures the resistance to flow imposed by the pulmonary vasculature without the influence of the left-sided filling pressure. PVR can also be measured according to the following equations:

PVR=TPG×80/CO [unit: dynes-sec-cm⁻⁵] OR PVR=(mPAP−PCWP)×80/CO [unit: dynes-sec-cm⁻⁵]

In some embodiments, the total PVR can be measured using the following equation:

TPR=mPAP/CO.

In some embodiments, the normal PVR is 20-180 dynes-sec-cm⁻⁵ or typically less than 0.5-2 Wood units. According to some embodiments, an elevated PVR may refer to a PVR above 2 Wood units, above 2.5 Wood units, above 3 Wood units or above 3.5 Wood units.

As yet another example, mPAP is related to dPAP and sPAP according to the following equation: mPAP=(⅔) dPAP+(⅓) sPAP.

In some embodiments, the pulmonary hemodynamic parameters are measured directly, such as during a right heart catheterization. In other embodiments, the pulmonary hemodynamic parameters are estimated and/or evaluated through other techniques such as magnetic resonance imaging (MRI) or echocardiography.

Exemplary pulmonary hemodynamic parameters include mPAP, PAWP, and PVR. The one or more pulmonary hemodynamic parameters may be measured by any appropriate procedures, such as by utilizing a right heart catheterization or echocardiography. Various hemodynamic characteristics of PH and pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are shown in Table 2.

TABLE 2
Hemodynamic Characteristics of Pulmonary Hypertension (PH)
and Pulmonary Hypertension Associated with Lung Disease

| | Hemodynamic Characteristics |
|---|---|
| Pulmonary | mPAP >20 mmHg |
| Hypertension | |
| Pulmonary | mPAP >20 mmHg |
| hypertension | PAWP ≤15 mmHg |
| associated with | PVR ≥3 Wood units |
| lung disease | |

The clinical classification or hemodynamic characteristics of PAH described herein and the associated diagnostic parameters may be updated or varied based on the availability of new or existing sources of data or when additional clinical entities are considered.

Characteristics of Pulmonary Hypertension Associated with Lung Disease

Pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) (WHO Group 3 PH) is the second most common form of pulmonary hypertension and is associated with increased morbidity and mortality. Patients with Group 3 pulmonary hypertension have a worse outcome than patients with Group 1 pulmonary hypertension. Similarly, patients with Group 1 pulmonary arterial hypertension and associated lung disease suffer from even worse outcomes relative to patients having only Group 1 pulmonary arterial hypertension.

A variety of factors contribute to the pathogenesis of pulmonary hypertension associated with lung disease. These factors vary based on the underlying lung disease. For example, in pulmonary hypertension caused by COPD, the most prominent etiology for pulmonary hypertension is hypoxic pulmonary vasoconstriction (HPVC) with remodeling of the pulmonary vascular bed. Initial changes during vascular remodeling include distal neomuscularization of the arterioles, intimal thickening, and medial hypertrophy. This remodeling eventually leads to fewer blood vessels and consequently increased peripheral vascular resistance seen in pulmonary hypertension. Additional mechanisms underlying ILD-associated pulmonary hypertension include vascular destruction due to progressive parenchymal fibrosis, vascular inflammation, perivascular fibrosis, thrombotic angiopathy, and endothelial dysfunction. More specifically, patients with pulmonary hypertension associated with idiopathic pulmonary fibrosis (IPF) may have an abnormal vascular phenotype, characterized by aberrant gene expression profiles that promote vascular remodeling.

Pulmonary hypertension associated with lung disease can be diagnosed on a mean pulmonary artery pressure (mPAP) of or above 25 mmHg. Pulmonary hypertension associated with lung disease can lead to persistent cough, productive cough, wheezing, exercise intolerance, respiratory infections, bronchiectasis, chronic infections, nasal polyps, hemoptysis, pneumothorax, respiratory failure, dyspnea, chest pain, hemoptysis, pneumothorax, pulmonary vascular remodeling, pulmonary fibrosis, pulmonary vascular endothelial dysfunction, hypoxia due to chronic pulmonary injury, hypoxic pulmonary vasoconstriction, inflammation, smooth muscle hypertrophy, and right ventricular hypertrophy. The lung diseases associated with pulmonary hypertension may be classified as either obstructive lung disease or restrictive lung disease. Obstructive lung diseases (e.g., COPD, cystic fibrosis, asthma, emphysema, and chronic bronchitis) is marked by the difficulty in exhalation. Alternatively, restrictive lung diseases, which can be further separated into intrinsic (e.g., pulmonary fibrosis, interstitial lung disease, sarcoidosis, idiopathic pulmonary fibrosis) and extrinsic (obesity, scoliosis, Myasthenia gravis, and pleural effusion) disorders, is characterized by the restriction of full lung expansion.

Pulmonary hypertension associated with lung disease (e.g., pulmonary hypertension associated with lung disease) can be challenging to diagnose due to the heterogeneity of the underlying lung condition. Many symptoms of the lung disease mimic the symptoms of pulmonary hypertension. There are, however, several clinical features that prompt a diagnosis of pulmonary hypertension associated with lung disease (e.g., exertional dyspnea or hypoxemia not fully explained by parenchymal lung disease or a sleep disorder, rapid decline of arterial oxygenation upon exercise, any clinical features suggestive of right-sided heart failure, enlarged pulmonary arteries, attenuation of peripheral pulmonary vasculature, or right ventricular enlargement as demonstrated by high resolution computed tomography (HRCT), severe reductions in diffusing capacity as demonstrated by pulmonary function testing, and lung biopsy). Klings, E. S. (2021). Pulmonary hypertension due to lung disease and/or hypoxemia (group 3 pulmonary hypertension): Epidemiology, pathogenesis, and diagnostic evaluation in adults. UpToDate. Retrieved Apr. 6, 2021 from https://www.uptodate.com/contents/pulmonary-hypertension-due-to-lung-disease-and-or-hypoxemia-group-3-pulmonary-hypertension-epidemiology-pathogenesis-and-diagnostic-evaluation-in-adults.

While echocardiography is a standard test when investigating patients with suspected pulmonary hypertension of unknown etiology for underlying lung disease and/or sleep disordered breathing, echocardiography may not be as reliable for accurately diagnosing pulmonary hypertension in a patient having severe lung disease. In such cases, right heart catheterization (RHC) may give a more accurate assessment.

### Chronic Obstructive Lung Disease

In some embodiments, the disclosure relates to methods of treating pulmonary hypertension associated with chronic obstructive pulmonary disease (COPD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. Chronic obstructive lung disease (also, chronic obstructive pulmonary disease (COPD)), is an inflammatory lung disease that causes obstructed airflow from the lungs. Within this group of diseases are emphysema and chronic bronchitis. According to the Centers for Disease Control and Prevention, millions of people suffer with COPD, 16 million of which are in the United States of America.

The severity of COPD is determined using the Global Initiative for Chronic Obstructive Lung Disease (GOLD) staging or grading system, determined by spirometry results (GOLD 1: mild, GOLD 2: moderate, GOLD 3: severe, and GOLD 4: very severe). This system bases the stage of COPD on several factors (e.g., overall symptoms, number of times COPD has worsened, hospitalizations due to worsening COPD, and results from spirometry). The majority of patients with pulmonary hypertension caused by COPD present with severe or very severe airflow obstruction (GOLD spirometric stages 3 or 4, FEV-1<50% of predicted) or severe emphysema, and mild-to-moderate precapillary pulmonary hypertension. Current treatments for COPD include short-acting bronchodilators, long-acting bronchodilators, inhaled steroids, combination inhalers that include both bronchodilators and inhaled steroids or more than one type of bronchodilator, oral steroids, phosphodiesterase-4 inhibitors, theophylline, antibiotics, various types of lung therapies, and in-home non-invasive ventilation therapy.

### Interstitial Lung Diseases

In some embodiments, the disclosure relates to methods of treating pulmonary hypertension associated with interstitial lung disease (ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. Interstitial lung disease, or ILD, is a chronic lung disease that occurs as a result of damage between the air sacs in the lungs that leads to lung scarring, inflammation, and breathing problems. ILDs may be caused by infections, medicines, and inhalation of harmful particles in the air. The underlying cause of the ILD determines the course of treatment. ILDs overall decrease the quality of life of the person living with the disease, and may shorten the person's life altogether.

There are approximately five categories of ILDs based on their underlying causes: ILDs caused by exposure or occupational related (e.g., asbestosis, silicosis, hypersensitivity pneumonitis), ILDs related to medications and/or medical treatments (e.g., chemotherapy, radiation therapy), ILDs associated with autoimmune disorders and/or connective tissue diseases (e.g., lupus, scleroderma, poly or dermatomyositis, rheumatoid arthritis), sarcoidosis, and idiopathic ILDs. Outside of the five general categories remains ILDs such as idiopathic pulmonary fibrosis (IPF), bronchiolitis obliterans, histiocytosis X, chronic eosinophilic pneumonia, collagen vascular disease, granulomatous vasculitis, Goodpasture's syndrome, and pulmonary alveolar proteinosis.

The symptoms of ILDs may vary from person to person, as well as based on the particular ILD, however the common link between the various forms of ILD is that all ILDs begin with an inflammation in the bronchioles (e.g., bronchiolitis), alveoli (e.g., alveolitis), or capillaries (vasculitis). The most common symptoms of ILDs, such as shortness of breath (especially with exertion), fatigue and weakness, loss of appetite, loss of weight, dry cough that does not produce phlegm, discomfort in the chest, labored breathing, and hemorrhage in the lungs, may resemble other lung conditions or medical problems.

Fibrosis results in permanent destruction of air sacs, the lung tissue between and surrounding the airs sacs, and the lung capillaries. Disease progression may be gradual or rapid and present with very mild, moderate, or very severe symptoms. The course of ILDs is unpredictable, but may improve with medical intervention.

Interstitial lung diseases are diagnosed using pulmonary function tests (PFTs), chest X-rays, blood tests (e.g., analysis of arterial blood gas to determine the amount of carbon dioxide and oxygen is in the blood), high-resolution computed tomography (HRCT, CT, or CAT scan), bronchoscopy, bronchoalveolar lavage, and lung biopsy.

Treatment plans for ILDs are typically determined based on a person's age, overall health, and medical history, extent of the disease, a person's tolerance for specific medications, procedures, and/or therapies, expectations for the course of the disease, as well as a person's opinion or preference. These treatment plans may include oral medications (e.g., corticosteroids), oxygen supplementation, and lung transplantation.

### Idiopathic Pulmonary Fibrosis and Other Idiopathic Interstitial Pneumonias

In some embodiments, the disclosure relates to methods of treating pulmonary hypertension associated with idiopathic pulmonary fibrosis (IPF), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. Idiopathic pulmonary fibrosis (IPF) (also, cryptogenic fibrosing alveolitis, chronic idiopathic fibrosing alveolitis, interstitial pneumonia) is one of the most frequently diagnosed interstitial lung diseases (ILDs), affecting approximately 13 to 20 per 100,000 people worldwide, with 30,000 to 40,000 new cases diagnosed each year. Though there are medical treatments for IPF available, the disease remains severe with an expectation of clinical decline.

There are several underlying factors that influence the progression of IPF, one of which is thought to be chronic and/or repetitive microinjuries of the alveolar epithelium (e.g., exposure to environmental pollutants, acid aspiration due to gastroesophageal reflux, and viral infections). Damage to the epithelium is followed by injury and/or activation of cells lining the vascular and interstitial compartments of the lung, epithelium of distal airways, ad resident macrophages. Genetic factors may contribute to IPF, which is suggested by the occurrence of IPF-like disease in patients with rare genetic disorders and by cases of familial idiopathic interstitial pneumonia.

There are currently no treatments that have proven effective at stopping the progression of the disease, but newer medications (e.g., pirfenidone and nintedanib) have been approved by the Food and Drug Administration to help slow the progression of the disease.

### Non-Idiopathic Pulmonary Fibrosis Interstitial Lung Disease

In some embodiments, the disclosure relates to methods of treating pulmonary hypertension associated with non-idiopathic pulmonary fibrosis interstitial lung disease (non-IPF ILD), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. Non-idiopathic pulmonary fibrosis interstitial lung disease (non-IPF) causes inflammation and fibrosis of the lung interstitium leading to impaired gas exchange. The estimated prevalence of non-IPF is estimated to range from 25 to 74 per 100,000 people. There are over 200 known causes for non-IPF that can typically be categorized as occupational and environmental exposures, organic substances causing hypersensitivity pneumonitis, drug-induced lung toxicity, connective tissue diseases, and systemic illnesses.

The pathogenesis of non-IPF is similar between non-IPFs resulting from any one of the over 200 known causes, involving phases of injury (e.g., recurrent and direct epithelial/endothelial injury to distal air spaces, and destruction of the alveolar-capillary basement membrane), inflammation caused by release of proinflammatory cytokines and chemokines by macrophages (e.g., transforming growth factor-beta), and repair (e.g., myofibroblast formation and secretion of fibrous proteins and ground substance that forms the extracellular matrix). This process, however, repeated over time results in continued thickening and irreversible fibrosis of the lung parenchyma.

Treatment and management of the disease involves supportive care, supplemental oxygen, and in certain conditions, corticosteroids. A lung transplant may be considered as an option in severe or progressive cases. Mortality rates can be as high as 100% during an acute exacerbation of non-IPF.

### Combined Pulmonary Fibrosis and Emphysema

In some embodiments, the disclosure relates to methods of treating pulmonary hypertension associated with combined pulmonary fibrosis and emphysema (CPFE), comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. Combined pulmonary fibrosis and emphysema (CPFE) is characterized by dyspnea, upper-lobe emphysema, lower-lobe fibrosis, and abnormalities of gas exchange. CPFE can be further complicated by pulmonary hypertension, acute lung injury, and lung cancer. Different pulmonary function tests (PFTs) are used to diagnose CPFE than the PFTs used to diagnose fibrosis or emphysema alone. Additionally, HRCT scanning can be used to detect the simultaneous occurrence of emphysema and pulmonary fibrosis.

CPFE has been linked to cigarette smoking, exposure to asbestos and mineral dust, hypersensitivity pneumonitis (or famer lung), as well as being male, and has significant mortality. Median survival has ranged from 2.1 to 8.5 years, and if pulmonary hypertension is present, the 1-year survival is only 60%. Despite this disparity, there is no specific treatment for CPFE, other than supportive care (e.g., smoking cessation).

Diagnosis of Pulmonary Hypertension Associated with Lung Disease

The diagnosis of pulmonary hypertension associated with lung disease (e.g., pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), including functional group, can be determined based on symptoms and physical examination using a review of a comprehensive set of parameters to determine if the hemodynamic and other criteria are met. Some of the criteria which may considered include the patient's clinical presentation (e.g., shortness of breath, fatigue, weakness, angina, syncope, dry-cough, exercise-induced nausea and vomiting), electrocardiogram (ECG) results, chest radiograph results, pulmonary function tests, arterial blood gases, echocardiography results, ventilation/perfusion lung scan results, high-resolution computed tomography results, contrast-enhanced computed tomography results, pulmonary angiography results, cardiac magnetic resonance imaging, blood tests (e.g., biomarkers such as BNP or NT-proBNP), immunology, abdominal ultrasound scan, right heart catheterization (RHC), vasoreactivity, and genetic testing. Galie N., et al Euro Heart J. (2016) 37, 67-119.

A diagnosis of pulmonary hypertension associated with lung disease (Group 3 pulmonary hypertension) is determined when chronic lung disease and/or hypoxemia is present in a person having pulmonary hypertension, and no alternative cause of pulmonary hypertension can be identified. Group 3 pulmonary hypertension can be made based clinical assessments and echocardiographic results, and definitively confirmed by right heart catheterization. Though pulmonary hypertension associated with lung disease has symptomatic and etiological overlap with other types of pulmonary hypertension, several features distinguish this group from the others (e.g., moderate to severe impairment (FEV₁<60% in patients with COPD, and FVC<70% in patients with pulmonary fibrosis), characteristic imaging of a lung disorder or polysomnographic findings of a sleep disorder, reduced breathing reserve, normal oxygen pulse, mixed venous oxygen saturation above the lower limit of normal, and an increase in the partial arterial pressure of carbon dioxide during exercise (especially in COPD), and presence of mild to moderate pulmonary hypertension on echocardiography or right heart catheterization).

### Measurements of Group 3 PH

Various pulmonary hemodynamic parameters are helpful in assessing disease progression and a patient's responsiveness to treatment protocols. Typically, these parameters describe or evaluate the blood flow through the heart and pulmonary vasculature. Examples of pulmonary hemodynamic parameters include, but are not limited to, mean pulmonary artery pressure (mPAP), diastolic pulmonary artery pressure (dPAP) [also known as pulmonary artery diastolic pressure (PADP)], systolic pulmonary artery pressure (sPAP) [also known as pulmonary artery systolic pressure (PASP)], mean right atrial pressure (mRAP), pulmonary capillary wedge pressure (PCWP) [also known as pulmonary artery wedge pressure (PAWP)], pulmonary vascular resistance (PVR) and cardiac output (CO). In certain aspects, the disclosure relates to methods of treating pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method alters or modifies one or more of the following parameters:
a) reduces the right ventricular systolic pressure (RVSP);
b) reduces mPAP
c) reduces mRAP;
d) decreases PVR;
e) decreases the diastolic pressure gradient (DPG);
f) decreases the BNP levels;
g) decreases the NT-proBNP levels;
h) decreases smooth muscle hypertrophy;
i) decreases a patient's CAMPHOR score;
j) improves ventricular function;
k) decreases right ventricular hypertrophy;
l) increases cardiac index;
m) increases the cardiac output;
n) decreases the composite physiologic index;
o) increases the arterial oxygen saturation;
p) increases exercise capacity;
q) increases forced expiratory volume;
r) increases forced vital capacity (FVC);
s) increases the DL_{CO};
t) reduces pulmonary fibrosis; and/or
u) increases transplant free survival in the patient.
   mPAP

Pulmonary blood pressure is normally a lot lower than systemic blood pressure. Normal pulmonary artery pressure is typically between 8-20 mm Hg at rest. If the pressure in the pulmonary artery is greater than 25 mm Hg at rest or 30 mm Hg during physical activity, it is abnormally high and is characterized as pulmonary hypertension.

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's mPAP is reduced by at least 10%. In some embodiments, the method relates to patients having an mPAP of at least 17 mmHg. In some embodiments, the method relates to patients having an mPAP of at least 20 mmHg. In some embodiments, the method relates to patients having an mPAP of at least 25 mmHg. In some embodiments, the method relates to patients having an mPAP between 25-34 mmHg. In some embodiments, the method relates to patients having an mPAP of at least 30 mmHg. In some embodiments, the method relates to patients having an mPAP of at least 35 mmHg. In some embodiments, the method relates to patients having an mPAP of at least 40 mmHg. In some embodiments, the method relates to patients having an mPAP of at least 45 mmHg. In some embodiments, the method relates to patients having an mPAP of at least 50 mmHg.

In some embodiments, the method relates to patients having an mPAP between 21-24 mmHg and a PVR of at least 3 Wood Units. In some embodiments, the method relates to patients having an mPAP of greater than 25 mmHg with a Cardiac Index (CI) of less than 2.0 L/min/m². In some embodiments, the method relates to patients having an mPAP of greater than 25 mmHg with a CI of less than 2.5 L/min/m².

In some embodiments, the method relates to reducing the mPAP in the patient by at least 10% (e.g., 10%, 15%, 20%, 25%, 30%, 35%, 40%, 45%, or at least 50%). In some embodiments, the method relates to reducing the mPAP in the patient by at least 15%. In some embodiments, the method relates to reducing the mPAP in the patient by at least 20%. In some embodiments, the method relates to reducing the mPAP in the patient by at least 25%. In some embodiments, the method relates to reducing the mPAP in the patient by at least 30%. In some embodiments, the method relates to reducing the mPAP in the patient by at least 35%. In some embodiments, the method relates to reducing the mPAP in the patient by at least 40%. In some embodiments, the method relates to reducing the mPAP in the patient by at least 45%. In some embodiments, the method relates to reducing the mPAP in the patient by at least 50%.

In some embodiments, the method relates to reducing the mPAP by at least 3 mmHg in the patient. In some embodiments, the method relates to reducing the mPAP by at least 5 mmHg. In some embodiments, the method relates to reducing the mPAP by at least 7 mmHg. In some embodiments, the method relates to reducing the mPAP by at least 10 mmHg. In some embodiments, the method relates to reducing the mPAP by at least 12 mmHg. In some embodiments, the method relates to reducing the mPAP by at least 15 mmHg. In some embodiments, the method relates to reducing the mPAP by at least 20 mmHg. In some embodiments, the method relates to reducing the mPAP by at least 25 mmHg. In some embodiments, the method relates to reducing the mPAP to less than 17 mmHg. In some embodiments, the method relates to reducing the mPAP to less than 20 mmHg. In some embodiments, the method relates to reducing the mPAP to less than 25 mmHg. In some embodiments, the method relates to reducing the mPAP to less than 30 mmHg.

mRAP

Right atrial pressure (RAP) is the blood pressure in the right atrium of the heart. RAP reflects the amount of blood returning to the heart and the ability of the heart to pump the blood into the arterial system. Normal right atrial pressure is typically between 2 mmHg and 6 mmHg. Elevated right atrial pressure reflects right ventricular (RV) overload and is an established risk factor.

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's mRAP is reduced by at least 10%.

In some embodiments, the patient has a mean right atrial pressure (mRAP) of at least 5 mmHg. In some embodiments, the patient has a mean right atrial pressure (mRAP) of at least 6 mmHg. In some embodiments, the patient has a mean right atrial pressure (mRAP) of at least 8 mmHg. In some embodiments, the patient has a mean right atrial pressure (mRAP) of at least 10 mmHg. In some embodiments, the patient has a mean right atrial pressure (mRAP) of at least 12 mmHg. In some embodiments, the patient has a mean right atrial pressure (mRAP) of at least 14 mmHg. In some embodiments, the patient has a mean right atrial pressure (mRAP) of at least 16 mmHg. In some embodiments, the method improves the mean right atrial pressure (mRAP) in the patient. In some embodiments, the improvement in the mRAP is a reduction in the mRAP.

In some embodiments, the method reduces the mRAP in the patient by at least 10%. In some embodiments, the method reduces the mRAP in the patient by at least 15%. In some embodiments, the method reduces the mRAP in the patient by at least 20%. In some embodiments, the method reduces the mRAP in the patient by at least 25%. In some embodiments, the method relates to reducing the mRAP in the patient by at least 30%. In some embodiments, the method relates to reducing the mRAP in the patient by at least 35%. In some embodiments, the method relates to reducing the mRAP in the patient by at least 40%. In some embodiments, the method relates to reducing the mRAP in the patient by at least 45%. In some embodiments, the method relates to reducing the mRAP in the patient by at least 50%.

In some embodiments, the method reduces the mRAP by at least 1 mmHg. In some embodiments, the method reduces the mRAP by at least 1 mmHg in the patient. In some embodiments, the method reduces the mRAP by at least 2 mmHg in the patient. In some embodiments, the method reduces the mRAP by at least 3 mmHg in the patient. In some embodiments, the method reduces the mRAP by at least 4 mmHg in the patient. In some embodiments, the method reduces the mRAP by at least 5 mmHg in the patient. In some embodiments, the method reduces the mRAP by at least 6 mmHg in the patient. In some embodiments, the method reduces the mRAP by at least 7 mmHg in the patient. In some embodiments, the method reduces the mRAP by at least 8 mmHg in the patient. In some embodiments, the method reduces the mRAP by at least 9 mmHg in the patient. In some embodiments, the method reduces the mRAP by at least 10 mmHg in the patient. In some embodiments, the method reduces the mRAP by at least 11 mmHg in the patient. In some embodiments, the method reduces the mRAP by at least 12 mmHg in the patient. In some embodiments, the method reduces the mRAP by at least 13 mmHg in the patient. In some embodiments, the method reduces the mRAP by at least 14 mmHg in the patient. In some embodiments, the method reduces the mRAP by at least 15 mmHg in the patient.

### PVR

Vascular resistance is the resistance that must be overcome to push blood through the circulatory system and create flow. Pulmonary vascular resistance is the resistance against blood flow from the pulmonary artery to the left atrium. Total blood flow represents the cardiac output (5 to 6 L/min). A normal value for pulmonary vascular resistance using conventional units is 0.25-1.6 mmHg·min/l. Pulmonary vascular resistance can also be represented in units of dynes/sec/cm5 (normal=37-250 dynes/sec/cm5). One of the factors that contributes to an increase in PVR is hypoxemia. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's PVR is decreased by at least 10%.

In some embodiments, the patient has a pulmonary vascular resistance (PVR) greater than or equal to 3 Wood Units. In some embodiments, the method decreases the PVR in the patient. In some embodiments, the method reduces the PVR in the patient by at least 10%. In some embodiments, the method reduces the PVR in the patient by at least 15%. In some embodiments, the method reduces the PVR in the patient by at least 20%. In some embodiments, the method reduces the PVR in the patient by at least 25%. In some embodiments, the method reduces the PVR in the patient by at least 30%. In some embodiments, the method reduces the PVR in the patient by at least 35%. In some embodiments, the method reduces the PVR in the patient by at least 40%. In some embodiments, the method reduces the PVR in the patient by at least 45%. In some embodiments, the method reduces the PVR in the patient by at least 50%. In some embodiments, the method reduces the PVR to less than 3 Woods Units.

### DPG

The pulmonary artery diastolic pressure gradient, DPG, has historically been used to determine the difference between diastolic pulmonary artery pressure and the wedge pressure. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's DPG is decreased by at least 10%.

In some embodiments, the patient has a diastolic pressure gradient (DPG) of greater than 7 mmHg. In some embodiments, the patient has a DPG of at least 7 mmHg. In some embodiments, the patient has a DPG of at least 10 mmHg. In some embodiments, the patient has a DPG of at least 15 mmHg. In some embodiments, the patient has a DPG of at least 20 mmHg. In some embodiments, the patient has a DPG of at least 25 mmHg. In some embodiments, the patient has a DPG of at least 30 mmHg. In some embodiments, the patient has a DPG of at least 35 mmHg. In some embodiments, the patient has a DPG of at least 40 mmHg. In some embodiments, the patient has a DPG of at least 45 mmHg. In some embodiments, the patient has a DPG of at least 50 mmHg.

In some embodiments, the method decreases the DPG in the patient. In some embodiments, the method decreases the DPG in the patient by at least 10%. In some embodiments, the method decreases the DPG in the patient by at least 15%. In some embodiments, the method decreases the DPG in the patient by at least 20%. In some embodiments, the method decreases the DPG in the patient by at least 25%. In some embodiments, the method decreases the DPG in the patient by at least 30%. In some embodiments, the method decreases the DPG in the patient by at least 35%. In some embodiments, the method decreases the DPG in the patient by at least 40%. In some embodiments, the method decreases the DPG in the patient by at least 45%. In some embodiments, the method decreases the DPG in the patient by at least 50%. In some embodiments, the method decreases the DPG in the patient to less than 7 mmHg.

### BNP

Brain natriuretic peptide (BNP) and NT-proBNP are markers of atrial and ventricular distension due to increased intracardiac pressure. The New York Heart Association (NYHA) developed a 4-stage functional classification system for congestive heart failure (CHF) based on the severity of symptoms. Studies have demonstrated that the measured concentrations of circulating BNP and NT-proBNP increase with the severity of CHF based on the NYHA classification. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's BNP levels are decreased.

In some embodiments, the patient has elevated BNP levels as compared to a healthy patient (e.g., a healthy person of similar age and sex). In some embodiments, the patient has normal BNP levels. In some embodiments, the patient has a BNP level of at least 100 pg/mL. In some embodiments, the patient has a BNP level of at least 150 pg/mL. In some embodiments, the patient has a BNP level of at least 200 pg/mL. In some embodiments, the patient has a BNP level of at least 300 pg/mL. In some embodiments, the patient has a BNP level of at least 400 pg/mL. In some embodiments, the patient has a BNP level of at least 500 pg/mL. In some embodiments, the patient has a BNP level of at least 1000 pg/mL. In some embodiments, the patient has a BNP level of at least 3000 pg/mL. In some embodiments, the patient has a BNP level of at least 5000 pg/mL. In some embodiments, the patient has a BNP level of at least 10,000 pg/mL. In some embodiments, the patient has a BNP level of at least 15,000 pg/mL. In some embodiments, the patient has a BNP level of at least 20,000 pg/mL.

In some embodiments, the method decreases BNP levels in the patient by at least 10%. In some embodiments, the method decreases BNP levels in the patient by at least 20%. In some embodiments, the method decreases BNP levels in the patient by at least 25%. In some embodiments, the method decreases BNP levels in the patient by at least 30%. In some embodiments, the method decreases BNP levels in the patient by at least 35%. In some embodiments, the method decreases BNP levels in the patient by at least 40%. In some embodiments, the method decreases BNP levels in the patient by at least 45%. In some embodiments, the method decreases BNP levels in the patient by at least 50%. In some embodiments, the method decreases BNP levels in the patient by at least 55%. In some embodiments, the method decreases BNP levels in the patient by at least 60%. In some embodiments, the method decreases BNP levels in the patient by at least 65%. In some embodiments, the method decreases BNP levels in the patient by at least 70%. In some embodiments, the method decreases BNP levels in the patient by at least 75%. In some embodiments, the method decreases BNP levels in the patient by at least 80%. In some embodiments, the method decreases BNP levels to normal levels (i.e., <100 pg/ml).

### NT-proBNP

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the patient's NT-proBNP levels are decreased.

In some embodiments, the patient has elevated NT-proBNP levels as compared to a healthy patient (e.g., a healthy person of similar age and sex). In some embodiments, the patient has normal NT-proBNP levels. In some embodiments, the patient has a NT-proBNP level of at least 100 pg/mL. In some embodiments, the patient has a NT-proBNP level of at least 150 pg/mL. In some embodiments, the patient has a NT-proBNP level of at least 200 pg/mL. In some embodiments, the patient has a NT-proBNP level of at least 300 pg/mL. In some embodiments, the patient has a NT-proBNP level of at least 400 pg/mL. In some embodiments, the patient has a NT-proBNP level of at least 500 pg/mL. In some embodiments, the patient has a NT-proBNP level of at least 1000 pg/mL. In some embodiments, the patient has a NT-proBNP level of at least 3000 pg/mL. In some embodiments, the patient has a NT-proBNP level of at least 5000 pg/mL. In some embodiments, the patient has a NT-proBNP level of at least 10,000 pg/mL. In some embodiments, the patient has a NT-proBNP level of at least 15,000 pg/mL. In some embodiments, the patient has a NT-proBNP level of at least 20,000 pg/mL.

In some embodiments, the method decreases NT-proBNP levels in the patient. In some embodiments, the method decreases NT-proBNP levels in the patient by at least 10%. In some embodiments, the method decreases NT-proBNP levels in the patient by at least 20%. In some embodiments, the method decreases NT-proBNP levels in the patient by at least 25%. In some embodiments, the method decreases NT-proBNP levels in the patient by at least 30%. In some embodiments, the method decreases NT-proBNP levels in the patient by at least 35%. In some embodiments, the method decreases NT-proBNP levels in the patient by at least 40%. In some embodiments, the method decreases NT-proBNP levels in the patient by at least 45%. In some embodiments, the method decreases NT-proBNP levels in the patient by at least 50%. In some embodiments, the method decreases NT-proBNP levels in the patient by at least 55%. In some embodiments, the method decreases NT-proBNP levels in the patient by at least 60%. In some embodiments, the method decreases NT-proBNP levels in the patient by at least 65%. In some embodiments, the method decreases NT-proBNP levels in the patient by at least 70%. In some embodiments, the method decreases NT-proBNP levels in the patient by at least 75%. In some embodiments, the method decreases NT-proBNP levels in the patient by at least 80%. In some embodiments, the method decreases NT-proBNP levels in the patient by at least 30%. In some embodiments, the method decreases NT-proBNP levels to normal levels. In some embodiments, the normal level of NT-proBNP is <100 pg/ml.

### Smooth Muscle Hypertrophy

Patients with COPD often experience airway wall remodeling, mostly in small airways, leading to airway wall thickening and airflow obstruction. Similarly, bronchial smooth muscle hypertrophy, characterized by an increase in the smooth muscle cells and thickening of the smooth muscle layer around airways, is a feature of airway wall remodeling in disease states resembling chronic asthma. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method decreases smooth muscle hypertrophy.

In some embodiments, the method decreases smooth muscle hypertrophy in the patient. In some embodiments, the method decreases smooth muscle hypertrophy in the patient by at least 10%. In some embodiments, the method decreases smooth muscle hypertrophy in the patient by at least 15%. In some embodiments, the method decreases smooth muscle hypertrophy in the patient by at least 20%. In some embodiments, the method decreases smooth muscle hypertrophy in the patient by at least 25%. In some embodiments, the method decreases smooth muscle hypertrophy in the patient by at least 30%. In some embodiments, the method decreases smooth muscle hypertrophy in the patient by at least 35%. In some embodiments, the method decreases smooth muscle hypertrophy in the patient by at least 40%. In some embodiments, the method decreases smooth muscle hypertrophy in the patient by at least 45%. In some embodiments, the method decreases smooth muscle hypertrophy in the patient by at least 50%.

### Quality of Life

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method improves a patient's quality of life.

In some embodiments, the patient's quality of life is measured using the Cambridge Pulmonary Hypertension Outcome Review (CAMPHOR). CAMPHOR is a disease specific patient-reported outcome measure which assesses the quality of life of patients with pulmonary hypertension. There are three dimension of CAMPHOR which assess symptoms, functioning, and quality of life. The quality of life (QoL) scale has twenty-five items which focus on socialization, role, acceptance, self-esteem, independence, and security. Similarly, the symptom dimension consists of twenty-five symptoms broken into three subscales: energy, breathlessness, and mood. The activity scale has fifteen items. Scores for QoL and symptoms range from 0-25, with higher scores indicating worse quality of life. Activity scores range from 0-30, with higher scores indicating more physical limitations. In some embodiments, the method decreases the patient's quality of life (QoL) score by at least 10%. In some embodiments, the method decreases the patient's quality of life (QoL) score by at least 2%. In some embodiments, the method decreases the patient's quality of life (QoL) score by at least 3%. In some embodiments, the method decreases the patient's quality of life (QoL) score by at least 4%. In some embodiments, the method decreases the patient's quality of life (QoL) score by at least 5%. In some embodiments, the method decreases the patient's quality of life (QoL) score by at least 10%. In some embodiments, the method decreases the patient's quality of life (QoL) score by at least 15%. In some embodiments, the method decreases the patient's quality of life (QoL) score by at least 20%. In some embodiments, the method decreases the patient's quality of life (QoL) score by at least 25%. In some embodiments, the method decreases the patient's quality of life (QoL) score by at least 30%. In some embodiments, the method decreases the patient's quality of life (QoL) score by at least 35%. In some embodiments, the method decreases the patient's quality of life (QoL) score by at least 40%. In some embodiments, the method decreases the patient's quality of life (QoL) score by at least 45%. In some embodiments, the method decreases the patient's quality of life (QoL) score by at least 50%. In some embodiments, the method decreases the patient's quality of life (QoL) score by at least 55%. In some embodiments, the method decreases the patient's quality of life (QoL) score by at least 60%. In some embodiments, the method decreases the patient's quality of life (QoL) score by at least 65%. In some embodiments, the method decreases the patient's quality of life (QoL) score by at least 70%. In some embodiments, the method decreases the patient's quality of life (QoL) score by at least 75%. In some embodiments, the method decreases the patient's quality of life (QoL) score by at least 80%. In some embodiments, the method decreases the patient's quality of life (QoL) score by at least 85%. In some embodiments, the method decreases the patient's quality of life (QoL) score by at least 90%. In some embodiments, the method decreases the patient's quality of life (QoL) score by at least 95%. In some embodiments, the method decreases the patient's quality of life (QoL) score by at least 100%. In some embodiments, the patient's quality of life is improved as measured using the Cambridge Pulmonary Hypertension Outcome Review (CAMPHOR).

### Ventricular Function

In certain aspects, the disclosure relates to methods of improving or maintaining ventricular function (e.g., left ventricular function or right ventricular function). In some embodiments, the method improves right ventricular function in the patient. In some embodiments, the improvement in right ventricular function is due to an increase in right ventricular fractional area change. In some embodiments, the improvement in right ventricular function is due to a decrease in right ventricular hypertrophy. In some embodiments, the improvement in ejection fraction. In some embodiments, the improvement in the right ventricular hypertrophy in the patient.

In certain aspects, the disclosure relates to diagnostic tests and methods for pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). Echocardiography is a useful noninvasive screening tool for determining the severity of pulmonary hypertension in a patient. Improvement or maintenance of ventricular function (e.g., left ventricular function or right ventricular function) can be assessed by many echocardiographic measurements. One such quantitative approach to assess ventricular function is the measurement of the tricuspid annular plane systolic excursion (TAPSE). The TAPSE estimates RV systolic function by measuring the level of systolic excursion of the lateral tricuspid valve annulus towards the apex. Other echocardiographic measurements that may be used to assess maintenance and/or improvements in ventricular function include, but are not limited to, right ventricular fractional area change (RVFAC), right ventricular end-diastolic area (RVEDA), right ventricular end-systolic area (RVESA), right ventricular free wall thickness (RVFWT), right ventricular ejection fraction (RVEF), right ventricular-pulmonary artery (RV-PA) coupling, pulmonary arterial systolic pressure (PASP), right ventricular systolic pressure (RVSP), pulmonary artery acceleration time (PAAT), tricuspid regurgitation velocity (TRV), left ventricular hypertrophy, and right ventricular hypertrophy.

### TAPSE

The tricuspid annular plane systolic excursion (TAPSE) can be obtained using echocardiography and represents a measure of RV longitudinal function. The TAPSE has previously been shown to have good correlations with parameters estimating RV global systolic function. A TAPSE<17 mm is highly suggestive of RV systolic dysfunction. In some embodiments of the methods disclosed herein, the patient has a TAPSE of less than 20 mm. In some embodiments, the patient has a TAPSE of less than 18 mm. In some embodiments, the patient has a TAPSE of less than 16 mm. In some embodiments, the patient has a TAPSE of less than 14 mm. In some embodiments, the patient has a TAPSE of less than 12 mm.

In some embodiments, the method increases the TAPSE to at least 20 mm. In some embodiments, the method increases the TAPSE to at least 22 mm. In some embodiments, the method increases the TAPSE to at least 24 mm. In some embodiments, the method increases the TAPSE to at least 26 mm. In some embodiments, the method increases the TAPSE to at least 28 mm. In some embodiments, the method increases the TAPSE to at least 30 mm.

### PASP and RVSP

In certain aspects, the disclosure relates to methods of treating pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method reduces the right ventricular systolic pressure (RVSP) by at least 10%.

In some embodiments, the PASP is a resting PASP. In some embodiments, the PASP is determined using the tricuspid regurgitation velocity (TRV) and right arterial (RA) pressure. In some embodiments, the PASP is determined using the following formula:

$$PASP = {TRV}^{2} \times 4 + RA pressure$$

TRV has been shown to correlate with PASP at rest and with exercise. The pressure gradient between the right ventricle and the right atrium can be calculated using the modified Bernoulli equation (Δp=4V²).

In some embodiments, the right ventricular systolic pressure (RVSP) is equal to PASP. In some embodiments, the RVSP is measured in the absence of right ventricular outflow tract obstruction. In some embodiments, the RVSP is determined using the following formula:

$$RVSP = 4 {V}^{2} + RAP$$

In the above formula, V represents the peak tricuspid regurgitant jet velocity and RAP is the mean right atrial pressure. RVSP is frequently used for estimating PASP.

In some embodiments, the patient has a right ventricular systolic pressure (RVSP) of greater than 35 mmHg. In some embodiments, the method decreases the RVSP in the patient. In some embodiments, the methods reduce the RVSP in the patient by at least 10%. In some embodiments, the method decreases the RVSP in the patient by at least 15%. In some embodiments, the methods reduce the RVSP in the patient by at least 20%. In some embodiments, the method decreases the RVSP in the patient by at least 25%. In some embodiments, the methods reduce the RVSP in the patient by at least 30%. In some embodiments, the method decreases the RVSP in the patient by at least 35%. In some embodiments, the methods reduce the RVSP in the patient by at least 40%. In some embodiments, the method decreases the RVSP in the patient by at least 45%. In some embodiments, the methods reduce the RVSP in the patient by at least 50%. In some embodiments, the methods reduce the RVSP in the patient to less than 25 mmHg.

In some embodiments, the patient has a pulmonary artery systolic pressure (PASP) of greater than 20 mmHg. In some embodiments, the patient has a PASP of greater than 25 mmHg. In some embodiments, the patient has a PASP of at least 35 mmHg. In some embodiments, the patient has a PASP of at least 40 mmHg. In some embodiments, the patient has a PASP of at least 50 mmHg. In some embodiments, the patient has a PASP of at least 55 mmHg. In some embodiments, the patient has a PASP of at least 60 mmHg.

In some embodiments, the method decreases the PASP in the patient. In some embodiments, the method reduces the PASP in the patient by at least 10%. In some embodiments, the method decreases the PASP in the patient by at least 15%. In some embodiments, the methods reduce the PASP in the patient by at least 20%. In some embodiments, the method decreases the PASP in the patient by at least 25%. In some embodiments, the methods reduce the PASP in the patient by at least 30%. In some embodiments, the method decreases the PASP in the patient by at least 35%. In some embodiments, the methods reduce the PASP in the patient by at least 40%. In some embodiments, the method decreases the PASP in the patient by at least 45%. In some embodiments, the methods reduce the PASP in the patient by at least 50%.

In some embodiments, the method reduces the PASP in the patient by at least 5 mmHg. In some embodiments, the method reduces the PASP in the patient by at least 10 mmHg. In some embodiments, the method reduces the PASP in the patient by at least 15 mmHg. In some embodiments, the method reduces the PASP in the patient by at least 20 mmHg. In some embodiments, the method reduces the PASP in the patient by at least 25 mmHg. In some embodiments, the method reduces the PASP in the patient to less than 25 mmHg. In some embodiments, the method reduces the PASP in the patient to less than 20 mmHg.

### Right Ventricular Hypertrophy

Right ventricular hypertrophy (RVH) is a pathological increase in muscle mass of the right ventricle in response to pressure overload, most commonly due to severe lung disease. Symptoms of RVH due to pulmonary hypertension include exertional chest pain, peripheral edema, exertional syncope, and right upper quadrant pain. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42.

In some embodiments, the methods decrease right ventricular hypertrophy in the patient. In some embodiments, the methods decrease right ventricular hypertrophy by at least 10%. In some embodiments, the methods decrease right ventricular hypertrophy by at least 15%. In some embodiments, the methods decrease right ventricular hypertrophy by at least 20%. In some embodiments, the methods decrease right ventricular hypertrophy by at least 25%. In some embodiments, the methods decrease right ventricular hypertrophy by at least 30%. In some embodiments, the methods decrease right ventricular hypertrophy by at least 35%. In some embodiments, the methods decrease right ventricular hypertrophy by at least 40%. In some embodiments, the methods decrease right ventricular hypertrophy by at least 45%. In some embodiments, the methods decrease right ventricular hypertrophy by at least 50%.

### Cardiac Index

Cardiac index (CI) is an assessment of cardiac output based on a patient's size. Both the cardiac output and cardiac index are important in determining whether the heart is pumping enough blood and delivering enough oxygen to cells. Cardiac index allows the comparison of cardiac function between individuals of different sizes. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method increases cardiac index.

In some embodiments, the patient has a cardiac index of less than 2.5 L/min/m². In some embodiments, the patient has a cardiac index of less than 2.0 L/min/m². In some embodiments, the patient has a cardiac index of less than 1.5 L/min/m². In some embodiments, the patient has a cardiac index of less than 1.0 L/min/m². In some embodiments, the method increases the CI in the patient by at least 10%. In some embodiments, the method increases the CI in the patient by at least 10%. In some embodiments, the method increases the CI in the patient by at least 10%. In some embodiments, the method increases the CI in the patient by at least 15%. In some embodiments, the method increases the CI in the patient by at least 20%. In some embodiments, the method increases the CI in the patient by at least 25%. In some embodiments, the method increases the CI in the patient by at least 30%. In some embodiments, the method increases the CI in the patient by at least 35%. In some embodiments, the method increases the CI in the patient by at least 40%. In some embodiments, the method increases the CI in the patient by at least 45%. In some embodiments, the method increases the CI in the patient by at least 50%. In some embodiments, the method increases the CI in the patient by at least 0.2 L/min/m². In some embodiments, the method increases the CI in the patient by at least 0.4 L/min/m². In some embodiments, the method increases the CI in the patient by at least 0.6 L/min/m². In some embodiments, the method increases the CI in the patient by at least 0.8 L/min/m². In some embodiments, the method increases the CI in the patient by at least 1 L/min/m². In some embodiments, the method increases the CI in the patient by at least 1.2 L/min/m². In some embodiments, the method increases the CI in the patient by at least 1.4 L/min/m². In some embodiments, the method increases the CI in the patient by at least 1.6 L/min/m². In some embodiments, the method increases the CI in the patient by at least 1.8 L/min/m². In some embodiments, the method increases the CI in the patient by at least 2 L/min/m². In some embodiments, the method increases the CI in the patient to at least 2.5 L/min/m².

### Cardiac Output

In general, normal cardiac output at rest is about 2.5-4.2 L/min/m2, and cardiac output can decline by almost 40% without deviating from the normal limits. A low cardiac index of less than about 2.5 L/min/m2 usually indicates a disturbance in cardiovascular performance. The cardiac output can be utilized to calculate the cardiac index (e.g., cardiac index=cardiac output/body surface area). The cardiac output can be also utilized to calculate the stroke volume (e.g., stroke volume=CO/heart rate). In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method increases the cardiac output.

In some embodiments, the patient has a cardiac output of less than 4 L/min. In some embodiments, the method increases the cardiac output in the patient by at least 10%. In some embodiments, the method increases the cardiac output in the patient by at least 15%. In some embodiments, the method increases the cardiac output in the patient by at least 20%. In some embodiments, the method increases the cardiac output in the patient by at least 25%. In some embodiment, the method increases the cardiac output in the patient by at least 30%. In some embodiments, the method increases the cardiac output in the patient by at least 35%. In some embodiments, the method increases the cardiac output in the patient by at least 40%. In some embodiments, the method increases the cardiac output in the patient by at least 45%. In some embodiments, the method increases the cardiac output in the patient by at least 50%. In some embodiments, the method increases the cardiac output in the patient by at least 0.5 L/min. In some embodiments, the method increases the cardiac output in the patient by at least 1 L/min. In some embodiments, the method increases the cardiac output in the patient by at least 1.5 L/min. In some embodiments, the method increases the cardiac output in the patient by at least 2 L/min. In some embodiments, the method increases the cardiac output in the patient by at least 2.5 L/min. In some embodiments, the method increases the cardiac output in the patient by at least 3 L/min. In some embodiments, the method increases the cardiac output in the patient by at least 3.5 L/min. In some embodiments, the method increases the cardiac output in the patient by at least 4 L/min.

### Composite Physiologic Index (CPI)

Composite physiologic index (CPI) can be used to determine the extent of pulmonary fibrosis. It is difficult to predict the clinical course of fibrotic lung diseases (e.g., idiopathic pulmonary fibrosis). CPI models can be used as a predictor of fibrotic disease progression. In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method decreases the composite physiologic index.

In some embodiments, the patient has a CPI greater than 15. In some embodiments of the methods herein, the patient has a CPI greater than 20. In some embodiments of the methods herein, the patient has a CPI greater than 25. In some embodiments of the methods herein, the patient has a CPI greater than 30. In some embodiments of the methods herein, the patient has a CPI greater than 35. In some embodiments of the methods herein, the patient has a CPI greater than 40. In some embodiments of the methods herein, the patient has a CPI greater than 45. In some embodiments of the methods herein, the patient has a CPI greater than 50. In some embodiments of the methods herein, the patient has a CPI greater than 55. In some embodiments of the methods herein, the patient has a CPI greater than 60. In some embodiments of the methods herein, the patient has a CPI greater than 65. In some embodiments of the methods herein, the patient has a CPI greater than 70. In some embodiments of the methods herein, the patient has a CPI greater than 75. In some embodiments of the methods herein, the patient has a CPI greater than 80. In some embodiments, the method decreases the CPI in the patient. In some embodiments, the method decreases the CPI in the patient by 10%. In some embodiments, the method decreases the CPI in the patient by 15%. In some embodiments, the method decreases the CPI in the patient by 20%. In some embodiments, the method decreases the CPI in the patient by 25%. In some embodiments, the method decreases the CPI in the patient by 30%. In some embodiments, the method decreases the CPI in the patient by 35%. In some embodiments, the method decreases the CPI in the patient by 40%. In some embodiments, the method decreases the CPI in the patient by 45%. In some embodiments, the method decreases the CPI in the patient by 50%. In some embodiments, the method decreases the CPI to less than 70. In some embodiments, the method decreases the CPI to less than 65. In some embodiments, the method decreases the CPI to less than 60. In some embodiments, the method decreases the CPI to less than 55. In some embodiments, the method decreases the CPI to less than 50. In some embodiments, the method decreases the CPI to less than 45. In some embodiments, the method decreases the CPI to less than 40. In some embodiments, the method decreases the CPI to less than 35. In some embodiments, the method decreases the CPI to less than 30. In some embodiments, the method decreases the CPI to less than 25. In some embodiments, the method decreases the CPI to less than 20. In some embodiments, the method decreases the CPI to less than 15. In some embodiments, the method decreases the CPI to less than 10. In some embodiments, the method decreases the CPI to less than 5.

### Oxygen Saturation at Rest

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the method increases the arterial oxygen saturation.

In some embodiments, the patient has an arterial oxygen saturation of less than 95%. In some embodiments of the methods disclosed herein, the patient has an arterial oxygen saturation of less than 90%. In some embodiments of the methods disclosed herein, the patient has an arterial oxygen saturation of less than 85%. In some embodiments of the methods disclosed herein, the patient has an arterial oxygen saturation of less than 80%. In some embodiments of the methods disclosed herein, the patient has an arterial oxygen saturation of less than 75%. In some embodiments of the methods disclosed herein, the patient has an arterial oxygen saturation of less than 70%. In some embodiments of the methods disclosed herein, the patient has an arterial oxygen saturation of less than 65%. In some embodiments of the methods disclosed herein, the patient has an arterial oxygen saturation of less than 60%. In some embodiments of the methods disclosed herein, the patient has an arterial oxygen saturation of less than 55%. In some embodiments of the methods disclosed herein, the patient has an arterial oxygen saturation of less than 50%. In some embodiments of the methods disclosed herein, the patient has an arterial oxygen saturation of less than 45%. In some embodiments of the methods disclosed herein, the patient has an arterial oxygen saturation of less than 40%. In some embodiments of the methods disclosed herein, the patient has an arterial oxygen saturation of less than 35%. In some embodiments of the methods disclosed herein, the patient has an arterial oxygen saturation of less than 30%. In some embodiments, the method increases the arterial oxygen saturation in a patient. In some embodiments, the method increases the arterial oxygen saturation in the patient by at least 5%.

In some embodiments, the method increases the arterial oxygen saturation in the patient by at least 10%. In some embodiments, the method increases the arterial oxygen saturation in the patient by at least 15%. In some embodiments, the method increases the arterial oxygen saturation in the patient by at least 20%. In some embodiments, the method increases the arterial oxygen saturation in the patient by at least 25%. In some embodiments, the method increases the arterial oxygen saturation in the patient by at least 30%. In some embodiments, the method increases the arterial oxygen saturation in the patient by at least 35%. In some embodiments, the method increases the arterial oxygen saturation in the patient by at least 40%. In some embodiments, the method increases the arterial oxygen saturation in the patient by at least 45%. In some embodiments, the method increases the arterial oxygen saturation in the patient by at least 50%. In some embodiments, the method increases the arterial oxygen saturation in the patient by at least 85%. In some embodiments, the method increases the arterial oxygen saturation in the patient by at least 90%. In some embodiments, the method increases the arterial oxygen saturation in the patient by at least 95%. In some embodiments, the arterial oxygen saturation is measured at rest.

### Exercise Capacity (6 MWD AND BDI)

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods increase exercise capacity in the patient.

Any suitable measure of exercise capacity can be used. For example, exercise capacity in a 6-minute walk test (6 MWT), which measures how far the subject can walk in 6 minutes, i.e., the 6-minute walk distance (6 MWD), is frequently used to assess pulmonary hypertension severity and disease progression. The BDI is a numerical scale for assessing perceived dyspnea (breathing discomfort), and may be used to measure exercise capacity. It measures the degree of breathlessness, for example, after completion of the 6 MWT, where a BDI of 0 indicates no breathlessness and 10 indicates maximum breathlessness. In some embodiments, the patient has a 6-minute walk distance (6 MWD) of less than 550 meters. In some embodiments, the patient has a 6-minute walk distance (6 MWD) of less than 550 meters. In some embodiments, the patient has a 6-minute walk distance (6 MWD) of less than 500 meters. In some embodiments, the patient has a 6-minute walk distance (6 MWD) of less than 450 meters. In some embodiments, the patient has a 6-minute walk distance (6 MWD) of less than 400 meters. In some embodiments, the patient has a 6-minute walk distance (6 MWD) of less than 350 meters. In some embodiments, the patient has a 6-minute walk distance (6 MWD) of less than 300 meters. In some embodiments, the patient has a 6-minute walk distance (6 MWD) of less than 250 meters. In some embodiments, the patient has a 6-minute walk distance (6 MWD) of less than 200 meters. In some embodiments, the patient has a 6-minute walk distance (6 MWD) of less than 150 meters. In some embodiments, the method increases the patient's 6 MWD by at least 10 meters. In some embodiments, the method increases the patient's 6 MWD by at least 15 meters. In some embodiments, the method increases the patient's 6 MWD by at least 20 meters. In some embodiments, the method increases the patient's 6 MWD by at least 25 meters. In some embodiments, the method increases the patient's 6 MWD by at least 30 meters. In some embodiments, the method increases the patient's 6 MWD by at least 35 meters. In some embodiments, the method increases the patient's 6 MWD by at least 40 meters. In some embodiments, the method increases the patient's 6 MWD by at least 45 meters. In some embodiments, the method increases the patient's 6 MWD by at least 50 meters. In some embodiments, the method increases the patient's 6 MWD by at least 55 meters. In some embodiments, the method increases the patient's 6 MWD by at least 60 meters. In some embodiments, the method increases the patient's 6 MWD by at least 65 meters. In some embodiments, the method increases the patient's 6 MWD by at least 70 meters. In some embodiments, the method increases the patient's 6 MWD by at least 75 meters. In some embodiments, the method increases the patient's 6 MWD by at least 80 meters. In some embodiments, the method increases the patient's 6 MWD by at least 85 meters. In some embodiments, the method increases the patient's 6 MWD by at least 90 meters. In some embodiments, the method increases the patient's 6 MWD by at least 95 meters. In some embodiments, the method increases the patient's 6 MWD by at least 100 meters. In some embodiments, the method increases the patient's 6 MWD by at least 125 meters. In some embodiments, the method increases the patient's 6 MWD by at least 150 meters. In some embodiments, the method increases the patient's 6 MWD by at least 175 meters. In some embodiments, the method increases the patient's 6 MWD by at least 200 meters. In some embodiments, the method increases the patient's 6 MWD by at least 250 meters. In some embodiments, the method increases the patient's 6 MWD by at least 300 meters. In some embodiments, the method increases the patient's 6 MWD by at least 400 meters.

In some embodiments, the method increases exercise capacity of the patient. In some embodiments, the patient has a Borg dyspnea index (BDI) at least 0.5 index points. In some embodiments, the patient has a Borg dyspnea index (BDI) at least 1 index points. In some embodiments, the patient has a Borg dyspnea index (BDI) at least 1.5 index points. In some embodiments, the patient has a Borg dyspnea index (BDI) at least 2 index points. In some embodiments, the patient has a Borg dyspnea index (BDI) at least 2.5 index points. In some embodiments, the patient has a Borg dyspnea index (BDI) at least 3 index points. In some embodiments, the patient has a Borg dyspnea index (BDI) at least 3.5 index points. In some embodiments, the patient has a Borg dyspnea index (BDI) at least 4 index points. In some embodiments, the patient has a Borg dyspnea index (BDI) at least 4.5 index points. In some embodiments, the patient has a Borg dyspnea index (BDI) at least 5 index points. In some embodiments, the patient has a Borg dyspnea index (BDI) at least 5.5 index points. In some embodiments, the patient has a Borg dyspnea index (BDI) at least 6 index points. In some embodiments, the patient has a Borg dyspnea index (BDI) at least 6.5 index points. In some embodiments, the patient has a Borg dyspnea index (BDI) at least 7 index points. In some embodiments, the patient has a Borg dyspnea index (BDI) at least 7.5 index points. In some embodiments, the patient has a Borg dyspnea index (BDI) at least 8 index points. In some embodiments, the patient has a Borg dyspnea index (BDI) at least 8.5 index points. In some embodiments, the patient has a Borg dyspnea index (BDI) at least 9 index points. In some embodiments, the patient has a Borg dyspnea index (BDI) at least 9.5 index points. In some embodiments, the patient has a Borg dyspnea index (BDI) at least 10 index points. In some embodiments, the method reduces the patient's Borg dyspnea index (BDI). In some embodiments, the method reduces the patient's BDI by at least 0.5 index points. In some embodiments, the method reduces the patient's BDI by at least 1 index points. In some embodiments, the method reduces the patient's BDI by at least 1.5 index points. In some embodiments, the method reduces the patient's BDI by at least 2 index points. In some embodiments, the method reduces the patient's BDI by at least 2.5 index points. In some embodiments, the method reduces the patient's BDI by at least 3 index points. In some embodiments, the method reduces the patient's BDI by at least 3.5 index points. In some embodiments, the method reduces the patient's BDI by at least 4 index points. In some embodiments, the method reduces the patient's BDI by at least 4.5 index points. In some embodiments, the method reduces the patient's BDI by at least 5 index points. In some embodiments, the method reduces the patient's BDI by at least 5.5 index points. In some embodiments, the method reduces the patient's BDI by at least 6 index points. In some embodiments, the method reduces the patient's BDI by at least 6.5 index points. In some embodiments, the method reduces the patient's BDI by at least 7 index points. In some embodiments, the method reduces the patient's BDI by at least 7.5 index points. In some embodiments, the method reduces the patient's BDI by at least 8 index points. In some embodiments, the method reduces the patient's BDI by at least 8.5 index points. In some embodiments, the method reduces the patient's BDI by at least 9 index points. In some embodiments, the method reduces the patient's BDI by at least 9.5 index points. In some embodiments, the method reduces the patient's BDI by at least 10 index points.

### Pulmonary Function Test

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods improve pulmonary function of the patient.

Spirometry, or measuring of breath, is a major pulmonary function test that can determine volume and/or speed (flow) of air that is inhaled and exhaled by a subject. A spirometer is used to measure forced vital capacity (FVC) (measured in liters and/or percentage of predicted) in a forced expiratory volume (FEV) test, among other characteristics. In an FEV test, a subject takes a deep breath, and exhales into a sensor as hard and for as long as possible (e.g., at least 6 seconds). Inhalation can also be tested using spirometry. An FEV test is typically repeated at least three times to ensure accuracy. “Normal” ranges for FVC are typically considered to be between 80% and 100% of predicted. “Of predicted” refers to reporting the subject's results as a percentage of the known predicted values for a healthy subject of similar characteristics (e.g., height, sex, age, race, weight). Other measurements that can be taken include, but are not limited to, FEV₁, wherein the FVC is measured within the first second of forced exhalation, and/or forced expiratory flow (FEF), which measures the flow of air coming out of the lung during the middle portion of forced expiration. An FEV₁/FVC ratio is also typically calculated.

In some embodiments of the methods disclosed herein, the patient has a forced expiratory volume in one second (FEV₁) of greater than 70%. In some embodiments of the methods disclosed herein, the patient has a forced expiratory volume in one second (FEV₁) of 60% to 69%. In some embodiments of the methods disclosed herein, the patient has a forced expiratory volume in one second (FEV₁) of 50% to 59%. In some embodiments of the methods disclosed herein, the patient has a forced expiratory volume in one second (FEV₁) of 35% to 49%. In some embodiments of the methods disclosed herein, the patient has a forced expiratory volume in one second (FEV₁) of less than 35%. In some embodiments, the method increases the FEV₁ in the patient. In some embodiments, the method increases the FEV₁ in the patient by at least 5%. In some embodiments, the method increases the FEV₁ in the patient by at least 10%. In some embodiments, the method increases the FEV₁ in the patient by at least 15%. In some embodiments, the method increases the FEV₁ in the patient by at least 20%. In some embodiments, the method increases the FEV₁ in the patient by at least 25%. In some embodiments, the method increases the FEV₁ in the patient by at least 30%. In some embodiments, the method increases the FEV₁ in the patient by at least 35%. In some embodiments, the method increases the FEV₁ in the patient by at least 40%. In some embodiments, the method increases the FEV₁ in the patient by at least 45%. In some embodiments, the method increases the FEV₁ in the patient by at least 50%. In some embodiments, the method increases the FEV₁ to at least 60%. In some embodiments, the method increases the FEV₁ to at least 65%. In some embodiments, the method increases the FEV₁ to at least 70%. In some embodiments, the method increases the FEV₁ to at least 75%. In some embodiments, the method increases the FEV₁ to at least 80%. In some embodiments, the method increases the FEV₁ to at least 85%. In some embodiments, the method increases the FEV₁ to at least 90%. In some embodiments, the method increases the FEV₁ to at least 95%.

In some embodiments, the patient has a forced vital capacity (FVC) of greater than 80%. In some embodiments, the patient has a forced vital capacity (FVC) of greater than 70%. In some embodiments, the patient has a forced vital capacity (FVC) of 60% to 69%. In some embodiments, the patient has a forced vital capacity (FVC) of 50% to 59%. In some embodiments, the patient has a forced vital capacity (FVC) of 35% to 49%. In some embodiments, the patient has a forced vital capacity (FVC) of less than 35%.

In some embodiments, the method increases the FVC in the patient. In some embodiments, the method increases the FVC in the patient by at least 5%. In some embodiments, the method increases the FVC in the patient by at least 10%. In some embodiments, the method increases the FVC in the patient by at least 15%. In some embodiments, the method increases the FVC in the patient by at least 20%. In some embodiments, the method increases the FVC in the patient by at least 25%. In some embodiments, the method increases the FVC in the patient by at least 30%. In some embodiments, the method increases the FVC in the patient by at least 35%. In some embodiments, the method increases the FVC in the patient by at least 40%. In some embodiments, the method increases the FVC in the patient by at least 45%. In some embodiments, the method increases the FVC in the patient by at least 50%. In some embodiments, the method increases the FVC to at least 60%. In some embodiments, the method increases the FVC to at least 65%. In some embodiments, the method increases the FVC to at least 70%. In some embodiments, the method increases the FVC to at least 75%. In some embodiments, the method increases the FVC to at least 80%. In some embodiments, the method increases the FVC to at least 85%. In some embodiments, the method increases the FVC to at least 90%. In some embodiments, the method increases the FVC to at least 95%.

### Carbon Monoxide Transfer Coefficient K_{CO}

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods increase diffusing capacity of carbon monoxide of the patient.

The diffusing capacity of carbon monoxide, or DL_{CO} can be used in conjunction with spirometry and lung volume assessment to diagnose underlying lung disease (e.g., normal spirometry and lung volumes associated with decreased DL_{CO} may suggest anemia, pulmonary vascular disorders, early ILD, or early emphysema). In some embodiments, the patient has a diffusing capacity of carbon monoxide (DL_{CO}) less than 60%. In some embodiments, the patient has a diffusing capacity of carbon monoxide (DL_{CO}) less than 55%. In some embodiments, the patient has a diffusing capacity of carbon monoxide (DL_{CO}) less than 50%. In some embodiments, the patient has a diffusing capacity of carbon monoxide (DL_{CO}) less than 45%. In some embodiments, the patient has a diffusing capacity of carbon monoxide (DL_{CO}) less than 40%. In some embodiments, the patient has a diffusing capacity of carbon monoxide (DL_{CO}) less than 35%. In some embodiments, the patient has a diffusing capacity of carbon monoxide (DL_{CO}) less than 30%. In some embodiments, the patient has a diffusing capacity of carbon monoxide (DL_{CO}) less than 25%. In some embodiments, the patient has a diffusing capacity of carbon monoxide (DL_{CO}) less than 20%.

In some embodiments, the method increases the DL_{CO} in the patient. In some embodiments, the method increases the DL_{CO} in the patient by at least 5%. In some embodiments, the method increases the DL_{CO} in the patient by at least 10%. In some embodiments, the method increases the DL_{CO} in the patient by at least 15%. In some embodiments, the method increases the DL_{CO} in the patient by at least 20%. In some embodiments, the method increases the DL_{CO} in the patient by at least 25%. In some embodiments, the method increases the DL_{CO} in the patient by at least 30%. In some embodiments, the method increases the DL_{CO} in the patient by at least 35%. In some embodiments, the method increases the DL_{CO} in the patient by at least 40%. In some embodiments, the method increases the DL_{CO} in the patient by at least 45%. In some embodiments, the method increases the DL_{CO} in the patient by at least 50%. In some embodiments, the method increases the DL_{CO} to at least 40%. In some embodiments, the method increases the DL_{CO} to at least 45%. In some embodiments, the method increases the DL_{CO} to at least 50%. In some embodiments, the method increases the DL_{CO} to at least 55%. In some embodiments, the method increases the DL_{CO} to at least 60%. In some embodiments, the method increases the DL_{CO} to at least 65%.

### Pulmonary Fibrosis

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods decrease pulmonary fibrosis in the patient. In some embodiments, the method reduces the pulmonary fibrosis in the patient by at least 10%. In some embodiments, the method reduces the pulmonary fibrosis in the patient by at least 15%. In some embodiments, the method reduces the pulmonary fibrosis in the patient by at least 20%. In some embodiments, the method reduces the pulmonary fibrosis in the patient by at least 25%. In some embodiments, the method reduces the pulmonary fibrosis in the patient by at least 30%. In some embodiments, the method reduces the pulmonary fibrosis in the patient by at least 35%. In some embodiments, the method reduces the pulmonary fibrosis in the patient by at least 40%. In some embodiments, the method reduces the pulmonary fibrosis in the patient by at least 45%. In some embodiments, the method reduces the pulmonary fibrosis in the patient by at least 50%.

### Transplant Free Survival

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods increase transplant free survival in the patient.

In some embodiments, the method increases transplant free survival in the patient by at least 10%. In some embodiments, the method increases transplant free survival in the patient by at least 15%. In some embodiments, the method increases transplant free survival in the patient by at least 20%. In some embodiments, the method increases transplant free survival in the patient by at least 25%. In some embodiments, the method increases transplant free survival in the patient by at least 30%. In some embodiments, the method increases transplant free survival in the patient by at least 35%. In some embodiments, the method increases transplant free survival in the patient by at least 40%. In some embodiments, the method increases transplant free survival in the patient by at least 45%. In some embodiments, the method increases transplant free survival in the patient by at least 50%.

### Death

In certain aspects, the disclosure relates to methods of treating, preventing, or reducing the progression rate and/or severity of one or more complications of pulmonary hypertension associated with lung disease, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42, wherein the methods reduce the risk of death.

In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 10%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 15%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 20%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 25%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 30%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 35%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 40%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 45%. In some embodiments, the method reduces the risk of death associated with pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) by at least 50%.

### Combination Therapies

Optionally, methods disclosed herein for treating, preventing, or reducing the progression rate and/or severity of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), particularly treating, preventing, or reducing the progression rate and/or severity of one or more complications of pulmonary hypertension associated with lung disease (e.g., pulmonary hypertension associated with chronic obstructive pulmonary disease (COPD), interstitial lung disease (ILD), or combined pulmonary fibrosis and emphysema (CPFE)), may further comprise administering to the patient one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). For example, the patient also may be administered one or more supportive therapies or active agents selected from the group consisting of: nitrates, hydralazine, pyridones (e.g., pirfenidone), small molecule tyrosine-kinase inhibitors (e.g., nintedanib), prostacyclin and derivatives thereof (e.g., epoprostenol, treprostinil, and iloprost); prostacyclin receptor agonists (e.g., selexipag); endothelin receptor antagonists (e.g., thelin, ambrisentan, macitentan, darusentan, and bosentan); calcium channel blockers (e.g., amlodipine, diltiazem, and nifedipine; anticoagulants (e.g., warfarin); diuretics; oxygen therapy; atrial septostomy; pulmonary thromboendarterectomy; phosphodiesterase type 5 inhibitors (e.g., sildenafil and tadalafil); activators of soluble guanylate cyclase (e.g., cinaciguat, vericiguat, and riociguat); ASK-1 inhibitors (e.g., CIIA; SCH79797; GS-4997; MSC2032964A; 3H-naphtho[1,2,3-de]quiniline-2,7-diones, NQDI-1; 2-thioxo-thiazolidines, 5-bromo-3-(4-oxo-2-thioxo-thiazolidine-5-ylidene)-1,3-dihydro-indol-2-one); NF-κB antagonists (e.g., dh404, CDDO-epoxide; 2.2-difluoropropionamide; C28 imidazole (CDDO-Im); 2-cyano-3,12-dioxoolean-1,9-dien-28-oic acid (CDDO); 3-Acetyloleanolic Acid; 3-Triflouroacetyloleanolic Acid; 28-Methyl-3-acetyloleanane; 28-Methyl-3-trifluoroacetyloleanane; 28-Methyloxyoleanolic Acid; SZC014; SCZ015; SZC017; PEGylated derivatives of oleanolic acid; 3-O-(beta-D-glucopyranosyl) oleanolic acid; 3-O-[beta-D-glucopyranosyl-(1-->3)-beta-D-glucopyranosyl] oleanolic acid; 3-O-[beta-D-glucopyranosyl-(1-->2)-beta-D-glucopyranosyl] oleanolic acid; 3-O-[beta-D-glucopyranosyl-(1-->3)-beta-D-glucopyranosyl] oleanolic acid 28-O-beta-D-glucopyranosyl ester; 3-O-[beta-D-glucopyranosyl-(1-->2)-beta-D-glucopyranosyl] oleanolic acid 28-O-beta-D-glucopyranosyl ester; 3-O-[a-L-rhamnopyranosyl-(1-->3)-beta-D-glucuronopyranosyl] oleanolic acid; 3-O-[alpha-L-rhamnopyranosyl-(1-->3)-beta-D-glucuronopyranosyl] oleanolic acid 28-O-beta-D-glucopyranosyl ester; 28-O-β-D-glucopyranosyl-oleanolic acid; 3-O-β-D-glucopyranosyl (1→3)-β-D-glucopyranosiduronic acid (CS1); oleanolic acid 3-O-β-D-glucopyranosyl (1→3)-β-D-glucopyranosiduronic acid (CS2); methyl 3,11-dioxoolean-12-en-28-olate (DIOXOL); ZCVI₄-2; Benzyl 3-dehydr-oxy-1,2,5-oxadiazolo[3′,4′:2,3]oleanolate), oxygen therapy, lung and/or heart transplantation. In some embodiments, the methods described herein may further comprise administering to the patient pirfenidone. In some embodiments, the methods described herein may further comprise administering to the patient nintedanib. In some embodiments, the methods described herein may further comprise administering to the patient parental prostacyclin. In some embodiments, the methods described herein may further comprise administering to the patient one additional supportive therapy or additional active agent (i.e., double therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). In some embodiments, the methods described herein may further comprise administering to the patient two additional supportive therapies or additional active agents (i.e., triple therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)). In some embodiments, the methods described herein may further comprise administering to the patient three additional supportive therapies or additional active agents (i.e., quadruple therapy) for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)).

In some embodiments, the methods described herein may further comprise administering to the patient an angiotensin antagonist (e.g., angiotensin receptor blocker, ARB). In some embodiments, a patient is further administered one or more ARBs selected from the group consisting of losartan, irbesartan, olmesartan, candesartan, valsartan, fimasartan, azilsartan, salprisartan, and telmisartan. In some embodiments, a patient is administered losartan. In some embodiments, a patient is administered irbesartan. In some embodiments, a patient is administered olmesartan. In some embodiments, a patient is administered candesartan. In some embodiments, a patient is administered valsartan. In some embodiments, a patient is administered fimasartan. In some embodiments, a patient is administered azilsartan. In some embodiments, a patient is administered salprisartan. In some embodiments, a patient is administered telmisartan.

In some embodiments, the methods described herein may further comprise administering to the patient one or more ACE inhibitors. In some embodiments, the one or more ACE inhibitors are selected from the group consisting of benazepril, captopril, enalapril, lisinopril, perindopril, ramipril (e.g., ramipen), trandolapril, and zofenopril. In some embodiments, a patient is administered benazepril. In some embodiments, a patient is administered captopril. In some embodiments, a patient is administered enalapril. In some embodiments, a patient is administered lisinopril. In some embodiments, a patient is administered perindopril. In some embodiments, a patient is administered ramipril. In some embodiments, a patient is administered trandolapril. In some embodiments, a patient is administered zofenopril. In some embodiments, the methods described herein may further comprise administering to the patient an ARB and an ACE inhibitor. In some embodiments, an alternative approach to angiotensin antagonism is to combine an ACE inhibitor and/or ARB with an aldosterone antagonist.

In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered prior to administration of the combination of an ActRII polypeptide and a TβRII polypeptide. In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered in combination with an ActRII polypeptide and a TβRII polypeptide. In some embodiments, the one or more supportive therapies or additional active agents for treating pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) are administered after the administration of the combination of an ActRII polypeptide and a TβRII polypeptide.

### Functional Classes

Pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) at baseline can be mild, moderate or severe, as measured for example by World Health Organization (WHO) functional class, which is a measure of disease severity in patients with pulmonary hypertension. The WHO functional classification is an adaptation of the New York Heart Association (NYHA) system and is routinely used to qualitatively assess activity tolerance, for example in monitoring disease progression and response to treatment (Rubin (2004) Chest 126:7-10). Four functional classes are recognized in the WHO system: Functional Class I: pulmonary hypertension without resulting limitation of physical activity; ordinary physical activity does not cause undue dyspnea or fatigue, chest pain or near syncope; Functional Class II: pulmonary hypertension resulting in slight limitation of physical activity; patient comfortable at rest; ordinary physical activity causes undue dyspnea or fatigue, chest pain or near syncope; Functional Class III: pulmonary hypertension resulting in marked limitation of physical activity; patient comfortable at rest; less than ordinary activity causes undue dyspnea or fatigue, chest pain or near syncope; Functional Class IV: pulmonary hypertension resulting in inability to carry out any physical activity without symptoms; patient manifests signs of right-heart failure; dyspnea and/or fatigue may be present even at rest; discomfort is increased by any physical activity. In some embodiments of the methods disclosed herein, the method prevents or reduces pulmonary hypertension Functional Class progression as recognized by the World Health Organization (WHO). In some embodiments, the method prevents or reduces pulmonary hypertension Functional Class progression from Functional Class I to Class II pulmonary hypertension as recognized by the WHO. In some embodiments, the method prevents or reduces pulmonary hypertension Functional Class progression from Functional Class II to Class III pulmonary hypertension as recognized by the WHO. In some embodiments, the method prevents or reduces pulmonary hypertension Functional Class progression from Functional Class III to Class IV pulmonary hypertension as recognized by the WHO. In some embodiments, the method promotes or increases pulmonary hypertension Functional Class regression as recognized by the WHO. In some embodiments, the method promotes or increases pulmonary hypertension Functional Class regression from Class IV to Class III pulmonary hypertension as recognized by the WHO. In some embodiments, the method promotes or increases pulmonary hypertension Functional Class regression from Class III to Class II pulmonary hypertension as recognized by the WHO. In some embodiments, the method promotes or increases pulmonary hypertension Functional Class regression from Class II to Class I pulmonary hypertension as recognized by the WHO.

In some embodiments, the disclosure relates to methods of preventing or reducing pulmonary hypertension Functional Class progression comprising administering to a patient in need thereof a combination of an effective amount of an ActRII polypeptide (e.g., an amino acid sequence that is at least 90% identical to an amino acid sequence corresponding to residues 30-110 of SEQ ID NO: 1) and an effective amount of a TβRII polypeptide (e.g., an amino acid sequence that is at least 90% identical to an amino acid sequence corresponding to residues 23-184 of SEQ ID NO: 42). In some embodiments, the reduction in Functional Class progression is a delay in Functional Class progression. In some embodiments, the method relates to preventing or decreasing pulmonary hypertension functional class progression as recognized by the WHO. In some embodiments, the method relates to a patient that has Functional Class I pulmonary hypertension as recognized by the WHO. In some embodiments, the method relates to preventing or reducing patient progression from Functional Class I pulmonary hypertension to Functional Class II pulmonary hypertension as recognized by the WHO. In some embodiments, the method relates to a patient that has Functional Class II pulmonary hypertension as recognized by the WHO. In some embodiments, the method relates to preventing or reducing patient progression from Functional Class II pulmonary hypertension to Functional Class III pulmonary hypertension as recognized by the WHO. In some embodiments, the method relates to a patient that has Functional Class III pulmonary hypertension as recognized by the WHO. In some embodiments, the method relates to preventing or reducing patient progression from Functional Class III pulmonary hypertension to Functional Class IV pulmonary hypertension as recognized by the WHO.

In certain aspects, the disclosure relates to methods of promoting or increasing pulmonary hypertension Functional Class regression in a pulmonary hypertension associated with lung disease (e.g., pulmonary hypertension associated with chronic obstructive pulmonary disease (COPD), interstitial lung disease (ILD), or combined pulmonary fibrosis and emphysema (CPFE)) patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the patient has Functional Class I, Functional Class II, Functional Class III, or Functional Class IV pulmonary hypertension as recognized by the WHO. In some embodiments, the method relates to a patient that has Functional Class II pulmonary hypertension as recognized by the WHO. In some embodiments, the method relates to promoting patient regression from Functional Class II pulmonary hypertension to Functional Class I pulmonary hypertension as recognized by the WHO. In some embodiments, the method relates to a patient that has Functional Class III pulmonary hypertension as recognized by the WHO. In some embodiments, the method relates to promoting patient regression from Functional Class III pulmonary hypertension to Functional Class II pulmonary hypertension as recognized by the WHO. In some embodiments, the method relates to promoting patient regression from Functional Class III pulmonary hypertension to Functional Class I pulmonary hypertension as recognized by the WHO. In some embodiments, the method relates to a patient that has Functional Class IV pulmonary hypertension as recognized by the WHO. In some embodiments, the method relates to promoting patient regression from Functional Class IV pulmonary hypertension to Functional Class III pulmonary hypertension as recognized by the WHO. In some embodiments, the method relates to promoting patient regression from Functional Class IV pulmonary hypertension to Functional Class II pulmonary hypertension as recognized by the WHO. In some embodiments, the method relates to promoting patient regression from Functional Class IV pulmonary hypertension to Functional Class I pulmonary hypertension as recognized by the WHO.

In some embodiments, functional class regression is tested after the patient has received 4 weeks of treatment utilizing a combination of an ActRII polypeptide and a TβRII polypeptide disclosed herein. In some embodiments, functional class regression is tested after the patient has received 8 weeks of treatment utilizing a combination of an ActRII polypeptide and a TβRII polypeptide disclosed herein. In some embodiments, functional class regression is tested after the patient has received 12 weeks of treatment utilizing a combination of an ActRII polypeptide and a TβRII polypeptide disclosed herein. In some embodiments, functional class regression is tested after the patient has received 16 weeks of treatment utilizing a combination of an ActRII polypeptide and a TβRII polypeptide disclosed herein. In some embodiments, functional class regression is tested after the patient has received 20 weeks of treatment utilizing a combination of an ActRII polypeptide and a TβRII polypeptide disclosed herein. In some embodiments, functional class regression is tested after the patient has received 22 weeks of treatment utilizing a combination of an ActRII polypeptide and a TβRII polypeptide disclosed herein. In some embodiments, functional class regression is tested after the patient has received 24 weeks of treatment utilizing a combination of an ActRII polypeptide and a TβRII polypeptide disclosed herein. In some embodiments, functional class regression is tested after the patient has received 26 weeks of treatment utilizing a combination of an ActRII polypeptide and a TβRII polypeptide disclosed herein. In some embodiments, functional class regression is tested after the patient has received 28 weeks of treatment utilizing a combination of an ActRII polypeptide and a TβRII polypeptide disclosed herein. In some embodiments, functional class regression is tested after the patient has received 48 weeks of treatment utilizing an ActRII polypeptide disclosed herein.

### Sustained Therapeutic Effect

In certain aspects, the disclosure relates to methods of treating, preventing, or reducing the progression rate and/or severity of one or more complications of pulmonary hypertension associated with lung disease in a sustained manner, comprising administering to a patient in need thereof: 1) an effective amount of a polypeptide comprising an amino acid sequence that is at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 21, 22, 23, 24, 25, 26, 27, 28, 29, or 30 of SEQ ID NO: 1 and ends at any one of amino acids 110, 111, 112, 113, 114, 115, 116, 117, 118, 119, 120, 121, 122, 123, 124, 125, 126, 127, 128, 129, 130, 131, 132, 133, 134, or 135 of SEQ ID NO: 1; and 2) an effective amount of a TβRII polypeptide comprising an amino acid sequence at least 70%, 75%, 80%, 85%, 86%, 87%, 88%, 89%, 90%, 91%, 92%, 93%, 94%, 95%, 96%, 97%, 98%, 99%, or 100% identical to an amino acid sequence that begins at any one of amino acids 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 of SEQ ID NO: 42 and ends at any one of amino acids 178, 179, 180, 181, 182, 183, or 184 of SEQ ID NO: 42. In some embodiments, the sustained manner comprises a persistent therapeutic effect following the reduction in administration of a combination of an ActRII polypeptide and a TβRII polypeptide described herein. In some embodiments, the sustained manner comprises a persistent therapeutic effect following the withdrawal of administration of a combination of an ActRII polypeptide and a TβRII polypeptide described herein. In some embodiments, the persistent therapeutic effect relates to maintaining functional or hematologic measurements over time. In some embodiments, the persistent therapeutic effect is measured as a sustained reduction in PVR. In some embodiments, the patient's PVR level does not increase for at least 1 week to at least 12 weeks following withdrawal of a combination of an ActRII polypeptide and a TβRII polypeptide treatment described herein. In some embodiments, the patient's PVR level does not increase for at least 1 week following withdrawal of a combination of an ActRII polypeptide and a TβRII polypeptide treatment described herein. In some embodiments, the patient's PVR level does not increase for at least 2 weeks following withdrawal of a combination of an ActRII polypeptide and a TβRII polypeptide treatment described herein. In some embodiments, the patient's PVR level does not increase for at least 3 weeks following withdrawal of a combination of an ActRII polypeptide and a TβRII polypeptide treatment described herein. In some embodiments, the patient's PVR level does not increase for at least 4 weeks following withdrawal of a combination of an ActRII polypeptide and a TβRII polypeptide treatment described herein. In some embodiments, the patient's PVR level does not increase for at least 5 weeks following withdrawal of a combination of an ActRII polypeptide and a TβRII polypeptide treatment described herein. In some embodiments, the patient's PVR level does not increase for at least 6 weeks following withdrawal of a combination of an ActRII polypeptide and a TβRII polypeptide treatment described herein. In some embodiments, the patient's PVR level does not increase for at least 1 month to at least 6 months following withdrawal of a combination of an ActRII polypeptide and a TβRII polypeptide treatment described herein.

In certain aspects, the disclosure relates to methods of treating or preventing cardiopulmonary remodeling associated with pulmonary hypertension associated with lung disease (e.g., pulmonary hypertension associated with COPD, ILD, or CPFE) in a patient, comprising administering to a patient in need thereof a combination of an effective amount of an ActRIIA polypeptide and a TβRII polypeptide, wherein said method slows down cardiac remodeling and/or reverses cardiac remodeling. In some embodiments, the reversal is a sustained reversal. In some embodiments, the cardiac remodeling is ventricle remodeling. In some embodiments, the ventricle remodeling is left ventricular remodeling. In some embodiments, the ventricle remodeling is right ventricular remodeling. In some embodiments, the cardiac remodeling is ventricular dilation. In some embodiments, the method decreases interventricular septal end diastole. In some embodiments, the method decreases posterior wall end diastole.

In some embodiments, echocardiographic measurements may be used to assess the persistent therapeutic effect. In some embodiments, the echocardiographic measurements include, but are not limited to, RV fractional area change (RVFAC), sPAP, tricuspid annular systolic velocity (TASV), and Tei index. In some embodiments, a patient treated with an ActRIIA polypeptide disclosed herein shows a persistent therapeutic effect. In some embodiments, the persistent therapeutic effect results in decreased intrusion of the ventral wall into the left ventricle. In some embodiments, the persistent therapeutic effect results in an increase in RVFAC.

### Measuring Various Parameters Over Time

In certain embodiments, one or more of the measurements of pulmonary hypertension (e.g., pulmonary hypertension associated with lung disease (e.g., pulmonary hypertension associated with COPD, ILD, or CPFE) described herein can be measured over various periods of treatment time. In some embodiments, one or more of the measurements of pulmonary hypertension described herein is measured after the patient has received 4 weeks of treatment utilizing a combination of an ActRII polypeptide and a TβRII polypeptide disclosed herein. In some embodiments, one or more of the measurements of pulmonary hypertension described herein is measured after the patient has received 8 weeks of treatment utilizing a combination of an ActRII polypeptide and a TβRII polypeptide disclosed herein. In some embodiments, one or more of the measurements of pulmonary hypertension described herein is measured after the patient has received 12 weeks of treatment utilizing a combination of an ActRII polypeptide and a TβRII polypeptide disclosed herein. In some embodiments, one or more of the measurements of pulmonary hypertension described herein is measured after the patient has received 16 weeks of treatment utilizing a combination of an ActRII polypeptide and a TβRII polypeptide disclosed herein. In some embodiments, one or more of the measurements of pulmonary hypertension described herein is measured after the patient has received 20 weeks of treatment utilizing a combination of an ActRII polypeptide and a TβRII polypeptide disclosed herein. In some embodiments, one or more of the measurements of pulmonary hypertension described herein is measured after the patient has received 22 weeks of treatment utilizing a combination of an ActRII polypeptide and a TβRII polypeptide disclosed herein. In some embodiments, one or more of the measurements of pulmonary hypertension described herein is measured after the patient has received 24 weeks of treatment utilizing a combination of an ActRII polypeptide and a TβRII polypeptide disclosed herein. In some embodiments, one or more of the measurements of pulmonary hypertension described herein is measured after the patient has received 26 weeks of treatment utilizing a combination of an ActRII polypeptide and a TβRII polypeptide disclosed herein. In some embodiments, one or more of the measurements of pulmonary hypertension described herein is measured after the patient has received 28 weeks of treatment utilizing a combination of an ActRII polypeptide and a TβRII polypeptide disclosed herein. In some embodiments, one or more of the measurements of pulmonary hypertension described herein is measured after the patient has received 48 weeks of treatment utilizing a combination of an ActRII polypeptide and a TβRII polypeptide disclosed herein.

### 8. Pharmaceutical Compositions & Modes of Administration

In certain embodiments, the therapeutic methods of the disclosure include administering the composition systemically, or locally as an implant or device. When administered, the therapeutic composition for use in this disclosure is in a substantially pyrogen-free, or pyrogen-free, physiologically acceptable form. Therapeutically useful agents other than the ActRII polypeptides which may also optionally be included in the composition as described above, may be administered simultaneously or sequentially with the subject compounds in the methods disclosed herein.

Typically, protein therapeutic agents disclosed herein will be administered parentally, and particularly intravenously or subcutaneously. Pharmaceutical compositions suitable for parenteral administration may comprise one or more ActRII polypeptides or TβRII polypeptides in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents. Examples of suitable aqueous and nonaqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants. The formulations can be presented in unit-dose or multi-dose sealed containers, such as ampules and vials, and can be stored in a freeze-dried (lyophilized) condition requiring only the addition of a sterile liquid excipient, for example, water, for injections, immediately prior to use. Extemporaneous injection solutions and suspensions can be prepared from sterile powders, granules, and tablets of the kind described herein.

The compositions and formulations may, if desired, be presented in a pack or dispenser device which may contain one or more unit dosage forms containing the active ingredient. The pack may for example comprise metal or plastic foil, such as a blister pack. The pack or dispenser device may be accompanied by instructions for administration.

Further, the composition may be encapsulated or injected in a form for delivery to a target tissue site. In certain embodiments, compositions of the invention include a matrix capable of delivering one or more therapeutic compounds (e.g., ActRII polypeptides, TβRII polypeptides) to a target tissue site, providing a structure for the developing tissue and optimally capable of being resorbed into the body. For example, the matrix may provide slow release of the ActRII or TβRII polypeptide. Such matrices may be formed of materials presently in use for other implanted medical applications.

The choice of matrix material is based on biocompatibility, biodegradability, mechanical properties, cosmetic appearance and interface properties. The particular application of the subject compositions will define the appropriate formulation. Potential matrices for the compositions may be biodegradable and chemically defined calcium sulfate, tricalcium phosphate, hydroxyapatite, polylactic acid and polyanhydrides. Other potential materials are biodegradable and biologically well defined, such as bone or dermal collagen. Further matrices are comprised of pure proteins or extracellular matrix components. Other potential matrices are non-biodegradable and chemically defined, such as sintered hydroxyapatite, bioglass, aluminates, or other ceramics. Matrices may be comprised of combinations of any of the above mentioned types of material, such as polylactic acid and hydroxyapatite or collagen and tricalcium phosphate. The bioceramics may be altered in composition, such as in calcium-aluminate-phosphate and processing to alter pore size, particle size, particle shape, and biodegradability.

In certain embodiments of the methods of the invention, the ActRII polypeptides and/or the TβRII polypeptides are administered orally, e.g., in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of an agent as an active ingredient. An agent may also be administered as a bolus, electuary or paste.

In solid dosage forms for oral administration (capsules, tablets, pills, dragees, powders, granules, and the like), one or more therapeutic compounds of the invention may be mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose, and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration include pharmaceutically acceptable emulsions, microemulsions, solutions, suspensions, syrups, and elixirs. In addition to the active ingredient, the liquid dosage forms may contain inert diluents commonly used in the art, such as water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor, and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming, and preservative agents.

Suspensions, in addition to the active compounds, may contain suspending agents such as ethoxylated isostearyl alcohols, polyoxyethylene sorbitol, and sorbitan esters, microcrystalline cellulose, aluminum metahydroxide, bentonite, agar-agar and tragacanth, and mixtures thereof.

The compositions of the invention may also contain adjuvants, such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption, such as aluminum monostearate and gelatin.

It is understood that the dosage regimen will be determined by the attending physician considering various factors which modify the action of the subject compounds of the disclosure (e.g., ActRII polypeptides). The various factors include, but are not limited to, the patient's age, sex, and diet, the severity disease, time of administration, and other clinical factors. Optionally, the dosage may vary with the type of matrix used in the reconstitution and the types of compounds in the composition. In some embodiments, a patient's hematologic parameters can be monitored by periodic assessments in order to determine if they have higher than normal red blood cell levels and/or hemoglobin levels (e.g., hemoglobin levels>16.0 g/dL or hemoglobin levels>18.0 g/dL). In some embodiments, patient's having higher than normal red blood cell levels and/or hemoglobin levels may receive a delayed or reduced dose until the levels have returned to a normal or acceptable level.

The probability of a patient having hemoglobin levels greater than 18 g/dL or increases in hemoglobin of greater than 2 g/dL may be higher during initial treatment with an ActRII polypeptide. In certain embodiments, a dosing regimen can be used to prevent, ameliorate, or decrease the adverse changes in hemoglobin levels. In some embodiments, ActRII polypeptides of the disclosure are administered using a dosing regimen. In some embodiments, the method comprises administering a dosing regimen of a therapeutically effective amount of an ActRII polypeptide as disclosed herein to a patient, comprising a first dose of between 0.1 mg/kg and 1.0 mg/kg of said polypeptide for a first period of time, and a second dose of between 0.1 mg/kg and 1.0 mg/kg of said polypeptide subsequently administered for a second period of time. In some embodiments, the method comprises administering a dosing regimen of therapeutically effective amount of an ActRII polypeptide as disclosed herein to a patient, comprising a first dose of between 0.1 mg/kg and 1.0 mg/kg of said polypeptide for a first period of time, a second dose of between 0.1 mg/kg and 1.0 mg/kg of said polypeptide administered for a second period of time, and a third dose of between 0.1 mg/kg and 1.0 mg/kg of said polypeptide subsequently administered for a third period of time. In some embodiments, the first dose of ActRII polypeptide is administered to a patient in an amount from about 0.2 mg/kg to about 0.4 mg/kg. In some embodiments, the first dose of ActRII polypeptide is administered to a patient at a dose of 0.3 mg/kg. In some embodiments, the second dose of ActRII polypeptide is administered to a patient in an amount from about 0.5 mg/kg to about 0.8 mg/kg. In some embodiments, the second dose of ActRII polypeptide is administered to a patient at a dose of 0.7 mg/kg. In some embodiments, the third dose of ActRII polypeptide is administered to a patient in an amount from about 0.2 mg/kg to about 0.4 mg/kg. In some embodiments, the third dose of ActRII polypeptide is administered to a patient at a dose of 0.3 mg/kg.

In some embodiments, the dosing regimen comprises administering a first dose of ActRII polypeptide to a patient in an amount of 0.3 mg/kg followed by administration of a second dose of ActRII polypeptide to the patient in an amount of 0.7 mg/kg. In some embodiments, the dosing regimen comprises administering a first dose of ActRI1 polypeptide to a patient in an amount of 0.3 mg/kg, administering a second dose of ActRII polypeptide to the patient in an amount of 0.7 mg/kg, and administering a third dose of ActRII polypeptide to the patient in an amount of 0.3 mg/kg. In some embodiments, the second dose exceeds the first dose. In some embodiments, the first dose exceeds the second dose. In some embodiments, the third dose exceeds the second dose. In some embodiments, the second dose exceeds the third dose. In some embodiments, the first period of time is at least 3 weeks. In some embodiments, the second period of time is at least 3 weeks. In some embodiments, the third period of time is at least 3 weeks. In some embodiments, the second period of time is at least 21 weeks. In some embodiments, the second period of time is at least 45 weeks. In some embodiments, the second period of time exceeds the first period of time. In some embodiments, the third period of time exceeds the first period of time. In some embodiments, the third period of time exceeds the second period of time.

In some embodiments, the change in dosing between the first dose and the second dose is determined by the attending physician considering various factors (e.g., hemoglobin levels). In some embodiments, the change in dosing between the second dose and the third dose is determined by the attending physician considering various factors (e.g., hemoglobin levels). In some embodiments, the various factors include, but are not limited to, the patient's change in hematologic parameters over a period of time. In some embodiments, a patient's hematologic parameters are monitored in order to determine if they have higher than normal red blood cell levels and/or hemoglobin levels (e.g., hemoglobin levels>16.0 g/dL or hemoglobin levels>18.0 g/dL). In some embodiments, a patient's hematologic parameters are monitored in order to determine if they have a higher than normal increase in hemoglobin levels over a period of time (e.g., hemoglobin level increase of >2 g/dL in less than 3 weeks). In some embodiments, the patient's dose of an ActRII polypeptide as disclosed herein will be decreased (e.g., decrease in dose from 0.7 mg/kg to 0.3 mg/kg) if one or more of the patient's hematologic parameters before or during treatment is abnormal. In some embodiments, the patient's dose of an ActRII polypeptide as disclosed herein will be maintained (e.g., maintained at 0.3 mg/kg or 0.7 mg/kg) if one or more of the patient's hematologic parameters before or during treatment is abnormal.

In some embodiments, the dosing regimen prevents, ameliorates, or decreases adverse effects of the ActRII polypeptide. In some embodiments, administration of an ActRII polypeptide in accordance with the dosage regimen as provided herein results in decreased adverse side effects. In some embodiments, administration of an ActRII polypeptide in accordance with the dosage regimen as provided herein decreases the probability of having hemoglobin levels greater than 18 g/dL during the first period of time. In some embodiments, administration of an ActRII polypeptide in accordance with the dosage regimen as provided herein decreases the probability of having hemoglobin levels greater than 18 g/dL in the first 3 weeks of treatment. In some embodiments, administration of an ActRII polypeptide in accordance with the dosage regimen as provided herein decreases the probability of increasing hemoglobin levels by greater than 2 g/dL during the first period of time. In some embodiments, administration of an ActRII polypeptide in accordance with the dosage regimen as provided herein decreases the probability of increasing hemoglobin levels by greater than 2 g/dL in the first 3 weeks of treatment.

In some embodiments, ActRII polypeptides of the disclosure are administered at a dosing range of 0.1 mg/kg to 2.0 mg/kg. In some embodiments, ActRII polypeptides of the disclosure are administered at 0.1 mg/kg. In some embodiments, ActRII polypeptides of the disclosure are administered at 0.2 mg/kg. In some embodiments, ActRII polypeptides of the disclosure are administered at 0.3 mg/kg. In some embodiments, ActRII polypeptides of the disclosure are administered at 0.4 mg/kg. In some embodiments, ActRII polypeptides of the disclosure are administered at 0.5 mg/kg. In some embodiments, ActRII polypeptides of the disclosure are administered at 0.6 mg/kg. In some embodiments, ActRII polypeptides of the disclosure are administered at 0.7 mg/kg. In some embodiments, ActRII polypeptides of the disclosure are administered at 0.8 mg/kg. In some embodiments, ActRII polypeptides of the disclosure are administered at 0.9 mg/kg. In some embodiments, ActRII polypeptides of the disclosure are administered at 1.0 mg/kg. In some embodiments, ActRII polypeptides of the disclosure are administered at 1.1 mg/kg. In some embodiments, ActRII polypeptides of the disclosure are administered at 1.2 mg/kg. In some embodiments, ActRII polypeptides of the disclosure are administered at 1.3 mg/kg. In some embodiments, ActRII polypeptides of the disclosure are administered at 1.4 mg/kg. In some embodiments, ActRII polypeptides of the disclosure are administered at 1.5 mg/kg. In some embodiments, ActRII polypeptides of the disclosure are administered at 1.6 mg/kg. In some embodiments, ActRII polypeptides of the disclosure are administered at 1.7 mg/kg. In some embodiments, ActRII polypeptides of the disclosure are administered at 1.8 mg/kg. In some embodiments, ActRII polypeptides of the disclosure are administered at 1.9 mg/kg. In some embodiments, ActRII polypeptides of the disclosure are administered at 2.0 mg/kg.

In certain embodiments, ActRII polypeptides of the disclosure are administered once a day. In certain embodiments, ActRII polypeptides of the disclosure are administered twice a day. In certain embodiments, ActRII polypeptides of the disclosure are administered once a week. In certain embodiments, ActRII polypeptides of the disclosure are administered twice a week. In certain embodiments, ActRII polypeptides of the disclosure are administered three times a week. In certain embodiments, ActRII polypeptides of the disclosure are administered every two weeks. In certain embodiments, ActRII polypeptides of the disclosure are administered every three weeks. In certain embodiments, ActRII polypeptides of the disclosure are administered every four weeks. In certain embodiments, ActRII polypeptides of the disclosure are administered every month.

In some embodiments, TβRII polypeptides of the disclosure are administered at a dosing range of 0.1 mg/kg to 2.0 mg/kg. In some embodiments, TβRII polypeptides of the disclosure are administered at 0.1 mg/kg. In some embodiments, TβRII polypeptides of the disclosure are administered at 0.2 mg/kg. In some embodiments, TβRII polypeptides of the disclosure are administered at 0.3 mg/kg. In some embodiments, TβRII polypeptides of the disclosure are administered at 0.4 mg/kg. In some embodiments, TβRII polypeptides of the disclosure are administered at 0.5 mg/kg. In some embodiments, TβRII polypeptides of the disclosure are administered at 0.6 mg/kg. In some embodiments, TβRII polypeptides of the disclosure are administered at 0.7 mg/kg. In some embodiments, TβRII polypeptides of the disclosure are administered at 0.8 mg/kg. In some embodiments, TβRII polypeptides of the disclosure are administered at 0.9 mg/kg. In some embodiments, TβRII polypeptides of the disclosure are administered at 1.0 mg/kg. In some embodiments, TβRII polypeptides of the disclosure are administered at 1.1 mg/kg. In some embodiments, TβRII polypeptides of the disclosure are administered at 1.2 mg/kg. In some embodiments, TβRII polypeptides of the disclosure are administered at 1.3 mg/kg. In some embodiments, TβRII polypeptides of the disclosure are administered at 1.4 mg/kg. In some embodiments, TβRII polypeptides of the disclosure are administered at 1.5 mg/kg. In some embodiments, TβRII polypeptides of the disclosure are administered at 1.6 mg/kg. In some embodiments, TβRII polypeptides of the disclosure are administered at 1.7 mg/kg. In some embodiments, TβRII polypeptides of the disclosure are administered at 1.8 mg/kg. In some embodiments, TβRII polypeptides of the disclosure are administered at 1.9 mg/kg. In some embodiments, TβRII polypeptides of the disclosure are administered at 2.0 mg/kg.

In certain embodiments, TβRII polypeptides of the disclosure are administered once a day. In certain embodiments, TβRII polypeptides of the disclosure are administered twice a day. In certain embodiments, TβRII polypeptides of the disclosure are administered once a week. In certain embodiments, TβRII polypeptides of the disclosure are administered twice a week. In certain embodiments, TβRII polypeptides of the disclosure are administered three times a week. In certain embodiments, TβRII polypeptides of the disclosure are administered every two weeks or about every two weeks. In certain embodiments, TβRII polypeptides of the disclosure are administered every three weeks or about every three weeks. In certain embodiments, TβRII polypeptides of the disclosure are administered every four weeks or about every four weeks. In certain embodiments, TβRII polypeptides of the disclosure are administered every month i.e., once per month during the treatment period.

In certain embodiments, the invention also provides gene therapy for the in vivo production of ActRII polypeptides and TβRII polypeptides. Such therapy would achieve its therapeutic effect by introduction of the ActRII polypeptide polynucleotide sequences or TβRII polypeptide polynucleotide sequences into cells or tissues having the disorders as listed above. Delivery of ActRII polypeptide polynucleotide sequences and TβRII polypeptide polynucleotide sequences can be achieved using a recombinant expression vector such as a chimeric virus or a colloidal dispersion system. Targeted liposomes may be used for therapeutic delivery of ActRII polypeptide polynucleotide sequences and TβRII polypeptide polynucleotide sequences.

Various viral vectors which can be utilized for gene therapy as taught herein include adenovirus, herpes virus, vaccinia, or, preferably, an RNA virus such as a retrovirus. Preferably, the retroviral vector is a derivative of a murine or avian retrovirus. Examples of retroviral vectors in which a single foreign gene can be inserted include, but are not limited to: Moloney murine leukemia virus (MoMuLV), Harvey murine sarcoma virus (HaMuSV), murine mammary tumor virus (MuMTV), and Rous Sarcoma Virus (RSV). A number of additional retroviral vectors can incorporate multiple genes. All of these vectors can transfer or incorporate a gene for a selectable marker so that transduced cells can be identified and generated. Retroviral vectors can be made target-specific by attaching, for example, a sugar, a glycolipid, or a protein. Targeting may be accomplished by using an antibody. Those of skill in the art will recognize that specific polynucleotide sequences can be inserted into the retroviral genome or attached to a viral envelope to allow target-specific delivery of the retroviral vector containing the ActRII polypeptide or the TβRII polypeptide. In one embodiment, the vector is targeted to bone or cartilage.

Alternatively, tissue culture cells can be directly transfected with plasmids encoding the retroviral structural genes gag, pol and env, by conventional calcium phosphate transfection. These cells are then transfected with the vector plasmid containing the genes of interest. The resulting cells release the retroviral vector into the culture medium.

Another targeted delivery system for ActRII polypeptide polynucleotides and TβRII polypeptide polynucleotides is a colloidal dispersion system. Colloidal dispersion systems include macromolecule complexes, nanocapsules, microspheres, beads, and lipid-based systems including oil-in-water emulsions, micelles, mixed micelles, and liposomes. A colloidal system useful for the invention is a liposome. Liposomes are artificial membrane vesicles which are useful as delivery vehicles in vitro and in vivo. RNA, DNA and intact virions can be encapsulated within the aqueous interior and be delivered to cells in a biologically active form (see e.g., Fraley, et al., Trends Biochem. Sci., 6:77, 1981). Methods for efficient gene transfer using a liposome vehicle, are known in the art, see e.g., Mannino, et al., Biotechniques, 6:682, 1988. The composition of the liposome is usually a combination of phospholipids, usually in combination with steroids, especially cholesterol. Other phospholipids or other lipids may also be used. The physical characteristics of liposomes depend on pH, ionic strength, and the presence of divalent cations.

Examples of lipids useful in liposome production include phosphatidyl compounds, such as phosphatidylglycerol, phosphatidylcholine, phosphatidylserine, phosphatidylethanolamine, sphingolipids, cerebrosides, and gangliosides. Illustrative phospholipids include egg phosphatidylcholine, dipalmitoylphosphatidylcholine, and distearoylphosphatidylcholine. The targeting of liposomes is also possible based on, for example, organ-specificity, cell-specificity, and organelle-specificity and is known in the art.

The disclosure provides formulations that may be varied to include acids and bases to adjust the pH; and buffering agents to keep the pH within a narrow range.

### 9. Exemplification

The disclosure above will be more readily understood by reference to the following examples, which are included merely for purposes of illustration of certain embodiments of the invention, and are not intended to limiting.

### Example 1: ActRIIA-Fc Fusion Proteins

A soluble ActRIIA fusion protein was constructed that has the extracellular domain of human ActRIIA fused to a human or mouse Fc domain with a minimal linker in between. The constructs are referred to as ActRIIA-hFec and ActRIIA-mFc, respectively.

ActRIIA-hFc is shown below as purified from CHO cell lines (SEQ ID NO: 23):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPGK

An additional ActRIIA-hFc lacking the C-terminal lysine is shown below as purified from CHO cell lines (SEQ ID NO: 41):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
EVTQPTSNPVTPKPPTGGGTHTCPPCPAPELLGGPSVFLFPPKPKDTLMI
SRTPEVTCVVVDVSHEDPEVKFNWYVDGVEVHNAKTKPREEQYNSTYRVV
SVLTVLHQDWLNGKEYKCKVSNKALPVPIEKTISKAKGQPREPQVYTLPP
SREEMTKNQVSLTCLVKGFYPSDIAVEWESNGQPENNYKTTPPVLDSDGS
FFLYSKLTVDKSRWQQGNVFSCSVMHEALHNHYTQKSLSLSPG

The ActRIIA-hFc and ActRIIA-mFc proteins were expressed in CHO cell lines. Three different leader sequences were considered:

(i) Honey bee melittin (HBML):
(SEQ ID NO: 24)
MKFLVNVALVFMVVYISYIYA
(ii) Tissue plasminogen activator (TPA):
(SEQ ID NO: 25)
MDAMKRGLCCVLLLCGAVFVSP
(iii) Native:
(SEQ ID NO: 26)
MGAAAKLAFAVFLISCSSGA

The selected form employs the TPA leader and has the following unprocessed amino acid sequence:

(SEQ ID NO: 27)
MDAMKRGLCCVLLLCGAVFVSPGAAILGRSETQECLFFNANWEKDRTNQT
GVEPCYGDKDKRRHCFATWKNISGSIEIVKQGCWLDDINCYDRTDCVEKK
DSPEVYFCCCEGNMCNEKFSYFPEMEVTQPTSNPVTPKPPTGGGTHTCPP
CPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKAL
PVPIEKTISKAKGQPREPQVYTLPPSREEMTKNQVSLTCLVKGFYPSDIA
VEWESNGQPENNYKTTPPVLDSDGSFFLYSKLTVDKSRWQQGNVFSCSVM
HEALHNHYTQKSLSLSPGK

This polypeptide is encoded by the following nucleic acid sequence:

(SEQ ID NO: 28)
ATGGATGCAATGAAGAGAGGGCTCTGCTGTGTGCTGCTGCTGTGTGGAGC
AGTCTTCGTTTCGCCCGGCGCCGCTATACTTGGTAGATCAGAAACTCAGG
AGTGTCTTTTTTTAATGCTAATTGGGAAAAAGACAGAACCAATCAAACTG
GTGTTGAACCGTGTTATGGTGACAAAGATAAACGGCGGCATTGTTTTGCT
ACCTGGAAGAATATTTCTGGTTCCATTGAATAGTGAAACAAGGTTGTTGG
CTGGATGATATCAACTGCTATGACAGGACTGATTGTGTAGAAAAAAAAGA
CAGCCCTGAAGTATATTTCTGTTGCTGTGAGGGCAATATGTGTAATGAAA
AGTTTTCTTATTTTCCGGAGATGGAAGTCACACAGCCCACTTCAAATCCA
GTTACACCTAAGCCACCCACCGGTGGTGGAACTCACACATGCCCACCGTG
CCCAGCACCTGAACTCCTGGGGGGACCGTCAGTCTTCCTCTTCCCCCCAA
AACCCAAGGACACCCTCATGATCTCCCGGACCCCTGAGGTCACATGCGTG
GTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGT
ACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCC
AGTCCCCATCGAGAAAACCATCTCCAAAGCCAAAGGGCAGCCCCGAGAAC
CACAGGTGTACACCCTGCCCCCATCCCGGGAGGAGATGACCAAGAACCAG
GTCAGCCTGACCTGCCTGGTCAAAGGCTTCTATCCCAGCGACATCGCCGT
GGAGTGGGAGAGCAATGGGCAGCCGGAGAACAACTACAAGACCACGCCTC
CCGTGCTGGACTCCGACGGCTCCTTCTTCCTCTATAGCAAGCTCACCGTG
GACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGG
GTAAATGAGAATTC

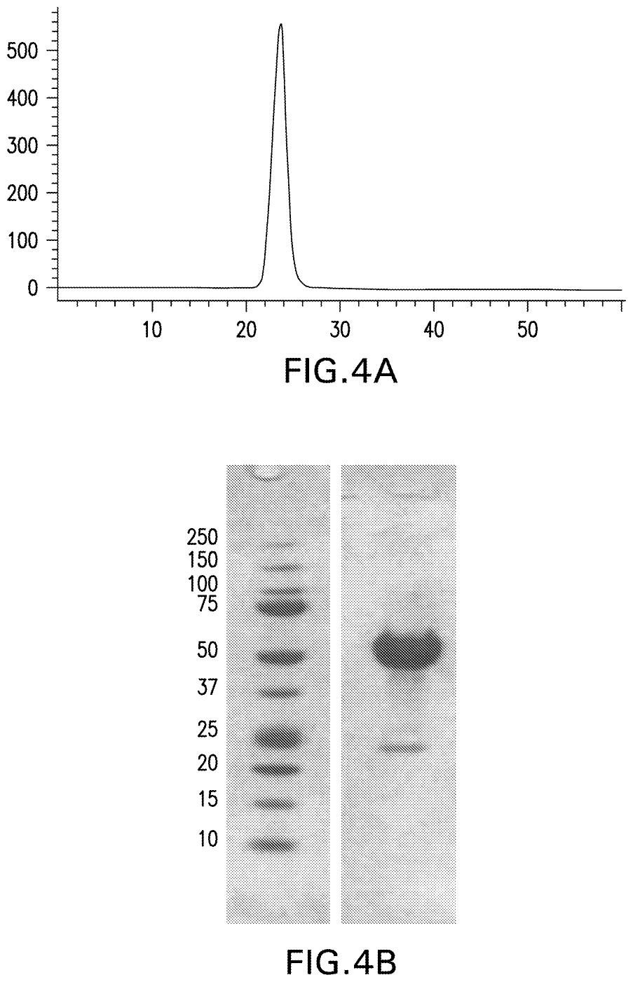

Both ActRIIA-hFc and ActRIIA-mFc were remarkably amenable to recombinant expression. As shown in FIGS. 4A and 4B, the protein was purified as a single, well-defined peak of protein. N-terminal sequencing revealed a single sequence of —ILGRSETQE (SEQ ID NO: 29). Purification could be achieved by a series of column chromatography steps, including, for example, three or more of the following, in any order: protein A chromatography, Q sepharose chromatography, phenylsepharose chromatography, size exclusion chromatography, and cation exchange chromatography. The purification could be completed with viral filtration and buffer exchange. The ActRIIA-hFc protein was purified to a purity of >98% as determined by size exclusion chromatography and >95% as determined by SDS PAGE.

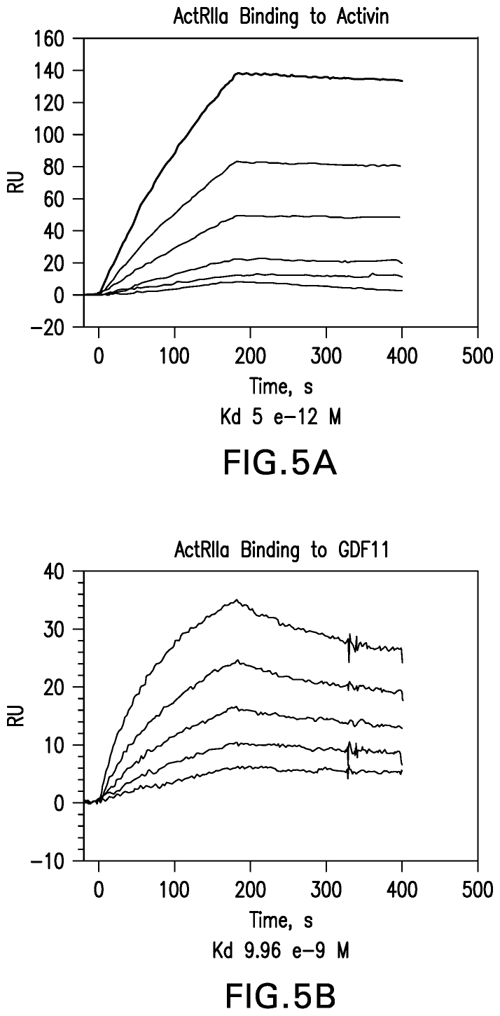

ActRIIA-hFc and ActRIIA-mFc showed a high affinity for ligands. GDF11 or activin A were immobilized on a Biacore™ CM5 chip using standard amine-coupling procedure. ActRIIA-hFc and ActRIIA-mFc proteins were loaded onto the system, and binding was measured. ActRIIA-hFc bound to activin with a dissociation constant (K_{D}) of 5×10⁻¹² and bound to GDF11 with a K_{D} of 9.96×10⁻⁹. See FIGS. 5A and 5B. Using a similar binding assay, ActRIIA-hFc was determined to have high to moderate affinity for other TGF-beta superfamily ligands including, for example, activin B, GDF8, BMP6, and BMP10. ActRIIA-mFc behaved similarly.

The ActRIIA-hFc was very stable in pharmacokinetic studies. Rats were dosed with 1 mg/kg, 3 mg/kg, or 10 mg/kg of ActRIIA-hFc protein, and plasma levels of the protein were measured at 24, 48, 72, 144 and 168 hours. In a separate study, rats were dosed at 1 mg/kg, 10 mg/kg, or 30 mg/kg. In rats, ActRIIA-hFc had an 11-14 day serum half-life, and circulating levels of the drug were quite high after two weeks (11 μg/ml, 110 μg/ml, or 304 μg/ml for initial administrations of 1 mg/kg, 10 mg/kg, or 30 mg/kg, respectively.) In cynomolgus monkeys, the plasma half-life was substantially greater than 14 days, and circulating levels of the drug were 25 μg/ml, 304 μg/ml, or 1440 μg/ml for initial administrations of 1 mg/kg, 10 mg/kg, or 30 mg/kg, respectively.

### Example 2: Characterization of an ActRIIA-hFc Protein

ActRIIA-hFc fusion protein was expressed in stably transfected CHO-DUKX B11 cells from a pAID4 vector (SV40 ori/enhancer, CMV promoter), using a tissue plasminogen leader sequence of SEQ ID NO: 25. The protein, purified as described above in Example 1, had a sequence of SEQ ID NO: 23. The Fc portion is a human IgG1 Fc sequence, as shown in SEQ ID NO: 23. Protein analysis reveals that the ActRIIA-hFc fusion protein is formed as a homodimer with disulfide bonding.

The CHO-cell-expressed material has a higher affinity for activin B ligand than that reported for an ActRIIA-hFc fusion protein expressed in human 293 cells [see, del Re et al. (2004) J Biol Chem. 279(51):53126-53135]. Additionally, the use of the TPA leader sequence provided greater production than other leader sequences and, unlike ActRIIA-Fc expressed with a native leader, provided a highly pure N-terminal sequence. Use of the native leader sequence resulted in two major species of ActRIIA-Fc, each having a different N-terminal sequence.

### Example 3: Alternative ActRIIA-Fc Proteins

A variety of ActRIIA variants that may be used according to the methods described herein are described in the International Patent Application published as WO2006/012627 (see e.g., pp. 55-58), incorporated herein by reference in its entirety. An alternative construct may have a deletion of the C-terminal tail (the final 15 amino acids of the extracellular domain of ActRIIA. The sequence for such a construct is presented below (Fc portion underlined) (SEQ ID NO: 30):

ILGRSETQECLFFNANWEKDRTNQTGVEPCYGDKDKRRHCFATWKNISGS
IEIVKQGCWLDDINCYDRTDCVEKKDSPEVYFCCCEGNMCNEKFSYFPEM
TGGGTHTCPPCPAPELLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPVPIEKTISKAKGQPREPQVYTLPPSREEMTKNQVSLTCL
VKGFYPSDIAVEWESNGQPENNYKTTPPVLDSDGSFFLYSKLTVDKSRWQ
QGNVFSCSVMHEALHNHYTOKSLSLSPGK

### Example 4: Effects of an ActRIIA-mFc on Group 3 Pulmonary Hypertension in Two Bleomycin-Induced Pulmonary Hypertension and Fibrosis Rat Model

The effects of an ActRIIA-mFc fusion protein (ActRIIA-mFc homodimer as described in Example 1) was examined in two rat models of Group 3 pulmonary hypertension (Grp3-PH) [Xiong et al., Hypertension 71(1):34-55 (2018); Schroll et al., Respir Physiol Neurobiol 170(1):32-36 (2010)].

In one model, twelve Wistar male rats were intratracheally administered with a single dose of bleomycin (Bleo, 0.6 U/rat) at day 0 and randomized into two treatment groups (6 rats per group): 1) treatment with monocrotaline (MCT, 60 mg/kg administered s.c. as a single dose at day 7 of study) and Tris buffered saline (s.c. as 1 ml/kg every three days) (vehicle treatment group), 2) treatment with MCT (60 mg/kg administered s.c. as a single dose at day 7 of study) and ActRIIA-mFc (5 mg/kg administered s.c. every three days). Rats were treated for 35 days. Body weights were recorded weekly throughout the study.

On day 42, rats were anesthetized with ˜3-4% isoflurane and placed on controlled heating pads. Right ventricular systolic pressure (RVSP) were measured by advancing a 2F curve tip pressure transducer catheter, (SPR-513, Millar Instruments) into the right ventricle (RV) via right jugular vein under ˜1.5-2% isofluorane anesthesia. RV hypertrophy was assessed by taking the weight ratio of RV free wall and LV+Septum (RV/LV+S, Fulton's Index). Lungs were collected, fixed in 10% formalin, embedded in paraffin, and sectioned for Masson's trichrome stain to assess fibrosis.

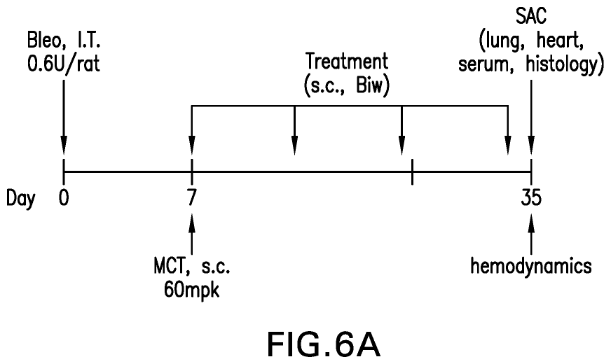

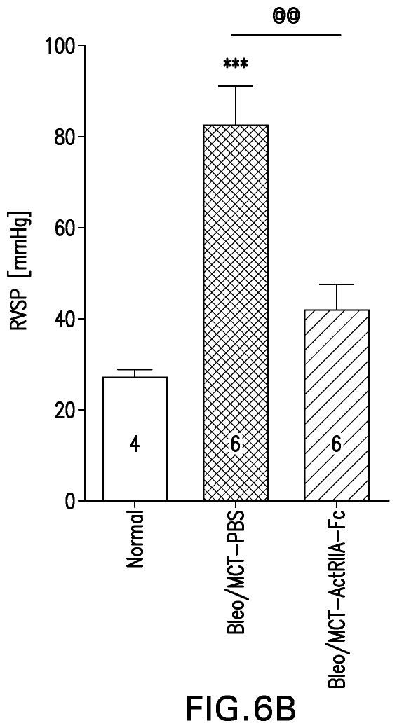

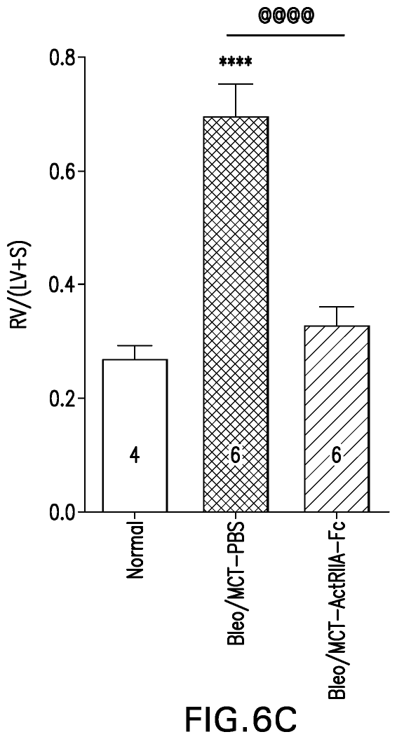

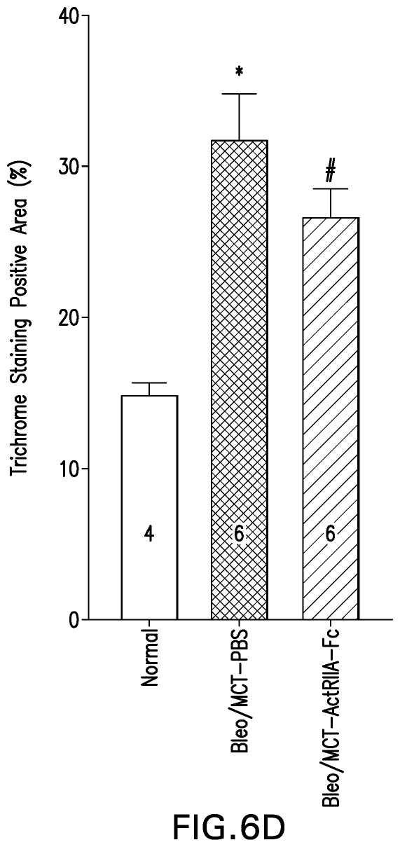

The effect of ActRIIA-mFc treatment of pulmonary hypertension and RV hypertrophy in Bleo-MCT PH-ILD rat model is shown in FIGS. 6A-6D. As shown in FIGS. 6B and 6C, Bleo-MCT treated rats (Bleo/MCT-PBS group) were observed to have elevated RVSP and right heart hypertrophy, indicating establishment of pulmonary hypertension and RV remodeling, compared to control animals. In addition, increased lung fibrosis was observed in Bleo-MCT rats (FIG. 6D). ActRIIA-mFc treatment significantly reduced increased RVSP (73%) and cardiac hypertrophy (87%). ActRIIA-mFc treatment also displayed a trend of decrease in lung fibrosis.

In another model, six Sprague-Dawley male rats were intratracheally administered with a single dose ofbleomycin (Bleo, 0.6 U/rat) at day 0 and randomized into two treatment groups: 1) treatment with semaxanib (20 mg/kg administered s.c. as a single dose at day 7 of study)/hypoxia and Tris buffered saline (administered s.c. as 1 ml/kg, every three days) (Bleo/Su/Hx-PBS group), 2) treatment with semaxanib (20 mg/kg administered s.c. as a single dose at day 7 of study)/hypoxia and ActRIIA-mFc (5 mg/kg administered s.c. every three days). Rats were treated for 35 days. Body weights were recorded weekly throughout the study.

On day 42, rats were anesthetized with ˜3-4% isoflurane and placed on controlled heating pads. Right ventricular systolic pressure (RVSP) was measured by advancing a 2F curve tip pressure transducer catheter, (SPR-513, Millar Instruments) into the right ventricle (RV) via right jugular vein under ˜1.5-2% isofluorane anesthesia. RV hypertrophy was assessed by taking the weight ratio of RV free wall and LV+Septum (RV/LV+S, Fulton's Index).

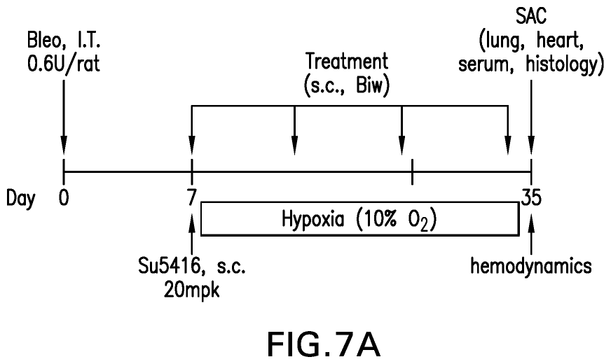

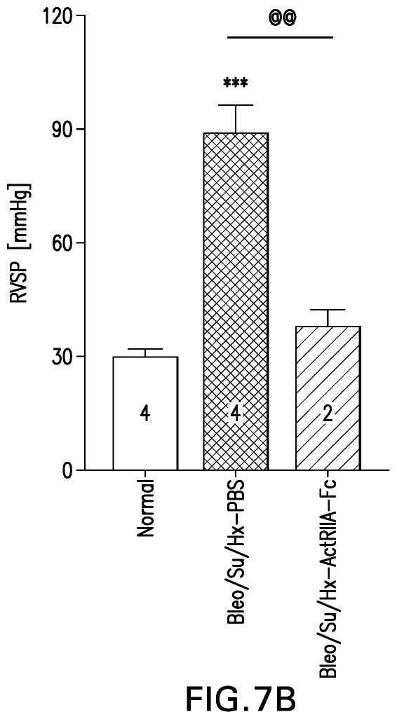

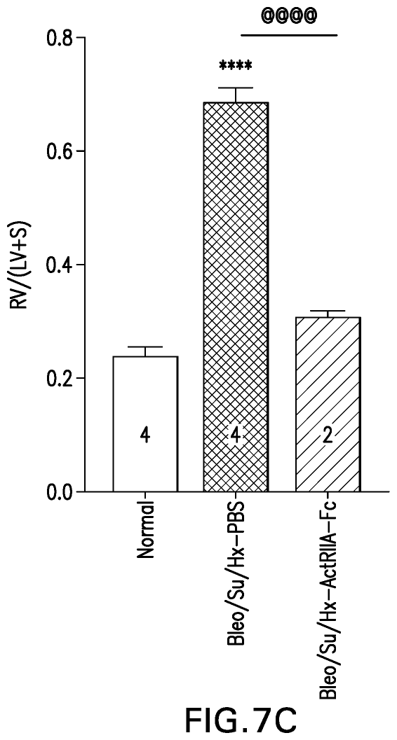

As shown in FIGS. 7A through 7C, Bleo-MCT treated rats (Bleo/Su/Hx-PBS group) were observed to have elevated RVSP and right heart hypertrophy, indicating establishment of pulmonary hypertension and RV remodeling, compared to control animals. ActRIIA-mFc treatment significantly reduced increased RVSP (87%) and cardiac hypertrophy (84%).

Together, these data demonstrate that ActRIIA-mFc is effective in ameliorating pulmonary hypertension in two bleomycin-induced Group 3 pulmonary hypertension models. In particular, ActRIIA-mFc had a significant effect in reducing RVSP and right heart hypertrophy. Furthermore, the data indicate that ActRIIA polypeptides, may be useful in the treatment of Group 3 PH, particularly in preventing or reducing the severity various complications of Group 3 PH.

### Example 5: TβRII Fusion Proteins

### TβRII ECD Variants

TβRII fusion proteins comprising a soluble extracellular portion of human TβRII and a human Fc portion were generated. For each fusion protein, a TβRII amino acid sequence having the amino acid sequence of SEQ ID NO: 46 was fused to an IgG Fc portion having the amino acid sequence of SEQ ID NO: 11 by means of one of several different linkers. Each of the fusion proteins also included a TPA leader sequence having the amino acid sequence of SEQ ID NO: 25 (below).

Tissue plasminogen activator (TPA):
(SEQ ID NO: 25)
MDAMKRGLCCVLLLCGAVEVSP

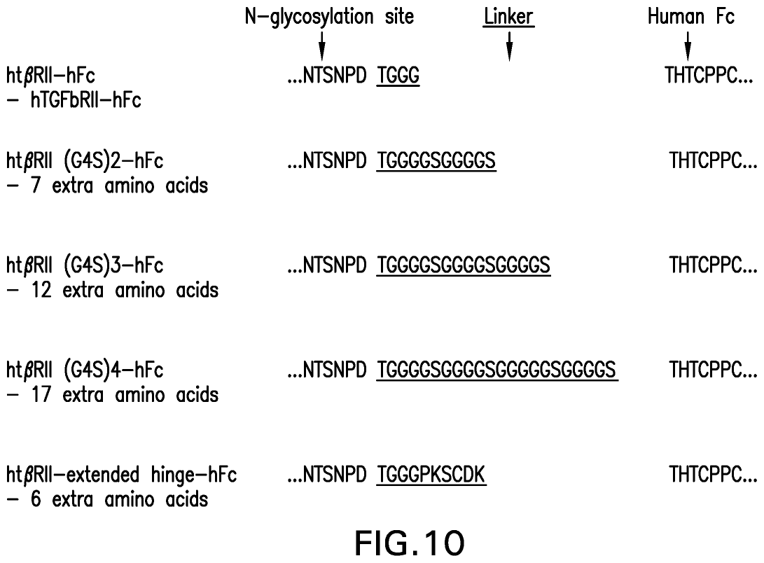

An illustration summary of several of the constructs designed is provided as FIG. 10. A table detailing the sequences for the different constructs tested in the Exemplification section is provided below:

| | Construct | |
|---|---|---|
| Construct | Amino Acid | |
| Name | Sequence | Linker Sequence |
| hTβRII-hFc | SEQ ID NO: | TGGG (SEQ ID NO: 20) |
| | 142 or 143 | |
| hTβRII (G4S)2- | SEQ ID NO: | TGGGGSGGGGS (SEQ ID |
| hFc | 73 or 77 | NO: 80) |
| hTβRII (G4S)3- | SEQ ID NO: | TGGGGSGGGGSGGGGS (SEQ |
| hFc | 71 or 75 | ID NO: 81) |
| hTβRII (G4S)4- | SEQ ID NO: | TGGGGSGGGGSGGGGSGGGGS |
| hFc | 72 or 76 | (SEQ ID NO: 82) |
| hTβRII | SEQ ID NO: | TGGGPKSCDK (SEQ ID NO: |
| extended hinge- | 74 or 78 | 86) |
| hFc | | |
| hTβRII (G4S)5- | SEQ ID NO: | TGGGGSGGGGSGGGGSGGGGS |
| hFc | 144 or 145 | GGGGS (SEQ ID NO: 83) |
| hTβRII (G4S)6- | SEQ ID NO: | TGGGGSGGGGSGGGGSGGGGS |
| hFc | 146 or 147 | GGGGSGGGGS (SEQ ID NO: |
| | | 84) |

The amino acid sequences for the construct components and each of the constructs, along with the nucleic acid sequence used to express these constructs, are provided below.

### TβRII Portion: Amino Acid Sequence

(SEQ ID NO: 46)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEYNTSN PD |

### Fc Portion: Amino Acid Sequence

(SEQ ID NO: 11)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WLNGKEYKCK |
| 101 | VSNKALPAPI EKTISKAKGQ PREPQVYTLP PSREEMTKNQ |
| | VSLTCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPGK |

The C-terminal lysine residue of the Fc domain can be deleted. The amino acid sequence of SEQ ID NO: 11 may optionally be provided with the lysine removed from the C-terminus (SEQ ID NO: 148)

(SEQ ID NO: 148)

| | |
|---|---|
| 1 | THTCPPCPAP ELLGGPSVFL FPPKPKDTLM ISRTPEVTCV |
| | VVDVSHEDPE |
| 51 | VKFNWYVDGV EVHNAKTKPR EEQYNSTYRV VSVLTVLHQD |
| | WINGKEYKCK |
| 101 | VSNKALPAPI EKTISKAKGQ PREPQVYTLP PSREEMTKNQ |
| | VSLTCLVKGF |
| 151 | YPSDIAVEWE SNGQPENNYK TTPPVLDSDG SFFLYSKLTV |
| | DKSRWQQGNV |
| 201 | FSCSVMHEAL HNHYTQKSLS LSPG |

hTβRII-hFc: Nucleic Acid Sequence

(SEQ ID NO: 149)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA ACTCACACAT GCCCACCGTG |
| | CCCAGCACCT |
| 601 | GAACTCCTGG GGGGACCGTC AGTCTTCCTC TTCCCCCCAA |
| | AACCCAAGGA |
| 651 | CACCCTCATG ATCTCCCGGA CCCCTGAGGT CACATGCGTG |
| | GTGGTGGACG |
| 701 | TGAGCCACGA AGACCCTGAG GTCAAGTTCA ACTGGTACGT |
| | GGACGGCGTG |
| 751 | GAGGTGCATA ATGCCAAGAC AAAGCCGCGG GAGGAGCAGT |
| | ACAACAGCAC |
| 801 | GTACCGTGTG GTCAGCGTCC TCACCGTCCT GCACCAGGAC |
| | TGGCTGAATG |
| 851 | GCAAGGAGTA CAAGTGCAAG GTCTCCAACA AAGCCCTCCC |
| | AGCCCCCATC |
| 901 | GAGAAAACCA TCTCCAAAGC CAAAGGGCAG CCCCGAGAAC |
| | CACAGGTGTA |
| 951 | CACCCTGCCC CCATCCCGGG AGGAGATGAC CAAGAACCAG |
| | GTCAGCCTGA |
| 1001 | CCTGCCTGGT CAAAGGCTTC TATCCCAGCG ACATCGCCGT |
| | GGAGTGGGAG |
| 1051 | AGCAATGGGC AGCCGGAGAA CAACTACAAG ACCACGCCTC |
| | CCGTGCTGGA |
| 1101 | CTCCGACGGC TCCTTCTTCC TCTATAGCAA GCTCACCGTG |
| | GACAAGAGCA |
| 1151 | GGTGGCAGCA GGGGAACGTC TTCTCATGCT CCGTGATGCA |
| | TGAGGCTCTG |
| 1201 | CACAACCACT ACACGCAGAA GAGCCTCTCC CTGTCTCCGG |
| | GTAAATGA |

hTβRII-hFc: Amino Acid Sequence

(SEQ ID NO: 142)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | THTCPPCPAP |
| 201 | ELLGGPSVFL FPPKPKDTLM ISRTPEVTCV VVDVSHEDPE |
| | VKENWYVDGV |
| 251 | EVHNAKTKPR EEQYNSTYRV VSVLIVLHQD WINGKEYKCK |
| | VSNKALPAPI |
| 301 | EKTISKAKGQ PREPQVYTLP PSREEMTKNQ VSLTCLVKGF |
| | YPSDIAVEWE |
| 351 | SNGQPENNYK TTPPVLDSDG SFFLYSKLTV DKSRWQQGNV |
| | FSCSVMHEAL |
| 401 | HNHYTQKSLS LSPGK |

The C-terminal lysine residue of the Fc domain can be deleted. The amino acid sequence of SEQ ID NO: 142 may optionally be provided with the lysine removed from the C-terminus (SEQ ID NO: 143):

(SEQ ID NO: 143)

| | |
|---|---|
| 1 | MDAMKRGLCC VLLLCGAVEV SPGATIPPHV QKSDVEMEAQ |
| | KDEIICPSCN |
| 51 | RTAHPLRHIN NDMIVIDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | THTCPPCPAP |
| 201 | ELLGGPSVFL FPPKPKDTLM ISRTPEVTCV VVDVSHEDPE |
| | VKENWYVDGV |
| 251 | EVHNAKTKPR EEQYNSTYRV VSVLTVLHQD WLNGKEYKCK |
| | VSNKALPAPI |
| 301 | EKTISKAKGQ PREPQVYTLP PSREEMTKNQ VSLTCLVKGF |
| | YPSDIAVEWE |
| 351 | SNGQPENNYK TTPPVLDSDG SFFLYSKLTV DKSRWQQGNV |
| | FSCSVMHEAL |
| 401 | HNHYTQKSLS LSPG |

hTβRII (G4S)3-hFc: Nucleic Acid Sequence

(SEQ ID NO: 61)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA GGAAGTGGTG GAGGTGGTTC |
| | TGGAGGTGGT |
| 601 | GGAAGTACTC ACACATGCCC ACCGTGCCCA GCACCTGAAC |
| | TCCTGGGGGG |
| 651 | ACCGTCAGTC TTCCTCTTCC CCCCAAAACC CAAGGACACC |
| | CTCATGATCT |
| 701 | CCCGGACCCC TGAGGTCACA TGCGTGGTGG TGGACGTGAG |
| | CCACGAAGAC |
| 751 | CCTGAGGTCA AGTTCAACTG GTACGTGGAC GGCGTGGAGG |
| | TGCATAATGC |
| 801 | CAAGACAAAG CCGCGGGAGG AGCAGTACAA CAGCACGTAC |
| | CGTGTGGTCA |
| 851 | GCGTCCTCAC CGTCCTGCAC CAGGACTGGC TGAATGGCAA |
| | GGAGTACAAG |
| 901 | TGCAAGGTCT CCAACAAAGC CCTCCCAGCC CCCATCGAGA |
| | AAACCATCTC |
| 951 | CAAAGCCAAA GGGCAGCCCC GAGAACCACA GGTGTACACC |
| | CTGCCCCCAT |
| 1001 | CCCGGGAGGA GATGACCAAG AACCAGGTCA GCCTGACCTG |
| | CCTGGTCAAA |
| 1051 | GGCTTCTATC CCAGCGACAT CGCCGTGGAG TGGGAGAGCA |
| | ATGGGCAGCC |
| 1101 | GGAGAACAAC TACAAGACCA CGCCTCCCGT GCTGGACTCC |
| | GACGGCTCCT |
| 1151 | TCTTCCTCTA TAGCAAGCTC ACCGTGGACA AGAGCAGGTG |
| | GCAGCAGGGG |
| 1201 | AACGTCTTCT CATGCTCCGT GATGCATGAG GCTCTGCACA |
| | ACCACTACAC |
| 1251 | GCAGAAGAGC CTCTCCCTGT CTCCGGGTAA ATGA |

hTβRII (G4S)3-hFc: Amino Acid Sequence

(SEQ ID NO: 71)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSTHTCPPCP APELLGGPSV FLFPPKPKDT LMISRTPEVT |
| | CVVVDVSHED |
| 251 | PEVKFNWYVD GVEVHNAKTK PREEQYNSTY RVVSVLTVLH |
| | QDWLNGKEYK |
| 301 | CKVSNKALPA PIEKTISKAK GOPREPQVYT LPPSREEMTK |
| | NQVSLTCLVK |
| 351 | GFYPSDIAVE WESNGQPENN YKTTPPVLDS DGSFFLYSKL |
| | TVDKSRWQQG |
| 401 | NVFSCSVMHE ALHNHYTQKS LSLSPGK |

The C-terminal lysine residue of the Fc domain can be deleted. The amino acid sequence of SEQ ID NO: 71 may optionally be provided with the lysine removed from the C-terminus (SEQ ID NO: 75):

(SEQ ID NO: 75)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVTDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NISNPDTGGG |
| | GSGGGGSGGG |
| 201 | GSTHTCPPCP APELLGGPSV FLFPPKPKDT LMISRTPEVT |
| | CVVVDVSHED |
| 251 | PEVKFNWYVD GVEVHNAKTK PREEQYNSTY RVVSVLTVLH |
| | QDWLNGKEYK |
| 301 | CKVSNKALPA PIEKTISKAK GQPREPQVYT LPPSREEMTK |
| | NQVSLTCLVK |
| 351 | GFYPSDIAVE WESNGQPENN YKTTPPVLDS DGSFFLYSKL |
| | TVDKSRWQQG |
| 401 | NVFSCSVMHE ALHNHYTQKS LSLSPG |

hTβRII (G4S)4-hFc: Nucleic Acid Sequence

(SEQ ID NO: 62)

| | |
|---|---|
| 1 | ATGGATGCAA TGAAGAGAGG GCTCTGCTGT GTGCTGCTGC |
| | TGTGTGGAGC |
| 51 | AGTCTTCGTT TCGCCCGGCG CCACGATCCC ACCGCACGTT |
| | CAGAAGTCGG |
| 101 | ATGTGGAAAT GGAGGCCCAG AAAGATGAAA TCATCTGCCC |
| | CAGCTGTAAT |
| 151 | AGGACTGCCC ATCCACTGAG ACATATTAAT AACGACATGA |
| | TAGTCACTGA |
| 201 | CAACAACGGT GCAGTCAAGT TTCCACAACT GTGTAAATTT |
| | TGTGATGTGA |
| 251 | GATTTTCCAC CTGTGACAAC CAGAAATCCT GCATGAGCAA |
| | CTGCAGCATC |
| 301 | ACCTCCATCT GTGAGAAGCC ACAGGAAGTC TGTGTGGCTG |
| | TATGGAGAAA |
| 351 | GAATGACGAG AACATAACAC TAGAGACAGT TTGCCATGAC |
| | CCCAAGCTCC |
| 401 | CCTACCATGA CTTTATTCTG GAAGATGCTG CTTCTCCAAA |
| | GTGCATTATG |
| 451 | AAGGAAAAAA AAAAGCCTGG TGAGACTTTC TTCATGTGTT |
| | CCTGTAGCTC |
| 501 | TGATGAGTGC AATGACAACA TCATCTTCTC AGAAGAATAT |
| | AACACCAGCA |
| 551 | ATCCTGACAC CGGTGGTGGA GGTTCTGGAG GTGGAGGAAG |
| | TGGTGGAGGT |
| 601 | GGTTCTGGAG GTGGTGGAAG TACTCACACA TGCCCACCGT |
| | GCCCAGCACC |
| 651 | TGAACTCCTG GGGGGACCGT CAGTCTTCCT CTTCCCCCCA |
| | AAACCCAAGG |
| 701 | ACACCCTCAT GATCTCCCGG ACCCCTGAGG TCACATGCGT |
| | GGTGGTGGAC |
| 751 | GTGAGCCACG AAGACCCTGA GGTCAAGTTC AACTGGTACG |
| | TGGACGGCGT |
| 801 | GGAGGTGCAT AATGCCAAGA CAAAGCCGCG GGAGGAGCAG |
| | TACAACAGCA |
| 851 | CGTACCGTGT GGTCAGCGTC CTCACCGTCC TGCACCAGGA |
| | CTGGCTGAAT |
| 901 | GGCAAGGAGT ACAAGTGCAA GGTCTCCAAC AAAGCCCTCC |
| | CAGCCCCCAT |
| 951 | CGAGAAAACC ATCTCCAAAG CCAAAGGGCA GCCCCGAGAA |
| | CCACAGGTGT |
| 1001 | ACACCCTGCC CCCATCCCGG GAGGAGATGA CCAAGAACCA |
| | GGTCAGCCTG |
| 1051 | ACCTGCCTGG TCAAAGGCTT CTATCCCAGC GACATCGCCG |
| | TGGAGTGGGA |
| 1101 | GAGCAATGGG CAGCCGGAGA ACAACTACAA GACCACGCCT |
| | CCCGTGCTGG |
| 1151 | ACTCCGACGG CTCCTTCTTC CTCTATAGCA AGCTCACCGT |
| | GGACAAGAGC |
| 1201 | AGGTGGCAGC AGGGGAACGT CTTCTCATGC TCCGTGATGC |
| | ATGAGGCTCT |
| 1251 | GCACAACCAC TACACGCAGA AGAGCCTCTC CCTGTCTCCG |
| | GGTAAATGA |

hTβRII (G4S)4-hFc: Amino Acid Sequence

(SEQ ID NO: 72)

| | |
|---|---|
| 1 | MDAMKRGLCC VLLLCGAVFV SPGATIPPHV QKSDVEMEAQ |
| | KDEIICPSCN |
| 51 | RTAHPLRHIN NDMIVIDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NTSNPDTGGG |
| | GSGGGGSGGG |
| 201 | GSGGGGSTHT CPPCPAPELL GGPSVFLFPP KPKDTLMISR |
| | TPEVTCVVVD |
| 251 | VSHEDPEVKF NWYVDGVEVH NAKTKPREEQ YNSTYRVVSV |
| | LTVLHQDWLN |
| 301 | GKEYKCKVSN KALPAPIEKT ISKAKGQPRE PQVYTLPPSR |
| | EEMTKNQVSL |
| 351 | TCLVKGFYPS DIAVEWESNG QPENNYKTTP PVLDSDGSFF |
| | LYSKLTVDKS |
| 401 | RWQQGNVFSC SVMHEALHNH YTQKSLSLSP GK |

The C-terminal lysine residue of the Fc domain can be deleted. The amino acid sequence of SEQ ID NO: 72 may optionally be provided with the lysine removed from the C-terminus (SEQ ID NO: 76):

(SEQ ID NO: 76)

| | |
|---|---|
| 1 | MDAMKRGLCC VLLLCGAVEV SPGATIPPHV QKSDVEMEAQ |
| | KDEIICPSCN |
| 51 | RTAHPLRHIN NDMIVTDNNG AVKFPQLCKF CDVRESTCDN |
| | QKSCMSNCSI |
| 101 | TSICEKPQEV CVAVWRKNDE NITLETVCHD PKLPYHDFIL |
| | EDAASPKCIM |
| 151 | KEKKKPGETF FMCSCSSDEC NDNIIFSEEY NISNPDIGGG |
| | GSGGGGSGGG |
| 201 | GSGGGGSTHT CPPCPAPELL GGPSVFLFPP KPKDTLMISR |
| | TPEVTCVVVD |
| 251 | VSHEDPEVKF NWYVDGVEVH NAKTKPREEQ YNSTYRVVSV |
| | LTVLHQDWLN |
| 301 | GKEYKCKVSN KALPAPIEKT ISKAKGQPRE PQVYTLPPSR |
| | EEMTKNQVSL |
| 351 | TCLVKGFYPS DIAVEWESNG QPENNYKTTP PVLDSDGSFF |
| | LYSKLTVDKS |
| 401 | RWQQGNVFSC SVMHEALHNH YTQKSLSLSP G |

hTβRII (G4S)4-hFc: Amino Acid Sequence Lacking Leader Sequence

(SEQ ID NO: 150)

| | |
|---|---|
| 1 | GATIPPHVQK SDVEMEAQKD EIICPSCNRT AHPLRHINND |
| | MIVTDNNGAV |
| 51 | KFPQLCKFCD VRESTCDNQK SCMSNCSITS ICEKPQEVCV |
| | AVWRKNDENI |
| 101 | TLETVCHDPK LPYHDFILED AASPKCIMKE KKKPGETFFM |
| | CSCSSDECND |
| 151 | NIIFSEEYNT SNPDTGGGGS GGGGSGGGGS GGGGSTHTCP |
| | PCPAPELLGG |
| 201 | PSVFLFPPKP KDTLMISRTP EVTCVVVDVS HEDPEVKENW |
| | YVDGVEVHNA |
| 251 | KIKPREEQYN STYRVVSVLT VLHQDWLNGK EYKCKVSNKA |
| | LPAPIEKTIS |
| 301 | KAKGQPREPQ VYTLPPSREE MTKNQVSLTC LVKGFYPSDI |
| | AVEWESNGQP |
| 351 | ENNYKTTPPV LDSDGSFFLY SKLTVDKSRW QQGNVFSCSV |
| | MHEALHNHYT |
| 401 | QKSLSLSPGK |

The C-terminal lysine residue of the Fc domain can be deleted. The amino acid sequence of SEQ ID NO: 150 may optionally be provided with the lysine removed from the C-terminus (SEQ ID NO: 151):

(SEQ ID NO: 151)

| | |
|---|---|
| 1 | GATIPPHVQK SDVEMEAQKD EIICPSCNRT AHPLRHINND |
| | MIVTDNNGAV |
| 51 | KFPQLCKFCD VRESTCDNQK SCMSNCSITS ICEKPQEVCV |
| | AVWRKNDENI |
| 101 | TLETVCHDPK LPYHDFILED AASPKCIMKE KKKPGETFFM |
| | CSCSSDECND |
| 151 | NIIFSEEYNT SNPDTGGGGS GGGGSGGGGS GGGGSTHTCP |
| | PCPAPELLGG |
| 201 | PSVFLFPPKP KDTLMISRTP EVTCVVVDVS HEDPEVKENW |
| | YVDGVEVHNA |
| 251 | KTKPREEQYN STYRVVSVLT VLHQDWLNGK EYKCKVSNKA |
| | LPAPIEKTIS |
| 301 | KAKGQPREPQ VYTLPPSREE MTKNQVSLTC LVKGFYPSDI |
| | AVEWESNGQP |
| 351 | ENNYKTTPPV LDSDGSFFLY SKLTVDKSRW QQGNVFSCSV |
| | MHEALHNHYT |
| 401 | QKSLSLSPG |

hTβRII (G4S)4-hFc: Amino Acid Sequence Lacking Leader Sequence and Lacking Glycine Prior to hTβRII Portion

(SEQ ID NO: 152)

| | |
|---|---|
| 1 | ATIPPHVQKS DVEMEAQKDE IICPSCNRTA HPLRHINNDM |
| | IVTDNNGAVK |
| 51 | FPQLCKFCDV RESTCDNQKS CMSNCSITSI CEKPQEVCVA |
| | VWRKNDENIT |
| 101 | LETVCHDPKL PYHDFILEDA ASPKCIMKEK KKPGETFFMC |
| | SCSSDECNDN |
| 151 | IIFSEEYNTS NPDTGGGGSG GGGSGGGGSG GGGSTHTCPP |
| | CPAPELLGGP |
| 201 | SVFLFPPKPK DTLMISRTPE VTCVVVDVSH EDPEVKENWY |
| | VDGVEVHNAK |
| 251 | TKPREEQYNS TYRVVSVLIV LHQDWLNGKE YKCKVSNKAL |
| | PAPIEKTISK |
| 301 | AKGQPREPQV YTLPPSREEM TKNQVSLTCL VKGFYPSDIA |
| | VEWESNGQPE |
| 351 | NNYKTTPPVL DSDGSFFLYS KLTVDKSRWQ QGNVFSCSVM |
| | HEALHNHYTQ |
| 401 | KSLSLSPGK |

The C-terminal lysine residue of the Fc domain can be deleted. The amino acid sequence of SEQ ID NO: 152 may optionally be provided with the lysine removed from the C-terminus (SEQ ID NO: 153):

(SEQ ID NO: 153)

| | |
|---|---|
| 1 | ATIPPHVQKS DVEMEAQKDE IICPSCNRTA HPLRHINNDM |
| | IVTDNNGAVK |
| 51 | FPQLCKFCDV RESTCDNQKS CMSNCSITSI CEKPQEVCVA |
| | VWRKNDENIT |
| 101 | LETVCHDPKL PYHDFILEDA ASPKCIMKEK KKPGETFFMC |
| | SCSSDECNDN |
| 151 | IIFSEEYNTS NPDTGGGGSG GGGSGGGGSG GGGSTHTCPP |
| | CPAPELLGGP |
| 201 | SVFLFPPKPK DTLMISRTPE VTCVVVDVSH EDPEVKENWY |
| | VDGVEVHNAK |
| 251 | TKPREEQYNS TYRVVSVLIV LHQDWLNGKE YKCKVSNKAL |
| | PAPIEKTISK |
| 301 | AKGQPREPQV YTLPPSREEM TKNQVSLTCL VKGFYPSDIA |
| | VEWESNGQPE |
| 351 | NNYKTTPPVL DSDGSFFLYS KLTVDKSRWQ QGNVFSCSVM |
| | HEALHNHYTQ |
| 401 | KSLSLSPG |

hTβRII (G4S)4-hFc: Amino Acid Sequence Lacking Leader Sequence and Lacking Glycine and Alanine Prior to hTβRII Portion

(SEQ ID NO: 68)

| | |
|---|---|
| 1 | TIPPHVQKSD VEMEAQKDEI ICPSCNRTAH PLRHINNDMI |
| | VTDNNGAVKF |
| 51 | PQLCKFCDVR FSTCDNQKSC MSNCSITSIC EKPQEVCVAV |
| | WRKNDENITL |
| 101 | ETVCHDPKLP YHDFILEDAA SPKCIMKEKK KPGETFFMCS |
| | CSSDECNDNI |
| 151 | IFSEEYNTSN PDTGGGGSGG GGSGGGGSGG GGSTHTCPPC |
| | PAPELLGGPS |
| 201 | VFLFPPKPKD TLMISRTPEV TCVVVDVSHE DPEVKENWYV |
| | DGVEVHNAKT |
| 251 | KPREEQYNST YRVVSVLIVL HQDWLNGKEY KCKVSNKALP |
| | APIEKTISKA |
| 301 | KGQPREPQVY TLPPSREEMT KNQVSLTCLV KGFYPSDIAV |
| | EWESNGQPEN |
| 351 | NYKTTPPVLD SDGSFFLYSK LTVDKSRWQQ GNVFSCSVMH |
| | EALHNHYTQK |
| 401 | SLSLSPGK |

The C-terminal lysine residue of the Fc domain can be deleted. The amino acid sequence of SEQ ID NO: 68 may optionally be provided with the lysine removed from the C-terminus (SEQ ID NO: 70):

```
(SEQ ID NO: 70)
    1 TIPPHVQKSD VEMEAQKDEI ICPSCNRTAH PLRHINNDMI
        VTDNNGAVKF
  51 PQLCKFCDVR FSTCDNQKSC MSNCSITSIC EKPQEVCVAV
        WRKNDENITL
101 ETVCHDPKLP YHDFILEDAA SPKCIMKEKK KPGETFFMCS
        CSSDECNDNI
151 IFSEEYNTSN PDTGGGGSGG GGSGGGGSGG GGSTHTCPPC
        PAPELLGGPS
201 VFLFPPKPKD TLMISRTPEV TCVVVDVSHE DPEVKFNWYV
        DGVEVHNAKT
251 KPREEQYNST YRVVSVLTVL HQDWLNGKEY KCKVSNKALP
        APIEKTISKA
301 KGQPREPQVY TLPPSREEMT KNQVSLTCLV KGFYPSDIAV
        EWESNGQPEN
351 NYKTTPPVLD SDGSFFLYSK LTVDKSRWQQ GNVFSCSVMH
        EALHNHYTQK
401 SLSLSPG
```

hTβRII (G4S)4-hFc: Amino Acid Sequence Lacking Leader Sequence and Lacking Glycine, Alanine, and Threonine Prior to hTβRII Portion

```
(SEQ ID NO: 154)
    1 IPPHVQKSDV EMEAQKDEII CPSCNRTAHP LRHINNDMIV
        TDNNGAVKFP
  51 QLCKFCDVRF STCDNQKSCM SNCSITSICE KPQEVCVAVW
        RKNDENITLE
101 TVCHDPKLPY HDFILEDAAS PKCIMKEKKK PGETFFMCSC
        SSDECNDNII
151 FSEEYNTSNP DTGGGGSGGG GSGGGGSGGG GSTHTCPPCP
        APELLGGPSV
201 FLFPPKPKDT LMISRTPEVT CVVVDVSHED PEVKFNWYVD
        GVEVHNAKTK
251 PREEQYNSTY RVVSVLIVLH QDWLNGKEYK CKVSNKALPA
        PIEKTISKAK
301 GQPREPQVYT LPPSREEMTK NQVSLTCLVK GFYPSDIAVE
        WESNGQPENN
351 YKTTPPVLDS DGSFFLYSKL TVDKSRWQQG NVFSCSVMHE
        ALHNHYTQKS
401 LSLSPGK
```

The C-terminal lysine residue of the Fc domain can be deleted. The amino acid sequence of SEQ ID NO: 154 may optionally be provided with the lysine removed from the C-terminus (SEQ ID NO: 155):

```
(SEQ ID NO: 155)
    1 IPPHVQKSDV EMEAQKDEII CPSCNRTAHP LRHINNDMIV
        TDNNGAVKFP
  51 QLCKFCDVRF STCDNQKSCM SNCSITSICE KPQEVCVAVW
        RKNDENITLE
101 TVCHDPKLPY HDFILEDAAS PKCIMKEKKK PGETFFMCSC
        SSDECNDNII
151 FSEEYNTSNP DTGGGGSGGG GSGGGGSGGG GSTHTCPPCP
        APELLGGPSV
201 FLFPPKPKDT LMISRTPEVT CVVVDVSHED PEVKFNWYVD
        GVEVHNAKTK
251 PREEQYNSTY RVVSVLTVLH QDWLNGKEYK CKVSNKALPA
        PIEKTISKAK
301 GQPREPQVYT LPPSREEMTK NQVSLTCLVK GFYPSDIAVE
        WESNGQPENN
351 YKTTPPVLDS DGSFFLYSKL TVDKSRWQQG NVFSCSVMHE
        ALHNHYTQKS
401 LSLSPG
```

hTβRII (G4S)4-hFc: Amino Acid Sequence Lacking Leader Sequence and Lacking Glycine, Alanine, Threonine, and Isoleucine Prior to hTβRII Portion

```
(SEQ ID NO: 156)
    1 PPHVQKSDVE MEAQKDEIIC PSCNRTAHPL RHINNDMIVT
        DNNGAVKFPQ
  51 LCKFCDVRFS TCDNQKSCMS NCSITSICEK PQEVCVAVWR
        KNDENITLET
101 VCHDPKLPYH DFILEDAASP KCIMKEKKKP GETFFMCSCS
        SDECNDNIIF
151 SEEYNTSNPD TGGGGSGGGG SGGGGSGGGG STHTCPPCPA
        PELLGGPSVF
201 LFPPKPKDTL MISRTPEVTC VVVDVSHEDP EVKENWYVDG
        VEVHNAKTKP
251 REEQYNSTYR VVSVLTVLHQ DWLNGKEYKC KVSNKALPAP
        IEKTISKAKG
301 QPREPQVYTL PPSREEMTKN QVSLTCLVKG FYPSDIAVEW
        ESNGQPENNY
351 KTTPPVLDSD GSFFLYSKLT VDKSRWQQGN VFSCSVMHEA
        LHNHYTQKSL
401 SLSPGK
```

The C-terminal lysine residue of the Fc domain can be deleted. The amino acid sequence of SEQ ID NO: 156 may optionally be provided with the lysine removed from the C-terminus (SEQ ID NO: 157):

```
(SEQ ID NO: 157)
    1 PPHVQKSDVE MEAQKDEIIC PSCNRTAHPL RHINNDMIVT
        DNNGAVKFPQ
  51 LCKFCDVRFS TCDNQKSCMS NCSITSICEK PQEVCVAVWR
        KNDENITLET
101 VCHDPKLPYH DFILEDAASP KCIMKEKKKP GETFFMCSCS
        SDECNDNIIF
151 SEEYNTSNPD TGGGGSGGGG SGGGGSGGGG STHTCPPCPA
        PELLGGPSVF
201 LFPPKPKDTL MISRTPEVTC VVVDVSHEDP EVKFNWYVDG
        VEVHNAKTKP
251 REEQYNSTYR VVSVLTVLHQ DWLNGKEYKC KVSNKALPAP
        IEKTISKAKG
301 QPREPQVYTL PPSREEMTKN QVSLTCLVKG FYPSDIAVEW
        ESNGQPENNY
351 KTTPPVLDSD GSFFLYSKLT VDKSRWQQGN VFSCSVMHEA
        LHNHYTQKSL
401 SLSPG
```

hTβRII (G4S)4-hFc: Amino Acid Sequence Lacking Leader Sequence and Lacking Glycine, Alanine, Threonine, Isoleucine, and Proline Prior to hTβRII Portion

```
(SEQ ID NO: 158)
    1 PHVQKSDVEM EAQKDEIICP SCNRTAHPLR HINNDMIVTD
        NNGAVKFPQL
  51 CKFCDVRFST CDNQKSCMSN CSITSICEKP QEVCVAVWRK
        NDENITLETV
101 CHDPKLPYHD FILEDAASPK CIMKEKKKPG ETFFMCSCSS
        DECNDNIIFS
151 EEYNTSNPDT GGGGSGGGGS GGGGSGGGGS THTCPPCPAP
        ELLGGPSVFL
201 FPPKPKDTLM ISRTPEVTCV VVDVSHEDPE VKFNWYVDGV
        EVHNAKTKPR
251 EEQYNSTYRV VSVLTVLHQD WLNGKEYKCK VSNKALPAPI
        EKTISKAKGQ
301 PREPQVYTLP PSREEMTKNQ VSLTCLVKGF YPSDIAVEWE
        SNGQPENNYK
351 TTPPVLDSDG SFFLYSKLTV DKSRWQQGNV FSCSVMHEAL
        HNHYTQKSLS
401 LSPGK
```

The C-terminal lysine residue of the Fc domain can be deleted. The amino acid sequence of SEQ ID NO: 158 may optionally be provided with the lysine removed from the C-terminus (SEQ ID NO: 159):

```
(SEQ ID NO: 159)
    1 PHVQKSDVEM EAQKDEIICP SCNRTAHPLR HINNDMIVTD
        NNGAVKFPQL
  51 CKFCDVRFST CDNQKSCMSN CSITSICEKP QEVCVAVWRK
        NDENITLETV
101 CHDPKLPYHD FILEDAASPK CIMKEKKKPG ETFFMCSCSS
        DECNDNIIFS
151 EEYNTSNPDT GGGGSGGGGS GGGGSGGGGS THTCPPCPAP
        ELLGGPSVFL
201 FPPKPKDTLM ISRTPEVTCV VVDVSHEDPE VKFNWYVDGV
        EVHNAKTKPR
251 EEQYNSTYRV VSVLTVLHQD WLNGKEYKCK VSNKALPAPI
        EKTISKAKGQ
301 PREPQVYTLP PSREEMTKNQ VSLTCLVKGF YPSDIAVEWE
        SNGQPENNYK
351 TTPPVLDSDG SFFLYSKLTV DKSRWQQGNV FSCSVMHEAL
        HNHYTQKSLS
401 LSPG
```

hTβRII (G4S)4-hFc: Amino Acid Sequence Lacking Leader Sequence and Lacking Glycine, Alanine, Threonine, Isoleucine, Proline, and Proline Prior to hTβRII Portion

```
(SEQ ID NO: 160)
    1 HVQKSDVEME AQKDEIICPS CNRTAHPLRH INNDMIVTDN
        NGAVKFPQLC
  51 KFCDVRFSTC DNQKSCMSNC SITSICEKPQ EVCVAVWRKN
        DENITLETVC
101 HDPKLPYHDF ILEDAASPKC IMKEKKKPGE TFFMCSCSSD
        ECNDNIIFSE
151 EYNTSNPDTG GGGSGGGGSG GGGSGGGGST HTCPPCPAPE
        LLGGPSVFLF
201 PPKPKDTLMI SRTPEVTCVV VDVSHEDPEV KFNWYVDGVE
        VHNAKTKPRE
251 EQYNSTYRVV SVLTVLHQDW LNGKEYKCKV SNKALPAPIE
        KTISKAKGQP
301 REPQVYTLPP SREEMTKNQV SLTCLVKGFY PSDIAVEWES
        NGQPENNYKT
351 TPPVLDSDGS FFLYSKLTVD KSRWQQGNVF SCSVMHEALH
        NHYTQKSLSL
401 SPGK
```

The C-terminal lysine residue of the Fc domain can be deleted. The amino acid sequence of SEQ ID NO: 160 may optionally be provided with the lysine removed from the C-terminus (SEQ ID NO: 161):

```
(SEQ ID NO: 161)
    1 HVQKSDVEME AQKDEIICPS CNRTAHPLRH INNDMIVTDN
        NGAVKFPQLC
  51 KFCDVRFSTC DNQKSCMSNC SITSICEKPQ EVCVAVWRKN
        DENITLETVC
101 HDPKLPYHDF ILEDAASPKC IMKEKKKPGE TFFMCSCSSD
        ECNDNIIFSE
151 EYNTSNPDTG GGGSGGGGSG GGGSGGGGST HTCPPCPAPE
        LLGGPSVFLF
201 PPKPKDTLMI SRTPEVTCVV VDVSHEDPEV KFNWYVDGVE
        VHNAKTKPRE
251 EQYNSTYRVV SVLTVLHQDW LNGKEYKCKV SNKALPAPIE
        KTISKAKGQP
301 REPQVYTLPP SREEMTKNQV SLTCLVKGFY PSDIAVEWES
        NGQPENNYKT
351 TPPVLDSDGS FFLYSKLTVD KSRWQQGNVF SCSVMHEALH
        NHYTQKSLSL
401 SPG
```

hTβRII (G4S)2-hFc: Nucleic Acid Sequence

```
(SEQ ID NO: 63)
      1 ATGGATGCAA TGAAGAGAGG GCTCTGCTGT GTGCTGCTGC
          TGTGTGGAGC
    51 AGTCTTCGTT TCGCCCGGCG CCACGATCCC ACCGCACGTT
          CAGAAGTCGG
  101 ATGTGGAAAT GGAGGCCCAG AAAGATGAAA TCATCTGCCC
          CAGCTGTAAT
  151 AGGACTGCCC ATCCACTGAG ACATATTAAT AACGACATGA
          TAGTCACTGA
  201 CAACAACGGT GCAGTCAAGT TTCCACAACT GTGTAAATTT
          TGTGATGTGA
  251 GATTTTCCAC CTGTGACAAC CAGAAATCCT GCATGAGCAA
          CTGCAGCATC
  301 ACCTCCATCT GTGAGAAGCC ACAGGAAGTC TGTGTGGCTG
          TATGGAGAAA
  351 GAATGACGAG AACATAACAC TAGAGACAGT TTGCCATGAC
          CCCAAGCTCC
  401 CCTACCATGA CTTTATTCTG GAAGATGCTG CTTCTCCAAA
          GTGCATTATG
  451 AAGGAAAAAA AAAAGCCTGG TGAGACTTTC TTCATGTGTT
          CCTGTAGCTC
  501 TGATGAGTGC AATGACAACA TCATCTTCTC AGAAGAATAT
          AACACCAGCA
  551 ATCCTGACAC CGGTGGAGGT GGTTCTGGAG GTGGTGGAAG
          TACTCACACA
  601 TGCCCACCGT GCCCAGCACC TGAACTCCTG GGGGGACCGT
          CAGTCTTCCT
  651 CTTCCCCCCA AAACCCAAGG ACACCCTCAT GATCTCCCGG
          ACCCCTGAGG
  701 TCACATGCGT GGTGGTGGAC GTGAGCCACG AAGACCCTGA
          GGTCAAGTTC
  751 AACTGGTACG TGGACGGCGT GGAGGTGCAT AATGCCAAGA
          CAAAGCCGCG
  801 GGAGGAGCAG TACAACAGCA CGTACCGTGT GGTCAGCGTC
          CTCACCGTCC
  851 TGCACCAGGA CTGGCTGAAT GGCAAGGAGT ACAAGTGCAA
          GGTCTCCAAC
  901 AAAGCCCTCC CAGCCCCCAT CGAGAAAACC ATCTCCAAAG
          CCAAAGGGCA
  951 GCCCCGAGAA CCACAGGTGT ACACCCTGCC CCCATCCCGG
          GAGGAGATGA
1001 CCAAGAACCA GGTCAGCCTG ACCTGCCTGG TCAAAGGCTT
          CTATCCCAGC
1051 GACATCGCCG TGGAGTGGGA GAGCAATGGG CAGCCGGAGA
          ACAACTACAA
1101 GACCACGCCT CCCGTGCTGG ACTCCGACGG CTCCTTCTTC
          CTCTATAGCA
1151 AGCTCACCGT GGACAAGAGC AGGTGGCAGC AGGGGAACGT
          CTTCTCATGC
1201 TCCGTGATGC ATGAGGCTCT GCACAACCAC TACACGCAGA
          AGAGCCTCTC
1251 CCTGTCTCCG GGTAAATGA
```

hTβRII (G4S)2-hFc: Amino Acid Sequence

```
(SEQ ID NO: 73)
    1 MDAMKRGLCC VLLLCGAVFV SPGATIPPHV QKSDVEMEAQ
        KDEIICPSCN
  51 RTAHPLRHIN NDMIVTDNNG AVKFPQLCKF CDVRESTCDN
        QKSCMSNCSI
101 TSICEKPQEV CVAVWRKNDE NITLETVCHD PKLPYHDFIL
        EDAASPKCIM
151 KEKKKPGETF FMCSCSSDEC NDNIIFSEEY NTSNPDTGGG
        GSGGGGSTHT
201 CPPCPAPELL GGPSVFLFPP KPKDTLMISR TPEVTCVVVD
        VSHEDPEVKF
```

251 NWYVDGVEVH NAKTKPREEQ YNSTYRVVSV LTVLHQDWLN
GKEYKCKVSN

```
301 KALPAPIEKT ISKAKGQPRE PQVYTLPPSR EEMTKNQVSL
        TCLVKGFYPS
351 DIAVEWESNG QPENNYKTTP PVLDSDGSFF LYSKLTVDKS
        RWQQGNVFSC
401 SVMHEALHNH YTQKSLSLSP GK
```

The C-terminal lysine residue of the Fc domain can be deleted. The amino acid sequence of SEQ ID NO: 73 may optionally be provided with the lysine removed from the C-terminus (SEQ ID NO: 77):

```
(SEQ ID NO: 77)
    1 MDAMKRGLCC VLLLCGAVFV SPGATIPPHV QKSDVEMEAQ
        KDEIICPSCN
  51 RTAHPLRHIN NDMIVTDNNG AVKFPQLCKF CDVRFSTCDN
        QKSCMSNCSI
101 TSICEKPQEV CVAVWRKNDE NITLETVCHD PKLPYHDFIL
        EDAASPKCIM
151 KEKKKPGETF FMCSCSSDEC NDNIIFSEEY NTSNPDTGGG
        GSGGGGSTHT
201 CPPCPAPELL GGPSVFLFPP KPKDTLMISR TPEVTCVVVD
        VSHEDPEVKF
251 NWYVDGVEVH NAKTKPREEQ YNSTYRVVSV LTVLHQDWLN
        GKEYKCKVSN
301 KALPAPIEKT ISKAKGQPRE PQVYTLPPSR EEMTKNQVSL
        TCLVKGFYPS
351 DIAVEWESNG QPENNYKTTP PVLDSDGSFF LYSKLTVDKS
        RWQQGNVFSC
401 SVMHEALHNH YTQKSLSLSP G
```

hTβRII Extended Hinge-hFc: Nucleic Acid Sequence

```
(SEQ ID NO: 64)
      1 ATGGATGCAA TGAAGAGAGG GCTCTGCTGT GTGCTGCTGC
          TGTGTGGAGC
    51 AGTCTTCGTT TCGCCCGGCG CCACGATCCC ACCGCACGTT
          CAGAAGTCGG
  101 ATGTGGAAAT GGAGGCCCAG AAAGATGAAA TCATCTGCCC
          CAGCTGTAAT
  151 AGGACTGCCC ATCCACTGAG ACATATTAAT AACGACATGA
          TAGTCACTGA
  201 CAACAACGGT GCAGTCAAGT TTCCACAACT GTGTAAATTT
          TGTGATGTGA
  251 GATTTTCCAC CTGTGACAAC CAGAAATCCT GCATGAGCAA
          CTGCAGCATC
  301 ACCTCCATCT GTGAGAAGCC ACAGGAAGTC TGTGTGGCTG
        TATGGAGAAA
  351 GAATGACGAG AACATAACAC TAGAGACAGT TTGCCATGAC
          CCCAAGCTCC
  401 CCTACCATGA CTTTATTCTG GAAGATGCTG CTTCTCCAAA
          GTGCATTATG
  451 AAGGAAAAAA AAAAGCCTGG TGAGACTTTC TTCATGTGTT
          CCTGTAGCTC
  501 TGATGAGTGC AATGACAACA TCATCTTCTC AGAAGAATAT
          AACACCAGCA
  551 ATCCTGACAC CGGTGGTGGA CCCAAATCTT GTGACAAAAC
          TCACACATGC
  601 CCACCGTGCC CAGCACCTGA ACTCCTGGGG GGACCGTCAG
          TCTTCCTCTT
  651 CCCCCCAAAA CCCAAGGACA CCCTCATGAT CTCCCGGACC
          CCTGAGGTCA
  701 CATGCGTGGT GGTGGACGTG AGCCACGAAG ACCCTGAGGT
          CAAGTTCAAC
  751 TGGTACGTGG ACGGCGTGGA GGTGCATAAT GCCAAGACAA
          AGCCGCGGGA
  801 GGAGCAGTAC AACAGCACGT ACCGTGTGGT CAGCGTCCTC
          ACCGTCCTGC
  851 ACCAGGACTG GCTGAATGGC AAGGAGTACA AGTGCAAGGT
          CTCCAACAAA
  901 GCCCTCCCAG CCCCCATCGA GAAAACCATC TCCAAAGCCA
          AAGGGCAGCC
  951 CCGAGAACCA CAGGTGTACA CCCTGCCCCC ATCCCGGGAG
          GAGATGACCA
1001 AGAACCAGGT CAGCCTGACC TGCCTGGTCA AAGGCTTCTA
          TCCCAGCGAC
1051 ATCGCCGTGG AGTGGGAGAG CAATGGGCAG CCGGAGAACA
          ACTACAAGAC
1101 CACGCCTCCC GTGCTGGACT CCGACGGCTC CTTCTTCCTC
          TATAGCAAGC
1151 TCACCGTGGA CAAGAGCAGG TGGCAGCAGG GGAACGTCTT
          CTCATGCTCC
1201 GTGATGCATG AGGCTCTGCA CAACCACTAC ACGCAGAAGA
          GCCTCTCCCT
1251 GTCCCCGGGT AAATGA
```

hTβRII Extended Hinge-hFc: Amino Acid Sequence

```
(SEQ ID NO: 74)
    1 MDAMKRGLCC VLLLCGAVEV SPGATIPPHV QKSDVEMEAQ
        KDEIICPSCN
  51 RTAHPLRHIN NDMIVTDNNG AVKFPQLCKF CDVRESTCDN
        QKSCMSNCSI
101 TSICEKPQEV CVAVWRKNDE NITLETVCHD PKLPYHDFIL
        EDAASPKCIM
151 KEKKKPGETF FMCSCSSDEC NDNIIFSEEY NTSNPDTGGG
        PKSCDKTHTC
201 PPCPAPELLG GPSVFLFPPK PKDTLMISRT PEVTCVVVDV
        SHEDPEVKEN
251 WYVDGVEVHN AKTKPREEQY NSTYRVVSVL TVLHQDWING
        KEYKCKVSNK
301 ALPAPIEKTI SKAKGQPREP QVYTLPPSRE EMTKNQVSLT
        CLVKGFYPSD
351 IAVEWESNGQ PENNYKTTPP VLDSDGSFFL YSKLTVDKSR
        WQQGNVFSCS
401 VMHEALHNHY TOKSLSLSPG K
```

The C-terminal lysine residue of the Fc domain can be deleted. The amino acid sequence of SEQ ID NO: 74 may optionally be provided with the lysine removed from the C-terminus (SEQ ID NO: 78):

```
(SEQ ID NO: 78)
    1 MDAMKRGLCC VLLLCGAVFV SPGATIPPHV QKSDVEMEAQ
        KDEIICPSCN
  51 RTAHPLRHIN NDMIVTDNNG AVKFPQLCKF CDVRESTCDN
        QKSCMSNCSI
101 TSICEKPQEV CVAVWRKNDE NITLETVCHD PKLPYHDFIL
        EDAASPKCIM
151 KEKKKPGETF FMCSCSSDEC NDNIIFSEEY NTSNPDTGGG
        PKSCDKTHTC
201 PPCPAPELLG GPSVFLFPPK PKDTLMISRT PEVTCVVVDV
        SHEDPEVKFN
251 WYVDGVEVHN AKTKPREEQY NSTYRVVSVL TVLHQDWING
        KEYKCKVSNK
301 ALPAPIEKTI SKAKGQPREP QVYTLPPSRE EMTKNQVSLT
        CLVKGFYPSD
351 IAVEWESNGQ PENNYKTTPP VLDSDGSFFL YSKLTVDKSR
        WQQGNVESCS
401 VMHEALHNHY TQKSLSLSPG
```

hTβRII (G4S)5-hFc: Amino Acid Sequence

(SEQ ID NO: 144)

| | |
|---|---|
| 1 | MDAMKRGLCC VLLLCGAVEV SPGATIPPHV |
| | QKSDVEMEAQ KDEIICPSCN |
| 51 | RTAHPLRHIN NDMIVTDNNG AVKFPQLCKF |
| | CDVRESTCDN QKSCMSNCSI |
| 101 | TSICEKPQEV CVAVWRKNDE NITLETVCHD |
| | PKLPYHDFIL EDAASPKCIM |
| 151 | KEKKKPGETF FMCSCSSDEC NDNIIFSEEY |
| | NTSNPDTGGG GSGGGGSGGG |
| 201 | GSGGGGSGGG GSTHTCPPCP APELLGGPSV |
| | FLFPPKPKDT LMISRTPEVT |
| 251 | CVVVDVSHED PEVKENWYVD GVEVHNAKTK |
| | PREEQYNSTY RVVSVLTVLH |
| 301 | QDWLNGKEYK CKVSNKALPA PIEKTISKAK |
| | GQPREPQVYT LPPSREEMTK |
| 351 | NQVSLTCLVK GFYPSDIAVE WESNGQPENN |
| | YKTTPPVLDS DGSFFLYSKL |
| 401 | TVDKSRWQQG NVFSCSVMHE ALHNHYTQKS |
| | LSLSPGK |

The C-terminal lysine residue of the Fc domain can be deleted. The amino acid sequence of SEQ ID NO: 144 may optionally be provided with the lysine removed from the C-terminus (SEQ ID NO: 145):

(SEQ ID NO: 145)

| | |
|---|---|
| 1 | MDAMKRGLCC VLLLCGAVEV SPGATIPPHV |
| | QKSDVEMEAQ KDEIICPSCN |
| 51 | RTAHPLRHIN NDMIVTDNNG AVKFPQLCKF |
| | CDVRESTCDN QKSCMSNCSI |
| 101 | TSICEKPQEV CVAVWRKNDE NITLETVCHD |
| | PKLPYHDFIL EDAASPKCIM |
| 151 | KEKKKPGETF FMCSCSSDEC NDNIIFSEEY |
| | NTSNPDTGGG GSGGGGSGGG |
| 201 | GSGGGGSGGG GSTHTCPPCP APELLGGPSV |
| | FLFPPKPKDT LMISRTPEVT |
| 251 | CVVVDVSHED PEVKENWYVD GVEVHNAKTK |
| | PREEQYNSTY RVVSVLTVLH |
| 301 | QDWLNGKEYK CKVSNKALPA PIEKTISKAK |
| | GQPREPQVYT LPPSREEMTK |
| 351 | NQVSLTCLVK GFYPSDIAVE WESNGQPENN |
| | YKTTPPVLDS DGSFFLYSKL |
| 401 | TVDKSRWQQG NVFSCSVMHE ALHNHYTQKS |
| | LSLSPG |

hTβRII (G4S)6-hFc: Amino Acid Sequence

(SEQ ID NO: 146)

| | |
|---|---|
| 1 | MDAMKRGLCC VLLLCGAVEV SPGATIPPHV |
| | QKSDVEMEAQ KDEIICPSCN |
| 51 | RTAHPLRHIN NDMIVTDNNG AVKFPQLCKF |
| | CDVRESTCDN QKSCMSNCSI |
| 101 | TSICEKPQEV CVAVWRKNDE NITLETVCHD |
| | PKLPYHDFIL EDAASPKCIM |
| 151 | KEKKKPGETF FMCSCSSDEC NDNIIFSEEY |
| | NTSNPDTGGG GSGGGGSGGG |
| 201 | GSGGGGSGGG GSGGGGSTHT CPPCPAPELL |
| | GGPSVFLFPP KPKDTLMISR |
| 251 | TPEVTCVVVD VSHEDPEVKF NWYVDGVEVH |
| | NAKTKPREEQ YNSTYRVVSV |
| 301 | LTVLHQDWLN GKEYKCKVSN KALPAPIEKT |
| | ISKAKGQPRE PQVYTLPPSR |
| 351 | EEMTKNQVSL TCLVKGFYPS DIAVEWESNG |
| | QPENNYKTTP PVLDSDGSFF |
| 401 | LYSKLTVDKS RWQQGNVFSC SVMHEALHNH |
| | YTQKSLSLSP GK |

The C-terminal lysine residue of the Fc domain can be deleted. The amino acid sequence of SEQ ID NO: 146 may optionally be provided with the lysine removed from the C-terminus (SEQ ID NO: 147):

(SEQ ID NO: 147)

| | |
|---|---|
| 1 | MDAMKRGLCC VLLLCGAVEV SPGATIPPHV |
| | QKSDVEMEAQ KDEIICPSCN |
| 51 | RTAHPLRHIN NDMIVTDNNG AVKFPQLCKF |
| | CDVRFSTCDN QKSCMSNCSI |
| 101 | TSICEKPQEV CVAVWRKNDE NITLETVCHD |
| | PKLPYHDFIL EDAASPKCIM |
| 151 | KEKKKPGETF FMCSCSSDEC NDNIIFSEEY |
| | NTSNPDTGGG GSGGGGSGGG |
| 201 | GSGGGGSGGG GSGGGGSTHT CPPCPAPELL |
| | GGPSVFLFPP KPKDTLMISR |
| 251 | TPEVTCVVVD VSHEDPEVKF NWYVDGVEVH |
| | NAKTKPREEQ YNSTYRVVSV |
| 301 | LTVLHQDWLN GKEYKCKVSN KALPAPIEKT |
| | ISKAKGQPRE PQVYTLPPSR |
| 351 | EEMTKNQVSL TCLVKGFYPS DIAVEWESNG |
| | QPENNYKTTP PVLDSDGSFF |
| 401 | LYSKLTVDKS RWQQGNVFSC SVMHEALHNH |
| | YTQKSLSLSP G |

hTβRII (G4S)5-hFc: Nucleotide Sequence

(SEQ ID NO: 162)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCACGATCCC |
| | ACCGCACGTT CAGAAGTCGG |
| 101 | ATGTGGAAAT GGAGGCCCAG AAAGATGAAA |
| | TCATCTGCCC CAGCTGTAAT |
| 151 | AGGACTGCCC ATCCACTGAG ACATATTAAT |
| | AACGACATGA TAGTCACTGA |
| 201 | CAACAACGGT GCAGTCAAGT TTCCACAACT |
| | GTGTAAATTT TGTGATGTGA |
| 251 | GATTTTCCAC CTGTGACAAC CAGAAATCCT |
| | GCATGAGCAA CTGCAGCATC |
| 301 | ACCTCCATCT GTGAGAAGCC ACAGGAAGTC |
| | TGTGTGGCTG TATGGAGAAA |
| 351 | GAATGACGAG AACATAACAC TAGAGACAGT |
| | TTGCCATGAC CCCAAGCTCC |
| 401 | CCTACCATGA CTTTATTCTG GAAGATGCTG |
| | CTTCTCCAAA GTGCATTATG |
| 451 | AAGGAAAAAA AAAAGCCTGG TGAGACTTTC |
| | TTCATGTGTT CCTGTAGCTC |
| 501 | TGATGAGTGC AATGACAACA TCATCTTCTC |
| | AGAAGAATAT AACACCAGCA |
| 551 | ATCCTGACAC CGGTGGAGGA GGTTCTGGTG |
| | GTGGAGGTTC TGGAGGTGGA |
| 601 | GGAAGTGGTG GAGGTGGTTC TGGAGGTGGT |
| | GGAAGTACTC ACACATGCCC |
| 651 | ACCGTGCCCA GCACCTGAAC TCCTGGGGGG |
| | ACCGTCAGTC TTCCTCTTCC |
| 701 | CCCCAAAACC CAAGGACACC CTCATGATCT |
| | CCCGGACCCC TGAGGTCACA |
| 751 | TGCGTGGTGG TGGACGTGAG CCACGAAGAC |
| | CCTGAGGTCA AGTTCAACTG |
| 801 | GTACGTGGAC GGCGTGGAGG TGCATAATGC |
| | CAAGACAAAG CCGCGGGAGG |
| 851 | AGCAGTACAA CAGCACGTAC CGTGTGGTCA |
| | GCGTCCTCAC CGTCCTGCAC |
| 901 | CAGGACTGGC TGAATGGCAA GGAGTACAAG |
| | TGCAAGGTCT CCAACAAAGC |
| 951 | CCTCCCAGCC CCCATCGAGA AAACCATCTC |
| | CAAAGCCAAA GGGCAGCCCC |
| 1001 | GAGAACCACA GGTGTACACC CTGCCCCCAT |
| | CCCGGGAGGA GATGACCAAG |
| 1051 | AACCAGGTCA GCCTGACCTG CCTGGTCAAA |
| | GGCTTCTATC CCAGCGACAT |
| 1101 | CGCCGTGGAG TGGGAGAGCA ATGGGCAGCC |
| | GGAGAACAAC TACAAGACCA |
| 1151 | CGCCTCCCGT GCTGGACTCC GACGGCTCCT |
| | TCTTCCTCTA TAGCAAGCTC |
| 1201 | ACCGTGGACA AGAGCAGGTG GCAGCAGGGG |
| | AACGTCTTCT CATGCTCCGT |
| 1251 | GATGCATGAG GCTCTGCACA ACCACTACAC |
| | GCAGAAGAGC CTCTCCCTGT |
| 1301 | CTCCGGGTAA ATGA |

hTβRII (G4S)6-hFc: Nucleotide Sequence

(SEQ ID NO: 163)

| | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCACGATCCC |
| | ACCGCACGTT CAGAAGTCGG |
| 101 | ATGTGGAAAT GGAGGCCCAG AAAGATGAAA |
| | TCATCTGCCC CAGCTGTAAT |
| 151 | AGGACTGCCC ATCCACTGAG ACATATTAAT |
| | AACGACATGA TAGTCACTGA |
| 201 | CAACAACGGT GCAGTCAAGT TTCCACAACT |
| | GTGTAAATTT TGTGATGTGA |
| 251 | GATTTTCCAC CTGTGACAAC CAGAAATCCT |
| | GCATGAGCAA CTGCAGCATC |
| 301 | ACCTCCATCT GTGAGAAGCC ACAGGAAGTC |
| | TGTGTGGCTG TATGGAGAAA |
| 351 | GAATGACGAG AACATAACAC TAGAGACAGT |
| | TTGCCATGAC CCCAAGCTCC |
| 401 | CCTACCATGA CTTTATTCTG GAAGATGCTG |
| | CTTCTCCAAA GTGCATTATG |
| 451 | AAGGAAAAAA AAAAGCCTGG TGAGACTTTC |
| | TTCATGTGTT CCTGTAGCTC |
| 501 | TGATGAGTGC AATGACAACA TCATCTTCTC |
| | AGAAGAATAT AACACCAGCA |
| 551 | ATCCTGACAC CGGTGGAGGT GGAAGTGGTG |
| | GAGGAGGTTC TGGTGGTGGA |
| 601 | GGITCTGGAG GTGGAGGAAG TGGTGGAGGT |
| | GGTTCTGGAG GTGGTGGAAG |
| 651 | TACTCACACA TGCCCACCGT GCCCAGCACC |
| | TGAACTCCTG GGGGGACCGT |
| 701 | CAGTCTTCCT CTTCCCCCCA AAACCCAAGG |
| | ACACCCTCAT GATCTCCCGG |
| 751 | ACCCCTGAGG TCACATGCGT GGTGGTGGAC |
| | GTGAGCCACG AAGACCCTGA |
| 801 | GGTCAAGTTC AACTGGTACG TGGACGGCGT |
| | GGAGGTGCAT AATGCCAAGA |
| 851 | CAAAGCCGCG GGAGGAGCAG TACAACAGCA |
| | CGTACCGTGT GGTCAGCGTC |
| 901 | CTCACCGTCC TGCACCAGGA CTGGCTGAAT |
| | GGCAAGGAGT ACAAGTGCAA |
| 951 | GGTCTCCAAC AAAGCCCTCC CAGCCCCCAT |
| | CGAGAAAACC ATCTCCAAAG |
| 1001 | CCAAAGGGCA GCCCCGAGAA CCACAGGTGT |
| | ACACCCTGCC CCCATCCCGG |
| 1051 | GAGGAGATGA CCAAGAACCA GGTCAGCCTG |
| | ACCTGCCTGG TCAAAGGCTT |
| 1101 | CTATCCCAGC GACATCGCCG TGGAGTGGGA |
| | GAGCAATGGG CAGCCGGAGA |
| 1151 | ACAACTACAA GACCACGCCT CCCGTGCTGG |
| | ACTCCGACGG CTCCTTCTTC |
| 1201 | CTCTATAGCA AGCTCACCGT GGACAAGAGC |
| | AGGTGGCAGC AGGGGAACGT |
| 1251 | CTTCTCATGC TCCGTGATGC ATGAGGCTCT |
| | GCACAACCAC TACACGCAGA |
| 1301 | AGAGCCTCTC CCTGTCTCCG GGTAAATGA |

The various constructs were successfully expressed in CHO cells and were purified to a high degree of purity as determined by analytical size-exclusion chromatography and SDS-PAGE. The hTβRII (G4S)2-hFc, hTβRII (G4S)3-hFc, hTβRII (G4S)4-hFc, hTβRII (G4S)5-hFc and hTβRII (G4S)6-hFc proteins displayed similarly strong stability as determined by SDS-PAGE analysis when maintained in PBS for 13 days at 37° C. The hTβRII (G4S)2-hFc, hTβRII (G4S)3-hFc, hTβRII (G4S)4-hFc proteins were also maintained in rat, mouse or human serum and displayed similarly strong stability.

### TβRII ECD Variants

In addition to the TβRII domains included in the fusion proteins described above (e.g., SEQ ID NO: 46), the disclosure also contemplates fusion proteins comprising alternative TβRII domains. For example, the fusion protein may comprise the wild-type hTβRIIₛₕₒᵣₜ(23-159) sequence shown below (SEQ ID NO: 45) or any of the other TβRII polypeptides disclosed below:

(SEQ ID NO: 45)

| | |
|---|---|
| 1 | TIPPHVQKSV NNDMIVTDNN GAVKFPQLCK |
| | FCDVRESTCD NQKSCMSNCS |
| 51 | ITSICEKPQE VCVAVWRKND ENITLETVCH |
| | DPKLPYHDFI LEDAASPKCI |
| 101 | MKEKKKPGET FFMCSCSSDE CNDNIIFSEE |
| | YNTSNPD |

(1) The hTβRIIₛₕₒᵣₜ(23-159/D110K) amino acid sequence shown below (SEQ ID NO: 57), in which the substituted residue is underlined.

(SEQ ID NO: 57)

| | |
|---|---|
| 1 | TIPPHVQKSV NNDMIVTDNN GAVKFPQLCK |
| | FCDVRESTCD NQKSCMSNCS |
| 51 | ITSICEKPQE VCVAVWRKND ENITLETVCH |
| | DPKLPYHKFI LEDAASPKCI |
| 101 | MKEKKKPGET FFMCSCSSDE CNDNIIFSEE |
| | YNTSNPD |

(2) The N-terminally truncated hTβRIIₛₕₒᵣₜ(29-159) amino acid sequence shown below (SEQ ID NO: 47).

(SEQ ID NO: 47)

| | |
|---|---|
| 1 | QKSVNNDMIV TDNNGAVKFP QLCKFCDVRF |
| | STCDNQKSCM SNCSITSICE |
| 51 | KPQEVCVAVW RKNDENITLE TVCHDPKLPY |
| | HDFILEDAAS PKCIMKEKKK |
| 101 | PGETFFMCSC SSDECNDNII FSEEYNTSNP |
| | D |

(3) The N-terminally truncated hTβRIIₛₕₒᵣₜ(35-159) amino acid sequence shown below (SEQ ID NO: 48).

(SEQ ID NO: 48)

| | |
|---|---|
| 1 | DMIVTDNNGA VKFPQLCKFC DVRESTCDNQ |
| | KSCMSNCSIT SICEKPQEVC |
| 51 | VAVWRKNDEN ITLETVCHDP KLPYHDFILE |
| | DAASPKCIMK EKKKPGETFF |
| 101 | MCSCSSDECN DNIIFSEEYN TSNPD |

(4) The C-terminally truncated hTβRIIₛₕₒᵣₜ(23-153) amino acid sequence shown below (SEQ ID NO: 49).

(SEQ ID NO: 49)

| | |
|---|---|
| 1 | TIPPHVQKSV NNDMIVIDNN GAVKFPQLCK |
| | FCDVRESTCD NQKSCMSNCS |
| 51 | ITSICEKPQE VCVAVWRKND ENITLETVCH |
| | DPKLPYHDFI LEDAASPKCI |
| 101 | MKEKKKPGET FFMCSCSSDE CNDNIIFSEE |
| | Y |

(5) The C-terminally truncated hTβRIIₛₕₒᵣₜ(23-153/N70D) amino acid sequence shown below (SEQ ID NO: 59), in which the substituted residue is underlined.

(SEQ ID NO: 59)

| | |
|---|---|
| 1 | TIPPHVQKSV NNDMIVTDNN GAVKFPQLCK |
| | FCDVRESTCD NQKSCMSDCS |
| 51 | ITSICEKPQE VCVAVWRKND ENITLETVCH |
| | DPKLPYHDFI LEDAASPKCI |
| 101 | MKEKKKPGET FFMCSCSSDE CNDNIIFSEE |
| | Y |

Applicants also envision five corresponding variants (SEQ ID NOs: 58, 52, 54, 60) based on the wild-type hTβRII_{long}(23-184) sequence shown above and below (SEQ ID NO: 67), in which the 25 amino-acid insertion is underlined. Note that splicing results in a conservative amino acid substitution (Val→Ile) at the flanking position C-terminal to the insertion.

(SEQ ID NO: 67)

| | |
|---|---|
| 1 | TIPPHVQKSD VEMEAQKDEI ICPSCNRTAH |
| | PLRHINNDMI VTDNNGAVKE |
| 51 | PQLCKFCDVR FSTCDNQKSC MSNCSITSIC |
| | EKPQEVCVAV WRKNDENITL |
| 101 | ETVCHDPKLP YHDFILEDAA SPKCIMKEKK |
| | KPGETFFMCS CSSDECNDNI |
| 151 | IFSEEYNTSN PD |

(1) The hTβRII_{long}(23-184/D135K) amino acid sequence shown below (SEQ ID NO: 58), in which the substituted residue is double underlined.

(SEQ ID NO: 58)

| | |
|---|---|
| 1 | TIPPHVQKSD VEMEAQKDEI ICPSCNRTAH |
| | PLRHINNDMI VTDNNGAVKE |
| 51 | PQLCKFCDVR FSTCDNQKSC MSNCSITSIC |
| | EKPQEVCVAV WRKNDENITL |
| 101 | ETVCHDPKLP YHKFILEDAA SPKCIMKEKK |
| | KPGETFFMCS CSSDECNDNI |
| 151 | IFSEEYNTSN PD |

(2) The N-terminally truncated hTβRII_{long}(29-184) amino acid sequence shown below (SEQ ID NO: 52).

(SEQ ID NO: 52)

| | |
|---|---|
| 1 | QKSDVEMEAQ KDEIICPSCN RTAHPLRHIN |
| | NDMIVTDNNG AVKFPQLCKE |
| 51 | CDVRFSTCDN QKSCMSNCSI TSICEKPQEV |
| | CVAVWRKNDE NITLETVCHD |
| 101 | PKLPYHDFIL EDAASPKCIM KEKKKPGETF |
| | FMCSCSSDEC NDNIIFSEEY |
| 151 | NTSNPD |

(3) The N-terminally truncated hTβRII_{long}(60-184) amino acid sequence shown below (same as SEQ ID NO: 48).

```
(same as SEQ ID NO: 48)
    1 DMIVTDNNGA VKFPQLCKFC DVRESTCDNQ
        KSCMSNCSIT SICEKPQEVC
  51 VAVWRKNDEN ITLETVCHDP KLPYHDFILE
        DAASPKCIMK EKKKPGETFF
101 MCSCSSDECN DNIIFSEEYN TSNPD
```

(4) The C-terminally truncated hTβRII_{long}(23-178) amino acid sequence shown below (SEQ ID NO: 54).

(SEQ ID NO: 54)

| | |
|---|---|
| 1 | TIPPHVQKSD VEMEAQKDEI ICPSCNRTAH |
| | PLRHINNDMI VTDNNGAVKF |
| 51 | PQLCKFCDVR FSTCDNQKSC MSNCSITSIC |
| | EKPQEVCVAV WRKNDENITL |
| 101 | ETVCHDPKLP YHDFILEDAA SPKCIMKEKK |
| | KPGETFFMCS CSSDECNDNI |
| 151 | IFSEEY |

(5) The C-terminally truncated hTβRII_{long}(23-178/N95D) amino acid sequence shown below (SEQ ID NO: 60), in which the substituted residue is double underlined.

(SEQ ID NO: 60)

| | |
|---|---|
| 1 | TIPPHVQKSD VEMEAQKDEI ICPSCNRTAH |
| | PLRHINNDMI VTDNNGAVKF |
| 51 | PQLCKFCDVR FSTCDNQKSC MSDCSITSIC |
| | EKPQEVCVAV WRKNDENITL |
| 101 | ETVCHDPKLP YHDFILEDAA SPKCIMKEKK |
| | KPGETFFMCS CSSDECNDNI |
| 151 | IFSEEY |

Additional TβRII ECD variants include:
(A) The N- and C-terminally truncated hTβRIIₛₕₒᵣₜ(35-153) or hTβRII_{long}(60-178) amino acid sequence shown below (SEQ ID NO: 51).

```
(SEQ ID NO: 51)
    1 DMIVTDNNGA VKFPQLCKFC DVRESTCDNQ
        KSCMSNCSIT SICEKPQEVC
  51 VAVWRKNDEN ITLETVCHDP KLPYHDFILE
        DAASPKCIMK EKKKPGETFF
101 MCSCSSDECN DNIIFSEEY
```

(B) The N- and C-terminally truncated hT RIIshorₜ(29-153) amino acid sequence shown below (SEQ ID NO: 50).

(SEQ ID NO: 50)

| | |
|---|---|
| 1 | QKSVNNDMIV TDNNGAVKFP QLCKFCDVRF |
| | STCDNQKSCM SNCSITSICE |
| 51 | KPQEVCVAVW RKNDENITLE TVCHDPKLPY |
| | HDFILEDAAS PKCIMKEKKK |
| 101 | PGETFFMCSC SSDECNDNII FSEEY |

(C) The N- and C-terminally truncated hTβRIIion_{g}(29-178) amino acid sequence shown below (SEQ ID NO: 55).

(SEQ ID NO: 55)

| | |
|---|---|
| 1 | QKSDVEMEAQ KDEIICPSCN RTAHPLRHIN |
| | NDMIVTDNNG AVKFPQLCKE |
| 51 | CDVRESTCDN QKSCMSNCSI TSICEKPQEV |
| | CVAVWRKNDE NITLETVCHD |
| 101 | PKLPYHDFIL EDAASPKCIM KEKKKPGETF |
| | FMCSCSSDEC NDNIIFSEEY |

Any of the above variants (SEQ ID NO: 57, 47, 48, 49, 59, 58, 52, 54, 60, 51, 50, and 55) could incorporate an insertion of 36 amino acids (SEQ ID NO: 65) between the pair of glutamate residues (positions 151 and 152 of SEQ ID NO: 43, or positions 176 and 177 of SEQ ID NO: 42) located near the C-terminus of the hTβRII ECD, as occurs naturally in the hTβRII isoform C (Konrad et al., BMC Genomics 8:318, 2007).

(SEQ ID NO: 65)
GRCKIRHIGS NNRLQRSTCQ NTGWESAHVM KTPGER

As an example, the paired glutamate residues flanking the optional insertion site are denoted below (underlined) for the hTβRIIₛₕₒᵣₜ(29-159) variant (SEQ ID NO: 47).

(SEQ ID NO: 47)

| | |
|---|---|
| 1 | QKSVNNDMIV TDNNGAVKFP QLCKFCDVRF |
| | STCDNQKSCM SNCSITSICE |
| 51 | KPQEVCVAVW RKNDENITLE TVCHDPKLPY |
| | HDFILEDAAS PKCIMKEKKK |
| 101 | PGETFFMCSC SSDECNDNII FSEEYNTSNP |
| | D |

### Fc Domain Variants

While the constructs described above were generated with an Fc domain having the amino acid sequence of SEQ ID NO: 11, the disclosure contemplates hTβRII-hFc fusion proteins comprising alternative Fc domains, including a human IgG2 Fc domain (SEQ ID NO: 12, below) or full-length human IgG1 Fc (hG1Fc) (SEQ ID NO: 164, below). Optionally, a polypeptide unrelated to an Fc domain could be attached in place of the Fc domain.

| (SEQ ID NO: 12) | |
|---|---|
| 1 | VECPPCPAPP VAGPSVFLFP PKPKDTLMIS |
| | RTPEVTCVVV DVSHEDPEVQ |
| 51 | FNWYVDGVEV HNAKTKPREE QFNSTERVVS |
| | VLTVVHQDWL NGKEYKCKVS |
| 101 | NKGLPAPIEK TISKTKGQPR EPQVYTLPPS |
| | REEMTKNQVS LTCLVKGFYP |
| 151 | SDIAVEWESN GQPENNYKTT PPMLDSDGSF |
| | FLYSKLTVDK SRWQQGNVES |
| 201 | CSVMHEALHN HYTQKSLSLS PGK |

The C-terminal lysine residue of the Fc domain can be deleted. The amino acid sequence of SEQ ID NO: 12 may optionally be provided with the lysine removed from the C-terminus (SEQ ID NO: 165):

| (SEQ ID NO: 165) | |
|---|---|
| 1 | VECPPCPAPP VAGPSVFLFP PKPKDTLMIS |
| | RTPEVTCVVV DVSHEDPEVQ |
| 51 | FNWYVDGVEV HNAKTKPREE QFNSTERVVS |
| | VLTVVHODWL NGKEYKCKVS |
| 101 | NKGLPAPIEK TISKTKGQPR EPQVYTLPPS |
| | REEMTKNQVS LTCLVKGFYP |
| 151 | SDIAVEWESN GQPENNYKTT PPMLDSDGSF |
| | FLYSKLTVDK SRWQQGNVES |
| 201 | CSVMHEALHN HYTQKSLSLS PG |
| (SEQ ID NO: 164) | |
| 1 | GGPKSCDKTH TCPPCPAPEL LGGPSVELFP |
| | PKPKDTLMIS RTPEVTCVVV |
| 51 | DVSHEDPEVK FNWYVDGVEV HNAKTKPREE |
| | QYNSTYRVVS VLTVLHQDWL |
| 101 | NGKEYKCKVS NKALPAPIEK TISKAKGQPR |
| | EPQVYTLPPS REEMTKNQVS |
| 151 | LTCLVKGFYP SDIAVEWESN GQPENNYKTT |
| | PPVLDSDGSF FLYSKLTVDK |
| 201 | SRWQQGNVFS CSVMHEALHN HYTQKSLSLS |
| | PGK |

The C-terminal lysine residue of the Fc domain can be deleted. The amino acid sequence of SEQ ID NO: 164 may optionally be provided with the lysine removed from the C-terminus (SEQ ID NO: 166):

| (SEQ ID NO: 166) | |
|---|---|
| 1 | GGPKSCDKTH TCPPCPAPEL LGGPSVELFP |
| | PKPKDTLMIS RTPEVTCVVV |
| 51 | DVSHEDPEVK FNWYVDGVEV HNAKTKPREE |
| | QYNSTYRVVS VLTVLHQDWL |
| 101 | NGKEYKCKVS NKALPAPIEK TISKAKGQPR |
| | EPQVYTLPPS REEMTKNQVS |
| 151 | LTCLVKGFYP SDIAVEWESN GQPENNYKTT |
| | PPVLDSDGSF FLYSKLTVDK |
| 201 | SRWQQGNVFS CSVMHEALHN HYTQKSLSLS |
| | PG |

### Leader Sequence Variants

While the generated constructs described above included the TPA leader sequence, alternative leader sequences may be used, such as the native leader sequence (SEQ ID NO: 167—below) or the honey bee melittin (SEQ ID NO: 24—below) leader sequences.

Native: MGRGLLRGLWPLHIVLWTRIAS (SEQ ID NO: 167)

Honey bee melittin (HBML): MKFLVNVALVFMVVYISYIYA (SEQ ID NO: 24)

mTβRII-mFc

mTβRII-mFc comprises murine TβRII extracellular domain and murine IgG2a Fc. The signal sequence is underlined. The linker in mTβRII-mFc is TGGG (SEQ ID NO: 142), bolded and underlined below. The human version (SEQ ID NO: 66) has a longer linker (SEQ ID NO: 82). The unprocessed sequence (SEQ ID NO: 168) is below:

| (SEQ ID NO: 168) | |
|---|---|
| 1 | MDAMKRGLCC VLLLCGAVFV SPGAIPPHVP |
| | KSDVEMEAQK DASIHLSCNR |
| 51 | TIHPLKHFNS DVMASDNGGA VKLPQLCKFC |
| | DVRLSTCDNQ KSCMSNCSIT |
| 101 | AICEKPHEVC VAVWRKNDKN ITLETVCHDP |
| | KLTYHGFTLE DAASPKCVMK |
| 151 | EKKRAGETFF MCACNMEECN DYIIFSEEYT |
| | TSSPDTGGGE PRVPITONPC |
| 201 | PPLKECPPCA APDLLGGPSV FIFPPKIKDV |
| | LMISLSPMVT CVVVDVSEDD |
| 251 | PDVQISWFVN NVEVHTAQTQ THREDYNSTL |
| | RVVSALPIQH QDWMSGKEFK |
| 301 | CKVNNRALPS PIEKTISKPR GPVRAPQVYV |
| | LPPPAEEMTK KEFSLTCMIT |
| 351 | GFLPAEIAVD WTSNGRTEQN YKNTATVLDS |
| | DGSYFMYSKL RVOKSTWERG |
| 401 | SLFACSVVHE GLHNHLTTKT ISRSLGK* |

The mature version of mTβRII-mFc is shown as SEQ ID NO: 169:

| (SEQ ID NO: 169) | |
|---|---|
| 1 | IPPHVPKSDV EMEAQKDASI HLSCNRTIHP |
| | LKHFNSDVMA SDNGGAVKLP |
| 51 | QLCKFCDVRL STCDNQKSCM SNCSITAICE |
| | KPHEVCVAVW RKNDKNITLE |
| 101 | TVCHDPKLTY HGFTLEDAAS PKCVMKEKKR |
| | AGETFFMCAC NMEECNDYII |
| 151 | FSEEYTTSSP DTGGGEPRVP ITONPCPPLK |
| | ECPPCAAPDL LGGPSVFIFP |
| 201 | PKIKDVLMIS LSPMVTCVVV DVSEDDPDVQ |
| | ISWFVNNVEV HTAQTQTHRE |
| 251 | DYNSTLRVVS ALPIQHQDWM SGKEFKCKVN |
| | NRALPSPIEK TISKPRGPVR |
| 301 | APQVYVLPPP AEEMTKKEFS LTCMITGFLP |
| | AEIAVDWTSN GRTEQNYKNT |
| 351 | ATVLDSDGSY FMYSKLRVOK STWERGSLFA |
| | CSVVHEGLHN HLTTKTISRS |
| 401 | LGK |

Furthermore, the C-terminal lysine residue of the Fc domain can be deleted. The amino acid sequence of SEQ ID NO: 168 may optionally be provided with the lysine removed from the C-terminus (SEQ ID NO: 170)

| (SEQ ID NO: 170) | |
|---|---|
| 1 | IPPHVPKSDV EMEAQKDASI HLSCNRTIHP |
| | LKHFNSDVMA SDNGGAVKLP |
| 51 | QLCKFCDVRL STCDNQKSCM SNCSITAICE |
| | KPHEVCVAVW RKNDKNITLE |
| 101 | TVCHDPKLTY HGFTLEDAAS PKCVMKEKKR |
| | AGETFFMCAC NMEECNDYII |
| 151 | FSEEYTTSSP DTGGGEPRVP ITQNPCPPLK |
| | ECPPCAAPDL LGGPSVFIFP |
| 201 | PKIKDVLMIS LSPMVTCVVV DVSEDDPDVQ |
| | ISWFVNNVEV HTAQTQTHRE |
| 251 | DYNSTLRVVS ALPIQHQDWM SGKEFKCKVN |
| | NRALPSPIEK TISKPRGPVR |
| 301 | APQVYVLPPP AEEMTKKEFS LTCMITGFLP |
| | AEIAVDWTSN GRTEQNYKNT |
| 351 | ATVLDSDGSY FMYSKLRVOK STWERGSLFA |
| | CSVVHEGLHN HLTTKTISRS |
| 401 | LG |

mTβRII-mFc nucleic acid sequence (SEQ ID NO: 171):

| (SEQ ID NO: 171) | |
|---|---|
| 1 | ATGGATGCAA TGAAGAGAGG GCTCTGCTGT |
| | GTGCTGCTGC TGTGTGGAGC |
| 51 | AGTCTTCGTT TCGCCCGGCG CCATCCCGCC |
| | GCACGTTCCC AAGTCGGATG |
| 101 | TGGAAATGGA AGCCCAGAAA GATGCATCCA |
| | TCCACCTAAG CTGTAATAGG |
| 151 | ACCATCCATC CACTGAAACA TTTTAACAGT |
| | GATGTCATGG CCAGCGACAA |
| 201 | TGGCGGTGCG GTCAAGCTTC CACAGCTGTG |
| | CAAGTTTTGC GATGTGAGAC |
| 251 | TGTCCACTTG CGACAACCAG AAGTCCTGCA |
| | TGAGCAACTG CAGCATCACG |
| 301 | GCCATCTGTG AGAAGCCGCA TGAAGTCTGC |
| | GTGGCCGTGT GGAGGAAGAA |
| 351 | CGACAAGAAC ATTACTCTGG AGACGGTTTG |
| | CCACGACCCC AAGCTCACCT |
| 401 | ACCACGGCTT CACTCTGGAA GATGCCGCTT |
| | CTCCCAAGTG TGTCATGAAG |
| 451 | GAAAAGAAAA GGGCGGGCGA GACTTTCTTC |
| | ATGTGTGCCT GTAACATGGA |
| 501 | AGAGTGCAAC GATTACATCA TCTTTTCGGA |
| | AGAATACACC ACCAGCAGTC |
| 551 | CCGACACCGG TGGGGGTGAG CCCAGAGTGC |
| | CCATAACACA GAACCCCTGT |
| 601 | CCTCCACTCA AAGAGTGTCC CCCATGCGCA |
| | GCTCCAGACC TCTTGGGTGG |
| 651 | ACCATCCGTC TTCATCTTCC CTCCAAAGAT |
| | CAAGGATGTA CTCATGATCT |
| 701 | CCCTGAGCCC CATGGTCACA TGTGTGGTGG |
| | TGGATGTGAG CGAGGATGAC |
| 751 | CCAGACGTCC AGATCAGCTG GTTTGTGAAC |
| | AACGTGGAAG TACACACAGC |
| 801 | TCAGACACAA ACCCATAGAG AGGATTACAA |
| | CAGTACTCTC CGGGTGGTCA |
| 851 | GTGCCCTCCC CATCCAGCAC CAGGACTGGA |
| | TGAGTGGCAA GGAGTTCAAA |
| 901 | TGCAAGGTCA ACAACAGAGC CCTCCCATCC |
| | CCCATCGAGA AAACCATCTC |
| 951 | AAAACCCAGA GGGCCAGTAA GAGCTCCACA |
| | GGTATATGTC TTGCCTCCAC |
| 1001 | CAGCAGAAGA GATGACTAAG AAAGAGTTCA |
| | GTCTGACCTG CATGATCACA |
| 1051 | GGCTTCTTAC CTGCCGAAAT TGCTGTGGAC |
| | TGGACCAGCA ATGGGCGTAC |
| 1101 | AGAGCAAAAC TACAAGAACA CCGCAACAGT |
| | CCTGGACTCT GATGGTTCTT |
| 1151 | ACTTCATGTA CAGCAAGCTC AGAGTACAAA |
| | AGAGCACTTG GGAAAGAGGA |
| 1201 | AGTCTTTTCG CCTGCTCAGT GGTCCACGAG |
| | GGTCTGCACA ATCACCTTAC |
| 1251 | GACTAAGACC ATCTCCCGGT CTCTGGGTAA |
| | ATGA |

### Example 6: Differential Ligand Inhibition by Receptor Fusion Protein Variants in Cell-Based Assay

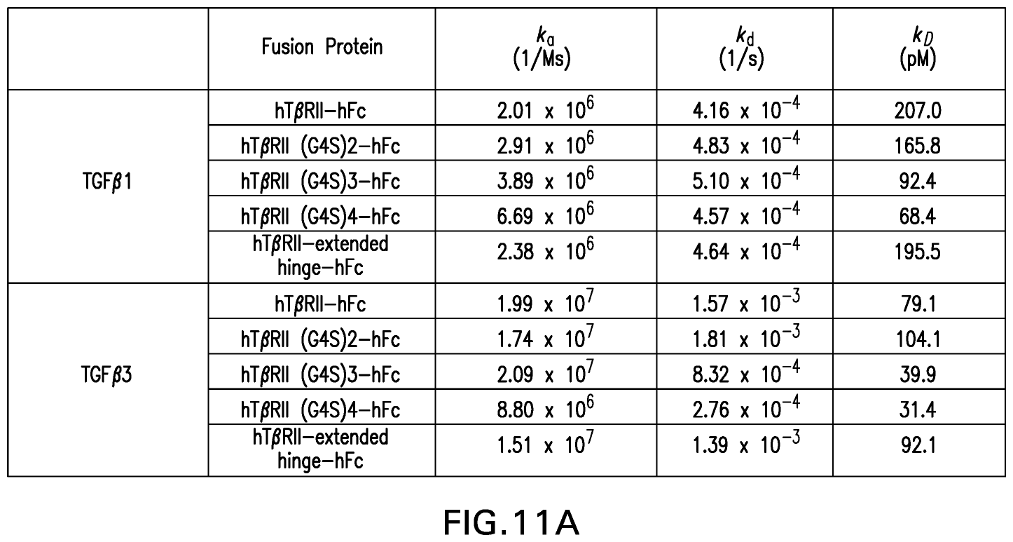

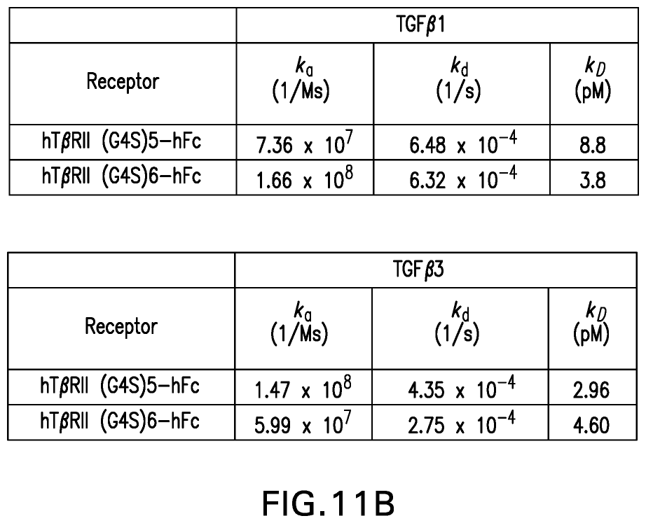

Affinities of TGFβ1, TGFβ2 and TGFβ3 for hTβRII (G4S)2-hFc; hTβRII (G4S)3-hFc; hTβRII (G4S)4-hFc; hTβRII-hFc; and hTβRII extended hinge-hFc proteins were evaluated in vitro with a Biacore™ instrument, and the results are summarized in FIGS. 11A and 11B. Each of the fusion proteins was capable of binding TGFβ1 and TGFβ3 with high affinity, but the constructs having linker lengths longer than or equal to (G4S)4 (SEQ ID NO: 172) were surprisingly capable of binding to both TGFβ1 and TGFβ3 with higher affinity than constructs having linker lengths shorter than (G4S)4 (SEQ ID NO: 172). Binding between TGFβ2 and any of the constructs was low or transient. Deglycosylation of the constructs did not change binding.

A reporter gene assay in A549 cells was used to determine the ability of hTβRII-hFc variants to inhibit activity of TGFβ1, TGFβ2 and TGFβ3. This assay is based on a human lung carcinoma cell line transfected with a pGL3(CAGA)12 reporter plasmid (Dennler et al, 1998, EMBO 17: 3091-3100) as well as a Renilla reporter plasmid (pRLCMV) to control for transfection efficiency. The CAGA motif is present in the promoters of TGFβ-responsive genes (for example, PAI-1), so this vector is of general use for factors signaling through SMAD2 and SMAD3.

On the first day of the assay, A549 cells (ATCC®: CCL-185™) were distributed in 48-well plates. On the second day, a solution containing pGL3(CAGA)12, pRLCMV, X-tremeGENE 9 (Roche Applied Science), and OptiMEM (Invitrogen) was preincubated, then added to Eagle's minimum essential medium (EMEM, ATCC®) supplemented with 0.1% BSA, which was applied to the plated cells for incubation overnight at 37° C., 5% CO₂. On the third day, medium was removed, and cells were incubated overnight at 37° C., 5% CO₂ with a mixture of ligands and inhibitors prepared as described below.

Serial dilutions of test articles were made in a 48-well plate in assay buffer (EMEM+0.1% BSA). An equal volume of assay buffer containing the test ligand was added to obtain a final ligand concentration equal to the EC50 determined previously. Human TGFβ1, human TGFβ2, and human TGFβ3 were obtained from PeproTech. Test solutions were incubated at 37° C. for 30 minutes, then a portion of the mixture was added to all wells. After incubation with test solutions overnight, cells were rinsed with phosphate-buffered saline, then lysed with passive lysis buffer (Promega E1941) and stored overnight at −70° C. On the fourth and final day, plates were warmed to room temperature with gentle shaking. Cell lysates were transferred in duplicate to a chemiluminescence plate (96-well) and analyzed in a luminometer with reagents from a Dual-Luciferase Reporter Assay system (Promega E1980) to determine normalized luciferase activity.

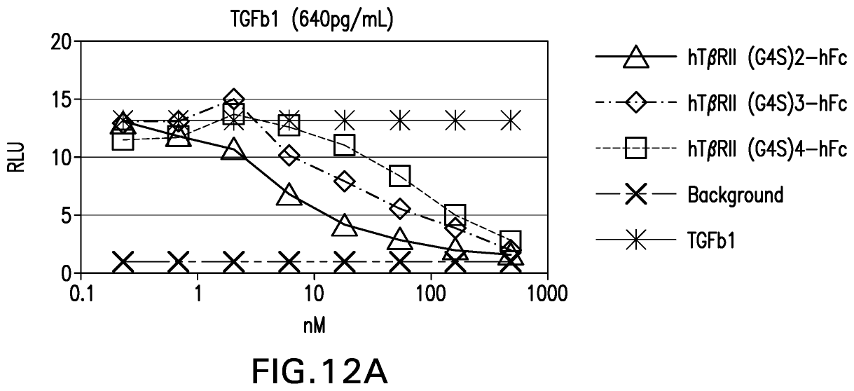

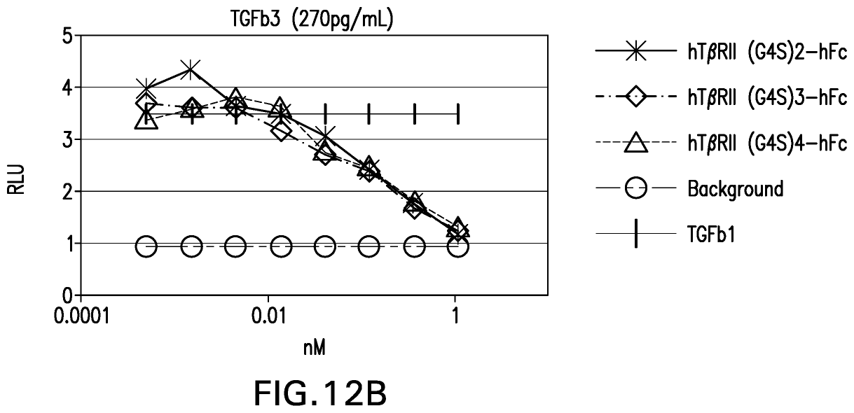

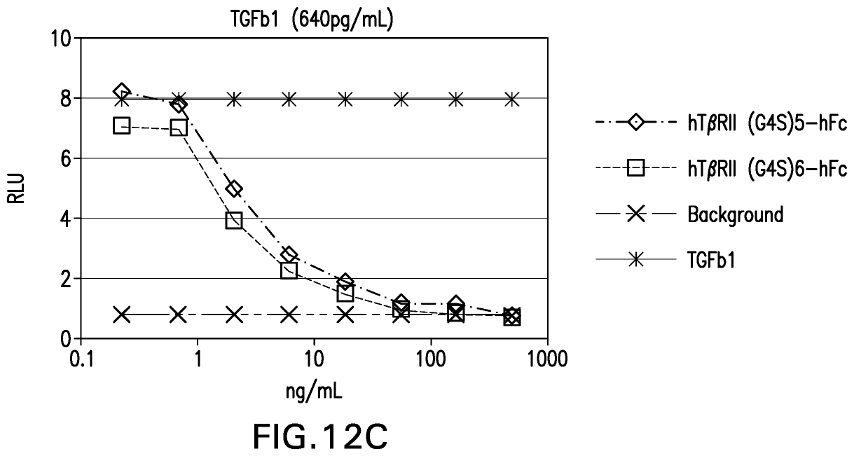

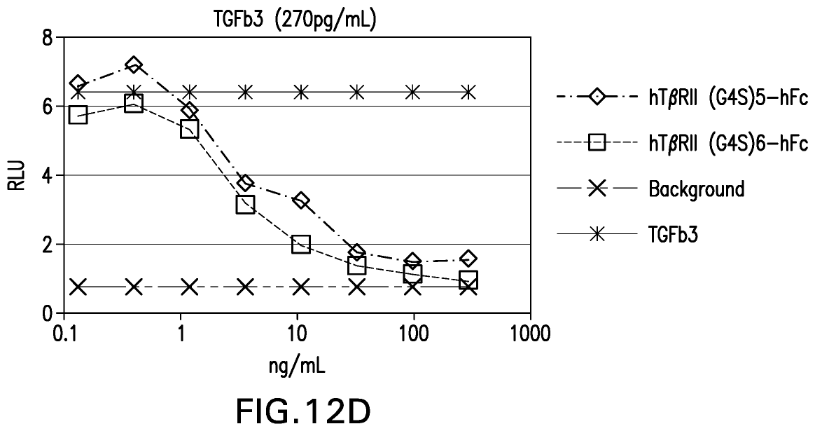

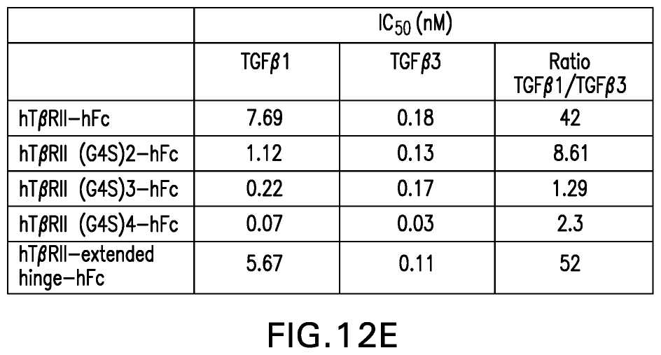

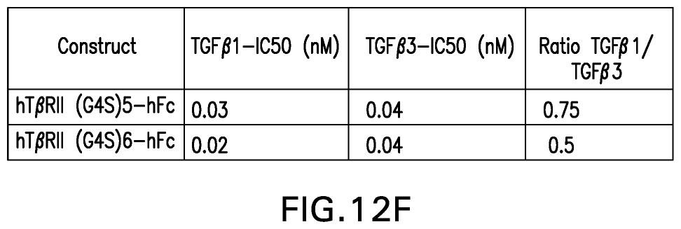

As illustrated in FIGS. 12A-12F, the hTβRII (G4S)2-hFc; hTβRII (G4S)3-hFc; hTβRII (G4S)4-hFc; hTβRII (G4S)5-hFc; hTβRII (G4S)6-hFc; hTβRII-hFc; and hTβRII extended hinge-hFc proteins all were capable of inhibiting both TGFβ1 and TGFβ3. Interestingly, while there was a correlation between improved TGFβ1 and TGFβ3 inhibition and linker length for the hTβRII (G4S)2-hFc; hTβRII (G4S)3-hFc and hTβRII (G4S)4-hFc constructs (FIG. 12E), this improvement trend appeared to have plateaued for hTβRII (G4S)5-hFc and hTβRII (G4S)6-hFc constructs (FIG. 12F).

### Example 7: Effects of a Combination Therapy on Group 3 Pulmonary Hypertension in Two Bleomycin-Induced Pulmonary Hypertension and Fibrosis Rat Model

The effects of a combination therapy comprising an ActRIIA-mFc fusion protein (ActRIIA-mFc homodimer as described in Example 1) and a TβRII-mFc fusion protein (TβRII-mFc homodimer as described in Example 5) will be examined in two rat models of Group 3 pulmonary hypertension (Grp3-PH).

Similar to the first model described in Example 4, twelve Wistar male rats will be intratracheally administered with a single dose of bleomycin (Bleo, 0.6 U/rat) at day 0 and randomized into two treatment groups (6 rats per group): 1) treatment with monocrotaline (MCT, 60 mg/kg administered s.c. as a single dose at day 7 of study) and Tris buffered saline (s.c. as 1 ml/kg every three days) (vehicle treatment group), 2) treatment with MCT (60 mg/kg administered s.c. as a single dose at day 7 of study) and a combination of ActRIIA-mFc (5 mg/kg administered s.c. every three days) and TβRII-mFc (5 mg/kg administered s.c. every three days). Rats will be treated for 35 days, and body weights will be recorded weekly.

Rats will be anesthetized on day 42 with ˜3-4% isoflurane and placed on heating pads. Right ventricular systolic pressure (RVSP) will be measured by advancing a 2F curve tip pressure transducer catheter, (SPR-513, Millar Instruments) into the right ventricle (RV) via right jugular vein under ˜1.5-2% isofluorane anesthesia. RV hypertrophy will be assessed by taking the weight ratio of RV free wall and LV+Septum (RV/LV+S, Fulton's Index). Lungs will be collected, fixed in 10% formalin, embedded in paraffin, and sectioned for Masson's trichrome stain to assess fibrosis.

In another model, six Sprague-Dawley male rats will be intratracheally administered with a single dose of bleomycin (Bleo, 0.6 U/rat) at day 0 and randomized into two treatment groups: 1) treatment with semaxanib (20 mg/kg administered s.c. as a single dose at day 7 of study)/hypoxia and Tris buffered saline (administered s.c. as 1 ml/kg, every three days) (Bleo/Su/Hx-PBS group), 2) treatment with semaxanib (20 mg/kg administered s.c. as a single dose at day 7 of study)/hypoxia and a combination of ActRIIA-mFc (5 mg/kg administered s.c. every three days) and TβRII-mFc (5 mg/kg administered s.c. every three days). Rats will be treated for 35 days, and body weights will be recorded weekly throughout the study.

On day 42, rats will be anesthetized with ˜3-4% isoflurane and placed on controlled heating pads. Right ventricular systolic pressure (RVSP) will be measured by advancing a 2F curve tip pressure transducer catheter, (SPR-513, Millar Instruments) into the right ventricle (RV) via right jugular vein under ˜1.5-2% isofluorane anesthesia. RV hypertrophy will be assessed by taking the weight ratio of RV free wall and LV+Septum (RV/LV+S, Fulton's Index).

1-235. (canceled)

236. A method of treating pulmonary hypertension associated with lung disease, comprising administering to a patient in need thereof:
1) an effective amount of an ActRIIA fusion protein comprising
a) an ActRIIA polypeptide comprising an amino acid sequence that is at least 95% identical to an amino acid sequence corresponding to residues 30-110 of SEQ ID NO: 1;
b) an Fc domain of an IgG1 immunoglobulin; and
c) a linker domain,
wherein the linker domain is positioned between the ActRIIA polypeptide and the Fc domain of the IgG1 immunoglobulin; and
2) an effective amount of a TβRII fusion protein comprising
a) a TβRII polypeptide comprising an amino acid sequence that is at least 95% identical to an amino acid sequence corresponding to SEQ ID NO: 45 or SEQ ID NO: 46;
b) an Fc domain of an IgG1 immunoglobulin; and
c) a linker domain,
wherein the linker domain is positioned between the TβRII polypeptide and the Fc domain of the IgG1 immunoglobulin.

237. The method of claim 236, wherein the ActRIIA polypeptide comprises an amino acid sequence of SEQ ID NO: 2.

238. The method of claim 236, wherein the ActRIIA fusion protein linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

239. The method of claim 236, wherein the ActRIIA fusion protein linker domain is TGGG (SEQ ID NO: 20).

240. The method of claim 236, wherein the ActRIIA fusion protein Fc domain comprises an amino acid sequence that is at least 95% identical to the amino acid sequence of SEQ ID NO: 32.

241. The method of claim 236, wherein the ActRIIA fusion protein Fc domain comprises an amino acid sequence that is at least 99% identical to the amino acid sequence of SEQ ID NO: 32.

242. The method of claim 236, wherein the ActRIIA fusion protein Fc domain comprises an amino acid sequence of SEQ ID NO: 32.

243. The method of claim 236, wherein the ActRIIA fusion protein comprises an amino acid sequence that is at least 95% identical to the amino acid sequence of SEQ ID NO: 23.

244. The method of claim 236, wherein the ActRIIA fusion protein comprises an amino acid sequence that is at least 99% identical to the amino acid sequence of SEQ ID NO: 23.

245. The method of claim 236, wherein the ActRIIA fusion protein comprises an amino acid sequence of SEQ ID NO: 23.

246. The method of claim 236, wherein the ActRII fusion protein comprises an amino acid sequence that is at least 95% identical to the amino acid sequence of SEQ ID NO: 41.

247. The method of claim 236, wherein the ActRIIA fusion protein comprises an amino acid sequence that is at least 99% identical to the amino acid sequence of SEQ ID NO: 41.

248. The method of claim 236, wherein the ActRIIA fusion protein comprises an amino acid sequence of SEQ ID NO: 41.

249. The method of claim 236, wherein the ActRIIA fusion protein comprises the amino acid sequence of SEQ ID NO: 41.

250. The method of claim 236, wherein the ActRIIA fusion protein is part of a homodimer protein complex.

251. The method of claim 250, wherein the ActRIIA fusion protein is glycosylated.

252. The method of claim 236, wherein the ActRIIA polypeptide binds to one or more ligands selected from the group consisting of: activin A, activin B, GDF11, BMP10, GDF8, and BMP6.

253. The method of claim 236, wherein the TβRII polypeptide comprises an amino acid sequence comprising SEQ ID NO: 46.

254. The method of claim 236, wherein the TβRII fusion protein linker domain is selected from the group consisting of GGGGSGGGGS (SEQ ID NO: 79), TGGGGSGGGGS (SEQ ID NO: 80), TGGGGSGGGGSGGGGS (SEQ ID NO: 81), TGGGGSGGGGSGGGGSGGGGS (SEQ ID NO: 82), TGGGGSGGGGSGGGGSGGGGSGGGGS (SEQ ID NO: 83), TGGGGSGGGGSGGGGSGGGGSGGGGSGGGGS (SEQ ID NO: 84), TGGGGSGGGGSGGGGSGGGGSGGGGSGGGGSGGGGS (SEQ ID NO: 85), GGGGSGGGGS (SEQ ID NO: 178), GGGGSGGGGSGGGGS (SEQ ID NO: 179), GGGGSGGGGSGGGGSGGGGS (SEQ ID NO: 180), GGGGSGGGGSGGGGSGGGGSGGGGS (SEQ ID NO: 181), GGGGSGGGGSGGGGSGGGGSGGGGSGGGGS (SEQ ID NO: 182), or GGGGSGGGGSGGGGSGGGGSGGGGSGGGGSGGGGS (SEQ ID NO: 183).

255. The method of claim 236, wherein the TβRII fusion protein linker domain is TGGGGSGGGGSGGGGSGGGGS (SEQ ID NO: 82).

256. The method of claim 236, wherein the TβRII fusion protein Fc domain comprises an amino acid sequence that is at least 95% identical to the amino acid sequence of SEQ ID NO: 11.

257. The method of claim 236, wherein the TβRII fusion protein Fc domain comprises an amino acid sequence that is at least 99% identical to the amino acid sequence of SEQ ID NO: 11.

258. The method of claim 236, wherein the TβRII fusion protein Fc domain comprises an amino acid sequence of SEQ ID NO: 11.

259. The method of claim 236, wherein the TβRII fusion protein comprises an amino acid sequence that is at least 95% identical to SEQ ID NO: 68.

260. The method of claim 236, wherein the TβRII fusion protein comprises an amino acid sequence that is at least 99% identical to SEQ ID NO: 68.

261. The method of claim 236, wherein the TβRII fusion protein comprises an amino acid sequence of SEQ ID NO: 68.

262. The method of claim 236, wherein the TβRII fusion protein comprises an amino acid sequence that is at least 95% identical to SEQ ID NO: 70.

263. The method of claim 236, wherein the TβRII fusion protein comprises an amino acid sequence that is at least 99% identical to SEQ ID NO: 70.

264. The method of claim 236, wherein the TβRII fusion protein comprises an amino acid sequence of SEQ ID NO: 70.

265. A method of treating pulmonary hypertension associated with obstructive lung disease, comprising administering to a patient in need thereof:
1) an effective amount of an ActRIIA fusion protein comprising
a) an ActRIIA polypeptide comprising an amino acid sequence that is at least 95% identical to an amino acid sequence corresponding to residues 30-110 of SEQ ID NO: 1;
b) an Fc domain of an IgG1 immunoglobulin; and
c) a linker domain,
wherein the linker domain is positioned between the ActRIIA polypeptide and the Fc domain of the IgG1 immunoglobulin; and
2) an effective amount of a TβRII fusion protein comprising
a) a TβRII polypeptide comprising an amino acid sequence that is at least 95% identical to an amino acid sequence corresponding to SEQ ID NO: 45 or SEQ ID NO: 46;
b) an Fc domain of an IgG1 immunoglobulin; and
c) a linker domain,
wherein the linker domain is positioned between the TβRII polypeptide and the Fc domain of the IgG1 immunoglobulin.

266. The method of claim 265, wherein the ActRIIA polypeptide comprises an amino acid sequence of SEQ ID NO: 2.

267. The method of claim 265, wherein the ActRIIA fusion protein linker domain is selected from the group consisting of: TGGG (SEQ ID NO: 20), TGGGG (SEQ ID NO: 18), SGGGG (SEQ ID NO: 19), GGGGS (SEQ ID NO: 22), GGG (SEQ ID NO: 16), GGGG (SEQ ID NO: 17), and SGGG (SEQ ID NO: 21).

268. The method of claim 265, wherein the ActRIIA fusion protein linker domain is TGGG (SEQ ID NO: 20).

269. The method of claim 265, wherein the ActRIIA fusion protein Fc domain comprises an amino acid sequence that is at least 90% identical to the amino acid sequence of SEQ ID NO: 32.

270. The method of claim 265, wherein the ActRIIA fusion protein Fc domain comprises an amino acid sequence that is at least 95% identical to the amino acid sequence of SEQ ID NO: 32.

271. The method of claim 265, wherein the ActRIIA fusion protein Fc domain comprises an amino acid sequence that is at least 99% identical to the amino acid sequence of SEQ ID NO: 32.

272. The method of claim 265, wherein the ActRIIA fusion protein comprises an amino acid sequence that is at least 95% identical to the amino acid sequence of SEQ ID NO: 23.

273. The method of claim 265, wherein the ActRIIA fusion protein comprises an amino acid sequence that is at least 99% identical to the amino acid sequence of SEQ ID NO: 23.

274. The method of claim 265, wherein the ActRIIA fusion protein comprises an amino acid sequence of SEQ ID NO: 23.

275. The method of claim 265, wherein the ActRIIA fusion protein comprises an amino acid sequence that is at least 95% identical to the amino acid sequence of SEQ ID NO: 41.

276. The method of claim 265, wherein the ActRIIA fusion protein comprises an amino acid sequence that is at least 99% identical to the amino acid sequence of SEQ ID NO: 41.

277. The method of claim 265, wherein the ActRIIA fusion protein comprises an amino acid sequence of SEQ ID NO: 41.

278. The method of claim 265, wherein the ActRIIA fusion protein is part of a homodimer protein complex.

279. The method of claim 278, wherein the ActRIIA fusion protein is glycosylated.

280. The method of claim 265, wherein the ActRIIA polypeptide binds to one or more ligands selected from the group consisting of: activin A, activin B, GDF11, BMP10, GDF8, and BMP6.

281. The method of claim 265, wherein the TβRII polypeptide comprises an amino acid sequence comprising SEQ ID NO: 46.

282. The method of claim 265, wherein the TβRII fusion protein linker domain is selected from the group consisting of GGGGSGGGGS (SEQ ID NO: 79), TGGGGSGGGGS (SEQ ID NO: 80), TGGGGSGGGGSGGGGS (SEQ ID NO: 81), TGGGGSGGGGSGGGGSGGGGS (SEQ ID NO: 82), TGGGGSGGGGSGGGGSGGGGSGGGGS (SEQ ID NO: 83), TGGGGSGGGGSGGGGSGGGGSGGGGSGGGGS (SEQ ID NO: 84), TGGGGSGGGGSGGGGSGGGGSGGGGSGGGGSGGGGS (SEQ ID NO: 85), GGGGSGGGGS (SEQ ID NO: 178), GGGGSGGGGSGGGGS (SEQ ID NO: 179), GGGGSGGGGSGGGGSGGGGS (SEQ ID NO: 180), GGGGSGGGGSGGGGSGGGGSGGGGS (SEQ ID NO: 181), GGGGSGGGGSGGGGSGGGGSGGGGSGGGGS (SEQ ID NO: 182), or GGGGSGGGGSGGGGSGGGGSGGGGSGGGGSGGGGS (SEQ ID NO: 183).

283. The method of claim 265, wherein the TβRII fusion protein linker domain is TGGGGSGGGGSGGGGSGGGGS (SEQ ID NO: 82).

284. The method of claim 265, wherein the TβRII fusion protein Fc domain comprises an amino acid sequence that is at least 95% identical to the amino acid sequence of SEQ ID NO: 11.

285. The method of claim 265, wherein the TβRII fusion protein Fc domain comprises an amino acid sequence that is at least 99% identical to the amino acid sequence of SEQ ID NO: 11.

286. The method of claim 265, wherein the TβRII fusion protein Fc domain comprises an amino acid sequence of SEQ ID NO: 11.

287. The method of claim 265, wherein the TβRII fusion protein comprises an amino acid sequence that is at least 95% identical to SEQ ID NO: 68.

288. The method of claim 265, wherein the TβRII fusion protein comprises an amino acid sequence that is at least 99% identical to SEQ ID NO: 68.

289. The method of claim 265, wherein the TβRII fusion protein comprises an amino acid sequence of SEQ ID NO: 68.

290. The method of claim 265, wherein the TβRII fusion protein comprises an amino acid sequence that is at least 95% identical to SEQ ID NO: 70.

291. The method of claim 265, wherein the TβRII fusion protein comprises an amino acid sequence that is at least 99% identical to SEQ ID NO: 70.

292. The method of claim 265, wherein the TβRII fusion protein comprises an amino acid sequence of SEQ ID NO: 70.

293. The method of claim 265, wherein the obstructive lung disease is selected from the group consisting of chronic obstructive pulmonary disease (COPD), cystic fibrosis, asthma, emphysema, lymphangioleiomyomatosis, and chronic bronchitis.


SEQUENCE LISTING (188 sequences)

SEQ ID NO: 1 - protein, 513 aa, Homo sapiens
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 2 - protein, 115 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPP

SEQ ID NO: 3 - protein, 100 aa, Homo sapiens
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEM

SEQ ID NO: 4 - DNA, 1539 bp, Homo sapiens
atgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtcta

SEQ ID NO: 5 - DNA, 345 bp, Homo sapiens
atacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccc

SEQ ID NO: 6 - protein, 115 aa, Homo sapiens
  MOD_RES (45)..(45): Any amino acid
ILGRSETQECLFFNANWEKDRTNQTQVEPCYGDKDKRRHCFATWXNISGSIEIVKQGCWLDDINCYDRTDCVEKKDSPEVYFCCCEGNMCNEKFSTFPEMEVTQPTSNPVTPKPP

SEQ ID NO: 7 - protein, 115 aa, Ovis aries
  MOD_RES (45)..(45): Any amino acid
  MOD_RES (114)..(114): Any amino acid
ILGRSETQECLFYNANWERDRTNRTGVESCYGDKDKRRHCFATWXNISGSIDIVKQGCWLDDINCYDRTDCIEKKDSPEVYFCCCEGNMCNERFSTFPEMEVTQPTSNPVTPKXP

SEQ ID NO: 8 - protein, 115 aa, Unknown
  Description of Unknown: Mole ActRIIA sequence
  MOD_RES (45)..(45): Any amino acid
  MOD_RES (113)..(113): Any amino acid
ILGRSETQECLFFNANWERDRTNQTGVEPCYGDKDKHRHCFATWXNISGSIEIVKQGCWLDDINCYDRTDCIEKKDSPEVYFCCCEGNMCNEKFSTFPEMEVTQPTSNPVTPXAP

SEQ ID NO: 9 - protein, 115 aa, Mus sp.
  MOD_RES (45)..(45): Any amino acid
  MOD_RES (68)..(68): Any amino acid
ILGRSETQECLFFNANWERDRTNQTGVEPCYGDKDKRRHCFATWXNISGSIEIVKQGCWLDDINCYDXTDCIEKKDSPEVYFCCCEGNMCNEKFSTFPEMEVTQPTSNPVTPKPP

SEQ ID NO: 10 - protein, 115 aa, Gallus gallus
  MOD_RES (45)..(45): Any amino acid
  MOD_RES (113)..(113): Any amino acid
ILGRSETQECLYYNANWEKDKTNRSGIEPCYGDKDKRRHCFATWXNISGSIEIVKQGCWLDDINCYDRNDCIEKKDSPEVFFCCCEGNMCNERFFTFPEMEVTQPTSNPVTPXPP

SEQ ID NO: 11 - protein, 225 aa, Homo sapiens
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 13 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 14 - protein, 279 aa, Homo sapiens
ELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 15 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - protein, 3 aa, Artificial Sequence
  Description of Artificial Sequence: Synthetic peptide
GGG

SEQ ID NO: 17 - protein, 4 aa, Artificial Sequence
  Description of Artificial Sequence: Synthetic peptide
GGGG

SEQ ID NO: 18 - protein, 5 aa, Artificial Sequence
  Description of Artificial Sequence: Synthetic peptide
TGGGG

SEQ ID NO: 19 - protein, 5 aa, Artificial Sequence
  Description of Artificial Sequence: Synthetic peptide
SGGGG

SEQ ID NO: 20 - protein, 4 aa, Artificial Sequence
  Description of Artificial Sequence: Synthetic peptide
TGGG

SEQ ID NO: 21 - protein, 4 aa, Artificial Sequence
  Description of Artificial Sequence: Synthetic peptide
SGGG

SEQ ID NO: 22 - protein, 5 aa, Artificial Sequence
  Description of Artificial Sequence: Synthetic peptide
GGGGS

SEQ ID NO: 23 - protein, 344 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 24 - protein, 21 aa, Apis cerana
MKFLVNVALVFMVVYISYIYA

SEQ ID NO: 25 - protein, 22 aa, Unknown
  Description of Unknown: Tissue plasminogen activator sequence
MDAMKRGLCCVLLLCGAVFVSP

SEQ ID NO: 26 - protein, 20 aa, Unknown
  Description of Unknown: Native ActRIIA leader sequence
MGAAAKLAFAVFLISCSSGA

SEQ ID NO: 27 - protein, 369 aa, Artificial Sequence
  Description of Artificial Sequence: Synthetic polypeptide
MDAMKRGLCCVLLLCGAVFVSPGAAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - DNA, 1114 bp, Artificial Sequence
  Description of Artificial Sequence: Synthetic polynucleotide
atggatgcaatgaagagagggctctgctgtgtgctgctgctgtgtggagcagtcttcgtttcgcccggcgccgctatacttggtagatcagaaactcaggagtgtctttttttaatgctaattgggaaaaagacagaaccaatcaaactggtgttgaaccgtgttatggtgacaaagataaacggcggcattgttttgctacctggaagaatatttctggttccattgaatagtgaaacaaggttgttggctggatgatatcaactgctatgacaggactgattgtgtagaaaaaaaagacagccctgaagtatatttctgttgctgtgagggcaatatgtgtaatgaaaagttttcttattttccggagatggaagtcacacagcccacttcaaatccagttacacctaagccacccaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagtccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatgagaattc

SEQ ID NO: 29 - protein, 9 aa, Artificial Sequence
  Description of Artificial Sequence: Synthetic peptide
ILGRSETQE

SEQ ID NO: 30 - protein, 329 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 115 aa, Homo sapiens
GRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPT

SEQ ID NO: 32 - protein, 225 aa, Homo sapiens; identical to SEQ ID NO: 11

SEQ ID NO: 33 - protein, 223 aa, Homo sapiens
VECPPCPAPPVAGPSVFLFPPKPKDTLMISRTPEVTCVVVDVSHEDPEVQFNWYVDGVEVHNAKTKPREEQFNSTFRVVSVLTVLHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 34 - protein, 232 aa, Homo sapiens
EPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYKTTPPMLDSDGSFFLYSKLTVDKSRWQQGNIFSCSVMHEALHNRFTQKSLSLSPGK

SEQ ID NO: 35 - protein, 229 aa, Homo sapiens; identical to SEQ ID NO: 15

SEQ ID NO: 36 - protein, 115 aa, Bos taurus
  MOD_RES (45)..(45): Any amino acid
  MOD_RES (113)..(113): Any amino acid
ILGRSETQECLFYNANWERDRTNRTGVESCYGDKDKRRHCFATWXNISGSIEIVKQGCWLDDINCYDRTDCIEKKDSPEVYFCCCEGNMCNERFSTFPEMEVTQPTSNPVTPXPP

SEQ ID NO: 37 - protein, 115 aa, Tyto alba; identical to SEQ ID NO: 10
  MOD_RES (45)..(45): Any amino acid
  MOD_RES (113)..(113): Any amino acid

SEQ ID NO: 38 - protein, 115 aa, Myotis davidii
  MOD_RES (45)..(45): Any amino acid
ILGRSETQECLFYNANWERDKTNRTGVELCYGDKDKHRHCFATWXNISGSIEIVKQGCWLDDINCYDKTDCIEKKDSPEVYFCCCEGNMCNERFSTFPEMEVTQPTSNPVTPKPP

SEQ ID NO: 39-40 - skipped (2 entries)

SEQ ID NO: 41 - protein, 343 aa, Artificial Sequence
  Description of Artificial Sequence: Synthetic polypeptide
ILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPVPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 592 aa, Homo sapiens
MGRGLLRGLWPLHIVLWTRIASTIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 43 - protein, 567 aa, Homo sapiens
MGRGLLRGLWPLHIVLWTRIASTIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDLLLVIFQVTGISLLPPLGVAISVIIIFYCYRVNRQQKLSSTWETGKTRKLMEFSEHCAIILEDDRSDISSTCANNINHNTELLPIELDTLVGKGRFAEVYKAKLKQNTSEQFETVAVKIFPYEEYASWKTEKDIFSDINLKHENILQFLTAEERKTELGKQYWLITAFHAKGNLQEYLTRHVISWEDLRKLGSSLARGIAHLHSDHTPCGRPKMPIVHRDLKSSNILVKNDLTCCLCDFGLSLRLDPTLSVDDLANSGQVGTARYMAPEVLESRMNLENVESFKQTDVYSMALVLWEMTSRCNAVGEVKDYEPPFGSKVREHPCVESMKDNVLRDRGRPEIPSFWLNHQGIQMVCETLTECWDHDPEARLTAQCVAERFSELEHLDRLSGRSCSEEKIPEDGSLNTTK

SEQ ID NO: 44 - skipped

SEQ ID NO: 45 - protein, 137 aa, Artificial Sequence
  Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 46 - protein, 162 aa, Homo sapiens
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 47 - protein, 131 aa, Artificial Sequence
  Description of Artificial Sequence: Synthetic polypeptide
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 48 - protein, 125 aa, Artificial Sequence
  Description of Artificial Sequence: Synthetic polypeptide
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 49 - protein, 131 aa, Artificial Sequence
  Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 50 - protein, 125 aa, Artificial Sequence
  Description of Artificial Sequence: Synthetic polypeptide
QKSVNNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 51 - protein, 119 aa, Artificial Sequence
  Description of Artificial Sequence: Synthetic polypeptide
DMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 52 - protein, 156 aa, Artificial Sequence
  Description of Artificial Sequence: Synthetic polypeptide
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPD

SEQ ID NO: 53 - skipped

SEQ ID NO: 54 - protein, 156 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 55 - protein, 150 aa, Artificial Sequence
  Description of Artificial Sequence: Synthetic polypeptide
QKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEY

SEQ ID NO: 56 - skipped

SEQ ID NO: 57 - protein, 137 aa, Artificial Sequence
  Description of Artificial Sequence: Synthetic polypeptide
TIPPHVQKSVNNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 58 - protein, 162 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHKFILEDAASPKCIMKEKKKPGETFFMCSCSSDECNDNIIFSEEYNTSNPD

SEQ ID NO: 59 - protein, 131 aa, Artificial Sequence
  Description of Artificial Sequence: Synthetic polypeptide
TIPPHVQKSVNNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 60 - protein, 156 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSDCSITSICEKPQEVCVAVWRKNDENITLETVCHDPKLPYHDFILEDAASPKCIMKEKKKPGETFFMCSCSSDECNDNIIFSEEY

SEQ ID NO: 61 - DNA, 1284 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 62 - DNA, 1299 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 63 - DNA, 1269 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 64 - DNA, 1266 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggacccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaatga

SEQ ID NO: 65 - protein, 36 aa, Artificial Sequence
  Description of Artificial Sequence: Synthetic polypeptide
GRCKIRHIGSNNRLQRSTCQNTGWESAHVMKTPGFR

SEQ ID NO: 66 - skipped

SEQ ID NO: 67 - protein, 162 aa, Artificial Sequence; identical to SEQ ID NO: 46
  Description of Artificial Sequence: Synthetic polypeptide

SEQ ID NO: 68 - protein, 408 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 69 - skipped

SEQ ID NO: 70 - protein, 407 aa, Artificial Sequence
  Description of Artificial Sequence: Synthetic polypeptide
TIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 71 - protein, 427 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 72 - protein, 432 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 73 - protein, 422 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 74 - protein, 421 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 75 - protein, 426 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 76 - protein, 431 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 77 - protein, 421 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 78 - protein, 420 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 79 - protein, 10 aa, Artificial Sequence
  Description of Artificial Sequence: Synthetic peptide
GGGGSGGGGS

SEQ ID NO: 80 - protein, 11 aa, Artificial Sequence
  Description of Artificial Sequence: Synthetic peptide
TGGGGSGGGGS

SEQ ID NO: 81 - protein, 16 aa, Artificial Sequence
  Description of Artificial Sequence: Synthetic peptide
TGGGGSGGGGSGGGGS

SEQ ID NO: 82 - protein, 21 aa, Artificial Sequence
  Description of Artificial Sequence: Synthetic peptide
TGGGGSGGGGSGGGGSGGGGS

SEQ ID NO: 83 - protein, 26 aa, Artificial Sequence
  Description of Artificial Sequence: Synthetic peptide
TGGGGSGGGGSGGGGSGGGGSGGGGS

SEQ ID NO: 84 - protein, 31 aa, Artificial Sequence
  Description of Artificial Sequence: Synthetic polypeptide
TGGGGSGGGGSGGGGSGGGGSGGGGSGGGGS

SEQ ID NO: 85 - protein, 36 aa, Artificial Sequence
  Description of Artificial Sequence: Synthetic polypeptide
TGGGGSGGGGSGGGGSGGGGSGGGGSGGGGSGGGGS

SEQ ID NO: 86 - protein, 10 aa, Artificial Sequence
  Description of Artificial Sequence: Synthetic peptide
TGGGPKSCDK

SEQ ID NO: 87 - protein, 4 aa, Artificial Sequence
  Description of Artificial Sequence: Synthetic peptide
GGGA

SEQ ID NO: 88 - protein, 4 aa, Artificial Sequence
  Description of Artificial Sequence: Synthetic peptide
GGGS

SEQ ID NO: 89 - protein, 4 aa, Artificial Sequence
  Description of Artificial Sequence: Synthetic peptide
GGGG

SEQ ID NO: 90 - protein, 5 aa, Artificial Sequence
  Description of Artificial Sequence: Synthetic peptide
GGGGA

SEQ ID NO: 91 - protein, 5 aa, Artificial Sequence
  Description of Artificial Sequence: Synthetic peptide
GGGGS

SEQ ID NO: 92 - protein, 5 aa, Artificial Sequence
  Description of Artificial Sequence: Synthetic peptide
GGGGG

SEQ ID NO: 93 - protein, 4 aa, Artificial Sequence
  Description of Artificial Sequence: Synthetic peptide
GGAG

SEQ ID NO: 94 - protein, 4 aa, Artificial Sequence
  Description of Artificial Sequence: Synthetic peptide
GGSG

SEQ ID NO: 95 - protein, 4 aa, Artificial Sequence
  Description of Artificial Sequence: Synthetic peptide
AGGG

SEQ ID NO: 96 - protein, 4 aa, Artificial Sequence
  Description of Artificial Sequence: Synthetic peptide
GAGA

SEQ ID NO: 97 - protein, 4 aa, Artificial Sequence
  Description of Artificial Sequence: Synthetic peptide
GSGS

SEQ ID NO: 98 - protein, 6 aa, Artificial Sequence
  Description of Artificial Sequence: Synthetic peptide
GAGAGA

SEQ ID NO: 99 - protein, 6 aa, Artificial Sequence
  Description of Artificial Sequence: Synthetic peptide
GSGSGS

SEQ ID NO: 100 - protein, 8 aa, Artificial Sequence
  Description of Artificial Sequence: Synthetic peptide
GAGAGAGA

SEQ ID NO: 101 - protein, 8 aa, Artificial Sequence
  Description of Artificial Sequence: Synthetic peptide
GSGSGSGS

SEQ ID NO: 102 - protein, 10 aa, Artificial Sequence
  Description of Artificial Sequence: Synthetic peptide
GAGAGAGAGA

SEQ ID NO: 103 - protein, 10 aa, Artificial Sequence
  Description of Artificial Sequence: Synthetic peptide
GSGSGSGSGS

SEQ ID NO: 104 - protein, 12 aa, Artificial Sequence
  Description of Artificial Sequence: Synthetic peptide
GAGAGAGAGAGA

SEQ ID NO: 105 - protein, 12 aa, Artificial Sequence
  Description of Artificial Sequence: Synthetic peptide
GSGSGSGSGSGS

SEQ ID NO: 106 - protein, 6 aa, Artificial Sequence
  Description of Artificial Sequence: Synthetic peptide
GGAGGA

SEQ ID NO: 107 - protein, 6 aa, Artificial Sequence
  Description of Artificial Sequence: Synthetic peptide
GGSGGS

SEQ ID NO: 108 - protein, 9 aa, Artificial Sequence
  Description of Artificial Sequence: Synthetic peptide
GGAGGAGGA

SEQ ID NO: 109 - protein, 9 aa, Artificial Sequence
  Description of Artificial Sequence: Synthetic peptide
GGSGGSGGS

SEQ ID NO: 110 - protein, 12 aa, Artificial Sequence
  Description of Artificial Sequence: Synthetic peptide
GGAGGAGGAGGA

SEQ ID NO: 111 - protein, 12 aa, Artificial Sequence
  Description of Artificial Sequence: Synthetic peptide
GGSGGSGGSGGS

SEQ ID NO: 112 - protein, 4 aa, Artificial Sequence
  Description of Artificial Sequence: Synthetic peptide
GGAG

SEQ ID NO: 113 - protein, 4 aa, Artificial Sequence
  Description of Artificial Sequence: Synthetic peptide
GGSG

SEQ ID NO: 114 - protein, 8 aa, Artificial Sequence
  Description of Artificial Sequence: Synthetic peptide
GGAGGGAG

SEQ ID NO: 115 - protein, 8 aa, Artificial Sequence
  Description of Artificial Sequence: Synthetic peptide
GGSGGGSG

SEQ ID NO: 116 - protein, 12 aa, Artificial Sequence
  Description of Artificial Sequence: Synthetic peptide
GGAGGGAGGGAG

SEQ ID NO: 117 - protein, 12 aa, Artificial Sequence
  Description of Artificial Sequence: Synthetic peptide
GGSGGGSGGGSG

SEQ ID NO: 118 - protein, 5 aa, Artificial Sequence
  Description of Artificial Sequence: Synthetic peptide
GGGGA

SEQ ID NO: 119 - protein, 5 aa, Artificial Sequence
  Description of Artificial Sequence: Synthetic peptide
GGGGS

SEQ ID NO: 120 - protein, 15 aa, Artificial Sequence
  Description of Artificial Sequence: Synthetic peptide
GGGGAGGGGAGGGGA

SEQ ID NO: 121 - protein, 15 aa, Artificial Sequence
  Description of Artificial Sequence: Synthetic peptide
GGGGSGGGGSGGGGS

SEQ ID NO: 122 - protein, 4 aa, Artificial Sequence
  Description of Artificial Sequence: Synthetic peptide
GGGA

SEQ ID NO: 123 - protein, 5 aa, Artificial Sequence
  Description of Artificial Sequence: Synthetic peptide
GGGAG

SEQ ID NO: 124 - protein, 6 aa, Artificial Sequence
  Description of Artificial Sequence: Synthetic peptide
GGGAGG

SEQ ID NO: 125 - protein, 7 aa, Artificial Sequence
  Description of Artificial Sequence: Synthetic peptide
GGGAGGG

SEQ ID NO: 126 - protein, 4 aa, Artificial Sequence
  Description of Artificial Sequence: Synthetic peptide
AAAL

SEQ ID NO: 127 - protein, 4 aa, Artificial Sequence
  Description of Artificial Sequence: Synthetic peptide
AAAK

SEQ ID NO: 128 - protein, 4 aa, Artificial Sequence
  Description of Artificial Sequence: Synthetic peptide
AAAR

SEQ ID NO: 129 - protein, 14 aa, Artificial Sequence
  Description of Artificial Sequence: Synthetic peptide
EGKSSGSGSESKST

SEQ ID NO: 130 - protein, 12 aa, Artificial Sequence
  Description of Artificial Sequence: Synthetic peptide
GSAGSAAGSGEF

SEQ ID NO: 131 - protein, 12 aa, Artificial Sequence
  Description of Artificial Sequence: Synthetic peptide
AEAAAKEAAAKA

SEQ ID NO: 132 - protein, 18 aa, Artificial Sequence
  Description of Artificial Sequence: Synthetic peptide
KESGSVSSEQLAQFRSLD

SEQ ID NO: 133 - protein, 10 aa, Artificial Sequence
  Description of Artificial Sequence: Synthetic peptide
GENLYFQSGG

SEQ ID NO: 134 - protein, 8 aa, Artificial Sequence
  Description of Artificial Sequence: Synthetic peptide
SACYCELS

SEQ ID NO: 135 - protein, 5 aa, Artificial Sequence
  Description of Artificial Sequence: Synthetic peptide
RSIAT

SEQ ID NO: 136 - protein, 17 aa, Artificial Sequence
  Description of Artificial Sequence: Synthetic peptide
RPACKIPNDLKQKVMNH

SEQ ID NO: 137 - protein, 36 aa, Artificial Sequence
  Description of Artificial Sequence: Synthetic polypeptide
GGSAGGSGSGSSGGSSGASGTGTAGGTGSGSGTGSG

SEQ ID NO: 138 - protein, 17 aa, Artificial Sequence
  Description of Artificial Sequence: Synthetic peptide
AAANSSIDLISVPVDSR

SEQ ID NO: 139 - protein, 36 aa, Artificial Sequence
  Description of Artificial Sequence: Synthetic polypeptide
GGSGGGSEGGGSEGGGSEGGGSEGGGSEGGGSGGGS

SEQ ID NO: 140 - protein, 5 aa, Artificial Sequence
  Description of Artificial Sequence: Synthetic peptide
EAAAK

SEQ ID NO: 141 - protein, 5 aa, Artificial Sequence
  Description of Artificial Sequence: Synthetic peptide
PAPAP

SEQ ID NO: 142 - protein, 415 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 143 - protein, 414 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 437 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 145 - protein, 436 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 146 - protein, 442 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 147 - protein, 441 aa, Artificial Sequence
  Description of Artificial Sequence: Synthetic polypeptide
MDAMKRGLCCVLLLCGAVFVSPGATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 224 aa, Artificial Sequence
  Description of Artificial Sequence: Synthetic polypeptide
THTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - DNA, 1248 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggtggaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 150 - protein, 410 aa, Artificial Sequence
  Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 409 aa, Artificial Sequence
  Description of Artificial Sequence: Synthetic polypeptide
GATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 152 - protein, 409 aa, Artificial Sequence
  Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 408 aa, Artificial Sequence
  Description of Artificial Sequence: Synthetic polypeptide
ATIPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 154 - protein, 407 aa, Artificial Sequence
  Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 155 - protein, 406 aa, Artificial Sequence
  Description of Artificial Sequence: Synthetic polypeptide
IPPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 156 - protein, 406 aa, Artificial Sequence
  Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 157 - protein, 405 aa, Artificial Sequence
  Description of Artificial Sequence: Synthetic polypeptide
PPHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 158 - protein, 405 aa, Artificial Sequence
  Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 404 aa, Artificial Sequence
  Description of Artificial Sequence: Synthetic polypeptide
PHVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 160 - protein, 404 aa, Artificial Sequence
  Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 161 - protein, 403 aa, Artificial Sequence
  Description of Artificial Sequence: Synthetic polypeptide
HVQKSDVEMEAQKDEIICPSCNRTAHPLRHINNDMIVTDNNGAVKFPQLCKFCDVRFSTCDNQKSCMSNCSITSICEKPQEVCVAVWRKNDENITLETVCHDPKLPYHDFILEDAASPKCIMKEKKKPGETFFMCSCSSDECNDNIIFSEEYNTSNPDTGGGGSGGGGSGGGGSGGGGSTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 162 - DNA, 1314 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 163 - DNA, 1329 bp, Artificial Sequence
  Description of Artificial Sequence: Synthetic polynucleotide
atggatgcaatgaagagagggctctgctgtgtgctgctgctgtgtggagcagtcttcgtttcgcccggcgccacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacaccggtggaggtggaagtggtggaggaggttctggtggtggaggttctggaggtggaggaagtggtggaggtggttctggaggtggtggaagtactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 164 - protein, 233 aa, Artificial Sequence
  Description of Artificial Sequence: Synthetic polypeptide
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 165 - protein, 222 aa, Artificial Sequence
  Description of Artificial Sequence: Synthetic polypeptide
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 166 - protein, 232 aa, Artificial Sequence
  Description of Artificial Sequence: Synthetic polypeptide
GGPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 167 - protein, 22 aa, Unknown
  Description of Unknown: Native leader sequence
MGRGLLRGLWPLHIVLWTRIAS

SEQ ID NO: 168 - protein, 427 aa, Artificial Sequence
  Description of Artificial Sequence: Synthetic polypeptide
MDAMKRGLCCVLLLCGAVFVSPGAIPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 169 - protein, 403 aa, Artificial Sequence
  Description of Artificial Sequence: Synthetic polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLGK

SEQ ID NO: 170 - protein, 402 aa, Artificial Sequence
  Description of Artificial Sequence: Synthetic polypeptide
IPPHVPKSDVEMEAQKDASIHLSCNRTIHPLKHFNSDVMASDNGGAVKLPQLCKFCDVRLSTCDNQKSCMSNCSITAICEKPHEVCVAVWRKNDKNITLETVCHDPKLTYHGFTLEDAASPKCVMKEKKRAGETFFMCACNMEECNDYIIFSEEYTTSSPDTGGGEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYFMYSKLRVQKSTWERGSLFACSVVHEGLHNHLTTKTISRSLG

SEQ ID NO: 171 - DNA, 1284 bp, Artificial Sequence
  Description of Artificial Sequence: Synthetic polynucleotide
atggatgcaatgaagagagggctctgctgtgtgctgctgctgtgtggagcagtcttcgtttcgcccggcgccatcccgccgcacgttcccaagtcggatgtggaaatggaagcccagaaagatgcatccatccacctaagctgtaataggaccatccatccactgaaacattttaacagtgatgtcatggccagcgacaatggcggtgcggtcaagcttccacagctgtgcaagttttgcgatgtgagactgtccacttgcgacaaccagaagtcctgcatgagcaactgcagcatcacggccatctgtgagaagccgcatgaagtctgcgtggccgtgtggaggaagaacgacaagaacattactctggagacggtttgccacgaccccaagctcacctaccacggcttcactctggaagatgccgcttctcccaagtgtgtcatgaaggaaaagaaaagggcgggcgagactttcttcatgtgtgcctgtaacatggaagagtgcaacgattacatcatcttttcggaagaatacaccaccagcagtcccgacaccggtgggggtgagcccagagtgcccataacacagaacccctgtcctccactcaaagagtgtcccccatgcgcagctccagacctcttgggtggaccatccgtcttcatcttccctccaaagatcaaggatgtactcatgatctccctgagccccatggtcacatgtgtggtggtggatgtgagcgaggatgacccagacgtccagatcagctggtttgtgaacaacgtggaagtacacacagctcagacacaaacccatagagaggattacaacagtactctccgggtggtcagtgccctccccatccagcaccaggactggatgagtggcaaggagttcaaatgcaaggtcaacaacagagccctcccatcccccatcgagaaaaccatctcaaaacccagagggccagtaagagctccacaggtatatgtcttgcctccaccagcagaagagatgactaagaaagagttcagtctgacctgcatgatcacaggcttcttacctgccgaaattgctgtggactggaccagcaatgggcgtacagagcaaaactacaagaacaccgcaacagtcctggactctgatggttcttacttcatgtacagcaagctcagagtacaaaagagcacttgggaaagaggaagtcttttcgcctgctcagtggtccacgagggtctgcacaatcaccttacgactaagaccatctcccggtctctgggtaaatga

SEQ ID NO: 172 - protein, 20 aa, Artificial Sequence
  Description of Artificial Sequence: Synthetic peptide
GGGGSGGGGSGGGGSGGGGS

SEQ ID NO: 173 - protein, 17 aa, Artificial Sequence
  Description of Artificial Sequence: Synthetic peptide
NTSNPDTGGGTHTCPPC

SEQ ID NO: 174 - protein, 24 aa, Artificial Sequence
  Description of Artificial Sequence: Synthetic peptide
NTSNPDTGGGGSGGGGSTHTCPPC

SEQ ID NO: 175 - protein, 29 aa, Artificial Sequence
  Description of Artificial Sequence: Synthetic peptide
NTSNPDTGGGGSGGGGSGGGGSTHTCPPC

SEQ ID NO: 176 - protein, 35 aa, Artificial Sequence
  Description of Artificial Sequence: Synthetic polypeptide
NTSNPDTGGGGSGGGGSGGGGGSGGGGSTHTCPPC

SEQ ID NO: 177 - protein, 23 aa, Artificial Sequence
  Description of Artificial Sequence: Synthetic peptide
NTSNPDTGGGPKSCDKTHTCPPC

SEQ ID NO: 178 - protein, 10 aa, Artificial Sequence
  Description of Artificial Sequence: Synthetic peptide
GGGGSGGGGS

SEQ ID NO: 179 - protein, 15 aa, Artificial Sequence
  Description of Artificial Sequence: Synthetic peptide
GGGGSGGGGSGGGGS

SEQ ID NO: 180 - protein, 20 aa, Artificial Sequence
  Description of Artificial Sequence: Synthetic peptide
  See specification as filed for detailed description of substitutions and preferred embodiments
GGGGSGGGGSGGGGSGGGGS

SEQ ID NO: 181 - protein, 25 aa, Artificial Sequence
  Description of Artificial Sequence: Synthetic peptide
GGGGSGGGGSGGGGSGGGGSGGGGS

SEQ ID NO: 182 - protein, 30 aa, Artificial Sequence
  Description of Artificial Sequence: Synthetic polypeptide
GGGGSGGGGSGGGGSGGGGSGGGGSGGGGS

SEQ ID NO: 183 - protein, 35 aa, Artificial Sequence
  Description of Artificial Sequence: Synthetic polypeptide
GGGGSGGGGSGGGGSGGGGSGGGGSGGGGSGGGGS

SEQ ID NO: 184 - protein, 6 aa, Artificial Sequence
  Description of Artificial Sequence: Synthetic 6xHis tag
HHHHHH

SEQ ID NO: 185 - protein, 10 aa, Artificial Sequence
  Description of Artificial Sequence: Synthetic peptide
GGGGSGGGGS

SEQ ID NO: 186 - protein, 20 aa, Artificial Sequence
  Description of Artificial Sequence: Synthetic peptide
GGGGSGGGGSGGGGSGGGGS

SEQ ID NO: 187 - protein, 5 aa, Artificial Sequence
  Description of Artificial Sequence: Synthetic peptide
GGGGS

SEQ ID NO: 188 - protein, 10 aa, Artificial Sequence
  Description of Artificial Sequence: Synthetic peptide
  MOD_RES (1)..(1): Any amino acid
  MOD_RES (3)..(3): Any amino acid
  MOD_RES (5)..(5): Any amino acid
  MOD_RES (7)..(7): Any amino acid
  MOD_RES (9)..(9): Any amino acid
  SITE (1)..(10): This sequence may encompass 1-5 “Xaa Pro” repeating units
XPXPXPXPXP